(19) United States
(12) Patent Application Publication
(10) Pub. No.: US 2003/0105051 A1
(43) Pub. Date: Jun. 5, 2003

(54) Nucleic acid treatment of diseases or conditions related to levels of HER2

(76) Inventor: James McSwiggen, Boulder, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/163,552

(22) Filed: Jun. 6, 2002

Related U.S. Application Data
(60) Provisional application No. 60/296,249, filed on Jun. 6, 2001.

(51) Int. Cl. A61K 48/00; C12N 9/22; C07H 21/04

(52) U.S. Cl. 514/44; 536/23.2; 435/199

(57) ABSTRACT

The present invention relates to enzymatic nucleic acid molecules, including DNAzymes (DNA enzymes, catalytic DNA), siRNA, antisense, aptamers and decoys, that modulate the expression of HER2 genes.

This patent application claims priority from McSwiggen U.S. Ser. No. 60/296,249 filed Jun. 6, 2001, entitled “Enzymatic Nucleic Acid Treatment of Diseases or Conditions Related to Levels of HER2”. This application is hereby incorporated by reference herein in its entirety including the drawings and tables.

### DESCRIPTION

1. Technical Field of the Invention

The present invention relates to novel nucleic acid compounds for the treatment or diagnosis of diseases or conditions related to HER2 gene expression.

2. Background of the Invention

HER2 (also known as neu, erbB2 and c-erbB2) is an oncogene that encodes a 185-kDa transmembrane tyrosine kinase receptor. HER2 is a member of the epidermal growth factor receptor (EGFR) family and shares partial homology with other family members. In normal adult tissues HER2 expression is low. However, HER2 is overexpressed in at least 25-30% of breast (McGuire, H. C. and Greene, M. I. (1989) The neu (c-erbB-2) oncogene. Semin. Oncol. 16: 148-155) and ovarian cancers (Berchuck, A. Kamel, A., Whitaker, R. et al. (1990)). Overexpression of her-2/neu is associated with poor survival in advanced epithelial ovarian cancer. Cancer Research 50: 4087-4091). Furthermore, overexpression of HER2 in malignant breast tumors has been correlated with increased metastasis, chemoresistance and poor survival rates (Slamon et al., 1987 Science 235: 177-182). Because HER2 expression is high in aggressive human breast and ovarian cancers, but low in normal adult tissues, it is an attractive target for enzymatic nucleic acid-mediated therapy. McSwiggen et al., International PCT Publication No. WO 01/16312 and Beigelman et al., International PCT Publication No. WO 99/55857 describe enzymatic nucleic acid molecules targeting HER2. Thompson and Draper, U.S. Pat. No. 5,599,704, describes enzymatic nucleic acid molecules targeting HER2 (erbB2/neu) gene expression.

### SUMMARY OF THE INVENTION

The present invention features nucleic acid molecules, including, for example, antisense oligonucleotides, siRNA, aptamers, decoys and enzymatic nucleic acid molecules such as DNAzyme molecules which modulate expression of nucleic acid molecules encoding HER2.

In one embodiment, the invention features an enzymatic nucleic acid molecule comprising a sequence having SEQ ID NOs: 989-1976 and 1982-1986.

In another embodiment, the invention features an enzymatic nucleic acid molecule comprising at least one binding arm having a sequence complementary to a sequence selected from the group consisting of SEQ ID NOs: 1-988 and 1977-1981.

In another embodiment, the invention features a siRNA molecule having complementarity to a sequence selected from the group consisting of SEQ ID NOs: 1-988 and 1977-1981.

In another embodiment, the invention features an antisense molecule having complementarity to a sequence selected from the group consisting of SEQ ID NOs: 1-988 and 1977-1981.

In another aspect of the invention, the nucleic acid of the invention is adapted to treat cancer.

In another embodiment, an enzymatic nucleic acid molecule of the invention has an endonuclease activity to cleave RNA having HER2 sequence.

In one embodiment, a siRNA molecule of the invention comprises a double stranded RNA wherein one strand of the RNA is complementary to the RNA of HER2 gene. In another embodiment, a siRNA molecule of the invention comprises a double stranded RNA wherein one strand of the RNA comprises a portion of a sequence of RNA having of HER2 gene sequence. In yet another embodiment, a siRNA molecule of the invention comprises a double stranded RNA wherein both strands of RNA are connected by a non-nucleotide linker. Alternately, a siRNA molecule of the invention comprises a double stranded RNA wherein both strands of RNA are connected by a nucleotide linker, such as a loop or stem loop structure.

In one embodiment, a single strand component of a siRNA molecule of the invention is from about 14 to about 50 nucleotides in length. In another embodiment, a single strand component of a siRNA molecule of the invention is about 14, 15, 16, 17, 18, 19, 20, 21, 22, 23, 24, 25, 26, 27, or 28 nucleotides in length. In yet another embodiment, a single strand component of a siRNA molecule of the invention is about 23 nucleotides in length. In one embodiment, a siRNA molecule of the invention is from about 28 to about 56 nucleotides in length. In another embodiment, a siRNA molecule of the invention is about 40, 41, 42, 43, 44, 45, 46, 47, 48, 49, 50, 51, or 52 nucleotides in length. In yet another embodiment, a siRNA molecule of the invention is about 46 nucleotides in length.

In one embodiment, a DNAzyme molecule of the invention is in a “10-23” configuration. In another embodiment, a DNAzyme of the invention comprises a sequence complementary to a sequence having SEQ ID NOs: 1-988 and 1977-1981. In yet another embodiment, a DNAzyme molecule of the invention comprises a sequence having SEQ ID NOs: 989-1976 and 1982-1986.

In another embodiment, a nucleic acid molecule of the invention comprises between 12 and 100 bases complementary to a nucleic acid molecule having HER2 sequence. In yet another embodiment, a nucleic acid molecule of the invention comprises between 14 and 24 bases complementary to a nucleic acid molecule having HER2 sequence.

In yet another embodiment, an enzymatic nucleic acid molecule of the invention is chemically synthesized. An enzymatic nucleic acid molecule of the invention can comprise at least one 2′-sugar modification, at least one nucleic acid base modification, and/or at least one phosphate backbone modification.

In one embodiment, the invention features a mammalian cell comprising a nucleic acid molecule of the invention. In another embodiment, the mammalian cell of the invention is a human cell.

In another embodiment, the invention features a method of reducing HER2 activity in a cell, comprising contacting the cell with the nucleic acid molecule of the invention, under conditions suitable for the reduction of HER2 activity.

In another embodiment, the invention features a method of treatment of a subject having a condition associated with the level of HER2, comprising contacting cells of the subject with the enzymatic nucleic acid molecule of the invention, under conditions suitable for the treatment.

In one embodiment, a method of treatment of the invention further comprises the use of one or more drug therapies under conditions suitable for the treatment.

In another embodiment, the invention features a method of cleaving RNA having HER2 sequence comprising contacting an enzymatic nucleic acid molecule of the invention with the RNA under conditions suitable for the cleavage, for example, where the cleavage is carried out in the presence of a divalent cation, such as Mg2+.

In one embodiment, a nucleic acid molecule of the invention comprises a cap structure, for example a 3′,3′-linked or 5′,5′-linked deoxyabasic ribose derivative, wherein the cap structure is at the 5′-end, 3′-end, or both the 5′-end and the 3′-end of the enzymatic nucleic acid molecule.

In another embodiment, the invention features an expression vector comprising a nucleic acid sequence encoding at least one enzymatic nucleic acid molecule of the invention, for example a DNAzyme or siRNA molecule, in a manner that allows expression of the enzymatic nucleic acid molecule.

In yet another embodiment, the invention features a mammalian cell, for example a human cell, comprising an expression vector of the invention.

In another embodiment, an expression vector of the invention further comprises a sequence for a nucleic acid molecule complementary to a nucleic acid molecule having HER2 sequence.

In one embodiment, an expression vector of the invention comprises a nucleic acid sequence encoding two or more nucleic acid molecules, which can be the same or different. In another embodiment, an expression vector of the invention further comprises a sequence encoding an antisense nucleic acid molecule complementary to a nucleic acid molecule having a HER2 sequence.

In another embodiment, the invention features a method for treating cancer, for example breast cancer or ovarian cancer, comprising administering to a subject a nucleic acid molecule of the invention under conditions suitable for the treatment. A method of treatment of cancer of the invention can further comprise administering to a subject one or more other therapies, for example monoclonal antibody therapy, such as Herceptin (trastuzumab); chemotherapy, such as paclitaxel (Taxol), docetaxel, cisplatin, Leucovorin, Irinotecan (CAMPTOSAR® or CPT-11 or Camptothecin-11 or Campto), Carboplatin, methotrexate, cyclophosphamide, doxorubin, fluorouracil carboplatin, edatrexate, gemcitabine, or vinorelbine; radiation therapy, or analgesic therapy and/or any combination thereof.

In another embodiment, the invention features a composition comprising a nucleic acid molecule of the invention in a pharmaceutically acceptable carrier.

In one embodiment, the invention features a method of administering to a cell, for example a mammalian cell or human cell, a nucleic acid molecule of the invention comprising contacting the cell with the nucleic acid molecule under conditions suitable for administration. The method of administration can be in the presence of a delivery reagent, for example a lipid, cationic lipid, phospholipid, or liposome.

### DETAILED DESCRIPTION OF THE INVENTION

First the drawings will be described briefly.

### DRAWINGS

FIG. 1 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig et al., International PCT Publication No. WO 98/58058 and U.S. patent application Ser. No. 08/878,640); G-Cleaver, represents G-cleaver ribozyme motif (Kore et al., 1998, Nucleic Acids Research 26, 4116-4120, Eckstein et al., U.S. Pat. No. 6,127,173). N or n, represent independently a nucleotide which can be the same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

FIG. 2 shows an example of the Amberzyme ribozyme motif that is chemically stabilized (see for example Beigelman et al., International PCT publication No. WO 99/55857 and U.S. patent application Ser. No. 09/476,387.).

FIG. 3 shows an example of a Zinzyme A ribozyme motif that is chemically stabilized (see for example Beigelman et al., International PCT publication No. WO 99/55857 and U.S. patent application Ser. No. 09/918,728).

FIG. 4 shows an example of a DNAzyme motif described by Santoro et al., 1997, PNAS, 94, 4262 and Joyce et al., U.S. Pat. No. 5,807,718.

The invention features novel nucleic acid molecules, including antisense oligonucleotides, siRNA and enzymatic nucleic acid molecules, and methods to modulate gene expression, for example, genes encoding HER2. In particular, the instant invention features nucleic-acid based molecules and methods to down-regulate the expression of HER2 gene sequences.

The invention features novel nucleic acid molecules, siRNA molecules and methods to modulate gene expression, for example, genes encoding HER2. In particular, the instant invention features nucleic-acid based molecules and methods to inhibit the expression of HER2.

The invention features one or more nucleic acid-based molecules and methods that independently or in combination modulate the expression of a gene or genes encoding HER2. In particular embodiments, the invention features nucleic acid-based molecules and methods that modulate the expression of HER2 gene, for example, Genbank Accession No. NM_{—}004448.

The description below of the various aspects and embodiments is provided with reference to an exemplary HER2 gene, referred to herein as HER2 but also known as ERB2, ERB-B2, NEU, NGL, and v-ERB-B2. However, the various aspects and embodiments are also directed to other genes that encode HER2 proteins and similar proteins to HER2. Those additional genes can be analyzed for target sites using the methods described for HER2. Thus, the inhibition and the effects of such inhibition of the other genes can be performed as described herein.

In one embodiment, the invention features the use of an enzymatic nucleic acid molecule, preferably in the hammerhead, NCH, G-cleaver, amberzyme, zinzyme and/or DNAzyme motif, to down-regulate the expression of HER2 genes or inhibit HER2 activity.

By “inhibit” or “down-regulate” it is meant that the expression of the gene, or level of RNAs or equivalent RNAs encoding one or more protein subunits or components, or activity of one or more protein subunits or components, such as HER2 protein or proteins, is reduced below that observed in the absence of the nucleic acid molecules of the invention. In one embodiment, inhibition or down-regulation with enzymatic nucleic acid molecule preferably is below that level observed in the presence of an enzymatically inactive or attenuated molecule that is able to bind to the same site on the target RNA, but is unable to cleave that RNA. In another embodiment, inhibition or down-regulation with antisense or siRNA oligonucleotides is preferably below that level observed in the presence of, for example, an oligonucleotide with scrambled sequence or with mismatches. In another embodiment, inhibition or down-regulation of HER2 expression and/or activity with the nucleic acid molecule of the instant invention is greater in the presence of the nucleic acid molecule than in its absence.

By “up-regulate” is meant that the expression of the gene, or level of RNAs or equivalent RNAs encoding one or more protein subunits or components, or activity of one or more protein subunits or components, such as HER2 protein or proteins, is greater than that observed in the absence of the nucleic acid molecules of the invention. For example, the expression of a gene, such as HER2 gene, can be increased in order to treat, prevent, ameliorate, or modulate a pathological condition caused or exacerbated by an absence or low level of gene expression.

By “modulate” is meant that the expression of the gene, or level of RNAs or equivalent RNAs encoding one or more protein subunits or components, or activity of one or more proteins is up-regulated or down-regulated, such that the expression, level, or activity is greater than or less than that observed in the absence of the nucleic acid molecules of the invention.

By “enzymatic nucleic acid molecule” as used herein, is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

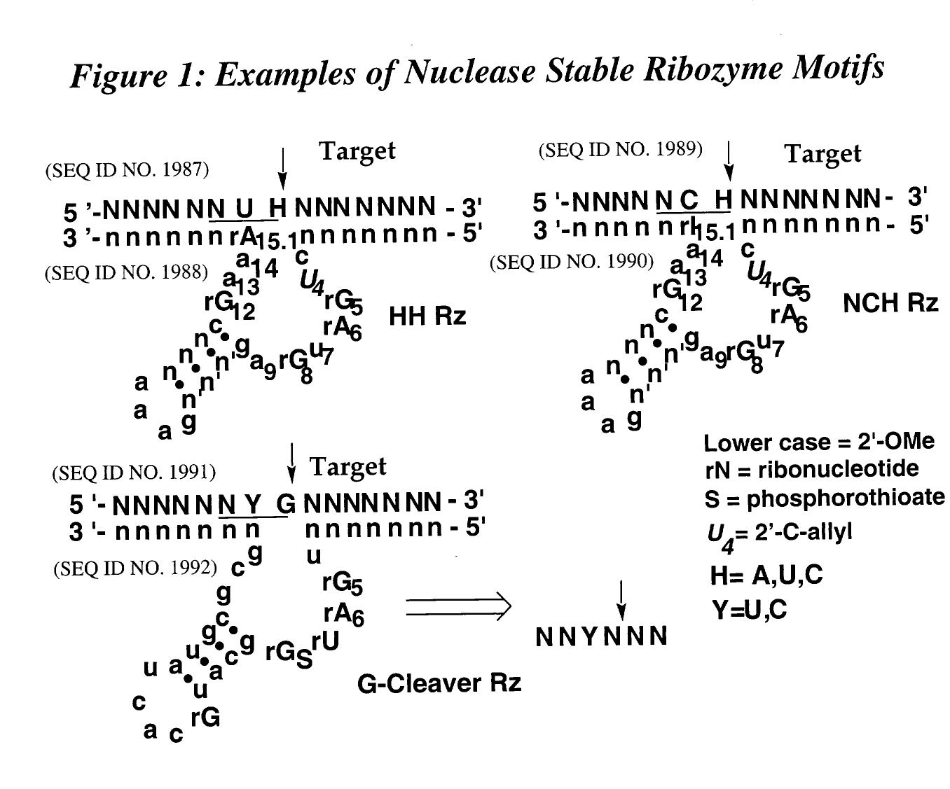

By “enzymatic portion” or “catalytic domain” is meant that portion/region of the enzymatic nucleic acid molecule essential for cleavage of a nucleic acid substrate (for example see FIGS. 1-4).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within an enzymatic nucleic acid that are intended to bring enzymatic nucleic acid and target RNA together through complementary base-pairing interactions. The enzymatic nucleic acid of the invention can have binding arms that are contiguous or non-contiguous and can be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra; Hampel et al., EP0360257; Berzal-Herranz et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif or configuration is meant, an enzymatic nucleic acid molecule comprising a motif as is generally described as NCH Rz in FIG. 1 and in Ludwig et al., International PCT Publication No. WO 98/58058 and U.S. patent application Ser. No. 08/878,640. Inozymes possess endonuclease activity to cleave nucleic acid substrates having a cleavage triplet NCH/, where N is a nucleotide, C is cytidine and H is adenosine, uridine or cytidine, and “/” represents the cleavage site. H is used interchangeably with X. Inozymes can also possess endonuclease activity to cleave nucleic acid substrates having a cleavage triplet NCN/, where N is a nucleotide, C is cytidine, and “/” represents the cleavage site. “I” in FIG. 1 represents an Inosine nucleotide, preferably a ribo-Inosine or xylo-Inosine nucleoside.

By “G-cleaver” motif or configuration is meant, an enzymatic nucleic acid molecule comprising a motif as is generally described as G-cleaver Rz in FIG. 1 and in Eckstein et al., U.S. Pat. No. 6,127,173. G-cleavers possess endonuclease activity to cleave nucleic acid substrates having a cleavage triplet NYN/, where N is a nucleotide, Y is uridine or cytidine and “/” represents the cleavage site. G-cleavers can be chemically modified as is generally shown in FIG. 1.

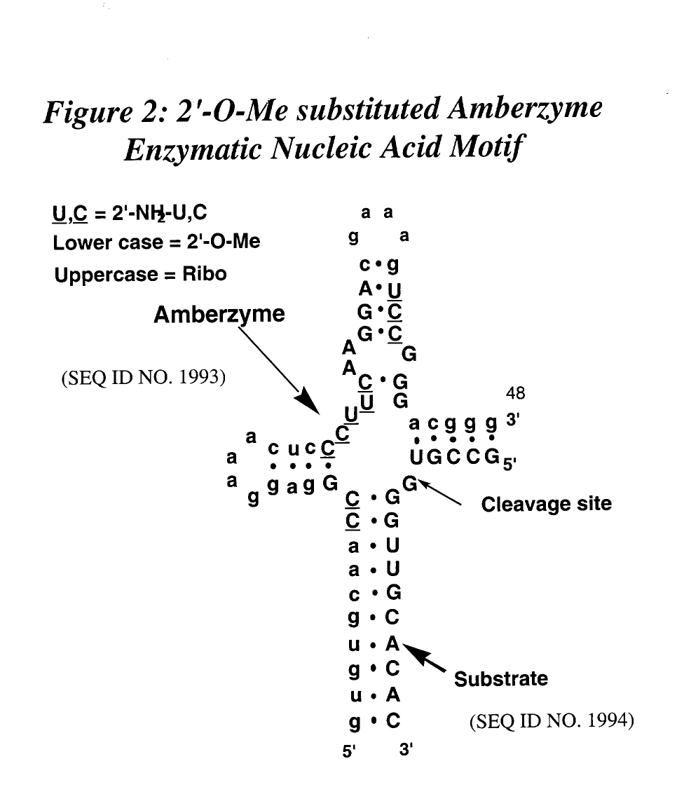

By “amberzyme” motif or configuration is meant, an enzymatic nucleic acid molecule comprising a motif as is generally described in FIG. 2 and in Beigelman et al., International PCT publication No. WO 99/55857 and U.S. patent application Ser. No. 09/476,387. Amberzymes possess endonuclease activity to cleave nucleic acid substrates having a cleavage triplet NG/N, where N is a nucleotide, G is guanosine, and “/” represents the cleavage site. Amberzymes can be chemically modified to increase nuclease stability through substitutions as are generally shown in FIG. 2. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

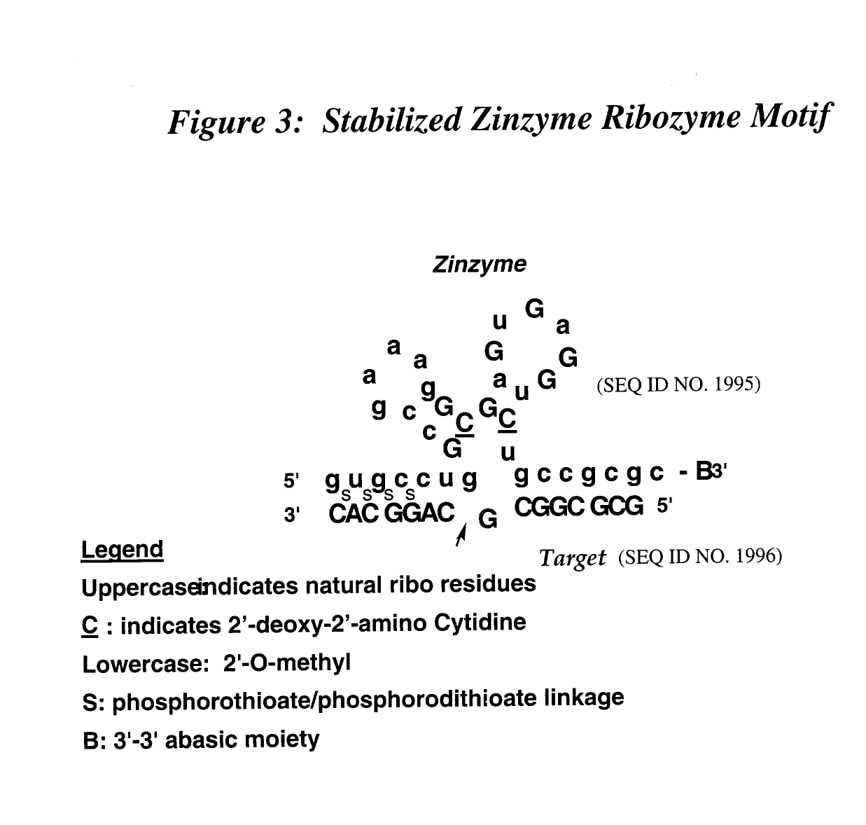

By “zinzyme” motif or configuration is meant, an enzymatic nucleic acid molecule comprising a motif as is generally described in FIG. 3 and in Beigelman et al., International PCT publication No. WO 99/55857 and U.S. patent application Ser. No. 09/918,728. Zinzymes possess endonuclease activity to cleave nucleic acid substrates having a cleavage triplet including but not limited to YG/Y, where Y is uridine or cytidine, and G is guanosine and “/” represents the cleavage site. Zinzymes can be chemically modified to increase nuclease stability through substitutions as are generally shown in FIG. 3, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

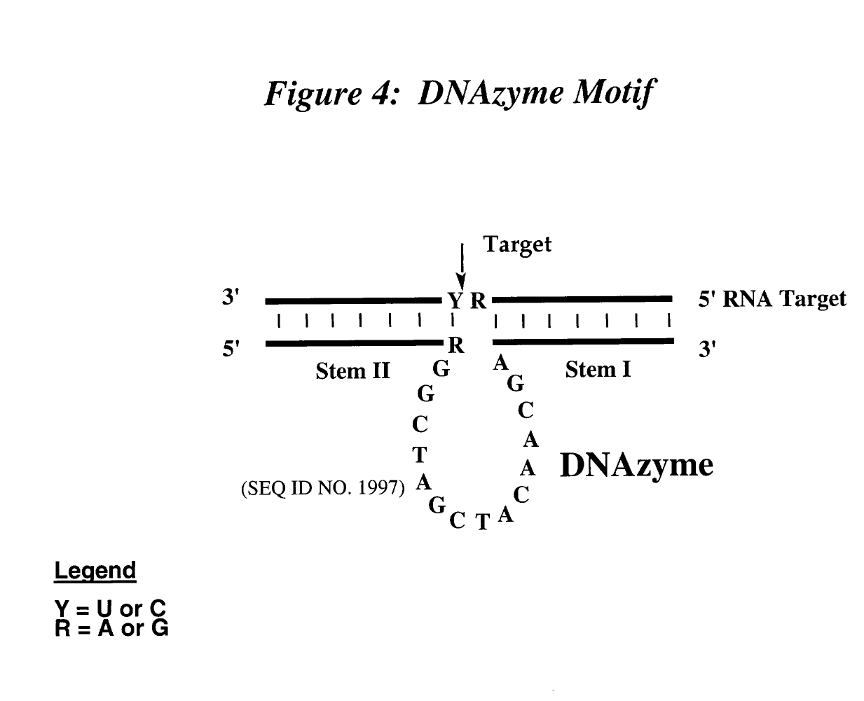

By ‘DNAzyme’ is meant, an enzymatic nucleic acid molecule that does not require the presence of a 2′-OH group within its own nucleic acid sequence for activity. In particular embodiments the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 4 and is generally reviewed in Usman et al., U.S. Pat. No., 6,159,714; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection, see Santoro et al., supra and as generally described in Joyce et al., U.S. Pat. No. 5,807,718. Additional DNAzyme motifs can be selected by using techniques similar to those described in these references, and hence, are within the scope of the present invention. DNAzymes of the invention can comprise nucleotides modified at the nucleic acid base, sugar, or phosphate backbone. Non-limiting examples of sugar modifications that can be used in DNAzymes of the invention include 2′-O-alkyl modifications such as 2′-O-methyl or 2′-O-allyl, 2′-C-alkyl modifications such as 2′-C-allyl, 2′-deoxy-2′-amino, 2′-halo modifications such as 2′-fluoro, 2′-chloro, or 2′-bromo, isomeric modifications such as arabinofuranose or xylofuranose based nucleic acids, and other sugar modifications such as 4′-thio or 4′-carbocyclic nucleic acids. Non-limiting examples of nucleic acid based modifications that can be used in DNAzymes of the invention include modified purine heterocycles, G-clamp heterocycles, and various modified pyrimidine cycles. Non-limiting examples of backbone modifications that can be used in DNAzymes of the invention include phosphorothioate, phosphorodithioate, phosphoramidate, and methylphosphonate internucleotide linkages. DNAzymes of the invention can comprise naturally occurring nucleic acids, chimeras of chemically modified and naturally occurring nucleic acids, or completely modified nucleic acids.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule.

By “stably interact” is meant interaction of oligonucleotides with target nucleic acid molecules (e.g., by forming hydrogen bonds with complementary nucleotides in the target under physiological conditions) that is sufficient to the intended purpose (e.g., cleavage of target RNA by an enzyme).

By “equivalent” RNA to HER2 is meant to include those naturally occurring RNA molecules having homology (partial or complete) to HER2 nucleic acids or encoding for proteins with similar function as HER2 proteins in various organisms, including humans, rodents, primates, rabbits, pigs, protozoans, fungi, plants, and other microorganisms and parasites. The equivalent RNA sequence also includes, in addition to the coding region, regions such as a 5′-untranslated region, a 3′-untranslated region, introns, a intron-exon junction and the like.

By “homology” is meant the nucleotide sequence of two or more nucleic acid molecules is partially or completely identical.

By “component” of HER2 is meant a peptide or protein subunit expressed from a HER2 gene.

By “antisense nucleic acid”, is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to a substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). An RNase H enzyme binds to a nucleic acid molecule-target RNA complex and cleaves the target RNA sequence. A RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, a RNase H activating region can also comprise a variety of sugar chemistries. For example, a RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of an RNase H activating region and the instant invention.

By “aptamer” or “nucleic acid aptamer” as used herein is meant a nucleic acid molecule that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand binding domain of a protein, thereby preventing interaction of the naturally occurring ligand with the protein. Similarly, the nucleic acid molecules of the instant invention can bind to Her-2 encoded RNA or proteins receptors to block activity of the activity of target protein or nucleic acid. This is a non-limiting example and those in the art will recognize that other embodiments can be readily generated using techniques generally known in the art, see for example Gold et al., U.S. Pat. Nos. 5,475,096 and 5,270,163; Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

The term “short interfering RNA” or “siRNA” as used herein refers to a double stranded nucleic acid molecule capable of RNA interference “RNAi”, see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. As used herein, siRNA molecules need not be limited to those molecules containing only RNA, but further encompasses chemically modified nucleotides and non-nucleotides.

By “gene” is meant a nucleic acid that encodes a RNA, for example, nucleic acid sequences including but not limited to structural genes encoding a polypeptide.

“Complementarity” refers to the ability of a nucleic acid to form hydrogen bond or bonds with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “decoy” is meant a nucleic acid molecule, for example RNA or DNA, or aptamer that is designed to preferentially bind to a predetermined ligand. Such binding can result in the inhibition or activation of a target molecule. A decoy or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy can be designed to bind to HER2 and block the binding of HER2 or a decoy can be designed to bind to HER2 and prevent interaction with the HER2 protein.

Several varieties of naturally occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target-binding portion of a enzymatic nucleic acid that is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

Nucleic acid molecules that modulate expression of HER2-specific RNAs represent a therapeutic approach to treat cancer, including, but not limited to breast and ovarian cancer and any other cancer, disease or condition that responds to the modulation of HER2 expression.

In one embodiment of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; Chowrira & McSwiggen, U.S. Pat. No. 5,631,359; of the hepatitis delta virus motif is described by Perrotta and Been, 1992 Biochemistry 31, 16; of the RNase P motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071 and of DNAzymes by Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262, and Beigelman et al., International PCT publication No. WO 99/55857. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs such as the Aptazyme (Breaker et al., WO 98/43993), Amberzyme (Class I motif; FIG. 2; Beigelman et al., U.S. Ser. No. 09/301,511) and Zinzyme (FIG. 3) (Beigelman et al., U.S. Ser. No. 09/301,511), all included by reference herein including drawings, can also be used in the present invention. These specific motifs or configurations are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In one embodiment of the present invention, a nucleic acid molecule of the instant invention can be between about 10 and 100 nucleotides in length. Exemplary enzymatic nucleic acid molecules of the invention are shown in Tables III and IV. For example, enzymatic nucleic acid molecules of the invention are preferably between about 15 and 50 nucleotides in length, more preferably between about 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between about 15 and 40 nucleotides in length, more preferably between about 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between about 15 and 75 nucleotides in length, more preferably between about 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between about 10 and 40 nucleotides in length, more preferably between about 12 and 25 nucleotides in length, e.g., 18, 19, 20, or 21 nucleotides in length (see for example Maher et al., 1990, Biochemistry, 29, 8820-8826; Strobel and Dervan, 1990, Science, 249, 73-75). Those skilled in the art will recognize that all that is required is for a nucleic acid molecule to be of length and conformation sufficient and suitable for the nucleic acid molecule to interact with its target and/or catalyze a reaction contemplated herein. The length of nucleic acid molecules of the instant invention are not limiting within the general limits stated.

Preferably, a nucleic acid molecule that modulates, for example down-regulates, HER2 expression, comprises between 12 and 100 bases complementary to a RNA molecule of HER2. Even more preferably, a nucleic acid molecule that modulates HER2 expression comprises between 14 and 24 bases complementary to a RNA molecule of HER2.

The invention provides a method for producing a class of nucleic acid-based gene modulating agents that exhibit a high degree of specificity for RNA of a desired target. For example, an enzymatic nucleic acid molecule is preferably targeted to a highly conserved sequence region of target RNAs encoding HER2 (and specifically a HER2 gene) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., enzymatic nucleic acid molecules, siRNA, antisense, and/or DNAzymes) can be expressed from DNA and/or RNA vectors that are delivered to specific cells.

As used in herein “cell” is used in its usual biological sense, and does not refer to an entire multicellular organism. A cell can, for example, be in vitro, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

By “HER2 proteins” is meant, a peptide or protein comprising HER2/ERB2/NEU tyrosine kinase-type cell surface receptor or a peptide or protein encoded by a HER2/ERB2/NEU gene.

By “highly conserved sequence region” is meant, a nucleotide sequence of one or more regions in a target gene that does not vary significantly from one generation to the other or from one biological system to the other.

Nucleic acid-based inhibitors of HER2 expression are useful for the prevention and/or treatment of cancer, including but not limited to breast cancer and ovarian cancer and any other disease or condition that respond to the modulation of HER2 expression.

By “related” is meant that the reduction of HER2 (and specifically a HER2 gene) RNA levels and thus reduction in the level of the respective protein relieves, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection or infusion pump, with or without their incorporation in biopolymers. In preferred embodiments, the enzymatic nucleic acid inhibitors comprise sequences that are complementary to the substrate sequences in Tables III and IV. Examples of such enzymatic nucleic acid molecules also are shown in Tables III and IV. Examples of such enzymatic nucleic acid molecules consist essentially of sequences defined in these tables.

In another embodiment, the invention features siRNA, antisense nucleic acid molecules and 2-5A chimera including sequences complementary to the substrate sequences shown in Tables III and IV. Such nucleic acid molecules can include sequences as shown for the binding arms of the enzymatic nucleic acid molecules in Tables III and IV. Similarly, triplex molecules can be targeted to corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to a substrate such that the substrate molecule forms a loop, and/or an antisense molecule can bind such that the antisense molecule forms a loop. Thus, the antisense molecule can be complementary to two (or even more) non-contiguous substrate sequences. In addition, two (or even more) non-contiguous sequence portions of an antisense molecule can be complementary to a target sequence.

By “consists essentially of” is meant that the active nucleic acid molecule of the invention, for example, an enzymatic nucleic acid molecule, contains an enzymatic center or core equivalent to those in the examples, and binding arms able to bind RNA such that cleavage at the target site occurs. Other sequences can be present that do not interfere with such cleavage. Thus, a core region of an enzymatic nucleic acid molecule can, for example, include one or more loop, stem-loop structure, or linker that does not prevent enzymatic activity. Thus, various regions in the sequences in Table III and IV can be such a loop, stem-loop, nucleotide linker, and/or non-nucleotide linker and can be represented generally as sequence “X”. The nucleic acid molecules of the instant invention, such as Hammerhead, Inozyme, G-cleaver, amberzyme, zinzyme, DNAzyme, antisense, 2-5A antisense, triplex forming nucleic acid, and decoy nucleic acids, can contain other sequences or non-nucleotide linkers that do not interfere with the function of the nucleic acid molecule.

Sequence X can be a linker of ≧2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, sequence X can be a non-nucleotide linker. In yet another embodiment, the nucleotide linker X can be a nucleic acid aptamer, such as an ATP aptamer, HER2 Rev aptamer (RRE), HER2 Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, a non-nucleotide linker X is as defined herein. Non-nucleotides can include abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in one embodiment, the invention features an enzymatic nucleic acid molecule having one or more non-nucleotide moieties, and having enzymatic activity to cleave an RNA or DNA molecule.

In another aspect of the invention, enzymatic nucleic acid molecules, siRNA molecules or antisense molecules that interact with target RNA molecules and modulate HER2 (and specifically a HER2 gene) activity or expression are expressed from transcription units inserted into DNA or RNA vectors. The recombinant vectors are preferably DNA plasmids or viral vectors. Enzymatic nucleic acid molecule, siRNA or antisense expressing viral vectors can be constructed based on, but not limited to, adeno-associated virus, retrovirus, adenovirus, or alphavirus as well as others known in the art. Preferably, recombinant vectors capable of expressing enzymatic nucleic acid molecules or antisense are delivered as described below, and persist in target cells. Alternatively, viral vectors can be used that provide for transient expression of enzymatic nucleic acid molecules or antisense. Such vectors can be repeatedly administered as necessary. Once expressed, the siRNA, enzymatic nucleic acid molecules or antisense bind to target RNA and modulate its function or expression. Delivery of enzymatic nucleic acid molecule or antisense expressing vectors can be systemic, such as by intravenous or intramuscular administration, by administration to target cells ex-planted from the subject followed by reintroduction into the subject, or by any other means that allows for introduction into a desired target cell. Antisense DNA and DNAzymes can be expressed via the use of a single stranded DNA intracellular expression vector.

By “vectors” is meant any nucleic acid- and/or viral-based technique used to deliver a desired nucleic acid.

By “subject” or “patient” is meant an organism that is a donor or recipient of explanted cells or the cells of the organism. “Subject” or “patient” also refers to an organism to which the nucleic acid molecules of the invention can be administered. Preferably, a subject or patient is a mammal or mammalian cells. More preferably, a subject or patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme, for example with a nucleic acid molecule comprising chemical modifications. In some cases, the activity or stability of the nucleic acid molecule can be decreased (i.e., less than ten-fold), but the overall activity of the nucleic acid molecule is enhanced, in vivo.

Nucleic acid molecules of the instant invention, individually, or in combination or in conjunction with other drugs, can be used to treat diseases or conditions discussed above. For example, to treat a disease or condition associated with the levels of HER2, a subject can be treated, or other appropriate cells can be treated, as is evident to those skilled in the art, individually or in combination with one or more drugs under conditions suitable for the treatment.

In a further embodiment, the described molecules, such as siRNA, antisense or enzymatic nucleic acid molecules, can be used in combination with other known treatments to treat conditions or diseases discussed above. For example, the described molecules can be used in combination with one or more known therapeutic agents to treat cancer, for example ovarian cancer and/or breast cancer, and any other disease or condition that respond to the modulation of HER2 expression.

In another embodiment, the invention features nucleic acid-based inhibitors (e.g., enzymatic nucleic acid molecules, including DNAzymes, ribozymes, and siRNA; antisense nucleic acids; 2-5A antisense chimeras; triplex DNA; antisense nucleic acids containing RNA cleaving chemical groups) and methods for their use to modulate the expression of genes (e.g., HER2 genes) capable of progression and/or maintenance of cancer and/or other disease states that respond to the modulation of HER2 expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

Other features and advantages of the invention will be apparent from the following description of the preferred embodiments thereof, and from the claims.

Mechanism of Action of Nucleic Acid Molecules of the Invention as is Known in the Art

Antisense: Antisense molecules can be modified or unmodified RNA, DNA, or mixed polymer oligonucleotides and primarily function by specifically binding to matching sequences resulting in inhibition of peptide synthesis (Wu-Pong, Nov 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). Backbone modified DNA chemistry which have thus far been shown to act as substrates for RNase H are phosphorothioates, phosphorodithioates, and borontrifluoridates. In addition, 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174, filed on Sep. 21, 1998). All of these references are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

RNA Interference: RNA interference refers to the process of sequence specific post transcriptional gene silencing in animals mediated by short interfering RNAs (siRNA) (Fire et al., 1998, Nature, 391, 806). The corresponding process in plants is commonly referred to as post transcriptional gene silencing or RNA silencing and is also referred to as quelling in fungi. The process of post transcriptional gene silencing is thought to be an evolutionarily conserved cellular defense mechanism used to prevent the expression of foreign genes which is commonly shared by diverse flora and phyla (Fire et al., 1999, Trends Genet., 15, 358). Such protection from foreign gene expression may have evolved in response to the production of double stranded RNAs (dsRNA) derived from viral infection or the random integration of transposon elements into a host genome via a cellular response that specifically destroys homologous single stranded RNA or viral genomic RNA. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNA) (Berstein et al., 2001, Nature, 409, 363). Short interfering RNAs derived from dicer activity are typically about 21-23 nucleotides in length and comprise about 19 base pair duplexes. Dicer has also been implicated in the excision of 21 and 22 nucleotide small temporal RNAs (stRNA) from precursor RNA of conserved structure that are implicated in translational control (Hutvagner et al., 2001, Science, 293, 834). The RNAi response also features an endonuclease complex containing a siRNA, commonly referred to as an RNA-induced silencing complex (RISC), which mediates cleavage of single stranded RNA having sequence homologous to the siRNA. Cleavage of the target RNA takes place in the middle of the region complementary to the guide sequence of the siRNA duplex (Elbashir et al., 2001, Genes Dev., 15, 188).

Short interfering RNA mediated RNAi has been studied in a variety of systems. Fire et al., 1998, Nature, 391, 806, were the first to observe RNAi in C. Elegans. Wianny and Goetz, 1999, Nature Cell Biol., 2, 70, describes RNAi mediated by dsRNA in mouse embryos. Hammond et al., 2000, Nature, 404, 293, describe RNAi in Drosophila cells transfected with dsRNA. Elbashir et al., 2001, Nature, 411, 494, describe RNAi induced by introduction of duplexes of synthetic 21-nucleotide RNAs in cultured mammalian cells including human embryonic kidney and HeLa cells. Recent work in Drosophila embryonic lysates has revealed certain requirements for siRNA length, structure, chemical composition, and sequence that are essential to mediate efficient RNAi activity. These studies have shown that 21 nucleotide siRNA duplexes are most active when containing two nucleotide 3′-overhangs. Furthermore, substitution of one or both siRNA strands with 2′-deoxy or 2′-O-methyl nucleotides abolishes RNAi activity, whereas substitution of 3′-terminal siRNA nucleotides with deoxy nucleotides was shown to be tolerated. Mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end (Elbashir et al., 2001, EMBO J., 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309), however siRNA molecules lacking a 5′-phosphate are active when introduced exogenously, suggesting that 5′-phosphorylation of siRNA constructs may occur in vivo.

Enzymatic Nucleic Acid: Several varieties of naturally occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention can modulate, e.g., down-regulate HER2 protein expression and can be used to treat disease or diagnose disease associated with the levels of HER2. Enzymatic nucleic acid sequences targeting HER2 RNA and sequences that can be targeted with nucleic acid molecules of the invention to down-regulate HER2 expression are shown in Tables III and IV.

The enzymatic nature of an enzymatic nucleic acid molecule allows the concentration of enzymatic nucleic acid molecule necessary to affect a therapeutic treatment to be lower than a nucleic acid molecule lacking enzymatic activity, such as an antisense nucleic acid. This reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a enzymatic nucleic acid molecule.

Nucleic acid molecules having an endonuclease enzymatic activity are able to repeatedly cleave other separate RNA molecules in a nucleotide base sequence-specific manner. With proper design and construction, such enzymatic nucleic acid molecules can be targeted to virtually any RNA transcript, and achieve efficient cleavage in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving enzymatic nucleic acid molecules can be used as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

Enzymatic nucleic acid molecules of the invention that are allosterically regulated (“allozymes”) can be used to modulate, including down-regulate HER2 expression. These allosteric enzymatic nucleic acids or allozymes (see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842) are designed to respond to a signaling agent, for example, mutant HER2 protein, wild-type HER2 protein, mutant HER2 RNA, wild-type HER2 RNA, other proteins and/or RNAs involved in HER2 activity, compounds, metals, polymers, molecules and/or drugs that are targeted to HER2 expressing cells etc., which in turn modulates the activity of the enzymatic nucleic acid molecule. In response to interaction with a predetermined signaling agent, the allosteric enzymatic nucleic acid molecule is activated or inhibited such that the expression of a particular target is selectively regulated, including down-regulated. The target can comprise wild-type HER2, mutant HER2, a component of HER2, and/or a predetermined cellular component that modulates HER2 activity. For example, allosteric enzymatic nucleic acid molecules that are activated by interaction with a RNA encoding HER2 protein can be used as therapeutic agents in vivo. The presence of RNA encoding the HER2 protein activates the allosteric enzymatic nucleic acid molecule that subsequently cleaves the RNA encoding HER2 protein resulting in the inhibition of HER2 protein expression. In this manner, cells that express the HER2 protein are selectively targeted.

In another non-limiting example, an allozyme can be activated by a HER2 protein, peptide, or mutant polypeptide that causes the allozyme to inhibit the expression of HER2 gene, by, for example, cleaving RNA encoded by HER2 gene. In this non-limiting example, the allozyme acts as a decoy to inhibit the function of HER2 and also inhibit the expression of HER2 once activated by the HER2 protein.

The nucleic acid molecules of the instant invention are also referred to as GeneBloc reagents, which are essentially nucleic acid molecules (eg; ribozymes, antisense) capable of down-regulating gene expression.

Target Sites

Targets for useful enzymatic nucleic acid molecules and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, and hereby incorporated by reference herein in totality. Other examples include the following PCT applications, which concern inactivation of expression of disease-related genes: WO 95/23225, WO 95/13380, WO 94/02595, incorporated by reference herein. Rather than repeat the guidance provided in those documents here, below are provided specific non-limiting examples of such methods, not limiting to those in the art. Enzymatic nucleic acid molecules to such targets are designed as described in the above applications and synthesized to be tested in vitro and in vivo, as also described. The sequences of human HER2 RNAs were screened for optimal enzymatic nucleic acid target sites using a computer-folding algorithm. Nucleic acid molecule binding/cleavage sites were identified. These sites are shown in Tables III and IV (all sequences are 5′ to 3′ in the tables). The nucleotide base position is noted in the Tables as that site to be cleaved by the designated type of enzymatic nucleic acid molecule. Human sequences can be screened and enzymatic nucleic acid molecule and/or antisense thereafter designed, as discussed in Stinchcomb et al., WO 95/23225. In addition, mouse targeted nucleic acid molecules can be used to test efficacy of action of the enzymatic nucleic acid molecule, siRNA and/or antisense prior to testing in humans.

In addition, enzymatic nucleic acid, siRNA, and antisense nucleic acid molecule binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions, such as between, for example the binding arms and the catalytic core of an enzymatic nucleic acid, are eliminated from consideration. Varying binding arm lengths can be chosen to optimize activity.

Antisense, hammerhead, DNAzyme, NCH, amberzyme, zinzyme or G-Cleaver enzymatic nucleic acid molecule, siRNA, and antisense nucleic acid binding/cleavage sites were identified and were designed to anneal to various sites in the RNA target. The enzymatic nucleic acid binding arms or siRNA and antisense nucleic acid sequences are complementary to the target site sequences described above. The nucleic acid molecules are chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic Acid Molecules

Synthesis of nucleic acids greater than 100 nucleotides in length can be difficult using automated methods, and currently the therapeutic cost of such molecules can be prohibitive. In this invention, small nucleic acid motifs (“small refers to nucleic acid motifs less than about 100 nucleotides in length, preferably less than about 80 nucleotides in length, and more preferably less than about 50 nucleotides in length; e.g., DNAzymes) are currently preferred for exogenous delivery. The simple structure of these molecules increases the ability of the nucleic acid to invade targeted regions of RNA structure. Exemplary molecules of the instant invention are chemically synthesized as described herein, and others can similarly be synthesized.

Oligonucleotides (e.g., DNAzymes, antisense) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small-scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNAzymes is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for RNA and chemically modified RNA or DNA, including certain enzymatic nucleic acid molecules and siRNA molecules, follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM 1₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA•3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA•3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive nucleic acid molecules or binding attenuated control (BAC) oligonucleotides can be synthesized by substituting one or more nucleotides in the nucleic acid molecule to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Nucleic acid molecules are purified by gel electrophoresis using known methods or are purified by high-pressure liquid chromatography (HPLC; See Wincott et al., Supra, the totality of which is hereby incorporated herein by reference) and are re-suspended in water.

The sequences of the nucleic acid molecules, including enzymatic nucleic acid molecules and antisense, that are chemically synthesized, are shown in Table IV. The sequences of the enzymatic nucleic acid and antisense constructs that are chemically synthesized, are complementary to the Target sequences shown in Table IV. Those in the art will recognize that these sequences are representative only of many more such sequences where the enzymatic portion of the ribozyme (all but the binding arms) is altered to affect activity. The enzymatic nucleic acid sequences listed in Tables III and IV can be formed of deoxyribonucleotides or other nucleotides or non-nucleotides. Such enzymatic nucleic acid molecules with enzymatic activity are equivalent to the enzymatic nucleic acid molecules described specifically in the Tables.

Optimizing Activity of the Nucleic Acid Molecule of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples of sugar, base and phosphate modifications that can be introduced into nucleic acid molecules with significant enhancement in their nuclease stability and efficacy. For example, oligonucleotides can be modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules are also known to increase efficacy (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). The publications describe general methods and strategies to determine the location of incorporation of sugar, base and/or phosphate modifications and the like into enzymatic nucleic acid molecules without inhibiting catalysis. Similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, excessive modifications can cause some toxicity. Therefore when designing nucleic acid molecules the amount of these internucleotide linkages should be minimized. The reduction in the concentration of these linkages can lower toxicity resulting in increased efficacy and higher specificity of the therapeutic nucleic acid molecules.

Nucleic acid molecules having chemical modifications that maintain or enhance activity are provided. Such nucleic acid molecules are also generally more resistant to nucleases than unmodified nucleic acid molecules. Thus, the in vitro and/or in vivo activity should not be significantly lowered. Therapeutic nucleic acid molecules delivered exogenously are optimally stable within cells until translation of the target RNA has been inhibited long enough to reduce the levels of the undesirable protein. This period of time varies between hours to days depending upon the disease state. Nucleic acid molecules are preferably resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein)) have expanded the ability to modify nucleic acid molecules by introducing nucleotide modifications to enhance their nuclease stability as described above.

In one embodiment, nucleic acid molecules of the invention include one or more G-clamp nucleotides. A G-clamp nucleotide is a modified cytosine analog wherein modifications result in the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substation within an oligonucleotide can result in substantially enhanced helical thermal stability and mismatch discrimination when hybridized to complementary oligonucleotides. The inclusion of such nucleotides in nucleic acid molecules of the invention can enable both enhanced affinity and specificity to nucleic acid targets.

In another embodiment, the invention features conjugates and/or complexes of nucleic acid molecules targeting HER2 genes. Compositions and conjugates are used to facilitate delivery of molecules into a biological system, such as cells. The conjugates provided by the instant invention can impart therapeutic activity by transferring therapeutic compounds across cellular membranes, altering the pharmacokinetics, and/or modulating the localization of nucleic acid molecules of the invention. The present invention encompasses the design and synthesis of novel agents for the delivery of molecules, including but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or 20 nucleotides in length, or can comprise a single nucleotide with a phosphorus based linkage, for example, a phosphoramidate or phosphodiester linkage. The biodegradable nucleic acid linker molecule can also comprise nucleic acid backbone, nucleic acid sugar, or nucleic acid base modifications.

The term “biodegradable” as used herein, refers to degradation in a biological system, for example, enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siRNA, dsRNA, allozymes, aptamers, decoys and analogs thereof. Biologically active molecules of the invention also include molecules capable of modulating the pharmacokinetics and/or pharmacodynamics of other biologically active molecules, for example lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus containing group and saturated or unsaturated alkyl group, optionally substituted with OH, COOH, oxo, amine, or substituted or unsubstituted aryl groups.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of subjects with nucleic acid molecules can also include combinations of different types of nucleic acid molecules.

In the case that down-regulation of the target is desired, therapeutic nucleic acid molecules (e.g., DNAzymes) delivered exogenously are optimally stable within cells until translation of the target RNA has been inhibited long enough to reduce the levels of the targeted protein. This period of time varies between hours to days depending upon the disease state. These nucleic acid molecules should be resistant to nucleases in order to function as effective intracellular therapeutic agents. Improvements in the chemical synthesis of nucleic acid molecules described in the instant invention and others known in the art have expanded the ability to modify nucleic acid molecules by introducing nucleotide modifications to enhance their nuclease stability as described above.

In another embodiment, nucleic acid catalysts having chemical modifications that maintain or enhance enzymatic activity are provided. Such nucleic acids are also generally more resistant to nucleases than unmodified nucleic acid. Thus, the in vitro and/or in vivo the activity of the nucleic acid should not be significantly lowered. As exemplified herein, such enzymatic nucleic acids are useful for in vitro and/or in vivo techniques even if activity over all is reduced 10 fold (Burgin et al., 1996, Biochemistry, 35, 14090). Such enzymatic nucleic acids herein are said to “maintain” the enzymatic activity of an all RNA ribozyme or all DNA DNAzyme.

In another aspect the nucleic acid molecules comprise a 5′ and/or a 3′- cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In another embodiment the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

The term “alkyl” as used herein refers to a saturated aliphatic hydrocarbon, including straight-chain, branched-chain “isoalkyl”, and cyclic alkyl groups. The term “alkyl” also comprises alkoxy, alkyl-thio, alkyl-thio-alkyl, alkoxyalkyl, alkylamino, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Preferably, the alkyl group has 1 to 12 carbons. More preferably it is a lower alkyl of from about 1 to 7 carbons, more preferably about 1 to 4 carbons. The alkyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkenyl groups containing at least one carbon-carbon double bond, including straight-chain, branched-chain, and cyclic groups. Preferably, the alkenyl group has about 2 to 12 carbons. More preferably it is a lower alkenyl of from about 2 to 7 carbons, more preferably about 2 to 4 carbons. The alkenyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkynyl groups containing at least one carbon-carbon triple bond, including straight-chain, branched-chain, and cyclic groups. Preferably, the alkynyl group has about 2 to 12 carbons. More preferably it is a lower alkynyl of from about 2 to 7 carbons, more preferably about 2 to 4 carbons. The alkynyl group can be substituted or unsubstituted. When substituted the substituted group(s) preferably comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Alkyl groups or moieties of the invention can also include aryl, alkylaryl, carbocyclic aryl, heterocyclic aryl, amide and ester groups. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from about 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NR—R, where R is either alkyl, aryl, alkylaryl or hydrogen. An “ester” refers to an —C(O)—OR′, where R is either alkyl, aryl, alkylaryl or hydrogen.

The term “alkoxyalkyl” as used herein refers to an alkyl-O-alkyl ether, for example methoxyethyl or ethoxymethyl.

The term “alkyl-thio-alkyl” as used herein refers to an alkyl-S-alkyl thioether, for example methylthiomethyl or methylthioethyl.

The term “amino” as used herein refers to a nitrogen containing group as is known in the art derived from ammonia by the replacement of one or more hydrogen radicals by organic radicals. For example, the terms “aminoacyl” and “aminoalkyl” refer to specific N-substituted organic radicals with acyl and alkyl substituent groups respectively.

The term “amination” as used herein refers to a process in which an amino group or substituted amine is introduced into an organic molecule.

The term “exocyclic amine protecting moiety” as used herein refers to a nucleobase amino protecting group compatible with oligonucleotide synthesis, for example an acyl or amide group.

The term “alkenyl” as used herein refers to a straight or branched hydrocarbon of a designed number of carbon atoms containing at least one carbon-carbon double bond. Examples of “alkenyl” include vinyl, allyl, and 2-methyl-3-heptene.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkenyl” as used herein refers to a C3-C8 cyclic hydrocarbon containing at least one carbon-carbon double bond. Examples of cycloalkenyl include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloalkyl” as used herein refers to a C3-C8 cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C3-C7 cycloalkyl group attached to the parent molecular moiety through an alkyl group, as defined above. Examples of cycloalkylalkyl groups include cyclopropylmethyl and cyclopentylethyl.

The terms “halogen” or “halo” as used herein refers to indicate fluorine, chlorine, bromine, and iodine.

The term “heterocycloalkyl,” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “C1-C6 hydrocarbyl” as used herein refers to straight, branched, or cyclic alkyl groups having 1-6 carbon atoms, optionally containing one or more carbon-carbon double or triple bonds. Examples of hydrocarbyl groups include, for example, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, vinyl, 2-pentene, cyclopropylmethyl, cyclopropyl, cyclohexylmethyl, cyclohexyl and propargyl. When reference is made herein to C1-C6 hydrocarbyl containing one or two double or triple bonds it is understood that at least two carbons are present in the alkyl for one double or triple bond, and at least four carbons for two double or triple bonds.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein. There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified enzymatic nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” is meant sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O-NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., DNAzyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of the disease progression by affording the possibility of combination therapies (e.g., multiple enzymatic nucleic acid molecules targeted to different genes, enzymatic nucleic acid molecules coupled with known small molecule inhibitors, or intermittent treatment with combinations of enzymatic nucleic acid molecules (including different enzymatic nucleic acid molecule motifs) and/or other chemical or biological molecules). The treatment of subjects with nucleic acid molecules can also include combinations of different types of nucleic acid molecules. Therapies can be devised which include a mixture of enzymatic nucleic acid molecules (including different enzymatic nucleic acid molecule motifs), antisense and/or 2-5A chimera molecules to one or more targets to alleviate symptoms of a disease.

Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995, which are both incorporated herein by reference. Sullivan et al., PCT WO 94/02595, further describes the general methods for delivery of enzymatic RNA molecules. These protocols can be utilized for the delivery of virtually any nucleic acid molecule. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. Alternatively, the nucleic acid/vehicle combination is locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches include the use of various transport and carrier systems, for example though the use of conjugates and biodegradable polymers. For a comprehensive review on drug delivery strategies including CNS delivery, see Ho et al., 1999, Curr. Opin. Mol. Ther., 1, 336-343 and Jain, Drug Delivery Systems: Technologies and Commercial Opportunities, Decision Resources, 1998 and Groothuis et al., 1997, J. Neuro Virol., 3, 387-400. More detailed descriptions of nucleic acid delivery and administration are provided in Sullivan et al., supra, Draper et al., PCT WO93/23569, Beigelman et al., PCT WO99/05094, and Klimuk et al., PCT WO99/04819, all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a subject.

The polynucleotides of the invention can be administered (e.g., RNA, DNA or protein) and introduced into a subject by any standard means described herein and known in the art, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies, including CNS delivery of the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058. All these references are hereby incorporated herein by reference.

The invention also features the use of the composition comprising surface-modified liposomes containing poly (ethylene glycol) lipids (PEG-modified, or long-circulating liposomes or stealth liposomes). Nucleic acid molecules of the invention can also comprise covalently attached PEG molecules of various molecular weights. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes, which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

In another aspect of the invention, nucleic acid molecules of the present invention are preferably expressed from transcription units (see for example Couture et al., 1996, TIG., 12, 510, Skillern et al., International PCT Publication No. WO 00/22113, Conrad, International PCT Publication No. WO 00/22114, and Conrad, U.S. Pat. No. 6,054,299) inserted into DNA or RNA vectors. The recombinant vectors are preferably DNA plasmids or viral vectors. Enzymatic nucleic acid expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecule binds to the target mRNA. Delivery of nucleic acid molecule expressing vectors can be systemic, such as by intravenous or intra-muscular administration, by administration to target cells ex-planted from the subject followed by reintroduction into the subject, or by any other means that would allow for introduction into the desired target cell (for a review see Couture et al., 1996, TIG., 12, 510).

One aspect of the invention features an expression vector comprising a nucleic acid sequence encoding at least one of the nucleic acid molecules of the instant invention. The nucleic acid sequence encoding the nucleic acid molecule of the instant invention is operably linked in a manner that allows expression of that nucleic acid molecule.

In another aspect, the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner that allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res.., 21, 2867-72; Lieber et al., 1993, Methods Enzymol., 217, 47-66; Zhou et al., 1990, Mol. Cell. Biol., 10, 4529-37). All of these references are incorporated by reference herein. Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

Another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner that allows expression and/or delivery of said nucleic acid molecule.

In another embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of DNAzyme molecules and binding/cleavage sites within HER2 RNA.

### Example 1

### Identification of Potential Target Sites in Human HER2 RNA

The sequence of human HER2 genes were screened for accessible sites using a computer-folding algorithm. Regions of the RNA that do not form secondary folding structures and contained potential enzymatic nucleic acid molecule and/or antisense binding/cleavage sites were identified. The sequences of these binding/cleavage sites are shown in Tables III and IV.

### Example 2

### Selection of Enzymatic Nucleic Acid Cleavage Sites in Human HER2 RNA

Enzymatic nucleic acid molecule target sites were chosen by analyzing sequences of Human HER2 (Genbank accession No: X03363) and prioritizing the sites on the basis of folding. Enzymatic nucleic acid molecules were designed that can bind each target and were individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core were eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of DNAzymes for Efficient Cleavage and/or Blocking of HER2 RNA

DNAzyme molecules were designed to anneal to various sites in the RNA message. The binding arms of the DNAzyme molecules were complementary to the target site sequences described above. The DNAzymes were chemically synthesized. The method of synthesis used followed the procedure for nucleic acid synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%. The sequences of the chemically synthesized DNAzyme molecules used in this study are shown below in Table IV.

### Example 4

### DNAzyme Cleavage of HER2 RNA Target in vitro

DNAzymes targeted to the human HER2 RNA were designed and synthesized as described above. These enzymatic nucleic acid molecules are tested for cleavage activity in vitro, for example, using the following procedure. The target sequences and the nucleotide location within the HER2 RNA are given in Tables III and IV.

Cleavage Reactions:

Ribozymes and substrates were synthesized in 96-well format using 0.2 μmol scale. Substrates were 5′-³²P labeled and gel purified using 7.5% polyacrylamide gels, and eluting into water. Assays were done by combining trace substrate with 500 nM Ribozyme or greater, and initiated by adding final concentrations of 40 mM Mg⁺², and 50 mM Tris-Cl pH 8.0. For each ribozyme/substrate combination a control reaction was done to ensure cleavage was not the result of non-specific substrate degradation. A single three hour time point was taken and run on a 15% polyacrylamide gel to asses cleavage activity. Gels were dried and scanned using a Molecular Dynamics Phosphorimager and quantified using Molecular Dynamics ImageQuant software. Percent cleaved was determined by dividing values for cleaved substrate bands by full-length (uncleaved) values plus cleaved values and multiplying by 100 (% cleaved=[C/(U+C)]*100).

### Example 4

### DNAzyme Cleavage of HER2 RNA Target in vivo

Cell Culture Review

The greatest HER2 specific effects have been observed in cancer cell lines that express high levels of HER2 protein (as measured by ELISA). Specifically, in one study that treated five human breast cancer cell lines with the HER2 antibody (anti-erbB2-sFv), the greatest inhibition of cell growth was seen in three cell lines (MDA-MB-361, SKBR-3 and BT-474) that express high levels of HER2 protein. No inhibition of cell growth was observed in two cell lines (MDA-MB-231 and MCF-7) that express low levels of HER2 protein (Wright, M., Grim, J., Deshane, J., Kim, M., Strong, T. V., Siegel, G. P., Curiel, D. T. (1997) An intracellular anti-erbB-2 single-chain antibody is specifically cytotoxic to human breast carcinoma cells overexpressing erbB-2. Gene Therapy 4: 317-322). Another group successfully used SKBR-3 cells to show HER2 antisense oligonucleotide-mediated inhibition of HER2 protein expression and HER2 RNA knockdown (Vaughn, J. P., Iglehart, J. D., Deimrdji, S., Davis, P., Babiss, L. E., Caruthers, M. H., Marks, J. R. (1995) Antisense DNA downregulation of the ERBB2 oncogene measured by a flow cytometric assay. Proc Natl Acad Sci USA 92: 8338-8342). Other groups have also demonstrated a decrease in the levels of HER2 protein, HER2 mRNA and/or cell proliferation in cultured cells using anti-HER2 DNAzymes or antisense molecules (Suzuki T., Curcio, L. D., Tsai, J. and Kashani-Sabet M. (1997) Anti-c-erb-B-2 Ribozyme for Breast Cancer. In Methods in Molecular Medicine, Vol. 11, Therapeutic Applications of Ribozmes, Human Press, Inc., Totowa, N J; Weichen, K., Zimmer, C. and Dietel, M. (1997) Selection of a high activity c-erbB-2 ribozyme using a fusion gene of c-erbB-2 and the enhanced green fluorescent protein. Cancer Gene Therapy 5: 45-51; Czubayko, F., Downing, S. G., Hsieh, S. S., Goldstein, D. J., Lu P. Y., Trapnell, B. C. and Wellstein, A. (1997) Adenovirus-mediated transduction of ribozymes abrogates HER-2/neu and pleiotrophin expression and inhibits tumor cell proliferation. Gene Ther. 4: 943-949; Colomer, R., Lupu, R., Bacus, S. S. and Gelmann, E. P. (1994) erbB-2 antisense oligonucloetides inhibit the proliferation of breast carcinoma cells with erbB-2 oncogene amplification. British J. Cancer 70: 819-825; Betram et al., 1994). Because cell lines that express higher levels of HER2 have been more sensitive to anti-HER2 agents, we prefer using several medium to high expressing cell lines, including SKBR-3 and T47D, for DNAzyme screens in cell culture.

A variety of endpoints have been used in cell culture models to look at HER2-mediated effects after treatment with anti-HER2 agents. Phenotypic endpoints include inhibition of cell proliferation, apoptosis assays and reduction of HER2 protein expression. Because overexpression of HER2 is directly associated with increased proliferation of breast and ovarian tumor cells, a proliferation endpoint for cell culture assays will preferably be used as the primary screen. There are several methods by which this endpoint is measured. Following treatment of cells with DNAzymes, cells are allowed to grow (typically 5 days) after which either the cell viability, the incorporation of [³H] thymidine into cellular DNA and/or the cell density is be measured. The assay of cell density is very straightforward and can be done in a 96-well format using commercially available fluorescent nucleic acid stains (such as Syto® 13 or CyQuant®). The assay using CyQuant® is described herein and is currently being employed to screen ˜100 DNAzymes targeting HER2 (details below).

As a secondary, confirmatory endpoint a DNAzyme-mediated decrease in the level of HER2 protein expression is evaluated using a HER2-specific ELISA.

Validation of Cell Lines and DNAzyme Treatment Conditions

Two human breast cancer cell lines (T47D and SKBR-3) that are known to express medium to high levels of HER2 protein, respectively, are considered for DNAzyme screening. In order to validate these cell lines for HER2-mediated sensitivity, both cell lines are treated with the HER2 specific antibody, Herceptin® (Genentech) and its effect on cell proliferation is determined. Herceptin® is added to cells at concentrations ranging from 0-8 μM in medium containing either no serum (OptiMem), 0.1% or 0.5% FBS and efficacy is determined via cell proliferation. Maximal inhibition of proliferation (˜50%) in both cell lines is typically observed after addition of Herceptin® at 0.5 nM in medium containing 0.1% or no FBS. The fact that both cell lines are sensitive to an anti-HER2 agent (Herceptin®) supports their use in experiments testing anti-HER2 DNAzymes.

Prior to DNAzyme screening, the choice of the optimal lipid(s) and conditions for DNAzyme delivery is determined empirically for each cell line. Applicant has established a panel of cationic lipids (lipids as described in PCT application WO99/05094) that can be used to deliver DNAzymes to cultured cells and are very useful for cell proliferation assays that are typically 3-5 days in length. (Additional description of useful lipids is provided above, and those skilled in the art are also familiar with a variety of lipids that can be used for delivery of oligonucleotide to cells in culture.) Initially, this panel of lipid delivery vehicles is screened in SKBR-3 and T47D cells using previously established control oligonucleotides. Specific lipids and conditions for optimal delivery are selected for each cell line based on these screens. These conditions are used to deliver HER2 specific DNAzymes to cells for primary (inhibition of cell proliferation) and secondary (decrease in HER2 protein) efficacy endpoints.

Primary Screen: Inhibition of Cell Proliferation

DNAzyme screens are performed using an automated, high throughput 96-well cell proliferation assay. Cell proliferation is measured over a 5-day treatment period using the nucleic acid stain CyQuant® for determining cell density. The growth of cells treated with DNAzyme/lipid complexes is compared to both untreated cells and to cells treated with Scrambled-arm Attenuated core Controls (“SACs”). SACs can no longer bind to the target site due to the scrambled arm sequence and have nucleotide changes in the core that greatly diminish DNAzyme cleavage. These SACs are used to determine non-specific inhibition of cell growth caused by DNAzyme chemistry (i.e. multiple 2′ O-Me modified nucleotides and a 3′ inverted abasic). Lead DNAzymes are chosen from the primary screen based on their ability to inhibit cell proliferation in a specific manner. Dose response assays are carried out on these leads and a subset was advanced into a secondary screen using the level of HER2 protein as an endpoint.

Secondary Screen: Decrease in HER2 Protein and/or RNA

A secondary screen that measures the effect of anti-HER2 DNAzymes on HER2 protein and/or RNA levels is used to affirm preliminary findings. A robust HER2 ELISA for both T47D and SKBR-3 cells has been established and is available for use as an additional endpoint. In addition, a real time RT-PCR assay (TaqMan assay) has been developed to assess HER2 RNA reduction compared to an actin RNA control. Dose response activity of nucleic acid molecules of the instant invention is used to assess both HER2 protein and RNA reduction endpoints.

DNAzyme Mechanism Assays

A TaqMan® assay for measuring the DNAzyme-mediated decrease in HER2 RNA has also been established. This assay is based on PCR technology and can measure in real time the production of HER2 mRNA relative to a standard cellular mRNA such as GAPDH. This RNA assay is used to establish proof that lead DNAzymes are working through an RNA cleavage mechanism and result in a decrease in the level of HER2 mRNA, thus leading to a decrease in cell surface HER2 protein receptors and a subsequent decrease in tumor cell proliferation.

Animal Models

Evaluating the efficacy of anti-HER2 agents in animal models is an important prerequisite to human clinical trials. As in cell culture models, the most HER2 sensitive mouse tumor xenografts are those derived from human breast carcinoma cells that express high levels of HER2 protein. In a recent study, nude mice bearing BT-474 xenografts were sensitive to the anti-HER2 humanized monoclonal antibody Herceptin®, resulting in an 80% inhibition of tumor growth at a 1 mg kg dose (ip, 2×week for 4-5 weeks). Tumor eradication was observed in 3 of 8 mice treated in this manner (Baselga, J., Norton, L. Albanell, J., Kim, Y. M. and Mendelsohn, J. (1998) Recombinant humanized anti-HER2 antibody (Herceptin) enhances the antitumor activity of paclitaxel and doxorubicin against HER2/neu overexpressing human breast cancer xenografts. Cancer Res. 15: 2825-2831). This same study compared the efficacy of Herceptin® alone or in combination with the commonly used chemotherapeutics, paclitaxel or doxorubicin. Although, all three anti-HER2 agents caused modest inhibition of tumor growth, the greatest antitumor activity was produced by the combination of Herceptin® and paclitaxel (93% inhibition of tumor growth vs 35% with paclitaxel alone). The above studies provide proof that inhibition of HER2 expression by anti-HER2 agents causes inhibition of tumor growth in animals. Lead anti-HER2 DNAzymes chosen from in vitro assays are further tested in mouse xenograft models. DNAzymes are first tested alone and then in combination with standard chemotherapies.

Animal Model Development

Three human breast tumor cell lines (T47D, SKBR-3 and BT-474) were characterized to establish their growth curves in mice. These three cell lines have been implanted into the mammary papillae of both nude and SCID mice and primary tumor volumes are measured 3 times per week. Growth characteristics of these tumor lines using a Matrigel implantation format can also be established. The use of two other breast cell lines that have been engineered to express high levels of HER2 can also be used in the described studies. The tumor cell line(s) and implantation method that supports the most consistent and reliable tumor growth is used in animal studies testing the lead HER2 DNAzyme(s). DNAzymes are administered by daily subcutaneous injection or by continuous subcutaneous infusion from Alzet mini osmotic pumps beginning 3 days after tumor implantation and continuing for the duration of the study. Group sizes of at least 10 animals are employed. Efficacy is determined by statistical comparison of tumor volume of DNAzyme-treated animals to a control group of animals treated with saline alone. Because the growth of these tumors is generally slow (45-60 days), an initial endpoint is the time in days it takes to establish an easily measurable primary tumor (i.e. 50-100 mm³) in the presence or absence of DNAzyme treatment.

Clinical Summary

Overview

Breast cancer is a common cancer in women and also occurs in men to a lesser degree. The incidence of breast cancer in the United States is ˜180,000 cases per year and ˜46,000 die each year of the disease. In addition, 21,000 new cases of ovarian cancer per year lead to ˜13,000 deaths (data from Hung, M. -C., Matin, A., Zhang, Y., Xing, X., Sorgi, F., Huang, L. and Yu, D. (1995) HER-2/neu-targeting gene therapy—a review. Gene 159: 65-71 and the Surveillance, Epidemiology and End Results Program, NCI Surveillance, Epidemiology and End Results Program (SEER) Cancer Statistics Review: http://www.seer.ims.nci.nih.gov/Publications/CSR1973_{—}1996/). Ovarian cancer is a potential secondary indication for anti-HER2 DNAzyme therapy.

Breast cancer is evaluated or “staged” on the basis of tumor size, and whether it has spread to lymph nodes and/or other parts of the body. In Stage I breast cancer, the cancer is no larger than 2 centimeters and has not spread outside of the breast. In Stage II, the subject's tumor is 2-5 centimeters but cancer may have spread to the axillary lymph nodes. By Stage III, metastasis to the lymph nodes is typical, and tumors are ≧5 centimeters. Additional tissue involvement (skin, chest wall, ribs, muscles etc.) may also be noted. Once cancer has spread to additional organs of the body, it is classed as Stage IV.

Almost all breast cancers (>90%) are detected at Stage I or II, but 31% of these are already lymph node positive. The 5-year survival rate for node negative subjects (with standard surgery/radiation/chemotherapy/hormone regimens) is 97%; however, involvement of the lymph nodes reduces the 5-year survival to only 77%. Involvement of other organs (≧Stage III) drastically reduces the overall survival, to 22% at 5 years. Thus, chance of recovery from breast cancer is highly dependent on early detection. Because up to 10% of breast cancers are hereditary, those with a family history are considered to be at high risk for breast cancer and should be monitored very closely.

Therapy

Breast cancer is highly treatable and often curable when detected in the early stages. (For a complete review of breast cancer treatments, see the NCI PDQ for Breast Cancer.) Common therapies include surgery, radiation therapy, chemotherapy and hormonal therapy. Depending upon many factors, including the tumor size, lymph node involvement and location of the lesion, surgical removal varies from lumpectomy (removal of the tumor and some surrounding tissue) to mastectomy (removal of the breast, lymph nodes and some or all of the underlying chest muscle). Even with successful surgical resection, as many as 21% of the subjects may ultimately relapse (10-20 years). Thus, once local disease is controlled by surgery, adjuvant radiation treatments, chemotherapies and/or hormonal therapies are typically used to reduce the rate of recurrence and improve survival. The therapy regimen employed depends not only on the stage of the cancer at its time of removal, but other variables such the type of cancer (ductal or lobular), whether lymph nodes were involved and removed, age and general health of the subject and if other organs are involved.

Common chemotherapies include various combinations of cytotoxic drugs to kill the cancer cells. These drugs include paclitaxel (Taxol), docetaxel, cisplatin, methotrexate, cyclophosphamide, doxorubin, fluorouracil etc. Significant toxicities are associated with these cytotoxic therapies. Well-characterized toxicities include nausea and vomiting, myelosuppression, alopecia and mucosity. Serious cardiac problems are also associated with certain of the combinations, e.g. doxorubin and paclitaxel, but are less common.

Testing for estrogen and progesterone receptors helps to determine whether certain anti-hormone therapies might be helpful in inhibiting tumor growth. If either or both receptors are present, therapies to interfere with the action of the hormone ligands, can be given in combination with chemotherapy and are generally continued for several years. These adjuvant therapies are called SERMs, selective estrogen receptor modulators, and they can give beneficial estrogen-like effects on bone and lipid metabolism while antagonizing estrogen in reproductive tissues. Tamoxifen is one such compound. The primary toxic effect associated with the use of tamoxifen is a 2 to 7-fold increase in the rate of endometrial cancer. Blood clots in the legs and lung and the possibility of stroke are additional side effects. However, tamoxifen has been determined to reduce breast cancer incidence by 49% in high-risk subjects and an extensive, somewhat controversial, clinical study is underway to expand the prophylactic use of tamoxifen. Another SERM, raloxifene, was also shown to reduce the incidence of breast cancer in a large clinical trial where it was being used to treat osteoporosis. In additional studies, removal of the ovaries and/or drugs to keep the ovaries from working are being tested.

Bone marrow transplantation is being studied in clinical trials for breast cancers that have become resistant to traditional chemotherapies or where >3 lymph nodes are involved. Marrow is removed from the subject prior to high-dose chemotherapy to protect it from being destroyed, and then replaced after the chemotherapy. Another type of “transplant” involves the exogenous treatment of peripheral blood stem cells with drugs to kill cancer cells prior to replacing the treated cells in the bloodstream.

One biological treatment, a humanized monoclonal anti-HER2 antibody, Herceptin® (Genentech) has been approved by the FDA as an additional treatment for HER2 positive tumors. Herceptin® binds with high affinity to the extracellular domain of HER2 and thus blocks its signaling action. Herceptin® can be used alone or in combination with chemotherapeutics (i.e. paclitaxel, docetaxel, cisplatin, etc.) (Pegram, M. D., Lipton, A., Hayes, D. F., Weber, B. L., Baselga, J. M., Tripathy, D., Baly, D., Baughman, S. A., Twaddell, T., Glaspy, J. A. and Slamon, D. J. (1998) Phase II study of receptor-enhanced chemosensitivity using recombinant humanized anti-p185HER2/neu monoclonal antibody plus cisplatin in subjects with HER2/neu-overexpressing metastatic breast cancer refractory to chemotherapy treatment. J. Clin. Oncol. 16: 2659-2671). In Phase III studies, Herceptin® significantly improved the response rate to chemotherapy as well as improving the time to progression (Ross, J. S. and Fletcher, J. A. (1998) The HER-2/neu oncogene in breast cancer: Prognostic factor, predictive factor and target for therapy. Oncologist 3: 1998). The most common side effects attributed to Herceptin® are fever and chills, pain, asthenia, nausea, vomiting, increased cough, diarrhea, headache, dyspnea, infection, rhinitis, and insomnia. Herceptin® in combination with chemotherapy (paclitaxel) can lead to cardiotoxicity (Sparano, J. A. (1999) Doxorubicin/taxane combinations: Cardiac toxicity and pharmacokinetics. Semin. Oncol. 26: 14-19), leukopenia, anemia, diarrhea, abdominal pain and infection.

HER2 Protein Levels for Subject Screening and as a Potential Endpoint

Because elevated HER2 levels can be detected in at least 30% of breast cancers, breast cancer subjects can be pre-screened for elevated HER2 prior to admission to initial clinical trials testing an anti-HER2 DNAzyme. Initial HER2 levels can be determined (by ELISA) from tumor biopsies or resected tumor samples.

During clinical trials, it may be possible to monitor circulating HER2 protein by ELISA (Ross and Fletcher, 1998). Evaluation of serial blood/serum samples over the course of the anti-HER2 DNAzyme treatment period could be useful in determining early indications of efficacy. In fact, the clinical course of Stage IV breast cancer was correlated with shed HER2 protein fragment following a dose-intensified paclitaxel monotherapy. In all responders, the HER2 serum level decreased below the detection limit (Luftner, D., Schnabel. S. and Possinger, K. (1999) c-erbB-2 in serum of subjects receiving fractionated paclitaxel chemotherapy. Int. J. Biol. Markers 14: 55-59).

Two cancer-associated antigens, CA27.29 and CA15.3, can also be measured in the serum. Both of these glycoproteins have been used as diagnostic markers for breast cancer. CA27.29 levels are higher than CA15.3 in breast cancer subjects; the reverse is true in healthy individuals. Of these two markers, CA27.29 was found to better discriminate primary cancer from healthy subjects. In addition, a statistically significant and direct relationship was shown between CA27.29 and large vs small tumors and node postive vs node negative disease (Gion, M., Mione, R., Leon, A. E. and Dittadi, R. (1999) Comparison of the diagnostic accuracy of CA27.29 and CA15.3 in primary breast cancer. Clin. Chem. 45: 630-637). Moreover, both cancer antigens were found to be suitable for the detection of possible metastases during follow-up (Rodriguez de Paterna, L., Arnaiz, F., Estenoz, J. Ortuno, B. and Lanzos E. (1999) Study of serum tumor markers CEA, CA15.3, CA27.29 as diagnostic parameters in subjects with breast carcinoma. Int. J. Biol. Markers 10: 24-29). Thus, blocking breast tumor growth may be reflected in lower CA27.29 and/or CA15.3 levels compared to a control group. FDA submissions for the use of CA27.29 and CA15.3 for monitoring metastatic breast cancer subjects have been filed (reviewed in Beveridge, R. A. (1999) Review of clinical studies of CA27.29 in breast cancer management. Int. J. Biol. Markers 14: 36-39). Fully automated methods for measurement of either of these markers are commercially available.

Indications

Particular degenerative and disease states that can be associated with HER2 expression modulation include but are not limited to cancer, for example breast cancer and ovarian cancer and/or any other diseases or conditions that are related to or will respond to the levels of HER2 in a cell or tissue, alone or in combination with other therapies

The present body of knowledge in HER2 research indicates the need for methods to assay HER2 activity and for compounds that can regulate HER2 expression for research, diagnostic, and therapeutic use.

The use of monoclonal antibodies, chemotherapy, radiation therapy, and analgesics, are all non-limiting examples of methods that can be combined with or used in conjunction with the nucleic acid molecules (e.g. DNAzymes) of the instant invention. Common chemotherapies that can be combined with nucleic acid molecules of the instant invention include various combinations of cytotoxic drugs to kill cancer cells. These drugs include but are not limited to paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the nucleic acid molecules of the instant invention (e.g. DNAzyme molecules) are hence within the scope of the instant invention.

Diagnostic Uses

The nucleic acid molecules of this invention (e.g., enzymatic nucleic acid molecules) can be used as diagnostic tools to examine genetic drift and mutations within diseased cells or to detect the presence of HER2 RNA in a cell. The close relationship between enzymatic nucleic acid molecule activity and the structure of the target RNA allows the detection of mutations in any region of the molecule that alters the base-pairing and three-dimensional structure of the target RNA. By using multiple enzymatic nucleic acid molecules described in this invention, one can map nucleotide changes which are important to RNA structure and function in vitro, as well as in cells and tissues. Cleavage of target RNAs with enzymatic nucleic acid molecules can be used to inhibit gene expression and define the role (essentially) of specified gene products in the progression of disease. In this manner, other genetic targets can be defined as important mediators of the disease. These experiments can lead to better treatment of the disease progression by affording the possibility of combinational therapies (e.g., multiple enzymatic nucleic acid molecules targeted to different genes, enzymatic nucleic acid molecules coupled with known small molecule inhibitors, or intermittent treatment with combinations of enzymatic nucleic acid molecules and/or other chemical or biological molecules). Other in vitro uses of enzymatic nucleic acid molecules of this invention are well known in the art, and include detection of the presence of mRNAs associated with HER2-related condition. Such RNA is detected by determining the presence of a cleavage product after treatment with an enzymatic nucleic acid molecule using standard methodology.

In a specific example, enzymatic nucleic acid molecules that cleave only wild-type or mutant forms of the target RNA are used for the assay. The first enzymatic nucleic acid molecule is used to identify wild-type RNA present in the sample and the second enzymatic nucleic acid molecule is used to identify mutant RNA in the sample. As reaction controls, synthetic substrates of both wild-type and mutant RNA are cleaved by both enzymatic nucleic acid molecules to demonstrate the relative enzymatic nucleic acid molecule efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acid molecules, two substrates and one unknown sample which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HER2) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively. The use of enzymatic nucleic acid molecules in diagnostic applications contemplated by the instant invention is more fully described in George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

Additional Uses

Potential uses of sequence-specific enzymatic nucleic acid molecules of the instant invention can have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to modulate gene expression of target genes in bacterial, microbial, fungal, viral, and eukaryotic systems including plant or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations, which is not specifically disclosed herein. Thus, for example, in each instance herein any of the terms “comprising”, “consisting essentially of” and “consisting of” can be replaced with either of the other two terms. The terms and expressions that have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed can be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the claims that follow.

### TABLE I

Characteristics of Naturally Occurring Ribozymes

Group I Introns

Size: ˜150 to >1000 nucleotides.

Requires a U in the target sequence immediately 5′ of the cleavage site.

Binds 4-6 nucleotides at the 5′-side of the cleavage site.

Reaction mechanism: attack by the 3′-OH of guanosine to generate cleavage products with 3′-OH and 5′-guanosine.

Additional protein cofactors required in some cases to help folding and maintenance of the active structure.

Over 300 known members of this class. Found as an intervening sequence in Tetrahymena thermophila rRNA, fungal mitochondria, chloroplasts, phage T4, blue-green algae, and others.

Major structural features largely established through phylogenetic comparisons, mutagenesis, and biochemical studies [^{I},^{II}].

Complete kinetic framework established for one ribozyme [^{III},^{IV},^{V},^{VI}].

Studies of ribozyme folding and substrate docking underway [^{VII},^{VIII},^{IX}].

Chemical modification investigation of important residues well established [^{X},^{XI}].

The small (4-6 nt) binding site may make this ribozyme too non-specific for targeted RNA cleavage, however, the Tetrahymena group I intron has been used to repair a “defective” β-galactosidase message by the ligation of new β-galactosidase sequences onto the defective message [^{XII}].

RNAse P RNA (M1 RNA)

Size: ˜290 to 400 nucleotides.

RNA portion of a ubiquitous ribonucleoprotein enzyme.

Cleaves tRNA precursors to form mature tRNA [^{XIII}].

Reaction mechanism: possible attack by M²⁺-OH to generate cleavage products with 3′-OH and 5′-phosphate.

RNAse P is found throughout the prokaryotes and eukaryotes. The RNA subunit has been sequenced from bacteria, yeast, rodents, and primates.

Recruitment of endogenous RNAse P for therapeutic applications is possible through hybridization of an External Guide Sequence (EGS) to the target RNA [^{XIV},^{XV}]

Important phosphate and 2′ OH contacts recently identified [^{XVI},^{XVII}]

Group II Introns

Size: >1000 nucleotides.

Trans cleavage of target RNAs recently demonstrated [^{XViII},^{XIX}].

Sequence requirements not fully determined.

Reaction mechanism: 2′-OH of an internal adenosine generates cleavage products with 3′-OH and a “lariat” RNA containing a 3′-5′ and a 2′-5′ branch point.

Only natural ribozyme with demonstrated participation in DNA cleavage [^{XX},^{XXI}] in addition to RNA cleavage and ligation.

Major structural features largely established through phylogenetic comparisons [^{XXII}].

Important 2′ OH contacts beginning to be identified [^{XXIII}]

Kinetic framework under development [^{XXIV}]

Neurospora VS RNA

Size: ˜144 nucleotides.

Trans cleavage of hairpin target RNAs recently demonstrated [^{XXV}].

Sequence requirements not fully determined.

Reaction mechanism: attack by 2′-OH 5′ to the scissile bond to generate cleavage products with 2′,3′-cyclic phosphate and 5′-OH ends.

Binding sites and structural requirements not fully determined.

Only 1 known member of this class. Found in Neurospora VS RNA.

Hammerhead Ribozyme (see text for references)

Size: ˜13 to 40 nucleotides.

Requires the target sequence UH immediately 5′ of the cleavage site.

Binds a variable number nucleotides on both sides of the cleavage site.

Reaction mechanism: attack by 2′-OH 5′ to the scissile bond to generate cleavage products with 2′,3′-cyclic phosphate and 5′-OH ends.

14 known members of this class. Found in a number of plant pathogens (virusoids) that use RNA as the infectious agent.

Essential structural features largely defined, including 2 crystal structures [^{XXVI},^{XXVII}]

Minimal ligation activity demonstrated (for engineering through in vitro selection) [^{XXVIII}]

Complete kinetic framework established for two or more ribozymes [^{XXIX}].

Chemical modification investigation of important residues well established [^{XXX}].

Hairpin Ribozyme

Size: ˜50 nucleotides.

Requires the target sequence GUC immediately 3′ of the cleavage site.

Binds 4-6 nucleotides at the 5′-side of the cleavage site and a variable number to the 3′-side of the cleavage site.

Reaction mechanism: attack by 2′-OH 5′ to the scissile bond to generate cleavage products with 2′,3′-cyclic phosphate and 5′-OH ends.

3 known members of this class. Found in three plant pathogen (satellite RNAs of the tobacco ringspot virus, arabis mosaic virus and chicory yellow mottle virus) which uses RNA as the infectious agent.

Essential structural features largely defined [^{xxxi},^{XXXII},^{XXXIII},^{XXXIV}]

Ligation activity (in addition to cleavage activity) makes ribozyme amenable to engineering through in vitro selection [^{XXXV}]

Complete kinetic framework established for one ribozyme [^{XXXVI}].

Chemical modification investigation of important residues begun [^{XXXVII},^{XXXVIII}].

Hepatitis Delta Virus (HDV) Ribozyme

Size: ˜60 nucleotides.

Trans cleavage of target RNAs demonstrated [^{XXXIX}].

Binding sites and structural requirements not fully determined, although no sequences 5′ of cleavage site are required. Folded ribozyme contains a pseudoknot structure [^{xI}].

Reaction mechanism: attack by 2′-OH 5′ to the scissile bond to generate cleavage products with 2′,3′-cyclic phosphate and 5′-OH ends.

Only 2 known members of this class. Found in human HDV.

Circular form of HDV is active and shows increased nuclease stability [^{xIi}]

ⁱ. Michel, Francois; Westhof, Eric. Slippery substrates. Nat. Struct. Biol. (1994), 1(1), 5-7.

ⁱⁱ. Lisacek, Frederique; Diaz, Yolande; Michel, Francois. Automatic identification of group I intron cores in genomic DNA sequences. J. Mol. Biol. (1994), 235(4), 1206-17.

ⁱⁱⁱ. Herschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.

^{iv}. Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.

^{v}. Knitt, Deborah S.; Herschlag, Daniel. pH Dependencies of the Tetrahymena Ribozyme Reveal an Unconventional Origin of an Apparent pKa. Biochemistry (1996), 35(5), 1560-70.

^{vi}. Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.

^{vil}. Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.

^{vill}. Banerjee, Aloke Raj; Turner, Douglas H.. The time dependence of chemical modification reveals slow steps in the folding of a group I ribozyme. Biochemistry (1995), 34(19), 6504-12.

^{ix}. Zarrinkar, Patrick P.; Williamson, James R.. The P9.1-P9.2 peripheral extension helps guide folding of the Tetrahymena ribozyme. Nucleic Acids Res. (1996), 24(5), 854-8.

^{x}. Strobel, Scott A.; Cech, Thomas R.. Minor groove recognition of the conserved G.cntdot.U pair at the Tetrahymena ribozyme reaction site. Science (Washington, D.C.) (1995), 267(5198), 675-9.

^{xi}. Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.

^{xil}. Sullenger, Bruce A.; Cech, Thomas R.. Ribozyme-mediated repair of defective mRNA by targeted trans-splicing. Nature (London) (1994), 371(6498), 619-22.

^{xill}. Robertson, H. D.; Altman, S.; Smith, J. D. J. Biol. Chem., 247 5243-5251 (1972).

^{xiv}. Forster, Anthony C.; Altman, Sidney. External guide sequences for an RNA enzyme. Science (Washington, D.C., 1883-) (1990), 249(4970), 783-6.

^{xv}. Yuan, Y.; Hwang, E. S.; Altman, S. Targeted cleavage of mRNA by human RNase P. Proc. Natl. Acad. Sci. USA (1992) 89, 8006-10.

^{xvl}. Harris, Michael E.; Pace, Norman R.. Identification of phosphates involved in catalysis by the ribozyme RNase P RNA. RNA (1995), 1(2), 210-18.

^{xvll}. Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U. S. A. (1995), 92(26), 12510-14.

^{xvlll}. Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.

^{xix}. Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.

^{xx}. Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.

^{xxl}. Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.

^{xxll}. Michel, Francois; Ferat, Jean Luc. Structure and activities of group II introns. Annu. Rev. Biochem. (1995), 64, 435-61.

^{XXIII}. Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.

^{XXIV}. Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.

^{XXV}. Guo, Hans C. T.; Collins, Richard A.. Efficient trans-cleavage of a stem-loop RNA substrate by a ribozyme derived from Neurospora VS RNA. EMBO J. (1995), 14(2), 368-76.

^{XXVI}. Scott, W. G., Finch, J. T., Aaron, K. The crystal structure of an all RNA hammerhead ribozyme: A proposed mechanism for RNA catalytic cleavage. Cell, (1995), 81, 991-1002.

^{XXVII}. McKay, Structure and function of the hammerhead ribozyme: an unfinished story. RNA, (1996), 2, 395-403.

^{XXVIII}. Long, D., Uhlenbeck, O., Hertel, K. Ligation with hammerhead ribozymes. U.S. Pat. No. 5,633,133.

^{XXIX}. Hertel, K. J., Herschlag, D., Uhlenbeck, O. A kinetic and thermodynamic framework for the hammerhead ribozyme reaction. Biochemistry, (1994) 33, 3374-3385.Beigelman, L., et al., Chemical modifications of hammerhead ribozymes. J. Biol. Chem., (1995) 270, 25702-25708.

^{XXX}. Beigelman, L., et al., Chemical modifications of hammerhead ribozymes. J. Biol. Chem., (1995) 270, 25702-25708.

^{XXXI}. Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.

^{XXXII}. Chowrira, Bharat M.; Berzal-Herranz, Alfredo; Burke, John M.. Novel guanosine requirement for catalysis by the hairpin ribozyme. Nature (London) (1991), 354(6351), 320-2.

^{XXXIII}. Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.

^{XXXIV}. Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.

^{XXXV}. Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.

^{XXXVI}. Hegg, Lisa A.; Fedor, Martha J.. Kinetics and Thermodynamics of Intermolecular Catalysis by Hairpin Ribozymes. Biochemistry (1995), 34(48), 15813-28.

^{XXXVII}. Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.

^{XXXVIII}. Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.

^{XXXIX}. Perrotta, Anne T.; Been, Michael D.. Cleavage of oligoribonucleotides by a ribozyme derived from the hepatitis delta. virus RNA sequence. Biochemistry (1992), 31(1), 16-21.

^{XI}. Perrotta, Anne T.; Been, Michael D.. A pseudoknot-like structure required for efficient self-cleavage of hepatitis delta virus RNA. Nature (London) (1991), 350(6317), 434-6.

^{xli}. Puttaraju, M.; Perrotta, Anne T.; Been, Michael D.. A circular trans-acting hepatitis delta virus ribozyme. Nucleic Acids Res. (1993), 21(18), 4253-8.

TABLE II
A. 2.5 μmol Synthesis Cycle ABI 394 Instrument

| | | | | | |
|---|---|---|---|---|---|
| | | | | Wait | |
| | | | Wait | Time* | Wait |
| | | | Time* | 2′-O- | Time |
| Reagent | Equivalents | Amount | DNA | methyl | RNA |

| | | | | | | | | | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Sythesis Cycle ABI 394 Instrument

| | | | | | |
|---|---|---|---|---|---|
| | | | | Wait | |
| | | | Wait | Time* | Wait |
| | | | Time* | 2′-O- | Time |
| Reagent | Equivalents | Amount | DNA | methyl | RNA |

| | | | | | | | | | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | | | | | |
|---|---|---|---|---|---|
| | | | | Wait | |
| | | | Wait | Time | Wait |
| | Equivalents:DNA/ | Amount:DNA/ | Time* | 2′-O- | Time |
| Reagent | 2′-O-methyl/Ribo | 2′-O-methyl/Ribo | DNA | methyl | Ribo |

| | | | | | | | | | |
|---|---|---|---|---|---|---|---|---|---|
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

TABLE III
Human HER2 DNAzyme and Substrate Sequence

| | | | | |
|---|---|---|---|---|
| | | Seq | | Seq |
| Pos | Substrate | ID | DNAzyme | ID |

| | | | | |
|---|---|---|---|---|
| 9 | AAGGGGAG G UAACCCUG | 1 | CAGGTTTA GGCTAGCTACAACGA CTCCCCTT | 989 |
| 12 | GGGAGGUA A CCCUGGCC | 2 | GGCCAGGG GGCTAGCTACAACGA TACCTCCC | 990 |
| 18 | UAACCCUG G CCCCUUUG | 3 | GGCCAGGG GGCTAGCTACAACGA TACCTCCC | 991 |
| 27 | CCCCUUUG G UCGGGGCC | 4 | GCCCGGGG GGCTAGCTACAACGA CCCGACCA | 992 |
| 33 | UGGUCGGG G CCCCGGGC | 5 | GCCCGGGG GGCTAGCTACAACGA CCCGACCA | 993 |
| 40 | GGCCCCGG G CAGCCGCG | 6 | CGCGGCTG GGCTAGCTACAACGA CCGGGGCC | 994 |
| 43 | CCCGGGCA G CCGCGCGC | 7 | GCGCGCGG GGCTAGCTACAACGA TGCCCGGG | 995 |
| 46 | GGGCAGCC G CGCGCCCC | 8 | GGGGCGCG GGCTAGCTACAACGA GGCTGCCC | 996 |
| 48 | GCAGCCGC G CGCCCCUU | 9 | AAGGGGCG GGCTAGCTACAACGA GCGGCTGC | 997 |
| 50 | AGCCGCGC G CCCCUUCC | 10 | GGAAGGGG GGCTAGCTACAACGA GCGCGGCT | 998 |
| 60 | CCCUUCCC A CGGGGCCC | 11 | GGGCCCCG GGCTAGCTACAACGA GGGAAGGG | 999 |
| 65 | CCCACGGG G CCCUUUAC | 12 | GTAAAGGG GGCTAGCTACAACGA GGGAAGGG | 1000 |
| 72 | GGCCCUUU A CUGCGCCG | 13 | CGGCGCAG GGCTAGCTACAACGA AAAGGGCC | 1001 |
| 75 | CCUUUACU G CGCCGCGC | 14 | GCGCGGCG GGCTAGCTACAACGA AGTAAAGG | 1002 |
| 77 | UUUACUGC G CCGCGCGC | 15 | GCGCGCGG GGCTAGCTACAACGA GCAGTAAA | 1003 |
| 80 | ACUGCGCC G CGCGCCCG | 16 | CGGGCGCG GGCTAGCTACAACGA GGCGCAGT | 1004 |
| 82 | UGCGCCGC G CGCCCGGC | 17 | GCCGGGCG GGCTAGCTACAACGA GCGGCGCA | 1005 |
| 84 | CGCCGCGC G CCCGGCCC | 18 | GGGCCGGG GGCTAGCTACAACGA GCGCGGCG | 1006 |
| 89 | CGCGCCCG G CCCCCACC | 19 | GGTGGGGG GGCTAGCTACAACGA CGGGCGCG | 1007 |
| 95 | CGGCCCCC A CCCCUCGC | 20 | GCGAGGGG GGCTAGCTACAACGA GGGGGCCG | 1008 |
| 102 | CACCCCUC G CAGCACCC | 21 | GGGTGCTG GGCTAGCTACAACGA GAGGGGTG | 1009 |
| 105 | CCCUCGCA G CACCCCGC | 22 | GCGGGGTG GGCTAGCTACAACGA TGCGAGGG | 1010 |
| 107 | CUCGCAGC A CCCCGCGC | 23 | GCGCGGGG GGCTAGCTACAACGA GCTGCCAG | 1011 |
| 112 | AGCACCCC G CGCCCCGC | 24 | GCGGGGCG GGCTAGCTACAACGA GGGGTGCT | 1012 |
| 114 | CACCCCGC G CCCCGCGC | 25 | GCGCGGGG GGCTAGCTACAACGA GCGGGGTG | 1013 |
| 119 | CGCGCCCC G CGCCCUCC | 26 | GGAGGGCG GGCTAGCTACAACGA GGGGCGCG | 1014 |
| 121 | CGCCCCGC G CCCUCCCA | 27 | TGGGAGGG GGCTAGCTACAACGA GCGGGGCG | 1015 |
| 130 | CCCUCCCA G CCGGGUCC | 28 | GGACCCGG GGCTAGCTACAACGA TGGGAGGG | 1016 |
| 135 | CCAGCCGG G UCCAGCCG | 29 | CGGCTGGA GGCTAGCTACAACGA CCGGCTGG | 1017 |
| 140 | CGGGUCCA G CCGGAGCC | 30 | GGCTCCGG GGCTAGCTACAACGA TGGACCCG | 1018 |
| 146 | CAGCCGGA G CCAUGGGG | 31 | CCCCATGG GGCTAGCTACAACGA TCCGGCTG | 1019 |
| 149 | CCGGAGCC A UGGGGCCG | 32 | CGGCCCCA GGCTAGCTACAACGA GGCTCCGG | 1020 |
| 154 | GCCAUGGG G CCGGAGCC | 33 | GGCTCCGG GGCTAGCTACAACGA CCCATGGC | 1021 |
| 160 | GGGCCGGA G CCGCAGUG | 34 | CACTGCGG GGCTAGCTACAACGA TCCGGCCC | 1022 |
| 163 | CCGGAGCC G CAGUGAGC | 35 | GCTCACTG GGCTAGCTACAACGA GGCTCCGG | 1023 |
| 166 | GAGCCGCA G UGAGCACC | 36 | GGTGCTCA GGCTAGCTACAACGA TGCGGCTC | 1024 |
| 170 | CGCAGUGA G CACCAUGG | 37 | CCATGGTG GGCTAGCTACAACGA TCACTGCG | 1025 |
| 172 | CAGUGUGA A CCAUGGAG | 38 | CTCCATGG GGCTAGCTACAACGA GCTCACTG | 1026 |
| 175 | UGAGCACC A UGGAGCUG | 39 | CAGCTCCA GGCTAGCTACAACGA GGTGCTCA | 1027 |
| 180 | ACCAUGGA G CUGGCGGC | 40 | GCCGCCAG GGCTAGCTACAACGA TCCATGGT | 1028 |
| 184 | UGGAGCUG G CGGCCUUG | 41 | CAAGGCCG GGCTAGCTACAACGA CAGCTCCA | 1029 |
| 187 | AGCUGGCG G CCUUGUGC | 42 | GCACAAGG GGCTAGCTACAACGA CGCCAGCT | 1030 |
| 192 | GCGGCCUU G UGCCGCUG | 43 | CAGCGGCA GGCTAGCTACAACGA AAGGCCGC | 1031 |
| 194 | GGCCUUGU G CCGCUGGG | 44 | CCCAGCGG GGCTAGCTACAACGA ACAAGGCC | 1032 |
| 197 | CUUGUGCC G CUGGGGGC | 45 | GCCCCCAG GGCTAGCTACAACGA GGCACAAG | 1033 |
| 204 | CGCUGGGG G CUCCUCCU | 46 | AGGAGGAG GGCTAGCTACAACGA CCCCAGCG | 1034 |
| 214 | UCCUCCUC G CCCUCUUG | 47 | CAAGAGGG GGCTAGCTACAACGA GAGGAGGA | 1035 |
| 222 | GCCCUCUU G CCCCCCGG | 48 | CCGGGGGG GGCTAGCTACAACGA AAGAGGGC | 1036 |
| 232 | CCCCCGGA G CCGCGAGC | 49 | GCTCGCGG GGCTAGCTACAACGA TCCTTTTT | 1037 |
| 235 | CCGGAGCC G CGAGCACC | 50 | GGTGCTCG GGCTAGCTACAACGA GGCTCCGG | 1038 |
| 239 | AGCCGCGA G CACCCAAG | 51 | CTTGGGTG GGCTAGCTACAACGA TCGCGGCT | 1039 |
| 241 | CCGCGAGC A CCCAAGUG | 52 | CACTTGGG GGCTAGCTACAACGA GCTCGCGG | 1040 |
| 247 | GCACCCAA G UGUGCACC | 53 | GGTGCACA GGCTAGCTACAACGA TTGGGTGC | 1041 |
| 249 | ACCCAAGU G UGCACCGG | 54 | CCGGTGCA GGCTAGCTACAACGA ACTTGGGT | 1042 |
| 251 | CCAAGUGU G CACCGGCA | 55 | TGCCGGTG GGCTAGCTACAACGA ACACTTGG | 1043 |
| 253 | AAGUGUGC A CCGGCACA | 56 | TGTGCCGG GGCTAGCTACAACGA GCACACTT | 1044 |
| 257 | GUGCACCG G CACAGACA | 57 | TGTCTGTG GGCTAGCTACAACGA CGGTGCAC | 1045 |
| 259 | GCACCGGC A CAGACAUG | 58 | CATGTCTG GGCTAGCTACAACGA GCCGGTGC | 1046 |
| 263 | CGGCACAG A CAUGAAGC | 59 | GCTTCATG GGCTAGCTACAACGA CTGTGCCG | 1047 |
| 265 | GCACAGAC A UGAAGCUG | 60 | CAGCTTCA GGCTAGCTACAACGA GTCTGTGC | 1048 |
| 270 | GACAUGAA G CUGCGGCU | 61 | AGCCGCAG GGCTAGCTACAACGA TTCATGTC | 1049 |
| 273 | AUGAAGCU G CGGCUCCC | 62 | GGGAGCCG GGCTAGCTACAACGA AGCTTCAT | 1050 |
| 276 | AAGCUGCG G CUCCCUGC | 63 | GCAGGGAG GGCTAGCTACAACGA CGCAGCTT | 1051 |
| 283 | GGCUCCCU G CCAGUCCC | 64 | GGGACTGG GGCTAGCTACAACGA AGGGAGCC | 1052 |
| 287 | CCCUGCCA G UCCCGAGA | 65 | TCTCGGGA GGCTAGCTACAACGA TGGCAGGG | 1053 |
| 295 | GUCCCGAG A CCCACCUG | 66 | CAGGTGGG GGCTAGCTACAACGA CTCGGGAC | 1054 |
| 299 | CGAGACCC A CCUGGACA | 67 | TGTCCAGG GGCTAGCTACAACGA GGGTCTCG | 1055 |
| 305 | CCACCUGG A CAUGCUCC | 68 | GGAGCATG GGCTAGCTACAACGA CCAGGTGG | 1056 |
| 307 | ACCUGGAC A UGCUCCGC | 69 | GCGGAGCA GGCTAGCTACAACGA GTCCAGGT | 1057 |
| 309 | CUGGACAU G CUCCGCCA | 70 | TGGCGGAG GGCTAGCTACAACGA ATGTCCAG | 1058 |
| 314 | CAUGCUCC G CCACCUCU | 71 | AGAGGTGG GGCTAGCTACAACGA GGAGCATG | 1059 |
| 317 | GCUCCGCC A CCUCUACC | 72 | GGTAGAGG GGCTAGCTACAACGA GGCGGAGC | 1060 |
| 323 | CCACCUCU A CCAGGGCU | 73 | AGCCCTGG GGCTAGCTACAACGA AGAGGTGG | 1061 |
| 329 | CUACCAGG G CUGCCAGG | 74 | CCTGGCAG GGCTAGCTACAACGA CCTGGTAG | 1062 |
| 332 | CCAGGGCU G CCAGGUGG | 75 | CCACCTGG GGCTAGCTACAACGA AGCCCTGG | 1063 |
| 337 | GCUGCCAG G UGGUGCAG | 76 | CTGCACCA GGCTAGCTACAACGA CTGGCAGC | 1064 |
| 340 | GCCAGGUG G UGCAGGGA | 77 | TCCCTGCA GGCTAGCTACAACGA CACCTGCC | 1065 |
| 342 | CAGGUGGU G CAGGGAAA | 78 | TTTCCCTG GGCTAGCTACAACGA ACCACCTG | 1066 |
| 350 | GCAGGGAA A CCUGGAAC | 79 | GTTCCAGG GGCTAGCTACAACGA TTCCCTGC | 1067 |
| 357 | AACCUGGA A CUCACCUA | 80 | TAGGTGAG GGCTAGCTACAACGA TCCAGGTT | 1068 |
| 361 | UGGAACUC A CCUACCUG | 81 | TGGGCAGG GGCTAGCTACAACGA GAGTTCCA | 1069 |
| 365 | ACUCACCU A CCUGCCCA | 82 | TGGGCAGG GGCTAGCTACAACGA AGGTGAGT | 1070 |
| 369 | ACCUACCU G CCCACCAA | 83 | TTGGTGGG GGCTAGCTACAACGA AGGTAGGT | 1071 |
| 373 | ACCUGCCC A CCAAUGCC | 84 | GGCATTGG GGCTAGCTACAACGA GGGCAGGT | 1072 |
| 377 | GCCCACCA A UGCCAGCC | 85 | GGCTGGCA GGCTAGCTACAACGA TGGTGGGC | 1073 |
| 379 | CCACCAAU G CCAGCCUG | 86 | CAGGCTGG GGCTAGCTACAACGA ATTGGTGG | 1074 |
| 383 | CAAUGCCA G CCUGUCCU | 87 | AGGACAGG GGCTAGCTACAACGA TGGCATTG | 1075 |
| 387 | GCCAGCCU G UCCUUCCU | 88 | AGGAAGGA GGCTAGCTACAACGA AGGCTGGC | 1076 |
| 396 | UCCUUCCU G CAGGAUAU | 89 | ATATCCTG GGCTAGCTACAACGA AGGAAGGA | 1077 |
| 401 | CCUGCAGG A UAUCCAGG | 90 | CCTGGATA GGCTAGCTACAACGA CCTGCAGG | 1078 |
| 403 | UGCAGGAU A UCCAGGAG | 91 | CTCCTGGA GGCTAGCTACAACGA ATCCTGCA | 1079 |
| 412 | UCCAGGAG G UGCAGGGC | 92 | GCCCTGCA GGCTAGCTACAACGA CTCCTGGA | 1080 |
| 414 | CAGGAGGU G CAGGGCUA | 93 | TAGCCCTG GGCTAGCTACAACGA ACCTCCTG | 1081 |
| 419 | GGUGCAGG G CUACGUGC | 94 | GCACGTAG GGCTAGCTACAACGA CCTGCACC | 1082 |
| 422 | GCAGGGCU A CGUGCUCA | 95 | TGAGCACG GGCTAGCTACAACGA AGCCCTGC | 1083 |
| 424 | AGGGCUAC G UGCUCAUC | 96 | GATGAGCA GGCTAGCTACAACGA GTAGCCCT | 1084 |
| 426 | GGCUACGU G CUCAUCGC | 97 | GCGATGAG GGCTAGCTACAACGA ACGTAGCC | 1085 |
| 430 | ACGUGCUC A UCGCUCAC | 98 | GTGAGCGA GGCTAGCTACAACGA GAGCACGT | 1086 |
| 433 | UGCUCAUC G CUCACAAC | 99 | GTTGTGAG GGCTAGCTACAACGA GATGAGCA | 1087 |
| 437 | CAUCGCUC A CAACCAAG | 100 | CTTGGTTG GGCTAGCTACAACGA GAGCGATG | 1088 |
| 440 | CGCUCACA A CCAAGUGA | 101 | TCACTTGG GGCTAGCTACAACGA TGTGAGCG | 1089 |
| 445 | ACAACCAA G UGAGGCAG | 102 | CTGCCTCA GGCTAGCTACAACGA TTGGTTGT | 1090 |
| 450 | CAAGUGAG G CAGGUCCC | 103 | GGGACCTG GGCTAGCTACAACGA CTCACTTG | 1091 |
| 454 | UGAGGCAG G UCCCACUG | 104 | CAGTGGGA GGCTAGCTACAACGA CTGCCTCA | 1092 |
| 459 | CAGGUCCC A CUGCAGAG | 105 | CTCTGCAG GGCTAGCTACAACGA GGGACCTG | 1093 |
| 462 | GUCCCACU G CAGAGGCU | 106 | AGCCTCTG GGCTAGCTACAACGA AGTGGGAC | 1094 |
| 468 | CUGCAGAG G CUGCGGAU | 107 | ATCCGCAG GGCTAGCTACAACGA CTCTGCAG | 1095 |
| 471 | CAGAGGCU G CGGAUUGU | 108 | ACAATCCG GGCTAGCTACAACGA AGCCTCTG | 1096 |
| 475 | GGCUGCGG A UUGUGCGA | 109 | TCGCACAA GGCTAGCTACAACGA CCGCAGCC | 1097 |
| 478 | UGCGGAUU G UGCGAGGC | 110 | GCCTCGCA GGCTAGCTACAACGA AATCCGCA | 1098 |
| 480 | CGGAUUGU G CGAGGCAC | 111 | GTGCCTCG GGCTAGCTACAACGA ACAATCCG | 1099 |
| 485 | UGUGCGAG G CACCCAGC | 112 | GCTGGGTG GGCTAGCTACAACGA CTCGCACA | 1100 |
| 487 | UGCGAGGC A CCCAGCUC | 113 | GAGCTGGG GGCTAGCTACAACGA GCCTCGCA | 1101 |
| 492 | GGCACCCA G CUCUUUGA | 114 | TCAAAGAG GGCTAGCTACAACGA TGGGTGCC | 1102 |
| 503 | CUUUGAGG A CAACUAUG | 115 | CATAGTTG GGCTAGCTACAACGA CTTCAAAG | 1103 |
| 506 | UGAGGACA A CUAUGCCC | 116 | GGGCATAG GGCTAGCTACAACGA TGTCCTCA | 1104 |
| 509 | GGACAACU A UGCCCUGG | 117 | CCAGGGCA GGCTAGCTACAACGA AGTTGTCC | 1105 |
| 511 | ACAACUAU G CCCUGGCC | 118 | GGCCAGGG GGCTAGCTACAACGA ATAGTTGT | 1106 |
| 517 | AUGCCCUG G CCGUGCUA | 119 | TAGCACGG GGCTAGCTACAACGA CAGGGCAT | 1107 |
| 520 | CCCUGGCC G UGCUAGAC | 120 | GTCTAGCA GGCTAGCTACAACGA GGCCAGGG | 1108 |
| 522 | CUGGCCGU G CUAGACAA | 121 | TTGTCTAG GGCTAGCTACAACGA ACGGCCAG | 1109 |
| 527 | CGUGCUAG A CAAUGGAG | 122 | CTCCATTG GGCTAGCTACAACGA CTAGCACG | 1110 |
| 530 | GCUAGACA A UGGAGACC | 123 | GGTCTCCA GGCTAGCTACAACGA TGTCTAGC | 1111 |
| 536 | CAAUGGAG A CCCGCUGA | 124 | TCAGCGGG GGCTAGCTACAACGA CTCCATTG | 1112 |
| 540 | GGAGACCC G CUGAACAA | 125 | TTGTTCAG GGCTAGCTACAACGA GGGTCTCC | 1113 |
| 545 | CCCGCUGA A CAAUACCA | 126 | TGGTAATG GGCTAGCTACAACGA TCAGCGGG | 1114 |
| 548 | GCUGAACA A UACCACCC | 127 | GGGTGGTA GGCTAGCTACAACGA TGTTCAGC | 1115 |
| 550 | UGAACAAU A CCACCCCU | 128 | AGGGGTGG GGCTAGCTACAACGA ATTGTTCA | 1116 |
| 553 | ACAAUACC A CCCCUGUC | 129 | GACAGGGG GGCTAGCTACAACGA GGTATTGT | 1117 |
| 559 | CCACCCCU G UCACAGGG | 130 | CCCTGTGA GGCTAGCTACAACGA AGGGGTGG | 1118 |
| 562 | CCCCUGUC A CAGGGGCC | 131 | GGCCCCTG GGCTAGCTACAACGA GACAGGGG | 1119 |
| 568 | UCACAGGG G CCUCCCCA | 132 | TGGGGAGG GGCTAGCTACAACGA CCCTGTGA | 1120 |
| 581 | CCCAGGAG G CCUGCGGG | 133 | CCCGCAGG GGCTAGCTACAACGA CTCCTGGG | 1121 |
| 585 | GGAGGCCU G CGGGAGCU | 134 | AGCTCCCG GGCTAGCTACAACGA AGGCCTCC | 1122 |
| 591 | CUGCGGGA G CUGCAGCU | 135 | AGCTGCAG GGCTAGCTACAACGA TCCCGCAG | 1123 |
| 594 | CGGGAGCU G CAGCUUCG | 136 | CGAAGCTG GGCTAGCTACAACGA AGCTCCCG | 1124 |
| 597 | GAGCUGCA G CUUCGAAG | 137 | CTTCGAAG GGCTAGCTACAACGA TGCAGCTC | 1125 |
| 605 | GCUUCGAA G CCUCACAG | 138 | CTGTGAGG GGCTAGCTACAACGA TTCGAAGC | 1126 |
| 610 | GAAGCCUC A CAGAGAUC | 139 | GATCTCTG GGCTAGCTACAACGA GAGGCTTC | 1127 |
| 616 | UCACAGAG A UCUUGAAA | 140 | TTTCAAGA GGCTAGCTACAACGA CTCTGTGA | 1128 |
| 631 | AAGGAGGG G UCUUGAUC | 141 | GATCAAGA GGCTAGCTACAACGA CCCTCCTT | 1129 |
| 637 | GGGUCUUG A UCCAGCGG | 142 | CCGCTGGA GGCTAGCTACAACGA CAAGACCC | 1130 |
| 642 | UUGAUCCA G CGGAACCC | 143 | GGGTTCCG GGCTAGCTACAACGA TGGATCAA | 1131 |
| 647 | CCAGCGGA A CCCCCAGC | 144 | GCTGGGGG GGCTAGCTACAACGA TCCGCTGG | 1132 |
| 654 | AACCCCCA G CUCUGCUA | 145 | TAGCAGAG GGCTAGCTACAACGA TGGGGGTT | 1133 |
| 659 | CCAGCUCU G CUACCAGG | 146 | CCTGGTAG GGCTAGCTACAACGA AGAGCTTG | 1134 |
| 662 | GCUCUGCU A CCAGGACA | 147 | TGTCCTGG GGCTAGCTACAACGA AGCAGAGC | 1135 |
| 668 | CUACCAGG A CACGAUUU | 148 | AAATCGTG GGCTAGCTACAACGA CCTGGTAG | 1136 |
| 670 | ACCAGGAC A CGAUUUUG | 149 | CAAAATCG GGCTAGCTACAACGA GTCCTGGT | 1137 |
| 673 | AGGACACG A UUUUGUGG | 150 | CCACAAAA GGCTAGCTACAACGA CGTGTCCT | 1138 |
| 678 | ACGAUUUU G UGGAAGGA | 151 | TCCTTCCA GGCTAGCTACAACGA AAAATCGT | 1139 |
| 686 | GUGGAAGG A CAUCUUCC | 152 | GGAAGATG GGCTAGCTACAACGA CCTTCCAC | 1140 |
| 688 | GGAAGGAC A UCUUCCAC | 153 | GTGGAAGA GGCTAGCTACAACGA GTCCTTCC | 1141 |
| 695 | CAUCUUCC A CAAGAACA | 154 | TGTTCTTG GGCTAGCTACAACGA GGAAGATG | 1142 |
| 701 | CCACAAGA A CAACCAGC | 155 | GCTGGTTG GGCTAGCTACAACGA TCTTGTGG | 1143 |
| 704 | CAAGAACA A CCAGCUGG | 156 | CCAGCTGG GGCTAGCTACAACGA TGTTCTTG | 1144 |
| 708 | AACAACCA G CUGGCUCU | 157 | AGAGCCAG GGCTAGCTACAACGA TGGTTGTT | 1145 |
| 712 | ACCAGCUG G CUCUCACA | 158 | TGTGAGAG GGCTAGCTACAACGA CAGCTGGT | 1146 |
| 718 | UGGCUCUC A CACUGAUA | 159 | TATCAGTG GGCTAGCTACAACGA GAGAGCCA | 1147 |
| 720 | GCUCUCAC A CUGAUAGA | 160 | TCTATCAG GGCTAGCTACAACGA GTGAGAGC | 1148 |
| 724 | UCACACUG A UAGACACC | 161 | GGTGTCTA GGCTAGCTACAACGA CAGTGTGA | 1149 |
| 728 | ACUGAUAG A CACCAACC | 162 | GGTTGGTG GGCTAGCTACAACGA CTATCAGT | 1150 |
| 730 | UGAUAGAC A CCAACCGC | 163 | GCGGTTGG GGCTAGCTACAACGA GTCTATCA | 1151 |
| 734 | AGACACCA A CCGCUCUC | 164 | GAGAGCGG GGCTAGCTACAACGA TGGTGTCT | 1152 |
| 737 | CACCAACC G CUCUCGGG | 165 | CCCGAGAG GGCTAGCTACAACGA GGTTGGTG | 1153 |
| 745 | GCUCUCGG G CCUGCCAC | 166 | GTGGCAGG GGCTAGCTACAACGA CCGAGAGC | 1154 |
| 749 | UCGGGCCU G CCACCCCU | 167 | AGGGGTGG GGCTAGCTACAACGA AGGCCCGA | 1155 |
| 752 | GGCCUGCC A CCCCUGUU | 168 | AACAGGGG GGCTAGCTACAACGA GGCAGGCC | 1156 |
| 758 | CCACCCCU G UUCUCCGA | 169 | TCGGAGAA GGCTAGCTACAACGA AGGGGTGG | 1157 |
| 766 | GUUCUCCG A UGUGUAAG | 170 | CTTACACA GGCTAGCTACAACGA CGGAGAAC | 1158 |
| 768 | UCUCCGAU G UGUAAGGG | 171 | CCCTTACA GGCTAGCTACAACGA ATCGGAGA | 1159 |
| 770 | UCCGAUGU G UAAGGGCU | 172 | AGCCCTTA GGCTAGCTACAACGA ACATCGGA | 1160 |
| 776 | GUGUAAGG G CUCCCGCU | 173 | AGCGGGAG GGCTAGCTACAACGA CCTTACAC | 1161 |
| 782 | GGGCUCCC G CUGCUGGG | 174 | CCCAGCAG GGCTAGCTACAACGA GGGAGCCC | 1162 |
| 785 | CUCCCGCU G CUGGGGAG | 175 | CTCCCCAG GGCTAGCTACAACGA AGCGGGAG | 1163 |
| 797 | GGGAGAGA G UUCUGAGG | 176 | CCTCAGAA GGCTAGCTACAACGA TCTCTCCC | 1164 |
| 806 | UUCUGAGG A UUGUCAGA | 177 | TCTGACAA GGCTAGCTACAACGA CCTCAGAA | 1165 |
| 809 | UGAGGAUU G UCAGAGCC | 178 | GGCTCTGA GGCTAGCTACAACGA AATCCTCA | 1166 |
| 815 | UUGUCAGA G CCUGACGC | 179 | GCGTCAGG GGCTAGCTACAACGA TCTGACAA | 1167 |
| 820 | AGAGCCUG A CGCGCACU | 180 | AGTGCGCG GGCTAGCTACAACGA CAGGCTCT | 1168 |
| 822 | AGCCUGAC G CGCACUGU | 181 | ACAGTGCG GGCTAGCTACAACGA GTCAGGCT | 1169 |
| 824 | CCUGACGC G CACUGUCU | 182 | AGACAGTG GGCTAGCTACAACGA GCGTCAGG | 1170 |
| 826 | UGACGCGC A CUGUCUGU | 183 | ACAGACAG GGCTAGCTACAACGA GCGCGTCA | 1171 |
| 829 | CGCGCACU G UCUGUGCC | 184 | GGCACAGA GGCTAGCTACAACGA AGTGCGCG | 1172 |
| 833 | CACUGUCU G UGCCGGUG | 185 | CACCGGCA GGCTAGCTACAACGA AGACAGTG | 1173 |
| 835 | CUGUCUGU G CCGGUGGC | 186 | GCCACCGG GGCTAGCTACAACGA ACAGACAG | 1174 |
| 839 | CUGUGCCG G UGGCUGUG | 187 | CACAGCCA GGCTAGCTACAACGA CGGCACAG | 1175 |
| 842 | UGCCGGUG G CUGUGCCC | 188 | GGGCACAG GGCTAGCTACAACGA CACCGGCA | 1176 |
| 845 | CGGUGGCU G UGCCCGCU | 189 | AGCGGGCA GGCTAGCTACAACGA AGCCACCG | 1177 |
| 847 | GUGGCUGU G CCCGCUGC | 190 | GCAGCGGG GGCTAGCTACAACGA ACAGCCAC | 1178 |
| 851 | CUGUGCCC G CUGCAAGG | 191 | CCTTGCAG GGCTAGCTACAACGA GGGCACAG | 1179 |
| 854 | UGCCCGCU G CAAGGGGC | 192 | GCCCCTTG GGCTAGCTACAACGA AGCGGGCA | 1180 |
| 861 | UGCAAGGG G CCACUGCC | 193 | GGCAGTGG GGCTAGCTACAACGA CCCTTGCA | 1181 |
| 864 | AAGGGGCC A CUGCCCAC | 194 | GTGGGCAG GGCTAGCTACAACGA GGCCCCTT | 1182 |
| 867 | GGGCCACU G CCCACUGA | 195 | TCAGTGGG GGCTAGCTACAACGA AGTGGCCC | 1183 |
| 871 | CACUGCCC A CUGACUGC | 196 | GCAGTCAG GGCTAGCTACAACGA GGGCAGTG | 1184 |
| 875 | GCCCACUG A CUGCUGCC | 197 | GGCAGCAG GGCTAGCTACAACGA CAGTGGGC | 1185 |
| 878 | CACUGACU G CUGCCAUG | 198 | CATGGCAG GGCTAGCTACAACGA AGTCAGTG | 1186 |
| 881 | UGACUGCU G CCAUGAGC | 199 | GCTCATGG GGCTAGCTACAACGA AGCAGTCA | 1187 |
| 884 | CUGCUGCC A UGAGCAGU | 200 | ACTGCTCA GGCTAGCTACAACGA GGCAGCAG | 1188 |
| 888 | UGCCAUGA G CAGUGUGC | 201 | GCACACTG GGCTAGCTACAACGA TCATGGCA | 1189 |
| 891 | CAUGAGCA G UGUGCUGC | 202 | GCAGCACA GGCTAGCTACAACGA TGCTCATG | 1190 |
| 893 | UGAGCAGU G UGCUGCCG | 203 | CGGCAGCA GGCTAGCTACAACGA ACTGCTCA | 1191 |
| 895 | AGCAGUGU G CUGCCGGC | 204 | GCCGGCAG GGCTAGCTACAACGA ACACTGCT | 1192 |
| 898 | AUGUGUCU G CCGGCUGC | 205 | GCAGCCGG GGCTAGCTACAACGA AGCACACT | 1193 |
| 902 | UGCUGCCG G CUGCACGG | 206 | CCGTGCAG GGCTAGCTACAACGA CGGCAGCA | 1194 |
| 905 | UGCCGGCU G CACGGGCC | 207 | GGCCCGTG GGCTAGCTACAACGA AGCCGGCA | 1195 |
| 907 | CCGGCUGC A CGGGCCCC | 208 | GGGGCCCG GGCTAGCTACAACGA GCAGCCGG | 1196 |
| 911 | CUGCACGG G CCCCAAGC | 209 | GCTTGGGG GGCTAGCTACAACGA CCGTGCAG | 1197 |
| 918 | GGCCCCAA G CACUCUGA | 210 | TCAGAGTG GGCTAGCTACAACGA TTGGGGCC | 1198 |
| 920 | CCCCAAGC A CUCUGACU | 211 | AGTCAGAG GGCTAGCTACAACGA GCTTGGGG | 1199 |
| 926 | GCACUCUG A CUGCCUGG | 212 | CCAGGCAG GGCTAGCTACAACGA CAGAGTGC | 1200 |
| 929 | CUCUGACU G CCUGGCCU | 213 | AGGCCAGG GGCTAGCTACAACGA AGTCAGAG | 1201 |
| 934 | ACUGCCUG G CCUGCCUC | 214 | GAGGCAGG GGCTAGCTACAACGA CAGGCAGT | 1202 |
| 938 | CCUGGCCU G CCUCCACU | 215 | AGTGGAGG GGCTAGCTACAACGA AGGCCAGG | 1203 |
| 944 | CUGCCUCC A CUUCAACC | 216 | GGTTGAAG GGCTAGCTACAACGA GGAGGCAG | 1204 |
| 950 | CCACUUCA A CCACAGUG | 217 | CACTGTGG GGCTAGCTACAACGA TGAAGTGG | 1205 |
| 953 | CUUCAACC A CAGUGGCA | 218 | TGCCACTG GGCTAGCTACAACGA GGTTGAAG | 1206 |
| 956 | CAACCACA G UGGCAUCU | 219 | AGATGCCA GGCTAGCTACAACGA TGTGGTTG | 1207 |
| 959 | CCACAGUG G CAUCUGUG | 220 | CACAGATG GGCTAGCTACAACGA CACTGTGG | 1208 |
| 961 | ACAGUGGC A UCUGUGAG | 221 | CTCACAGA GGCTAGCTACAACGA GCCACTGT | 1209 |
| 965 | UGGCAUCU G UGAGCUGC | 222 | GCAGCTCA GGCTAGCTACAACGA AGATGCCA | 1210 |
| 969 | AUCUGUGA G CUGCACUG | 223 | CAGTGCAG GGCTAGCTACAACGA TCACAGAT | 1211 |
| 972 | UGUGAGCU G CACUGCCC | 224 | GGGCAGTG GGCTAGCTACAACGA AGCTCACA | 1212 |
| 974 | UGAGCUGC A CUGCCCAG | 225 | CTGGGCAG GGCTAGCTACAACGA GCAGCTCA | 1213 |
| 977 | GCUGCACU G CCCAGCCC | 226 | GGGCTGGG GGCTAGCTACAACGA AGTGCAGC | 1214 |
| 982 | ACUGCCCA G CCCUGGUC | 227 | GACCAGGG GGCTAGCTACAACGA TGGGCAGT | 1215 |
| 988 | CAGCCCUG G UCACCUAC | 228 | GTAGGTGA GGCTAGCTACAACGA CAGGGCTG | 1216 |
| 991 | CCCUGGUC A CCUACAAC | 229 | GTTGTAGG GGCTAGCTACAACGA GACCAGGG | 1217 |
| 995 | GGUCACCU A CAACACAG | 230 | CTGTGTTG GGCTAGCTACAACGA AGGTGACC | 1218 |
| 998 | CACCUACA A CACAGACA | 231 | TGTCTGTG GGCTAGCTACAACGA TGTAGGTG | 1219 |
| 1000 | CCUACAAC A CAGACACG | 232 | CGTGTCTG GGCTAGCTACAACGA GTTGTAGG | 1220 |
| 1004 | CAACACAG A CACGUUUG | 233 | CAAACGTG GGCTAGCTACAACGA CTGTGTTG | 1221 |
| 1006 | ACACAGAC A CGUUUGAG | 234 | CTCAAACG GGCTAGCTACAACGA GTCTGTGT | 1222 |
| 1008 | ACAGACAC G UUUGAGUC | 235 | GACTCAAA GGCTAGCTACAACGA GTGTCTGT | 1223 |
| 1014 | ACGUUUGA G UCCAUGCC | 236 | GGCATGGA GGCTAGCTACAACGA TCAAACGT | 1224 |
| 1018 | UUGAGUCC A UGCCCAAU | 237 | ATTGGGCA GGCTAGCTACAACGA GGACTCAA | 1225 |
| 1020 | GAGUCCAU G CCCAAUCC | 238 | GGATTGGG GGCTAGCTACAACGA ATGGACTC | 1226 |
| 1025 | CAUGCCCA A UCCCGAGG | 239 | CCTCGGGA GGCTAGCTACAACGA TGGGCATG | 1227 |
| 1034 | UCCCGAGG G CCGGUAUA | 240 | TATACCGG GGCTAGCTACAACGA CCTCGGGA | 1228 |
| 1038 | GAGGGCCG G UAUACAUU | 241 | AATGTATA GGCTAGCTACAACGA CGGCCCTC | 1229 |
| 1040 | GGGCCGGU A UACAUUCG | 242 | CGAATGTA GGCTAGCTACAACGA ACCGGCCC | 1230 |
| 1042 | GCCGGUAU A CAUUCGGC | 243 | GCCGAATG GGCTAGCTACAACGA ATACCGGC | 1231 |
| 1044 | CGGUAUAC A UUCGGCGC | 244 | GCGCCGAA GGCTAGCTACAACGA GTATACCG | 1232 |
| 1049 | UACAUUCG G CGCCAGCU | 245 | AGCTGGCG GGCTAGCTACAACGA CGAATGTA | 1233 |
| 1051 | CAUUCGGC G CCAGCUGU | 246 | ACAGCTGG GGCTAGCTACAACGA GCCGAATG | 1234 |
| 1055 | CGGCGCCA G CUGUGUGA | 247 | TCACACAG GGCTAGCTACAACGA TGGCGCCG | 1235 |
| 1058 | CGCCAGCU G UGUGACUG | 248 | CAGTCACA GGCTAGCTACAACGA AGCTGGCG | 1236 |
| 1060 | CCAGCUGU G UGACUGCC | 249 | GGCAGTCA GGCTAGCTACAACGA ACAGCTGG | 1237 |
| 1063 | GCUGUGUG A CUGCCUGU | 250 | ACAGGCAG GGCTAGCTACAACGA CACACAGC | 1238 |
| 1066 | GUGUGACU G CCUGUCCC | 251 | GGGACAGG GGCTAGCTACAACGA AGTCACAC | 1239 |
| 1070 | GACUGCCU G UCCCUACA | 252 | TGTAGGGA GGCTAGCTACAACGA AGGCAGTC | 1240 |
| 1076 | CUGUCCCU A CAACUACC | 253 | GGTAGTTG GGCTAGCTACAACGA AGGGACAG | 1241 |
| 1079 | UCCCUACA A CUACCUUU | 254 | AAAGGTAG GGCTAGCTACAACGA TGTAGGGA | 1242 |
| 1082 | CUACAACU A CCUUUCUA | 255 | AAAGGTAG GGCTAGCTACAACGA TGTAGGGA | 1243 |
| 1090 | ACCUUUCU A CCUUUCUA | 256 | CACGTCCG GGCTAGCTACAACGA AGAAAGGT | 1244 |
| 1094 | UUCUACGG A CGUGGGAU | 257 | ATCCCACG GGCTAGCTACAACGA CCGTAGAA | 1245 |
| 1096 | CUACGGAC G UGGGAUCC | 258 | GGATCCCA GGCTAGCTACAACGA GTCCGTAG | 1246 |
| 1101 | GACGUGGG A UCCUGCAC | 259 | GTGCAGGA GGCTAGCTACAACGA CCCACGTC | 1247 |
| 1106 | GGGAUCCU G CACCCUCG | 260 | CGAGGGTG GGCTAGCTACAACGA AGGATCCC | 1248 |
| 1108 | GAUCCUGC A CCCUCGUC | 261 | GACGAGGG GGCTAGCTACAACGA GCAGGATC | 1249 |
| 1114 | GCACCCUC G UCUGCCCC | 262 | GGGGCAGA GGCTAGCTACAACGA GAGGGTGC | 1250 |
| 1118 | CCUCGUCU G CCCCCUGC | 263 | GCAGGGGG GGCTAGCTACAACGA AGACGAGG | 1251 |
| 1125 | UGCCCCCU G CACAACCA | 264 | TGGTTGTG GGCTAGCTACAACGA AGGGGGCA | 1252 |
| 1127 | CCCCCUGC A CAACCAAG | 265 | CTTGGTTG GGCTAGCTACAACGA GCAGGGGG | 1253 |
| 1130 | CCUGCACA A CCAAGAGG | 266 | CCTCTTGG GGCTAGCTACAACGA TGTGCAGG | 1254 |
| 1138 | ACCAAGAG G UGACAGCA | 267 | TGCTGTCA GGCTAGCTACAACGA CTCTTGGT | 1255 |
| 1141 | AAGAGGUG A CAGCAGAG | 268 | CTCTGCTG GGCTAGCTACAACGA CACCTCTT | 1256 |
| 1144 | AGGUGACA G CAGAGGAU | 269 | ATCCTCTG GGCTAGCTACAACGA TGTCACCT | 1257 |
| 1151 | AGCAGAGG A UGGAACAC | 270 | GTGTTCCA GGCTAGCTACAACGA CCTCTGCT | 1258 |
| 1156 | AGGAUGGA A CACAGCGG | 271 | CCGCTGTG GGCTAGCTACAACGA TCCATCCT | 1259 |
| 1158 | GAUGGAAC A CAGCGGUG | 272 | CACCGCTG GGCTAGCTACAACGA GTTCCATC | 1260 |
| 1161 | GGAACACA G CGGUGUGA | 273 | TCACACCG GGCTAGCTACAACGA TGTGTTCC | 1261 |
| 1164 | ACACAGCG G UGUGAGAA | 274 | TTCTCACA GGCTAGCTACAACGA CGCTGTGT | 1262 |
| 1166 | ACAGCGGU G UGAGAAGU | 275 | ACTTCTCA GGCTAGCTACAACGA ACCGCTGT | 1263 |
| 1173 | UGUGAGAA G UGCAGCAA | 276 | TTGCTGCA GGCTAGCTACAACGA TTCTCACA | 1264 |
| 1175 | UGAGAAGU G CAGCAAGC | 277 | GCTTGCTG GGCTAGCTACAACGA ACTTCTCA | 1265 |
| 1178 | GAAGUGCA G CAAGCCCU | 278 | AGGGCTTG GGCTAGCTACAACGA TGCACTTC | 1266 |
| 1182 | UGCAGCAA G CCCUGUGC | 279 | GCACAGGG GGCTAGCTACAACGA TTGCTGCA | 1267 |
| 1187 | CAAGCCCU G UGCCCGAG | 280 | CTCGGGCA GGCTAGCTACAACGA AGGGCTTG | 1268 |
| 1189 | AGCCCUGU G CCCGAGUG | 281 | CACTCGGG GGCTAGCTACAACGA ACAGGGCT | 1269 |
| 1195 | GUGCCCGA G UGUGCUAU | 282 | ATAGCACA GGCTAGCTACAACGA TCGGGCAC | 1270 |
| 1197 | GCCCGAGU G UGCUAUGG | 283 | CCATAGCA GGCTAGCTACAACGA ACTCGGGC | 1271 |
| 1199 | CCGAGUGU G CUAUGGUC | 284 | GACCATAG GGCTAGCTACAACGA ACACTCGG | 1272 |
| 1202 | AGUGUGCU A UGGUCUGG | 285 | CCAGACCA GGCTAGCTACAACGA AGCACACT | 1273 |
| 1205 | GUGCUAUG G UCUGGGCA | 286 | TGCCCAGA GGCTAGCTACAACGA CATAGCAC | 1274 |
| 1211 | UGGUCUGG G CAUGGAGC | 287 | GCTCCATG GGCTAGCTACAACGA CCAGACCA | 1275 |
| 1213 | GUCUGGGC A UGGAGCAC | 288 | GTGCTCCA GGCTAGCTACAACGA GCCCAGAC | 1276 |
| 1218 | GGCAUGGA G CACUUGCG | 289 | CGCAAGTG GGCTAGCTACAACGA TCCATGCC | 1277 |
| 1220 | CAUGGAGC A CUUGCGAG | 290 | CTCGCAAG GGCTAGCTACAACGA GCTCCATG | 1278 |
| 1224 | GAGCACUU G CGAGAGGU | 291 | ACCTCTCG GGCTAGCTACAACGA AAGTGCTC | 1279 |
| 1231 | UGCGAGAG G UGAGGGCA | 292 | TGCCCTCA GGCTAGCTACAACGA CTCTCGCA | 1280 |
| 1237 | AGGUGAGG G CAGUUACC | 293 | GGTAACTG GGCTAGCTACAACGA CTTCACCT | 1281 |
| 1240 | UGAGGGCA G UUACCAGU | 294 | ACTGGTAA GGCTAGCTACAACGA TGCCCTCA | 1282 |
| 1243 | GGGCAGUU A CCAGUGCC | 295 | GGCACTGG GGCTAGCTACAACGA AACTGCCC | 1283 |
| 1247 | AGUUACCA G UGCCAAUA | 296 | TATTGGCA GGCTAGCTACAACGA TGGTAACT | 1284 |
| 1249 | UUACCAGU G CCAAUAUC | 297 | GATATTGG GGCTAGCTACAACGA ACTGGTAA | 1285 |
| 1253 | CAGUGCCA A UAUCCAGG | 298 | CCTGGATA GGCTAGCTACAACGA TGGCACTG | 1286 |
| 1255 | GUGCCAAU A UCCAGGAG | 299 | CTCCTGGA GGCTAGCTACAACGA ATTGGCAC | 1287 |
| 1263 | AUCCAGGA G UUUGCUGG | 300 | CCAGCAAA GGCTAGCTACAACGA TCCTGGAT | 1288 |
| 1267 | AGGAGUUU G CUGGCUGC | 301 | GCAGCCAG GGCTAGCTACAACGA AAACTCCT | 1289 |
| 1271 | GUUUGCUG G CUGCAAGA | 302 | TCTTGCAG GGCTAGCTACAACGA CAGCAAAC | 1280 |
| 1274 | UGCUGGCU G CAAGAAGA | 303 | TCTTCTTG GGCTAGCTACAACGA AGCCAGCA | 1291 |
| 1282 | GCAAGAAG A UCUUUGGG | 304 | CCCAAAGA GGCTAGCTACAACGA CTTCTTGC | 1292 |
| 1292 | CUUUGGGA G CCUGGCAU | 305 | ATGCCAGG GGCTAGCTACAACGA TCCCAAAG | 1293 |
| 1297 | GGAGCCUG G CAUUUCUG | 306 | CAGAAATG GGCTAGCTACAACGA CAGGCTCC | 1294 |
| 1299 | AGCCUGGC A UUUCUGCC | 307 | GGCAGAAA GGCTAGCTACAACGA GCCAGGCT | 1295 |
| 1305 | GCAUUUCU G CCGGAGAG | 308 | CTCTCCGG GGCTAGCTACAACGA AGAAATGC | 1296 |
| 1313 | GCCGGAGA G CUUUGAUG | 309 | CATCAAAG GGCTAGCTACAACGA TCTCCGGC | 1297 |
| 1319 | GAGCUUUG A UGGGGACC | 310 | GGTCCCCA GGCTAGCTACAACGA CAAAGCTC | 1298 |
| 1325 | UGAUGGGG A CCCAGCCU | 311 | AGGCTGGG GGCTAGCTACAACGA CCCCATCA | 1299 |
| 1330 | GGGACCCA G CCUCCAAC | 312 | GTTGGAGG GGCTAGCTACAACGA TGGGTCCC | 1300 |
| 1337 | AGCCUCCA A CACUGCCC | 313 | GGGCAGTG GGCTAGCTACAACGA TGGAGGCT | 1301 |
| 1339 | CCUCCAAC A CUGCCCCG | 314 | CGGGGCAG GGCTAGCTACAACGA GTTGGAGG | 1302 |
| 1342 | CCAACACU G CCCCGCUC | 315 | GAGCGGGG GGCTAGCTACAACGA AGTGTTGG | 1303 |
| 1347 | ACUGCCCC G CUCCAGCC | 316 | GGCTGGAG GGCTAGCTACAACGA GGGGCAGT | 1304 |
| 1353 | CCGCUCCA G CCAGAGCA | 317 | TGCTCTGG GGCTAGCTACAACGA TGGAGCGG | 1305 |
| 1359 | CAGCCAGA G CAGCUCCA | 318 | TGGAGCTG GGCTAGCTACAACGA TCTGGCTG | 1306 |
| 1362 | CCAGAGCA G CUCCAAGU | 319 | ACTTGGAG GGCTAGCTACAACGA TGCTCTGG | 1307 |
| 1369 | AGCUCCAA G UGUUUGAG | 320 | CTCAAACA GGCTAGCTACAACGA TTGGAGCT | 1308 |
| 1371 | CUCCAAGU G UUUGAGAC | 321 | GTCTCAAA GGCTAGCTACAACGA ACTTGGAG | 1309 |
| 1378 | UGUUUGAG A CUCUGGAA | 322 | TTCCAGAG GGCTAGCTACAACGA CTCAAACA | 1310 |
| 1390 | UGGAAGAG A UCACAGGU | 323 | ACCTGTGA GGCTAGCTACAACGA CTCTTCCA | 1311 |
| 1393 | AAGAGAUC A CAGGUUAC | 324 | GTAACCTG GGCTAGCTACAACGA GATCTCTT | 1312 |
| 1397 | GAUCACAG G UUACCUAU | 325 | ATAGGTAA GGCTAGCTACAACGA CTGTGATC | 1313 |
| 1400 | CACAGGUU A CCUAUACA | 326 | TGTATAGG GGCTAGCTACAACGA AACCTGTG | 1314 |
| 1404 | GGUUACCU A UACAUCUC | 327 | GAGATGTA GGCTAGCTACAACGA AGGTAACC | 1315 |
| 1406 | UUACCUAU A CAUCUCAG | 328 | CTGAGATG GGCTAGCTACAACGA ATAGGTAA | 1316 |
| 1408 | ACCUAUAC A UCUCAGCA | 329 | TGCTGAGA GGCTAGCTACAACGA GTATAGGT | 1317 |
| 1414 | ACAUCUCA G CAUGGCCG | 330 | CGGCCATG GGCTAGCTACAACGA TGAGATGT | 1318 |
| 1416 | AUCUCAGC A UGGCCGGA | 331 | TCCGGCCA GGCTAGCTACAACGA GCTGAGAT | 1319 |
| 1419 | UCAGCAUG G CCGGACAG | 332 | CTGTCCGG GGCTAGCTACAACGA CATGCTGA | 1320 |
| 1424 | AUGGCCGG A CAGCCUGC | 333 | GCAGGCTG GGCTAGCTACAACGA CCGGCCAT | 1321 |
| 1427 | GCCGGACA G CCUGCCUG | 334 | CAGGCAGG GGCTAGCTACAACGA TGTCCGGC | 1322 |
| 1431 | GACAGCCU G CCUGACCU | 335 | AGGTCAGG GGCTAGCTACAACGA AGGCTGTC | 1323 |
| 1436 | CCUGCCUG A CCUCAGCG | 336 | CGCTGAGG GGCTAGCTACAACGA CAGGCAGG | 1324 |
| 1442 | UGACCUCA G CGUCUUCC | 337 | GGAAGACG GGCTAGCTACAACGA TGAGGTCA | 1325 |
| 1444 | ACCUCAGC G UCUUCCAG | 338 | CTGGAAGA GGCTAGCTACAACGA GCTGAGGT | 1326 |
| 1454 | CUUCCAGA A CCUGCAAG | 339 | CTTGCAGG GGCTAGCTACAACGA TCTGGAAG | 1327 |
| 1458 | CAGAACCU G CAAGUAAU | 340 | ATTACTTG GGCTAGCTACAACGA AGGTTCTG | 1328 |
| 1462 | ACCUGCAA G UAAUCCGG | 341 | CCGGATTA GGCTAGCTACAACGA TTGCAGGT | 1329 |
| 1465 | UGCAAGUA A UCCGGGGA | 342 | TCCCCGGA GGCTAGCTACAACGA TACTTGCA | 1330 |
| 1473 | AUCCGGGG A CGAAUUCU | 343 | AGAATTCG GGCTAGCTACAACGA CCCCGGAT | 1331 |
| 1477 | GGGGACGA A UUCUGCAC | 344 | GCGCAGAA GGCTAGCTACAACGA TCGTCCCC | 1332 |
| 1482 | CGAAUUCU G CACAAUGG | 345 | CCATTGTG GGCTAGCTACAACGA AGAATTCG | 1333 |
| 1484 | AAUUCUGC A CAAUGGCG | 346 | CGCCATTG GGCTAGCTACAACGA GCAGAATT | 1334 |
| 1487 | UCUGCACA A UGGCGCCU | 347 | AGGCGCCA GGCTAGCTACAACGA TGTGCAGA | 1335 |
| 1490 | GCACAAUG G CGCCUACU | 348 | AGTAGGCG GGCTAGCTACAACGA CATTGTGC | 1336 |
| 1492 | ACAAUGGC G CCUACUCG | 349 | CGAGTAGG GGCTAGCTACAACGA GCCATTGT | 1337 |
| 1496 | UGGCGCCU A CUCGCUGA | 350 | TCAGCGAG GGCTAGCTACAACGA AGGCGCCA | 1338 |
| 1500 | GCCUACUC G CUGACCCU | 351 | AGGGTCAG GGCTAGCTACAACGA GAGTAGGC | 1339 |
| 1504 | ACUCGCUG A CCCUGCAA | 352 | TTGCAGGG GGCTAGCTACAACGA CAGCGAGT | 1340 |
| 1509 | CUGACCCU G CAAGGGCU | 353 | AGCCCTTG GGCTAGCTACAACGA AGGGTCAG | 1341 |
| 1515 | CUGCAAGG G CUGGGCAU | 354 | ATGCCCAG GGCTAGCTACAACGA CCTTGCAG | 1342 |
| 1520 | AGGGCUGG G CAUCAGCU | 355 | AGCTGATG GGCTAGCTACAACGA CCAGCCCT | 1343 |
| 1522 | GGCUGGGC A UCAGCUGG | 356 | CCAGCTGA GGCTAGCTACAACGA GCCCAGCC | 1344 |
| 1526 | GGGCAUCA G CUGGCUGG | 357 | CCAGCCAG GGCTAGCTACAACGA TGATGCCC | 1345 |
| 1530 | AUCAGCUG G CUGGGGCU | 358 | AGCCCCAG GGCTAGCTACAACGA CAGCTGAT | 1346 |
| 1536 | UGGCUGGG G CUGCGCUC | 359 | GAGCGCAG GGCTAGCTACAACGA CCCAGCCA | 1347 |
| 1539 | CUGGGGCU G CGCUCACU | 360 | AGTGAGCG GGCTAGCTACAACGA AGCCCCAG | 1348 |
| 1541 | GGGGCUGC G CUCACUGA | 361 | TCAGTGAG GGCTAGCTACAACGA GCAGCCCC | 1349 |
| 1545 | CUGCGCUC A CUGAGGGA | 362 | TCCCTCAG GGCTAGCTACAACGA GAGCGCAG | 1350 |
| 1554 | CUGAGGGA A CUGGGCAG | 363 | CTGCCCAG GGCTAGCTACAACGA TCCCTCAG | 1351 |
| 1559 | GGAACUGG G CAGUGGAC | 364 | GTCCACTG GGCTAGCTACAACGA CCAGTTCC | 1352 |
| 1562 | ACUGGGCA G UGGACUGG | 365 | CCAGTCCA GGCTAGCTACAACGA TGCCCAGT | 1353 |
| 1566 | GGCAGUGG A CUGGCCCU | 366 | AGGGCCAG GGCTAGCTACAACGA CCACTGCC | 1354 |
| 1570 | GUGGACUG G CCCUCAUC | 367 | GATGAGGG GGCTAGCTACAACGA CAGTCCAC | 1355 |
| 1576 | UGGCCCUC A UCCACCAU | 368 | ATGGTGGA GGCTAGCTACAACGA GAGGGCCA | 1356 |
| 1580 | CCUCAUCC A CCAUAACA | 369 | TGTTATGG GGCTAGCTACAACGA GGATGAGG | 1357 |
| 1583 | CAUCCACC A UAACACCC | 370 | GGGTGTTA GGCTAGCTACAACGA GGTGGATG | 1358 |
| 1586 | CCACCAUA A CACCCACC | 371 | GGTGGGTG GGCTAGCTACAACGA TATGGTGG | 1359 |
| 1588 | ACCAUAAC A CCCACCUC | 372 | GAGGTGGG GGCTAGCTACAACGA GTTATGGT | 1360 |
| 1592 | UAACACCC A CCUCUGCU | 373 | AGCAGAGG GGCTAGCTACAACGA GGGTGTTA | 1361 |
| 1598 | CCACCUCU G CUUCGUGC | 374 | GCACGAAG GGCTAGCTACAACGA AGAGGTGG | 1362 |
| 1603 | UCUGCUUC G UGCACACG | 375 | CGTGTGCA GGCTAGCTACAACGA GAAGCAGA | 1363 |
| 1605 | UGCUUCGU G CACACGGU | 376 | ACCGTGTG GGCTAGCTACAACGA ACGAAGCA | 1364 |
| 1607 | CUUCGUGC A CACGGUGC | 377 | GCACCGTG GGCTAGCTACAACGA GCACGAAG | 1365 |
| 1609 | UCGUGCAC A CGGUGCCC | 378 | GGGCACCG GGCTAGCTACAACGA GTGCACGA | 1366 |
| 1612 | UGCACACG G UGCCCUGG | 379 | CCAGGGCA GGCTAGCTACAACGA CGTGTGCA | 1367 |
| 1614 | CACACGGU G CCCUGGGA | 380 | TCCCAGGG GGCTAGCTACAACGA ACCGTGTG | 1368 |
| 1622 | GCCCUGGG A CCAGCUCU | 381 | AGAGCTGG GGCTAGCTACAACGA CCCAGGGC | 1369 |
| 1626 | UGGGACCA G CUCUUUCG | 382 | CGAAAGAG GGCTAGCTACAACGA TGGTCCCA | 1370 |
| 1637 | CUUUCGGA A CCCGCACC | 383 | GGTGCGGG GGCTAGCTACAACGA TCCGAAAG | 1371 |
| 1641 | CGGAACCC G CACCAAGC | 384 | GCTTGGTG GGCTAGCTACAACGA GGGTTCCG | 1372 |
| 1643 | GAACCCGC A CCAAGCUC | 385 | GAGCTTGG GGCTAGCTACAACGA GCGGGTTC | 1373 |
| 1648 | CGCACCAA G CUCUGCUC | 386 | GAGCAGAG GGCTAGCTACAACGA TTGGTGCG | 1374 |
| 1653 | CAAGCUCU G CUCCACAC | 387 | GTGTGGAG GGCTAGCTACAACGA AGAGCTTG | 1375 |
| 1658 | UCUGCUCC A CACUGCCA | 388 | TGGCAGTG GGCTAGCTACAACGA GGAGCAGA | 1376 |
| 1660 | UGCUCCAC A CUGCCAAC | 389 | GTTGGCAG GGCTAGCTACAACGA GTGGAGCA | 1377 |
| 1663 | UCCACACU G CCAACCGG | 390 | CCGGTTGG GGCTAGCTACAACGA AGTGTGGA | 1378 |
| 1667 | CACUGCCA A CCGGCCAG | 391 | CTGGCCGG GGCTAGCTACAACGA TGGCAGTG | 1379 |
| 1671 | GCCAACCG G CCAGAGGA | 392 | TCCTCTGG GGCTAGCTACAACGA CGGTTGGC | 1380 |
| 1679 | GCCAGAGG A CGAGUGUG | 393 | CACACTCG GGCTAGCTACAACGA CCTCTGGC | 1381 |
| 1683 | GAGGACGA G UGUGUGGG | 394 | CCCACACA GGCTAGCTACAACGA TCGTCCTC | 1382 |
| 1685 | GGACGAGU G UGUGGGCG | 395 | CGCCCACA GGCTAGCTACAACGA ACTCGTCC | 1383 |
| 1687 | ACGAGUGU G UGGGCGAG | 396 | CTCGCCCA GGCTAGCTACAACGA ACACTCGT | 1384 |
| 1691 | GUGUGUGG G CGAGGGCC | 397 | GGCCCTCG GGCTAGCTACAACGA CCACACAC | 1385 |
| 1697 | GGGCGAGG G CCUGGCCU | 398 | AGGCCAGG GGCTAGCTACAACGA CCTCGCCC | 1386 |
| 1702 | AGGGCCUG G CCUGCCAC | 399 | GTGGCAGG GGCTAGCTACAACGA CAGGCCCT | 1387 |
| 1706 | CCUGGCCU G CCACCAGC | 400 | GCTGGTGG GGCTAGCTACAACGA AGGCCAGG | 1388 |
| 1709 | GGCCUGCC A CCAGCUGU | 401 | ACAGCTGG GGCTAGCTACAACGA GGCAGGCC | 1389 |
| 1713 | UGCCACCA G CUGUGCGC | 402 | GCGCACAG GGCTAGCTACAACGA TGGTGGCA | 1390 |
| 1716 | CACCAGCU G UGCGCCCG | 403 | CGGGCGCA GGCTAGCTACAACGA AGCTGGTG | 1391 |
| 1718 | CCAGCUGU G CGCCCGAG | 404 | CTCGGGCG GGCTAGCTACAACGA ACAGCTGG | 1392 |
| 1720 | AGCUGUGC G CCCGAGGG | 405 | CCCTCGGG GGCTAGCTACAACGA GCACAGCT | 1393 |
| 1728 | GCCCGAGG G CACUGCUG | 406 | CAGCAGTG GGCTAGCTACAACGA CCTCGGGC | 1394 |
| 1730 | CCGAGGGC A CUGCUGGG | 407 | CCCAGCAG GGCTAGCTACAACGA GCCCTCGG | 1395 |
| 1733 | AGGGCACU G CUGGGGUC | 408 | GACCCCAG GGCTAGCTACAACGA AGTGCCCT | 1396 |
| 1739 | AGGGCACU G UCCAGGGC | 409 | GCCCTGGA GGCTAGCTACAACGA CCCAGCAG | 1397 |
| 1746 | GGUCCAGG G CCCACCCA | 410 | TGGGTGGG GGCTAGCTACAACGA CCTGGACC | 1398 |
| 1750 | CAGGGCCC A CCCAGUGU | 411 | ACACTGGG GGCTAGCTACAACGA GGGCCCTG | 1399 |
| 1755 | CCCACCCA G UGUGUCAA | 412 | TTGACACA GGCTAGCTACAACGA TGGGTGGG | 1400 |
| 1757 | CACCCAGU G UGUCAACU | 413 | AGTTGACA GGCTAGCTACAACGA ACTGGGTG | 1401 |
| 1759 | CCCAGUGU G UCAACUGC | 414 | GCAGTTGA GGCTAGCTACAACGA ACACTGGG | 1402 |
| 1763 | GUGUGUCA A CUGCAGCC | 415 | GGCTGCAG GGCTAGCTACAACGA TGACACAC | 1403 |
| 1766 | UGUCAACU G CAGCCAGU | 416 | ACTGGCTG GGCTAGCTACAACGA AGTTGACA | 1404 |
| 1769 | CAACUGCA G CCAGUUCC | 417 | GGAACTGG GGCTAGCTACAACGA TGCAGTTG | 1405 |
| 1773 | UGCAGCCA G UUCCUUCG | 418 | CGAAGGAA GGCTAGCTACAACGA TGGCTGCA | 1406 |
| 1784 | CCUUCGGG G CCAGGAGU | 419 | ACTCCTGG GGCTAGCTACAACGA CCCGAAGG | 1407 |
| 1791 | GGCCAGGA G UGCGUGGA | 420 | TCCACGCA GGCTAGCTACAACGA TCCTGGCC | 1408 |
| 1793 | CCAGGAGU G CGUGGAGG | 421 | CCTCCACG GGCTAGCTACAACGA ACTCCTGG | 1409 |
| 1795 | AGGAGUGC G UGGAGGAA | 422 | TTCCTCCA GGCTAGCTACAACGA GCACTCCT | 1410 |
| 1803 | GUGGAGGA A UGCCGAGU | 423 | ACTCGGCA GGCTAGCTACAACGA TCCTCCAC | 1411 |
| 1805 | GGAGGAAU G CCGAGUAC | 424 | GTACTCGG GGCTAGCTACAACGA ATTCCTCC | 1412 |
| 1810 | AAUGCCGA G UACUGCAG | 425 | CTGCAGTA GGCTAGCTACAACGA TCGGCATT | 1413 |
| 1812 | UGCCGAGU A CUGCAGGG | 426 | CCCTGCAG GGCTAGCTACAACGA ACTCGGCA | 1414 |
| 1815 | CGAGUACU G CAGGGGCU | 427 | AGCCCCTG GGCTAGCTACAACGA AGTACTCG | 1415 |
| 1821 | CUGCAGGG G CUCCCCAG | 428 | CTGGGGAG GGCTAGCTACAACGA CCCTGCAG | 1416 |
| 1833 | CCCAGGGA G UAUGUGAA | 429 | TTCACATA GGCTAGCTACAACGA TCCCTGGG | 1417 |
| 1835 | CAGGGAGU A UGUGAAUG | 430 | CATTCACA GGCTAGCTACAACGA ACTCCCTG | 1418 |
| 1837 | GGGAGUAU G UGAAUGCC | 431 | GGCATTCA GGCTAGCTACAACGA ATACTCCC | 1419 |
| 1841 | GUAUGUGA A UGCCAGGC | 432 | GCCTGGCA GGCTAGCTACAACGA TCACATAC | 1420 |
| 1843 | AUGUGAAU G CCAGGCAC | 433 | GTGCCTGG GGCTAGCTACAACGA ATTCACAT | 1421 |
| 1848 | AAUGCCAG G CACUGUUU | 434 | AAACAGTG GGCTAGCTACAACGA CTGGCATT | 1422 |
| 1850 | UGCCAGGC A CUGUUUGC | 435 | GCAAACAG GGCTAGCTACAACGA GCCTGGCA | 1423 |
| 1853 | CAGGCACU G UUUGCCGU | 436 | ACGGCAAA GGCTAGCTACAACGA AGTGCCTG | 1424 |
| 1857 | CACUGUUU G CCGUGCCA | 437 | TGGCACGG GGCTAGCTACAACGA AAACAGTG | 1425 |
| 1860 | UGUUUGCC G UGCCACCC | 438 | GGGTGGCA GGCTAGCTACAACGA GGCAAACA | 1426 |
| 1862 | UUUGCCGU G CCACCCUG | 439 | CAGGGTGG GGCTAGCTACAACGA ACGGCAAA | 1427 |
| 1865 | GCCGUGCC A CCCUGAGU | 440 | ACTCAGGG GGCTAGCTACAACGA GGCACGGC | 1428 |
| 1872 | CACCCUGA G UGUCAGCC | 441 | GGCTGACA GGCTAGCTACAACGA TCAGGGTG | 1429 |
| 1874 | CCCUGAGU G UCAGCCCC | 442 | GGGGCTGA GGCTAGCTACAACGA ACTCAGGG | 1430 |
| 1878 | GAGUGUCA G CCCCAGAA | 443 | TTCTGGGG GGCTAGCTACAACGA TGACACTC | 1431 |
| 1886 | GCCCCAGA A UGGCUCAG | 444 | CTGAGCCA GGCTAGCTACAACGA TCTGGGGC | 1432 |
| 1889 | CCAGAAUG G CUCAGUGA | 445 | TCACTGAG GGCTAGCTACAACGA CATTCTGG | 1433 |
| 1894 | AUGGCUCA G UGACCUGU | 446 | ACAGGTCA GGCTAGCTACAACGA TGAGCCAT | 1434 |
| 1897 | GCUCAGUG A CCUGUUUU | 447 | AAAACAGG GGCTAGCTACAACGA CACTGAGC | 1435 |
| 1901 | AGUGACCU G UUUUGGAC | 448 | GTCCAAAA GGCTAGCTACAACGA AGGTCACT | 1436 |
| 1908 | UGUUUGGG A CCGGAGGC | 449 | GCCTCCGG GGCTAGCTACAACGA CCAAAACA | 1437 |
| 1915 | GACCGGAG G CUGACCAG | 450 | CTGGTCAG GGCTAGCTACAACGA CTCCGGTC | 1438 |
| 1919 | GGAGGCUG A CCAGUGUG | 451 | CACACTGG GGCTAGCTACAACGA CAGCCTCC | 1439 |
| 1923 | GCUGACCA G UGUGUGGC | 452 | GCCACACA GGCTAGCTACAACGA TGGTCAGC | 1440 |
| 1925 | UGACCAGU G UGUGGCCU | 453 | AGGCCACA GGCTAGCTACAACGA ACTGGTCA | 1441 |
| 1927 | ACCAGUGU G UGGCCUGU | 454 | ACAGGCCA GGCTAGCTACAACGA ACACTGGT | 1442 |
| 1930 | AGUGUGUG G CCUGUGCC | 455 | GGCACAGG GGCTAGCTACAACGA CACACACT | 1443 |
| 1934 | UGUGGCCU G UGCCCACU | 456 | AGTGGGCA GGCTAGCTACAACGA AGGCCACA | 1444 |
| 1936 | UGGCCUGU G CCCACUAU | 457 | ATAGTGGG GGCTAGCTACAACGA ACAGGCCA | 1445 |
| 1940 | CUGUGCCC A CUAUAAGG | 458 | CCTTATAG GGCTAGCTACAACGA GGGCACAG | 1446 |
| 1943 | UGCCCACU A UAAGGACC | 459 | GGTCCTTA GGCTAGCTACAACGA AGTGGGCA | 1447 |
| 1949 | CUAUAAGG A CCCUCCCU | 460 | AGGGAGGG GGCTAGCTACAACGA CCTTATAG | 1448 |
| 1961 | UCCCUUCU G CGUGGCCC | 461 | GGGCCACG GGCTAGCTACAACGA AGAAGGGA | 1449 |
| 1963 | CCUUCUGC G UGGCCCGC | 462 | GCGGGCCA GGCTAGCTACAACGA GCAGAAGG | 1450 |
| 1966 | UCUGCGUG G CCCGCUGC | 463 | GCAGCGGG GGCTAGCTACAACGA CACGCAGA | 1451 |
| 1970 | CGUGGCCC G CUGCCCCA | 464 | TGGGGCAG GGCTAGCTACAACGA GGGCCACG | 1452 |
| 1973 | GGCCCGCU G CCCCAGCG | 465 | CGCTGGGG GGCTAGCTACAACGA AGCGGGCC | 1453 |
| 1979 | CUGCCCCA G CGGUGUGA | 466 | TCACACCG GGCTAGCTACAACGA TGGGGCAG | 1454 |
| 1982 | CCCCAGCG G UGUGAAAC | 467 | GTTTCACA GGCTAGCTACAACGA CGCTGGGG | 1455 |
| 1984 | CCAGCGGU G UGAAACCU | 468 | AGGTTTCA GGCTAGCTACAACGA ACCGCTGG | 1456 |
| 1989 | GGUGUGAA A CCUGACCU | 469 | AGGTCAGG GGCTAGCTACAACGA TTCACACC | 1457 |
| 1994 | GAAACCUG A CCUCUCCU | 470 | AGGAGAGG GGCTAGCTACAACGA CAGGTTTC | 1458 |
| 2003 | CCUCUCCU A CAUGCCCA | 471 | TGGGCATG GGCTAGCTACAACGA AGGAGAGG | 1459 |
| 2005 | UCUCCAUC A UGCCCAUC | 472 | GATGGGCA GGCTAGCTACAACGA GTAGGAGA | 1460 |
| 2007 | UCCUACAU G CCCAUCUG | 473 | CAGATGGG GGCTAGCTACAACGA ATGTAGGA | 1461 |
| 2011 | ACAUGCCC A UCUGGAAG | 474 | CTTCCAGA GGCTAGCTACAACGA GGGCATGT | 1462 |
| 2019 | AUCUGGAA G UUUCCAGA | 475 | TCTGGAAA GGCTAGCTACAACGA TTCCAGAT | 1463 |
| 2027 | GUUUCCAG A UGAGGAGG | 476 | CCTCCTCA GGCTAGCTACAACGA CTGGAAAC | 1464 |
| 2036 | UGAGGAGG G CGCAUGCC | 477 | GGCATGCG GGCTAGCTACAACGA CCTCCTCA | 1465 |
| 2038 | AGGAGGGC G CAUGCCAG | 478 | CTGGCATG GGCTAGCTACAACGA GCCCTCCT | 1466 |
| 2040 | GAGGGCGC A UGCCAGCC | 479 | GGCTGGCA GGCTAGCTACAACGA GCGCCCTC | 1467 |
| 2042 | GGGCGCAU G CCAGCCUU | 480 | AAGGCTGG GGCTAGCTACAACGA ATGCGCCC | 1468 |
| 2046 | GGAUGCCA G CCUUGCCC | 481 | GGGCAAGG GGCTAGCTACAACGA TGGCATGC | 1469 |
| 2051 | CCAGCCUU G CCCCAUCA | 482 | TGATGGGG GGCTAGCTACAACGA AAGGCTGG | 1470 |
| 2056 | CUUGCCCC A UCAACUGC | 483 | GCAGTTGA GGCTAGCTACAACGA GGGGCAAG | 1471 |
| 2060 | CCCCAUCA A CUGCACCC | 484 | GGGTGCAG GGCTAGCTACAACGA TGATGGGG | 1472 |
| 2063 | CAUCAACU G CACCCACU | 485 | AGTGGGTG GGCTAGCTACAACGA AGTTGATG | 1473 |
| 2065 | UCAACUGC A CCCACUCC | 486 | GGAGTGGG GGCTAGCTACAACGA GCAGTTGA | 1474 |
| 2069 | CUGCACCC A CUCCUGUG | 487 | CACAGGAG GGCTAGCTACAACGA GGGTGCAG | 1475 |
| 2075 | CCACUCCU G UGUGGACC | 488 | GGTCCACA GGCTAGCTACAACGA AGGAGTGG | 1476 |
| 2077 | ACUCCUGU G UGGACCUG | 489 | CAGGTCCA GGCTAGCTACAACGA ACAGGAGT | 1477 |
| 2081 | CUGUGUGG A CCUGGAUG | 490 | CATCCAGG GGCTAGCTACAACGA CCACACAG | 1478 |
| 2087 | GGACCUGG A UGACAAGG | 491 | CCTTGTCA GGCTAGCTACAACGA CCAGGTCC | 1479 |
| 2090 | CCUGGAUG A CAAGGGCU | 492 | AGCCCTTG GGCTAGCTACAACGA CATCCAGG | 1480 |
| 2096 | UGACAAGG G CUGCCCCG | 493 | CGGGGCAG GGCTAGCTACAACGA CCTTGTCA | 1481 |
| 2099 | CAAGGGCU G CCCCGCCG | 494 | CGGCGGGG GGCTAGCTACAACGA AGCCCTTG | 1482 |
| 2104 | GCUGCCCC G CCGAGCAG | 495 | CTGCTCGG GGCTAGCTACAACGA GGGGCAGC | 1483 |
| 2109 | CCCGCCGA G CAGAGAGC | 496 | GCTCTCTG GGCTAGCTACAACGA TCGGCGGG | 1484 |
| 2116 | AGCAGAGA G CCAGCCCU | 497 | AGGGCTGG GGCTAGCTACAACGA TCTCTGCT | 1485 |
| 2120 | GAGAGCCA G CCCUCUGA | 498 | TCAGAGGG GGCTAGCTACAACGA TGGCTCTC | 1486 |
| 2128 | GCCCUCUG A CGUCCAUC | 499 | GATGGACG GGCTAGCTACAACGA CAGAGGGC | 1487 |
| 2130 | CCUCUGAC G UCCAUCAU | 500 | ATGATGGA GGCTAGCTACAACGA GTCAGAGG | 1488 |
| 2134 | UGACGUCC A UCAUCUCU | 501 | AGAGATGA GGCTAGCTACAACGA GGACGTCA | 1489 |
| 2137 | CGUCCAUC A UCUCUGCG | 502 | CGCAGAGA GGCTAGCTACAACGA GATGGACG | 1490 |
| 2143 | UCAUCUCU G CGGUGGUU | 503 | AACCACCG GGCTAGCTACAACGA AGAGATGA | 1491 |
| 2146 | UCUCUGCG G UGGUUGGC | 504 | GCCAACCA GGCTAGCTACAACGA CGCAGAGA | 1492 |
| 2149 | CUGCGGUG G UUGGCAUU | 505 | AATGCCAA GGCTAGCTACAACGA CACCGCAG | 1493 |
| 2153 | GGUGGUUG G CAUUCUGC | 506 | GCAGAATG GGCTAGCTACAACGA CAACCACC | 1494 |
| 2155 | UGGUUGGC A UUCUGCUG | 507 | CAGCAGAA GGCTAGCTACAACGA GCCAACCA | 1495 |
| 2160 | GGCAUUCU G CUGGUCGU | 508 | ACGACCAG GGCTAGCTACAACGA AGAATGCC | 1496 |
| 2164 | UUCUGCUG G UCGUGGUC | 509 | GACCACGA GGCTAGCTACAACGA CAGCAGAA | 1497 |
| 2167 | UGCUGGUC G UGGUCUUG | 510 | CAAGACCA GGCTAGCTACAACGA GACCAGCA | 1498 |
| 2170 | UGGUCGUG G UCUUGGGG | 511 | CCCCAAGA GGCTAGCTACAACGA CACGACCA | 1499 |
| 2179 | UCUUGGGG G UGGUCUUU | 512 | AAAGACCA GGCTAGCTACAACGA CCCCAAGA | 1500 |
| 2182 | UGGGGGUG G UCUUUGGG | 513 | CCCAAAGA GGCTAGCTACAACGA CACCCCCA | 1501 |
| 2191 | UCUUUGGG A UCCUCAUC | 514 | GATGAGGA GGCTAGCTACAACGA CCCAAAGA | 1502 |
| 2197 | GGAUCCUC A UCAAGCGA | 515 | TCGCTTGA GGCTAGCTACAACGA GAGGATCC | 1503 |
| 2202 | CUCAUCAA G CGACGGCA | 516 | TGCCGTCG GGCTAGCTACAACGA TTGATGAG | 1504 |
| 2205 | AUCAAGCG A CGGCAGCA | 517 | TGCTGCCG GGCTAGCTACAACGA CGCTTGAT | 1505 |
| 2208 | AAGCGACG G CAGCAGAA | 518 | TTCTGCTG GGCTAGCTACAACGA CGTCGCTT | 1506 |
| 2211 | CGACGGCA G CAGAAGAU | 519 | ATCTTCTG GGCTAGCTACAACGA TGCCGTCG | 1507 |
| 2218 | AGCAGAAG A UCCGGAAG | 520 | CTTCCGGA GGCTAGCTACAACGA CTTCTGCT | 1508 |
| 2226 | AUCCGGAA G UACACGAU | 521 | ATCGTGTA GGCTAGCTACAACGA TTCCGGAT | 1509 |
| 2228 | CCGGAAGU A CACGAUGC | 522 | GCATCGTG GGCTAGCTACAACGA ACTTCCGG | 1510 |
| 2230 | GGAAGUAC A CGAUGCGG | 523 | CCGCATCG GGCTAGCTACAACGA GTACTTCC | 1511 |
| 2233 | AGUACACG A UGCGGAGA | 524 | TCTCCGCA GGCTAGCTACAACGA CGTGTACT | 1512 |
| 2235 | UACACGAU G CGGAGACU | 525 | AGTCTCCG GGCTAGCTACAACGA ATCGTGTA | 1513 |
| 2241 | AUGCGGAG A CUGCUGCA | 526 | TGCAGCAG GGCTAGCTACAACGA CTCCGCAT | 1514 |
| 2244 | CGGAGACU G CUGCAGGA | 527 | TCCTGCAG GGCTAGCTACAACGA AGTCTCCG | 1515 |
| 2247 | AGACUGCU G CAGGAAAC | 528 | GTTTCCTG GGCTAGCTACAACGA AGCAGTCT | 1516 |
| 2254 | UGCAGGAA A CGGAGCUG | 529 | CAGCTCCG GGCTAGCTACAACGA TTCCTGCA | 1517 |
| 2259 | GAAACGGA G CUGGUGGA | 530 | TCCACCAG GGCTAGCTACAACGA TCCGTTTC | 1518 |
| 2263 | CGGAGCUG G UGGAGCCG | 531 | CGGCTCCA GGCTAGCTACAACGA CAGCTCCG | 1519 |
| 2268 | CUGGUGGA G CCGCUGAC | 532 | GTCAGCGG GGCTAGCTACAACGA TCCACCAG | 1520 |
| 2271 | GUGGAGCC G CUGACACC | 533 | GGTGTCAG GGCTAGCTACAACGA GGCTCCAC | 1521 |
| 2275 | AGCCGCUG A CACCUAGC | 534 | GCTAGGTG GGCTAGCTACAACGA CAGCGGCT | 1522 |
| 2277 | CCGCUGAC A CCUAGCGG | 535 | CCGCTAGG GGCTAGCTACAACGA GTCAGCGG | 1523 |
| 2282 | GACACCUA G CGGAGCGA | 536 | TCGCTCCG GGCTAGCTACAACGA TAGGTGTC | 1524 |
| 2287 | CUAGCGGA G CGAUGCCC | 537 | GGGCATCG GGCTAGCTACAACGA TCCGCTAG | 1525 |
| 2290 | GCGGAGCG A UGCCCAAC | 538 | GTTGGGCA GGCTAGCTACAACGA CGCTCCGC | 1526 |
| 2292 | GGAGCGAU G CCCAACCA | 539 | TGGTTGGG GGCTAGCTACAACGA ATCGCTCC | 1527 |
| 2297 | GAUGCCCA A CCAGGCGC | 540 | GCGCCTGG GGCTAGCTACAACGA TGGGCATC | 1528 |
| 2302 | CCAACCAG G CGCAGAUG | 541 | CATCTGCG GGCTAGCTACAACGA CTGGTTGG | 1529 |
| 2304 | AACCAGGC G CAGAUGCG | 542 | CGCATCTG GGCTAGCTACAACGA GCCTGGTT | 1530 |
| 2308 | AGGCGCAG A UGCGGAUC | 543 | GATCCGCA GGCTAGCTACAACGA CTGCGCCT | 1531 |
| 2310 | GCGCAGAU G CGGAUCCU | 544 | AGGATCCG GGCTAGCTACAACGA ATCTGCGC | 1532 |
| 2314 | AGAUGCGG A UCCUGAAA | 545 | TTTCAGGA GGCTAGCTACAACGA CCGCATCT | 1533 |
| 2326 | UGAAAGAG A CGGAGCUG | 546 | CAGCTCCG GGCTAGCTACAACGA CTCTTTCA | 1534 |
| 2331 | GAGACGGA G CUGAGGAA | 547 | TTCCTCAG GGCTAGCTACAACGA TCCGTCTC | 1535 |
| 2341 | UGAGGAAG G UGAAGGUG | 548 | CACCTTCA GGCTAGCTACAACGA CTTCCTCA | 1536 |
| 2347 | AGGUGAAG G UGCUUGGA | 549 | TCCAAGCA GGCTAGCTACAACGA CTTCACCT | 1537 |
| 2349 | GUGAAGGU G CUUGGAUC | 550 | GATCCAAG GGCTAGCTACAACGA ACCTTCAC | 1538 |
| 2355 | GUGCUUGG A UCUGGCGC | 551 | GCGCCAGA GGCTAGCTACAACGA CCAAGCAC | 1539 |
| 2360 | UGGAUCUG G CGCUUUUG | 552 | CAAAAGCG GGCTAGCTACAACGA CAGATCCA | 1540 |
| 2362 | GAUCUGGC G CUUUUGGC | 553 | GCCAAAAG GGCTAGCTACAACGA GCCAGATC | 1541 |
| 2369 | CGCUUUUG G CACAGUCU | 554 | AGACTGTG GGCTAGCTACAACGA CAAAAGCG | 1542 |
| 2371 | CUUUUGGC A CAGUCUAC | 555 | GTAGACTG GGCTAGCTACAACGA GCCAAAAG | 1543 |
| 2374 | UUGGCACA G UCUACAAG | 556 | CTTGTAGA GGCTAGCTACAACGA TGTGCCAA | 1544 |
| 2378 | CACAGUCU A CAAGGGCA | 557 | TGCCCTTG GGCTAGCTACAACGA AGACTGTG | 1545 |
| 2384 | CUACAAGG G CAUCUGGA | 558 | TCCAGATG GGCTAGCTACAACGA CCTTGTAG | 1546 |
| 2386 | ACAAGGGC A UCUGGAUC | 559 | GATCCAGA GGCTAGCTACAACGA GCCCTTGT | 1547 |
| 2392 | GCAUCUGG A UCCCUGAU | 560 | ATCAGGGA GGCTAGCTACAACGA CCAGATGC | 1548 |
| 2399 | GAUCCCUG A UGGGGAGA | 561 | TCTCCCCA GGCTAGCTACAACGA CAGGGATC | 1549 |
| 2408 | UGGGGAGA A UGUGAAAA | 562 | TTTTCACA GGCTAGCTACAACGA TCTCCCCA | 1550 |
| 2410 | GGGAGAAU G UGAAAAUU | 563 | AATTTTCA GGCTAGCTACAACGA ATTCTCCC | 1551 |
| 2416 | AUGUGAAA A UUCCAGUG | 564 | CACTGGAA GGCTAGCTACAACGA TTTCACAT | 1552 |
| 2422 | AAAUUCCA G UGGCCAUC | 565 | GATGGCCA GGCTAGCTACAACGA TGGAATTT | 1553 |
| 2425 | UUCCAGUG G CCAUCAAA | 566 | TTTGATGG GGCTAGCTACAACGA CACTGGAA | 1554 |
| 2428 | CAGUGGCC A UCAAAGUG | 567 | CACTTTGA GGCTAGCTACAACGA GGCCACTG | 1555 |
| 2434 | CCAUCAAA G UGUUGAGG | 568 | CCTCAACA GGCTAGCTACAACGA TTTGATGG | 1556 |
| 2436 | AUCAAAGU G UUGAGGGA | 569 | TCCCTCAA GGCTAGCTACAACGA ACTTTGAT | 1557 |
| 2447 | GAGGGAAA A CACAUCCC | 570 | GGGATGTG GGCTAGCTACAACGA TTTCCCTC | 1558 |
| 2449 | GGGAAAAC A CAUCCCCC | 571 | GGGGGATG GGCTAGCTACAACGA GTTTTCCC | 1559 |
| 2451 | GAAAACAC A UCCCCCAA | 572 | TTGGGGGA GGCTAGCTACAACGA GTGTTTTC | 1560 |
| 2461 | CCCCCAAA G CCAACAAA | 573 | TTTGTTGG GGCTAGCTACAACGA TTTGGGGG | 1561 |
| 2465 | CAAAGCCA A CAAAGAAA | 574 | TTTCTTTG GGCTAGCTACAACGA TGGCTTTG | 1562 |
| 2473 | ACAAAGAA A UCUUAGAC | 575 | GTCTAAGA GGCTAGCTACAACGA TTCTTTGT | 1563 |
| 2480 | AAUCUUAG A CGAAGCAU | 576 | ATGCTTCG GGCTAGCTACAACGA CTAAGATT | 1564 |
| 2485 | UAGACGAA G CAUACGUG | 577 | CACGTATG GGCTAGCTACAACGA TTCGTCTA | 1565 |
| 2487 | GACGAAGC A UACGUGAU | 578 | ATCACGTA GGCTAGCTACAACGA GCTTCGTC | 1566 |
| 2489 | CGAAGCAU A CGUGAUGG | 579 | CCATCACG GGCTAGCTACAACGA ATGCTTCG | 1567 |
| 2491 | AAGCAUAC G UGAUGGCU | 580 | AGCCATCA GGCTAGCTACAACGA GTATGCTT | 1568 |
| 2494 | CAUACGUG A UGGCUGGU | 581 | ACCAGCCA GGCTAGCTACAACGA CACGTATG | 1569 |
| 2497 | ACGUGAUG G CUGGUGUG | 582 | CACACCAG GGCTAGCTACAACGA CATCACGT | 1570 |
| 2501 | GAUGGCUG G UGUGGGCU | 583 | AGCCCACA GGCTAGCTACAACGA CAGCCATC | 1571 |
| 2503 | UGGCUGGU G UGGGCUCC | 584 | GGAGCCCA GGCTAGCTACAACGA ACCAGCCA | 1572 |
| 2507 | UGGUGUGG G CUCCCCAU | 585 | ATGGGGAG GGCTAGCTACAACGA CCACACCA | 1573 |
| 2514 | GGCUCCCC A UAUGUCUC | 586 | GAGACATA GGCTAGCTACAACGA GGGGAGCC | 1574 |
| 2516 | CUCCCCAU A UGUCUCCC | 587 | GGGAGACA GGCTAGCTACAACGA ATGGGGAG | 1575 |
| 2518 | CCCCAUAU G UCUCCCGC | 588 | GCGGGAGA GGCTAGCTACAACGA ATATGGGG | 1576 |
| 2525 | UGUCUCCC G CCUUCUGG | 589 | CCAGAAGG GGCTAGCTACAACGA GGGAGACA | 1577 |
| 2534 | CCUUCUGG G CAUCUGCC | 590 | GGCAGATG GGCTAGCTACAACGA CCAGAAGG | 1578 |
| 2536 | UUCUGGGC A UCUGCCUG | 591 | CAGGCAGA GGCTAGCTACAACGA GCCCAGAA | 1579 |
| 2540 | GGGCAUCU G CCUGACAU | 592 | ATGTCAGG GGCTAGCTACAACGA AGATGCCC | 1580 |
| 2545 | UCUGCCUG A CAUCCACG | 593 | CGTGGATG GGCTAGCTACAACGA CAGGCAGA | 1581 |
| 2547 | UGCCUGAC A UCCACGGU | 594 | ACCGTGGA GGCTAGCTACAACGA GTCAGGCA | 1582 |
| 2551 | UGACAUCC A CGGUGCAG | 595 | CTGCACCG GGCTAGCTACAACGA GGATGTCA | 1583 |
| 2554 | CAUCCACG G UGCAGCUG | 596 | CAGCTGCA GGCTAGCTACAACGA CGTGGATG | 1584 |
| 2556 | UCCACGGU G CAGCUGGU | 597 | ACCAGCTG GGCTAGCTACAACGA ACCGTGGA | 1585 |
| 2559 | ACGGUGCA G CUGGUGAC | 598 | GTCACCAG GGCTAGCTACAACGA TGCACCGT | 1586 |
| 2563 | UGCAGCUG G UGACACAG | 599 | CTGTGTCA GGCTAGCTACAACGA CAGCTGCA | 1587 |
| 2566 | AGCUGGUG A CACAGCUU | 600 | AAGCTGTG GGCTAGCTACAACGA CACCAGCT | 1588 |
| 2568 | CUGGUGAC A CAGCUUAU | 601 | ATAAGCTG GGCTAGCTACAACGA GTCACCAG | 1589 |
| 2571 | GUGACACA G CUUAUGCC | 602 | GGCATAAG GGCTAGCTACAACGA TGTGTCAC | 1590 |
| 2575 | CACAGCUU A UGCCCUAU | 603 | ATAGGGCA GGCTAGCTACAACGA AAGCTGTG | 1591 |
| 2577 | CAGCUUAU G CCCUAUGG | 604 | CCATAGGG GGCTAGCTACAACGA ATAAGCTG | 1592 |
| 2582 | UAUGCCCU A UGGCUGCC | 605 | GGCAGCCA GGCTAGCTACAACGA AGGGCATA | 1593 |
| 2585 | GCCCUAUG G CUGCCUCU | 606 | AGAGGCAG GGCTAGCTACAACGA CATAGGGC | 1594 |
| 2588 | CUAUGGCU G CCUCUUAG | 607 | CTAAGAAG GGCTAGCTACAACGA AGCCATAG | 1595 |
| 2597 | CCUCUUAG A CCAUGUCC | 608 | GGACATGG GGCTAGCTACAACGA CTAAGAGG | 1596 |
| 2600 | CUUAGACC A UGUCCGGG | 609 | CCCGGACA GGCTAGCTACAACGA GGTCTAAG | 1597 |
| 2602 | UAGACCAU G UCCGGGAA | 610 | TTCCCGGA GGCTAGCTACAACGA ATGGTCTA | 1598 |
| 2612 | CCGGGAAA A CCGCGGAC | 611 | GTCCGCGG GGCTAGCTACAACGA TTTCCCGG | 1599 |
| 2615 | GGAAAACC G CGGACGCC | 612 | GGCGTCCG GGCTAGCTACAACGA GGTTTTCC | 1600 |
| 2619 | AACCGCGG A CGCCUGGG | 613 | CCCAGGCG GGCTAGCTACAACGA CCGCGGTT | 1601 |
| 2621 | CCGCGGAC G CCUGGGCU | 614 | AGCCCAGG GGCTAGCTACAACGA GTCCGCGG | 1602 |
| 2627 | ACGCCUGG G CUCCCAGG | 615 | CCTGGGAG GGCTAGCTACAACGA CCAGGCGT | 1603 |
| 2636 | CUCCCAGG A CCUGCUGA | 616 | TCAGCAGG GGCTAGCTACAACGA CCTGGGAG | 1604 |
| 2640 | CAGGACCU G CUGAACUG | 617 | CAGTTCAG GGCTAGCTACAACGA AGGTCCTG | 1605 |
| 2645 | CCUGCUGA A CUGGUGUA | 618 | TACACCAG GGCTAGCTACAACGA TCAGCAGG | 1606 |
| 2649 | CUGAACUG G UGUAUGCA | 619 | TGCATACA GGCTAGCTACAACGA CAGTTCAG | 1607 |
| 2651 | GAACUGGU G UAUGCAGA | 620 | TCTGCATA GGCTAGCTACAACGA ACCAGTTC | 1608 |
| 2653 | ACUGGUGU A UGCAGAUU | 621 | AATCTGCA GGCTAGCTACAACGA ACACCAGT | 1609 |
| 2655 | UGGCGUAU G CAGAUUGC | 622 | GCAATCTG GGCTAGCTACAACGA ATACACCA | 1610 |
| 2659 | GUAUGCAG A UUGCCAAG | 623 | GTTGGCAA GGCTAGCTACAACGA CTGCATAC | 1611 |
| 2662 | UGCAGAUU G CCAAGGGG | 624 | CCCCTTGG GGCTAGCTACAACGA AATCTGCA | 1612 |
| 2671 | CCAAGGGG A UGAGCUAC | 625 | GTAGCTCA GGCTAGCTACAACGA CCCCTTGG | 1613 |
| 2675 | GGGGAUGA G CUACCUGG | 626 | CCAGGTAG GGCTAGCTACAACGA TCATCCCC | 1614 |
| 2678 | GAUGAGCU A CCUGGAGG | 627 | CCTCCAGG GGCTAGCTACAACGA AGCTCATC | 1615 |
| 2687 | CCUGGAGG A UGUGCGGC | 628 | GCCGCACA GGCTAGCTACAACGA CCTCCAGG | 1616 |
| 2689 | UGGAGGAU G UGCGGCUC | 629 | GAGCCGCA GGCTAGCTACAACGA ATCCTCCA | 1617 |
| 2691 | GAGGAUGU G CGGCUCGU | 630 | ACGAGCCG GGCTAGCTACAACGA ACATCCTC | 1618 |
| 2694 | GAUGUGCG G CUCGUACA | 631 | TGTACGAG GGCTAGCTACAACGA CGCACATC | 1619 |
| 2698 | UGCGGCUC G UACACAGG | 632 | CCTGTGTA GGCTAGCTACAACGA GAGCCGCA | 1620 |
| 2700 | CGGCUCGU A CACAGGGA | 633 | TCCCTGTG GGCTAGCTACAACGA ACGAGCCG | 1621 |
| 2702 | GCUCGUAC A CAGGGACU | 634 | AGTCCCTG GGCTAGCTACAACGA GTACGAGC | 1622 |
| 2708 | ACACAGGG A CUUGGCCG | 635 | CGGCCAAG GGCTAGCTACAACGA CCCTGTGT | 1623 |
| 2713 | GGGACUUG G CCGCUCGG | 636 | CCGAGCGG GGCTAGCTACAACGA CAAGTCCC | 1624 |
| 2716 | ACUUGGCC G CUCGGAAC | 637 | GTTCCGAG GGCTAGCTACAACGA GGCCAAGT | 1625 |
| 2723 | CGCUCGGA A CGUGCUGG | 638 | CCAGCACG GGCTAGCTACAACGA TCCGAGCG | 1626 |
| 2725 | CUCGGAAC G UGCUGGUC | 639 | GACCAGCA GGCTAGCTACAACGA GTTCCGAG | 1627 |
| 2727 | CGGAACGU G CUGGUCAA | 640 | TTGACCAG GGCTAGCTACAACGA ACGTTCCG | 1628 |
| 2731 | ACGUGCUG G UCAAGAGU | 641 | ACTCTTGA GGCTAGCTACAACGA CAGCACGT | 1629 |
| 2738 | GGUCAAGA G UCCCAACC | 642 | GGTTGGGA GGCTAGCTACAACGA TCTTGACC | 1630 |
| 2744 | GAGUCCCA A CCAUGUCA | 643 | TGACATGG GGCTAGCTACAACGA TGGGACTC | 1631 |
| 2747 | UCCCAACC A UGUCAAAA | 644 | TTTTGACA GGCTAGCTACAACGA GGTTGGGA | 1632 |
| 2749 | CCAACCAU G UCAAAAUU | 645 | AATTTTGA GGCTAGCTACAACGA ATGGTTGG | 1633 |
| 2755 | AUGUCAAA A UUACAGAC | 646 | GTCTGTAA GGCTAGCTACAACGA TTTGACAT | 1634 |
| 2758 | UCAAAAUU A CAGACUUC | 647 | GAAGTCTG GGCTAGCTACAACGA AATTTTGA | 1635 |
| 2762 | AAUUACAG A CUUCGGGC | 648 | GCCCGAAG GGCTAGCTACAACGA CTGTAATT | 1636 |
| 2769 | GACUUCGG G CUGGCUCG | 649 | CGAGCCAG GGCTAGCTACAACGA CCGAAGTC | 1637 |
| 2773 | UCGGGCUG G CUCGGCUG | 650 | CAGCCGAG GGCTAGCTACAACGA CAGCCCGA | 1638 |
| 2778 | CUGGCUCG G CUGCUGGA | 651 | TCCAGCAG GGCTAGCTACAACGA CGAGCCAG | 1639 |
| 2781 | GCUCGGCU G CUGGACAU | 652 | ATGTCCAG GGCTAGCTACAACGA AGCCGAGC | 1640 |
| 2786 | GCUGCUGG A CAUUGACG | 653 | CGTCAATG GGCTAGCTACAACGA CCAGCAGC | 1641 |
| 2788 | UGCUGGAC A UUGACGAG | 654 | CTCGTCAA GGCTAGCTACAACGA GTCCAGCA | 1462 |
| 2792 | GGACAUUG A CGAGACAG | 655 | CTGTCTCG GGCTAGCTACAACGA CAATGTCC | 1643 |
| 2797 | UUGACGAG A CAGAGUAC | 656 | GTACTCTG GGCTAGCTACAACGA CTCGTCAA | 1644 |
| 2802 | GAGACAGA G UACCAUGC | 657 | GCATGGTA GGCTAGCTACAACGA TCTGTCTC | 1645 |
| 2804 | GACAGAGU A CCAUGCAG | 658 | CTGCATGG GGCTAGCTACAACGA ACTCTGTC | 1646 |
| 2807 | AGAGUACC A UGCAGAUG | 659 | CATCTGCA GGCTAGCTACAACGA GGTACTCT | 1647 |
| 2809 | AGUACCAU G CAGAUGGG | 660 | CCCATCTG GGCTAGCTACAACGA ATGGTACT | 1648 |
| 2813 | CCAUGCAG A UGGGGGCA | 661 | TGCCCCCA GGCTAGCTACAACGA CTGCATGG | 1649 |
| 2819 | AGAUGGGG G CAAGGUGC | 662 | GCACCTTG GGCTAGCTACAACGA CCCCATCT | 1650 |
| 2824 | GGGGCAAG G UGCCCAUC | 663 | GATGGGCA GGCTAGCTACAACGA CTTGCCCC | 1651 |
| 2835 | CCCAUCAA G UGGAUGGC | 666 | GCCATCCA GGCTAGCTACAACGA TTGATGGG | 1654 |
| 2839 | UCAAGUGG A UGGCGCUG | 667 | CAGCGCCA GGCTAGCTACAACGA CCACTTGA | 1655 |
| 2842 | AGUGGAUG G CGCUGGAG | 668 | CTCCAGCG GGCTAGCTACAACGA CATCCACT | 1656 |
| 2844 | UGGAUGGC G CUGGAGUC | 669 | GACTCCAG GGCTAGCTACAACGA GCCATCCA | 1657 |
| 2850 | GCGCUGGA G UCCAUUCU | 670 | AGAATGGA GGCTAGCTACAACGA TCCAGCGC | 1658 |
| 2854 | UGGAGUCC A UUCUCCGC | 671 | GCGGAGAA GGCTAGCTACAACGA GGACTCCA | 1659 |
| 2861 | CAUUCUCC G CCGGCGGU | 672 | ACCGCCGG GGCTAGCTACAACGA GGAGAATG | 1660 |
| 2865 | CUCCGCCG G CGGUUCAC | 673 | GTGAACCG GGCTAGCTACAACGA CGGCGGAG | 1661 |
| 2868 | CGCCGGCG G UUCACCCA | 674 | TGGGTGAA GGCTAGCTACAACGA CGCCGGCG | 1662 |
| 2872 | GGCGGUUC A CCCACCAG | 675 | CTGGTGGG GGCTAGCTACAACGA GAACCGCC | 1663 |
| 2876 | GUUCACCC A CCAGAGUG | 676 | CACTCTGG GGCTAGCTACAACGA GGGTGAAC | 1664 |
| 2882 | CCACCAGA G UGAUGUGU | 677 | ACACATCA GGCTAGCTACAACGA TCTGGTGG | 1665 |
| 2885 | CCAGAGUG A UGUGUGGA | 678 | TCCACACA GGCTAGCTACAACGA CACTCTGG | 1666 |
| 2887 | AGAGUGAU G UGUGGAGU | 679 | ACTCCACA GGCTAGCTACAACGA ATCACTCT | 1667 |
| 2889 | AGUGAUGU G UGGAGUUA | 680 | TACCTCCA GGCTAGCTACAACGA ACATCACT | 1668 |
| 2894 | UGUGUGGA G UUAUGGUG | 681 | CACCATAA GGCTAGCTACAACGA TCCACACA | 1669 |
| 2897 | GUGGAGUU A UGGUGUGA | 682 | TCACACCA GGCTAGCTACAACGA AACTCCAC | 1670 |
| 2900 | GAGUUAUG G UGUGACUG | 683 | CAGTCACA GGCTAGCTACAACGA CATAACTC | 1671 |
| 2902 | GUUAUGGU G UGACUGUG | 684 | CACAGTCA GGCTAGCTACAACGA ACCATAAC | 1672 |
| 2905 | AUGGUGUG A CUGUGUGG | 685 | CCACACAG GGCTAGCTACAACGA CACACCAT | 1673 |
| 2908 | GUGUGACU G UGUGGGAG | 686 | CTCCCACA GGCTAGCTACAACGA AGTCACAC | 1674 |
| 2910 | GUGACUGU G UGGGAGCU | 687 | AGCTCCCA GGCTAGCTACAACGA ACAGTCAC | 1675 |
| 2916 | GUGUGGGA G CUGAUGAC | 688 | GTCATCAG GGCTAGCTACAACGA TCCCACAC | 1676 |
| 2920 | GGGAGCUG A UGACUUUU | 689 | AAAAGTCA GGCTAGCTACAACGA CAGCTCCC | 1677 |
| 2923 | AGCUGAUG A CUUUUGGG | 690 | CCCAAAAG GGCTAGCTACAACGA CATCAGCT | 1678 |
| 2932 | CUUUUGGG G CCAAACCU | 691 | AGGTTTGG GGCTAGCTACAACGA CCCAAAAG | 1679 |
| 2937 | GGGGCCAA A CCUUACGA | 692 | TCGTAAGG GGCTAGCTACAACGA TTGGCCCC | 1680 |
| 2942 | CAAACCUU A CGAUGGGA | 693 | TCCCATCG GGCTAGCTACAACGA AAGGTTTG | 1681 |
| 2945 | ACCUUACG A UGGGAUCC | 694 | GGATCCCA GGCTAGCTACAACGA CGTAAGGT | 1682 |
| 2950 | ACGAUGGG A UCCCAGCC | 695 | GGCTGGGA GGCTAGCTACAACGA CCCATCGT | 1683 |
| 2956 | GGAUCCCA G CCCGGGAG | 696 | CTCCCGGG GGCTAGCTACAACGA TGGGATCC | 1684 |
| 2965 | CCCGGGAG A UCCCUGAC | 697 | GTCAGGGA GGCTAGCTACAACGA CTCCCGGG | 1685 |
| 2972 | GAUCCCUG A CCUGCUGG | 698 | CCAGCAGG GGCTAGCTACAACGA CAGGGATC | 1686 |
| 2976 | CCUGACCU G CUGGAAAA | 699 | TTTTCCAG GGCTAGCTACAACGA AGGTCAGG | 1687 |
| 2991 | AAGGGGGA G CGGCUGCC | 700 | GGCAGCCG GGCTAGCTACAACGA TCCCCCTT | 1688 |
| 2994 | GGGGAGCG G CUGCCCCA | 701 | TGGGGCAG GGCTAGCTACAACGA CGCTCCCC | 1689 |
| 2997 | GAGCGGCU G CCCCAGCC | 702 | GGCTGGGG GGCTAGCTACAACGA AGCCGCTC | 1690 |
| 3003 | CUGCCCCA G CCCCCCAU | 703 | ATGGGGGG GGCTAGCTACAACGA TGGGGCAG | 1691 |
| 3010 | AGCCCCCC A UCUGCACC | 704 | GGTGCAGA GGCTAGCTACAACGA GGGGGGCT | 1692 |
| 3014 | CCCCAUCU G CACCAUUG | 705 | CAATGGTG GGCTAGCTACAACGA AGATGGGG | 1693 |
| 3016 | CCAUCUGC A CCAUUGAU | 706 | ATCAATGG GGCTAGCTACAACGA GCAGATGG | 1694 |
| 3019 | UCUGCACC A UUGAUGUC | 707 | GACATCAA GGCTAGCTACAACGA GGTGCAGA | 1695 |
| 3023 | CACCAUUG A UGUCUACA | 708 | TGTAGACA GGCTAGCTACAACGA CAATGGTG | 1696 |
| 3025 | CCAUUGAU G UCUACAUG | 709 | CATGTAGA GGCTAGCTACAACGA ATCAATGG | 1697 |
| 3029 | UGAUGUCU A CAUGAUCA | 710 | TGATCATG GGCTAGCTACAACGA AGACATCA | 1698 |
| 3031 | AUGUCUAC A UGAUCAUG | 711 | CATGATCA GGCTAGCTACAACGA GTAGACAT | 1699 |
| 3034 | UCUACAUG A UCAUGGUC | 712 | GACCATGA GGCTAGCTACAACGA CATGTAGA | 1700 |
| 3037 | ACAUGAUC A UGGUCAAA | 713 | TTTGACCA GGCTAGCTACAACGA GATCATGT | 1701 |
| 3040 | UGAUCAUG G UCAAAUGU | 714 | ACATTTGA GGCTAGCTACAACGA CATGATCA | 1702 |
| 3045 | AUGGUCAA A UGUUGGAU | 715 | ATCCAACA GGCTAGCTACAACGA TTGACCAT | 1703 |
| 3047 | GGUCAAAU G UUGGAUGA | 716 | TCATCCAA GGCTAGCTACAACGA ATTTGACC | 1704 |
| 3052 | AAUGUUGG A UGAUUGAC | 717 | GTCAATCA GGCTAGCTACAACGA CCAACATT | 1705 |
| 3055 | GUUGGAUG A UUGACUCU | 718 | AGAGTCAA GGCTAGCTACAACGA CATCCAAC | 1706 |
| 3059 | GAUGAUUG A CUCUGAAU | 719 | ATTCAGAG GGCTAGCTACAACGA CAATCATC | 1707 |
| 3066 | GACUCUGA A UGUCGGCC | 720 | GGCCGACA GGCTAGCTACAACGA TCAGAGTC | 1708 |
| 3068 | CUCUGAAU G UCGGCCAA | 721 | TTGGCCGA GGCTAGCTACAACGA ATTCAGAG | 1709 |
| 3072 | GAAUGUCG G CCAAGAUU | 722 | AATCTTGG GGCTAGCTACAACGA CGACATTC | 1710 |
| 3078 | CGGCCAAG A UUCCGGGA | 723 | TCCCGGAA GGCTAGCTACAACGA CTTGGCCG | 1711 |
| 3087 | UUCCGGGA G UUGGUGUC | 724 | GACACCAA GGCTAGCTACAACGA TCCCGGAA | 1712 |
| 3091 | GGGAGUUG G UGUCUGAA | 725 | TTCAGACA GGCTAGCTACAACGA CAACTCCC | 1713 |
| 3093 | GAGUUGGU G UCUGAAUU | 726 | AATTCAGA GGCTAGCTACAACGA ACCAACTC | 1714 |
| 3099 | GUGUCUGA A UUCUCCCG | 727 | CGGGAGAA GGCTAGCTACAACGA TCAGACAC | 1715 |
| 3107 | AUUCUCCC G CAUGGCCA | 728 | TGGCCATG GGCTAGCTACAACGA GGGAGAAT | 1716 |
| 3109 | UCUCCCGC A UGGCCAGG | 729 | CCTGGCCA GGCTAGCTACAACGA GCGGGAGA | 1717 |
| 3112 | CCCGCAUG G CCAGGGAC | 730 | GTCCCTGG GGCTAGCTACAACGA CATGCGGG | 1718 |
| 3119 | GGCCAGGG A CCCCCAGC | 731 | GCTGGGGG GGCTAGCTACAACGA CCCTGGCC | 1719 |
| 3126 | GACCCCCA G CGCUUUGU | 732 | ACAAAGCG GGCTAGCTACAACGA TGGGGGTC | 1720 |
| 3128 | CCCCCAGC G CUUUGUGG | 733 | CCACAAAG GGCTAGCTACAACGA GCTGGGGG | 1721 |
| 3133 | AGCGCUUU G UGGUCAUC | 734 | GATGACCA GGCTAGCTACAACGA AAAGCGCT | 1722 |
| 3136 | GCUUUGUG G UCAUCCAG | 735 | CTGGATGA GGCTAGCTACAACGA CACAAAGC | 1723 |
| 3139 | UUGUGGUC A UCCAGAAU | 736 | ATTCTGGA GGCTAGCTACAACGA GACCACAA | 1724 |
| 3146 | CAUCCAGA A UGAGGACU | 737 | AGTCCTCA GGCTAGCTACAACGA TCTGGATG | 1725 |
| 3152 | GAAUGAGG A CUUGGGCC | 738 | GGCCCAAG GGCTAGCTACAACGA CCTCATTC | 1726 |
| 3158 | GGACUUGG G CCCAGCCA | 739 | TGGCTGGG GGCTAGCTACAACGA CCAAGTCC | 1727 |
| 3163 | UGGGCCCA G CCAGUCCC | 740 | GGGACTGG GGCTAGCTACAACGA TGGGCCCA | 1728 |
| 3167 | CCCAGCCA G UCCCUUGG | 741 | CCAAGGGA GGCTAGCTACAACGA TGGCTGGG | 1729 |
| 3176 | UCCCUUGG A CAGCACCU | 742 | AGGTGCTG GGCTAGCTACAACGA CCAAGGGA | 1730 |
| 3179 | CUUGGACA G CACCUUCU | 743 | AGAAGGTG GGCTAGCTACAACGA TGTCCAAG | 1731 |
| 3181 | UGGACAGC A CCUUCUAC | 744 | GTAGAAGG GGCTAGCTACAACGA GCTGTCCA | 1732 |
| 3188 | CACCUUCU A CCGCUCAC | 745 | GTGAGCGG GGCTAGCTACAACGA AGAAGGTG | 1733 |
| 3191 | CUUCUACC G CUCACUGC | 746 | GCAGTGAG GGCTAGCTACAACGA GGTAGAAG | 1734 |
| 3195 | UACCGCUC A CUGCUGGA | 747 | TCCAGCAG GGCTAGCTACAACGA GAGCGGTA | 1735 |
| 3198 | CGCUCACU G CUGGAGGA | 748 | TCCTCCAG GGCTAGCTACAACGA AGTGAGCG | 1736 |
| 3206 | GCUGGAGG A CGAUGACA | 749 | TGTCATCG GGCTAGCTACAACGA CCTCCAGC | 1737 |
| 3209 | GGAGGACG A UGACAUGG | 750 | CCATGTCA GGCTAGCTACAACGA CGTCCTCC | 1738 |
| 3212 | GGACGAUG A CAUGGGGG | 751 | CCCCCATG GGCTAGCTACAACGA CATCGTCC | 1739 |
| 3214 | ACGAUGAC A UGGGGGAC | 752 | GTCCCCCA GGCTAGCTACAACGA GTCATCGT | 1740 |
| 3221 | CAUGGGGG A CCUGGUGG | 753 | CCACCAGG GGCTAGCTACAACGA CCCCCATG | 1741 |
| 3226 | GGGACCUG G UGGAUGCU | 754 | AGCATCCA GGCTAGCTACAACGA CAGGTCCC | 1742 |
| 3230 | CCUGGUGG A UGCUGAGG | 755 | CCTCAGCA GGCTAGCTACAACGA CCACCAGG | 1743 |
| 3232 | UGGUGGAU G CUGAGGAG | 756 | CTCCTCAG GGCTAGCTACAACGA ATCCACCA | 1744 |
| 3240 | GCUGAGGA G UAUCUGGU | 757 | ACCAGATA GGCTAGCTACAACGA TCCTCAGC | 1745 |
| 3242 | UGAGGAGU A UCUGGUAC | 758 | GTACCAGA GGCTAGCTACAACGA ACTCCTCA | 1746 |
| 3247 | AGUAUCUG G UACCCCAG | 759 | CTGGGGTA GGCTAGCTACAACGA CAGATACT | 1747 |
| 3279 | UAUCUGGU A CCCCAGCA | 760 | TGCTGGGG GGCTAGCTACAACGA ACCAGATA | 1748 |
| 3255 | GUACCCCA G CAGGGCUU | 761 | AAGCCCTG GGCTAGCTACAACGA TGGGGTAC | 1749 |
| 3260 | CCAGCAGG G CUUCUUCU | 762 | AGAAGAAG GGCTAGCTACAACGA CCTGCTGG | 1750 |
| 3269 | CUUCUUCU G UCCAGACC | 763 | GGTCTGGA GGCTAGCTACAACGA AGAAGAAG | 1751 |
| 3275 | CUGUCCAG A CCCUGCCC | 764 | GGGCAGGG GGCTAGCTACAACGA CTGGACAG | 1752 |
| 3280 | CAGACCCU G CCCCGGGC | 765 | GCCCGGGG GGCTAGCTACAACGA AGGGTCTG | 1753 |
| 3287 | UGCCCCGG G CGCUGGGG | 766 | CCCCAGCG GGCTAGCTACAACGA CCGGGGCA | 1754 |
| 3289 | CCCCGGGC G CUGGGGGC | 767 | GCCCCCAG GGCTAGCTACAACGA GCCCGGGG | 1755 |
| 3296 | CGCUGGGG G CAUGGUCC | 768 | GGACCATG GGCTAGCTACAACGA CCCCAGCG | 1756 |
| 3298 | CUGGGGGC A UGGUCCAC | 769 | GTGGACCA GGCTAGCTACAACGA GCCCCCAG | 1757 |
| 3301 | GGGGCAUG G UCCACCAC | 770 | GTGGTGGA GGCTAGCTACAACGA CATGCCCC | 1758 |
| 3305 | CAUGGUCC A CCACAGGC | 771 | GCCTGTGG GGCTAGCTACAACGA GGACCATG | 1759 |
| 3308 | GGUCCACC A CAGGCACC | 772 | GGTGCCTG GGCTAGCTACAACGA GGTGGACC | 1760 |
| 3312 | CACCACAG G CACCGCAG | 773 | CTGCGGTG GGCTAGCTACAACGA CTGTGGTG | 1761 |
| 3314 | CCACAGGC A CCGCAGCU | 774 | AGCTGCGG GGCTAGCTACAACGA GCCTGTGG | 1762 |
| 3317 | CAGGCACC G CAGCUCAU | 775 | ATGAGCTG GGCTAGCTACAACGA GGTGCCTG | 1763 |
| 3320 | GCACCGCA G CUCAUCUA | 776 | TAGATGAG GGCTAGCTACAACGA TGCGGTGC | 1764 |
| 3324 | CGCAGCUC A UCUACCAG | 777 | CTGGTAGA GGCTAGCTACAACGA GAGCTGCG | 1765 |
| 3328 | GCUCAUCU A CCAGGAGU | 778 | ACTCCTGG GGCTAGCTACAACGA AGATGAGC | 1766 |
| 3335 | UACCAGGA G UGGCGGUG | 779 | CACCGCCA GGCTAGCTACAACGA TCCTGGTA | 1767 |
| 3338 | CAGGAGUG G CGGUGGGG | 780 | CCCCACCG GGCTAGCTACAACGA CACTCCTG | 1768 |
| 3341 | GAGUGGCG G UGGGGACC | 781 | GGTCCCCA GGCTAGCTACAACGA CGCCACTC | 1769 |
| 3347 | CGGUGGGG A CCUGACAC | 782 | GTGTCAGG GGCTAGCTACAACGA CCCCACCG | 1770 |
| 3352 | GGGACCUG A CACUAGGG | 783 | CCCTAGTG GGCTAGCTACAACGA CAGGTCCC | 1771 |
| 3354 | GACCUGAC A CUAGGGCU | 784 | AGCCCTAG GGCTAGCTACAACGA GTCAGGTC | 1772 |
| 3360 | ACACUAGG G CUGGAGCC | 785 | GGCTCCAG GGCTAGCTACAACGA CCTAGTGT | 1773 |
| 3366 | GGGUCGGA G CCCUCUGA | 786 | TCAGAGGG GGCTAGCTACAACGA TCCAGCCC | 1774 |
| 3382 | AAGAGGAG G CCCCCAGG | 787 | CCTGGGGG GGCTAGCTACAACGA CTCCTCTT | 1775 |
| 3390 | GCCCCCAG G UCUCCACU | 788 | AGTGGAGA GGCTAGCTACAACGA CTGGGGGC | 1776 |
| 3396 | AGGUCUCC A CUGGCACC | 789 | GGTGCCAG GGCTAGCTACAACGA GGAGACCT | 1777 |
| 3400 | CUCCACUG G CACCCUCC | 790 | GGAGGGTG GGCTAGCTACAACGA CAGTGGAG | 1778 |
| 3402 | CCACUGGC A CCCUCCGA | 791 | TCGGAGGG GGCTAGCTACAACGA GCCAGTGG | 1779 |
| 3415 | CCGAAGGG G CUGGCUCC | 792 | GGAGCCAG GGCTAGCTACAACGA CCCTTCGG | 1780 |
| 3419 | AGGGGCUG G CUCCGAUG | 793 | CATCGGAG GGCTAGCTACAACGA CAGCCCCT | 1781 |
| 3425 | UGGCUCCG A UGUAUUUG | 794 | CAAATACA GGCTAGCTACAACGA CGGAGCCA | 1782 |
| 3427 | GCUCCGAU G UAUUUGAU | 795 | ATCAAATA GGCTAGCTACAACGA ATCGGAGC | 1783 |
| 3429 | UCCGAUGU A UUUGAUGG | 796 | CCATCAAA GGCTAGCTACAACGA ACATCGGA | 1784 |
| 3434 | UGUAUUUG A UGGUGACC | 797 | GGTCACCA GGCTAGCTACAACGA CAAATACA | 1785 |
| 3437 | AUUUGAUG G UGACCUGG | 798 | CCAGGTCA GGCTAGCTACAACGA CATCAAAT | 1786 |
| 3440 | UGAUGGUG A CCUGGGAA | 799 | TTCCCAGG GGCTAGCTACAACGA CACCATCA | 1787 |
| 3448 | ACCUGGGA A UGGGGGCA | 800 | TGCCCCCA GGCTAGCTACAACGA TCCCAGGT | 1788 |
| 3454 | GAAUGGGG G CAGCCAAG | 801 | CTTGGCTG GGCTAGCTACAACGA CCCCATTC | 1789 |
| 3457 | UGGGGGCA G CCAAGGGG | 802 | CCCCTTGG GGCTAGCTACAACGA TGCCCCCA | 1790 |
| 3465 | GCCAAGGG G CUGCAAAG | 803 | CTTTGCAG GGCTAGCTACAACGA CCCTTGGC | 1791 |
| 3468 | AAGGGGCU G CAAAGCCU | 804 | AGGCTTTG GGCTAGCTACAACGA AGCCCCTT | 1792 |
| 3473 | GCUGCAAA G CCUCCCCA | 805 | TGGGGAGG GGCTAGCTACAACGA TTTGCAGC | 1793 |
| 3481 | GCCUCCCC A CACAUGAC | 806 | GTCATGTG GGCTAGCTACAACGA GGGGAGGC | 1794 |
| 3483 | CUCCCCAC A CAUGACCC | 807 | GGGTCATG GGCTAGCTACAACGA GTGGGGAG | 1795 |
| 3485 | CCCCACAC A UGACCCCA | 808 | TGGGGTCA GGCTAGCTACAACGA GTGTGGGG | 1796 |
| 3488 | CACACAUG A CCCCAGCC | 809 | GGCTGGGG GGCTAGCTACAACGA CATGTGTG | 1797 |
| 3494 | UGACCCCA G CCCUCUAC | 810 | GTAGAGGG GGCTAGCTACAACGA TGGGGTCA | 1798 |
| 3501 | AGCCCUCU A CAGCGGUA | 811 | TACCGCTG GGCTAGCTACAACGA AGAGGGCT | 1799 |
| 3504 | CCUCUACA G CGGUACAG | 812 | CTGTACCG GGCTAGCTACAACGA TGTAGAGG | 1800 |
| 3507 | CUACAGCG G UACAGUGA | 813 | TCACTGTA GGCTAGCTACAACGA CGCTGTAG | 1801 |
| 3509 | ACAGCGGU A CAGUGAGG | 814 | CCTCACTG GGCTAGCTACAACGA ACCGCTGT | 1802 |
| 3512 | GCGGUACA G UGAGGACC | 815 | GGTCCTCA GGCTAGCTACAACGA TGTACCGC | 1803 |
| 3518 | CAGUGAGG A CCCCACAG | 816 | CTGTGGGG GGCTAGCTACAACGA CCTCACTG | 1804 |
| 3523 | AGGACCCC A CAGUACCC | 817 | GGGTACTG GGCTAGCTACAACGA GGGGTCCT | 1805 |
| 3526 | ACCCCACA G UACCCCUG | 818 | CAGGGGTA GGCTAGCTACAACGA TGTGGGGT | 1806 |
| 3528 | CCCACAGU A CCCCUGCC | 819 | GGCAGGGG GGCTAGCTACAACGA ACTGTGGG | 1807 |
| 3534 | GUACCCCU G CCCUCUGA | 820 | TCAGAGGG GGCTAGCTACAACGA AGGGGTAC | 1808 |
| 3544 | UGAGACUG A UGGCUACG | 822 | CGTAGCCA GGCTAGCTACAACGA CAGTCTCA | 1810 |
| 3548 | UGAGACUG A UGGCUACG | 822 | CGTAGCCA GGCTAGCTACAACGA CAGTCTCA | 1810 |
| 3551 | GACUGAUG G CUACGUUG | 823 | CAACGTAG GGCTAGCTACAACGA CATCAGTC | 1811 |
| 3554 | UGAUGGCU A CGUUGCCC | 824 | GGGCAACG GGCTAGCTACAACGA AGCCATCA | 1812 |
| 3556 | AUGGCUAC G UUGCCCCC | 825 | GGGGGCAA GGCTAGCTACAACGA GTAGCCAT | 1813 |
| 3559 | GCUACGUU G CCCCCCUG | 826 | CAGGGGGG GGCTAGCTACAACGA AACGTAGC | 1814 |
| 3568 | CCCCCCUG A CCUGCAGC | 827 | GCTGCAGG GGCTAGCTACAACGA CAGGGGGG | 1815 |
| 3572 | CCUGACCU G CAGCCCCC | 828 | GGGGGCTG GGCTAGCTACAACGA AGGTCAGG | 1816 |
| 3575 | GACCUGCA G CCCCCAGC | 829 | GCTGGGGG GGCTAGCTACAACGA TGCAGGTC | 1817 |
| 3582 | AGCCCCCA G CCUGAAUA | 830 | TATTCAGG GGCTAGCTACAACGA TGGGGGCT | 1818 |
| 3588 | CAGCCUGA A UAUGUGAA | 831 | TTCACATA GGCTAGCTACAACGA TCAGGCTG | 1819 |
| 3590 | GCCUGAAU A UGUGAACC | 832 | GGTTCACA GGCTAGCTACAACGA ATTCAGGC | 1820 |
| 3592 | CUGAAUAU G UGAACCAG | 833 | CTGGTTCA GGCTAGCTACAACGA ATATTCAG | 1821 |
| 3596 | AUAUGUGA A CCAGCCAG | 834 | CTGGCTGG GGCTAGCTACAACGA TCACATAT | 1822 |
| 3600 | GUGAACCA G CCAGAUGU | 835 | ACATCTGG GGCTAGCTACAACGA TGGTTCAC | 1823 |
| 3605 | CCAGCCAG A UGUUCGGC | 836 | GCCGAACA GGCTAGCTACAACGA CTGGCTGG | 1824 |
| 3607 | AGCCAGAU G UUCGGCCC | 837 | GGGCCGAA GGCTAGCTACAACGA ATCTGGCT | 1825 |
| 3612 | GAUGUUCG G CCCCAGCC | 838 | GGCTGGGG GGCTAGCTACAACGA CGAACATC | 1826 |
| 3618 | CGGCCCCA G CCCCCUUC | 839 | GAAGGGGG GGCTAGCTACAACGA TGGGGCCG | 1827 |
| 3627 | CCCCCUUC G CCCCGAGA | 840 | TCTCGGGG GGCTAGCTACAACGA GAAGGGGG | 1828 |
| 3638 | CCGAGAGG G CCCUCUGC | 841 | GCAGAGGG GGCTAGCTACAACGA CCTCTCGG | 1829 |
| 3645 | GGCCCUCU G CCUGCUGC | 842 | GCAGCAGG GGCTAGCTACAACGA AGAGGGCC | 1830 |
| 3649 | CUCUGCCU G CUGCCCGA | 843 | TCGGGCAG GGCTAGCTACAACGA AGGCAGAG | 1831 |
| 3652 | UGCCUGCU G CCCGACCU | 844 | AGGTCGGG GGCTAGCTACAACGA AGCAGGCA | 1832 |
| 3657 | GCUGCCCG A CCUGCUGG | 845 | CCAGCAGG GGCTAGCTACAACGA CGGGCAGC | 1833 |
| 3661 | CCCGACCU G CUGGUGCC | 846 | GGCACCAG GGCTAGCTACAACGA AGGTCGGG | 1834 |
| 3665 | ACCUGCUG G UGCCACUC | 847 | GAGTGGCA GGCTAGCTACAACGA CAGCAGGT | 1835 |
| 3667 | CUGCUGGU G CCACUCUG | 848 | CAGAGTGG GGCTAGCTACAACGA ACCAGCAG | 1836 |
| 3670 | CUGGUGCC A CUCUGGAA | 849 | TTCCAGAG GGCTAGCTACAACGA GGCACCAG | 1837 |
| 3681 | CUGGAAAG G CCCAAGAC | 850 | GTCTTGGG GGCTAGCTACAACGA CTTTCCAG | 1838 |
| 3688 | GGCCCAAG A CUCUCUCC | 851 | GGAGAGAG GGCTAGCTACAACGA CTTGGGCC | 1839 |
| 3707 | AGGGAAGA A UGGGGUCG | 852 | CGACCCCA GGCTAGCTACAACGA TCTTCCCT | 1840 |
| 3712 | AGAAUGGG G UCGUCAAA | 853 | TTTGACGA GGCTAGCTACAACGA CCCATTCT | 1841 |
| 3715 | AUGGGGUC G UCAAAGAC | 854 | GTCTTTGA GGCTAGCTACAACGA GACCCCAT | 1842 |
| 3722 | CGUCAAAG A CGUUUUUG | 855 | CAAAAACG GGCTAGCTACAACGA CTTTGACG | 1843 |
| 3724 | UCAAAGAC G UUUUUGCC | 856 | GGCAAAAA GGCTAGCTACAACGA GTCTTTGA | 1844 |
| 3730 | ACGUUUUU G CCUUUGGG | 857 | CCCAAAGG GGCTAGCTACAACGA AAAAACGT | 1845 |
| 3740 | CUUUGGGG G UGCCGUGG | 858 | CCACGGCA GGCTAGCTACAACGA CCCCAAAG | 1846 |
| 3742 | UUGGGGGU G CCGUGGAG | 859 | CTCCACGG GGCTAGCTACAACGA ACCCCCAA | 1847 |
| 3745 | GGGGUGCC G UGGAGAAC | 860 | GTTCTCCA GGCTAGCTACAACGA GGCACCCC | 1848 |
| 3752 | CGUGGAGA A CCCCGAGU | 861 | ACTCGGGG GGCTAGCTACAACGA TCTCCACG | 1849 |
| 3759 | AACCCCGA G UACUUGAC | 862 | GTCAAGTA GGCTAGCTACAACGA TCGGGGTT | 1850 |
| 3761 | CCCCGAGU A CUUGACAC | 863 | GTGTCAAG GGCTAGCTACAACGA ACTCGGGG | 1851 |
| 3766 | AGUACUUG A CACCCCAG | 864 | CTGGGGTG GGCTAGCTACAACGA CAAGTACT | 1852 |
| 3768 | UACUUGAC A CCCCAGGG | 865 | CCCTGGGG GGCTAGCTACAACGA GTCAAGTA | 1853 |
| 3781 | AGGGAGGA G CUGCCCCU | 866 | AGGGGCAG GGCTAGCTACAACGA TCCTCCCT | 1854 |
| 3784 | GAGGAGCU G CCCCUCAG | 867 | CTGAGGGG GGCTAGCTACAACGA AGCTCCTC | 1855 |
| 3792 | GCCCCUCA G CCCCACCC | 868 | GGGTGGGG GGCTAGCTACAACGA TGAGGGGC | 1856 |
| 3797 | UCAGCCCC A CCCUCCUC | 869 | GAGGAGGG GGCTAGCTACAACGA GGGGCTGA | 1857 |
| 3808 | CUCCUCCU G CCUUCAGC | 870 | GCTGAAGG GGCTAGCTACAACGA AGGAGGAG | 1858 |
| 3815 | UGCCUUCA G CCCAGCCU | 871 | AGGCTGGG GGCTAGCTACAACGA TGAAGGCA | 1859 |
| 3820 | UCAGCCCA G CCUUCGAC | 872 | GTCGAAGG GGCTAGCTACAACGA TGGGCTGA | 1860 |
| 3827 | AGCCUUCG A CAACCUCU | 873 | AGAGGTTG GGCTAGCTACAACGA CGAAGGCT | 1861 |
| 3830 | CUUCGACA A CCUCUAUU | 874 | AATAGAGG GGCTAGCTACAACGA TGTCGAAG | 1862 |
| 3836 | CAACCUCU A UUACUGGG | 875 | CCCAGTAA GGCTAGCTACAACGA AGAGGTTG | 1863 |
| 3839 | CCUCUAUU A CUGGGACC | 876 | GGTCCCAG GGCTAGCTACAACGA AATAGAGG | 1864 |
| 3845 | UUACUGGG A CCAGGACC | 877 | GGTCCTGG GGCTAGCTACAACGA CCCAGTAA | 1865 |
| 3851 | GGACCAGG A CCCACCAG | 878 | CTGGTGGG GGCTAGCTACAACGA CCTGGTCC | 1866 |
| 3855 | CAGGACCC A CCAGAGCG | 879 | CGCTCTGG GGCTAGCTACAACGA GGGTCCTG | 1867 |
| 3861 | CCACCAGA G CGGGGGGC | 880 | GCCCCCCG GGCTAGCTACAACGA TCTGGTGG | 1868 |
| 3868 | AGCGGGGG G CUCCACCC | 881 | GGGTGGAG GGCTAGCTACAACGA CCCCCGCT | 1869 |
| 3873 | GGGGCUCC A CCCAGCAC | 882 | GTGCTGGG GGCTAGCTACAACGA GGAGCCCC | 1870 |
| 3878 | UCCACCCA G CACCUUCA | 883 | TGAAGGTG GGCTAGCTACAACGA TGGGTGGA | 1871 |
| 3880 | CACCCAGC A CCUUCAAA | 884 | TTTGAAGG GGCTAGCTACAACGA GCTGGGTG | 1872 |
| 3892 | UCAAAGGG A CACCUACG | 885 | CGTAGGTG GGCTAGCTACAACGA CCCTTTGA | 1873 |
| 3894 | AAAGGGAC A CCUACGGC | 886 | GCCGTAGG GGCTAGCTACAACGA GTCCCTTT | 1874 |
| 3898 | GGACACCU A CGGCAGAG | 887 | CTCTGCCG GGCTAGCTACAACGA AGGTGTCC | 1875 |
| 3901 | CACCUACG G CAGAGAAC | 888 | GTTCTCTG GGCTAGCTACAACGA CGTAGGTG | 1876 |
| 3908 | GGCAGAGA A CCCAGAGU | 889 | ACTCTGGG GGCTAGCTACAACGA TCTCTGCC | 1877 |
| 3915 | AACCCAGA G UACCUGGG | 890 | CCCAGGTA GGCTAGCTACAACGA TCTGGGTT | 1878 |
| 3917 | CCCAGAGU A CCUGGGUC | 891 | GACCCAGG GGCTAGCTACAACGA ACTCTGGG | 1879 |
| 3923 | GUACCUGG G UCUGGACG | 892 | CGTCCAGA GGCTAGCTACAACGA CCAGGTAC | 1880 |
| 3929 | GGGUCUGG A CGUGCCAG | 893 | CTGGCACG GGCTAGCTACAACGA CCAGACCC | 1881 |
| 3931 | GUCUGGAC G UGCCAGUG | 894 | CACTGGCA GGCTAGCTACAACGA GTCCAGAC | 1882 |
| 3933 | CUGGACGU G CCAGUGUG | 895 | CACACTGG GGCTAGCTACAACGA ACGTCCAG | 1883 |
| 3937 | ACGUGCCA G UGUGAACC | 896 | GGTTCACA GGCTAGCTACAACGA TGGCACGT | 1884 |
| 3939 | GUGCCAGU G UGAACCAG | 897 | CTGGTTCA GGCTAGCTACAACGA ACTGGCAC | 1885 |
| 3943 | CAGUGUGA A CCAGAAGG | 898 | CCTTCTGG GGCTAGCTACAACGA TCACACTG | 1886 |
| 3951 | ACCAGAAG G CCAAGUCC | 899 | GGACTTGG GGCTAGCTACAACGA CTTCTGGT | 1887 |
| 3956 | AAGGCCAA G UCCGCAGA | 900 | TCTGCGGA GGCTAGCTACAACGA TTGGCCTT | 1888 |
| 3960 | CCAAGUCC G CAGAAGCC | 901 | GGCTTCTG GGCTAGCTACAACGA GGACTTGG | 1889 |
| 3966 | CCGCAGAA G CCCUGAUG | 902 | CATCAGGG GGCTAGCTACAACGA TTCTGCGG | 1890 |
| 3972 | AAGCCCUG A UGUGUCCU | 903 | AGGACACA GGCTAGCTACAACGA CAGGGCTT | 1891 |
| 3974 | GCCCUGAU G UGUCCUCA | 904 | TGAGGACA GGCTAGCTACAACGA ATCAGGGC | 1892 |
| 3976 | CCUGAUGU G UCCUCAGG | 905 | CCTGAGGA GGCTAGCTACAACGA ACATCAGG | 1893 |
| 3987 | CUCAGGGA G CAGGGAAG | 906 | CTTCCCTG GGCTAGCTACAACGA TCCCTGAG | 1894 |
| 3996 | CAGGGAAG G CCUGACUU | 907 | AAGTCAGG GGCTAGCTACAACGA CTTCCCTG | 1895 |
| 4001 | AAGGCCUG A CUUCUGCU | 908 | AGCAGAAG GGCTAGCTACAACGA CAGGCCTT | 1896 |
| 4007 | UGACUUCU G CUGGCAUC | 909 | GATGCCAG GGCTAGCTACAACGA AGAAGTCA | 1897 |
| 4011 | UUCUGCUG G CAUCAAGA | 910 | TCTTGATG GGCTAGCTACAACGA CAGCAGAA | 1898 |
| 4013 | CUGCUGGC A UCAAGAGG | 911 | CCTCTTGA GGCTAGCTACAACGA GCCAGCAG | 1899 |
| 4021 | AUCAAGAG G UGGGAGGG | 912 | CCCTCCCA GGCTAGCTACAACGA CTCTTGAT | 1900 |
| 4029 | GUGGGAGG G CCCUCCGA | 913 | TCGGAGGG GGCTAGCTACAACGA CCTCCCAC | 1901 |
| 4037 | GCCCUCCG A CCACUUCC | 914 | GGAAGTGG GGCTAGCTACAACGA CGGAGGGC | 1902 |
| 4040 | CUCCGACC A CUUCCAGG | 915 | CCTGGAAG GGCTAGCTACAACGA GGTCGGAG | 1903 |
| 4052 | CCAGGGGA A CCUGCCAU | 916 | ATGGCAGG GGCTAGCTACAACGA TCCCCTGG | 1904 |
| 4056 | GGGAACCU G CCAUGCCA | 917 | TGGCATGG GGCTAGCTACAACGA AGGTTCCC | 1905 |
| 4059 | AACCUGCC A UGCCAGGA | 918 | TCCTGGCA GGCTAGCTACAACGA GGCAGGTT | 1906 |
| 4061 | CCUGCCAU G CCAGGAAC | 919 | GTTCCTGG GGCTAGCTACAACGA ATGGCAGG | 1907 |
| 4068 | UGCCAGGA A CCUGUCCU | 920 | AGGACAGG GGCTAGCTACAACGA TCCTGGCA | 1908 |
| 4072 | AGGAACCU G UCCUAAGG | 921 | CCTTAGGA GGCTAGCTACAACGA AGGTTCCT | 1909 |
| 4082 | CCUAAGGA A CCUUCCUU | 922 | AAGGAAGG GGCTAGCTACAACGA TCCTTAGG | 1910 |
| 4094 | UCCUUCCU G CUUGAGUU | 923 | AACTCAAG GGCTAGCTACAACGA AGGAAGGA | 1911 |
| 4100 | CUGCUUGA G UUCCCAGA | 924 | TCTGGGAA GGCTAGCTACAACGA TCAAGCAG | 1912 |
| 4108 | GUUCCCAG A UGGCUGGA | 925 | TCCAGCCA GGCTAGCTACAACGA CTGGGAAC | 1913 |
| 4111 | CCCAGAUG G CUGGAAGG | 926 | CCTTCCAG GGCTAGCTACAACGA CATCTGGG | 1914 |
| 4121 | UGGAAGGG G UCCAGCCU | 927 | AGGCTGGA GGCTAGCTACAACGA CCCTTCCA | 1915 |
| 4126 | GGGGUCCA G CCUCGUUG | 928 | CAACGAGG GGCTAGCTACAACGA TGGACCCC | 1916 |
| 4131 | CCAGCCUC G UUGGAAGA | 929 | TCTTCCAA GGCTAGCTACAACGA GAGGCTGG | 1917 |
| 4143 | GAAGAGGA A CAGCACUG | 930 | CAGTGCTG GGCTAGCTACAACGA TCCTCTTC | 1918 |
| 4146 | GAGGAACA G CACUGGGG | 931 | CCCCAGTG GGCTAGCTACAACGA TGTTCCTC | 1919 |
| 4148 | GGAACAGC A CUGGGGAG | 932 | CTCCCCAG GGCTAGCTACAACGA GCTGTTCC | 1920 |
| 4156 | AGUGGGGA G UCUUUGUG | 933 | CACAAAGA GGCTAGCTACAACGA TCCCCAGT | 1921 |
| 4162 | GAGUCUUU G UGGAUUCU | 934 | AGAATCCA GGCTAGCTACAACGA AAAGACTC | 1922 |
| 4166 | CUUUGUGG A UUCUGAGG | 935 | CCTCAGAA GGCTAGCTACAACGA CCACAAAG | 1923 |
| 4174 | AUUCUGAG G CCCUGCCC | 936 | GGGCAGGG GGCTAGCTACAACGA CTCAGAAT | 1924 |
| 4179 | GAGGCCCU G CCCAAUGA | 937 | TCATTGGG GGCTAGCTACAACGA AGGGCCTC | 1925 |
| 4184 | CCUGCCCA A UGAGACUC | 928 | GAGTCTCA GGCTAGCTACAACGA TGGGCAGG | 1926 |
| 4189 | CCAAUGAG A CUCUAGGG | 939 | CCCTAGAG GGCTAGCTACAACGA CTCATTGG | 1927 |
| 4197 | ACUCUAGG G UCCAGUGG | 940 | CCACTGGA GGCTAGCTACAACGA CCTAGAGT | 1928 |
| 4202 | AGGGUCCA G UGGAUGCC | 941 | GGCATCCA GGCTAGCTACAACGA TGGACCCT | 1929 |
| 4206 | UCCAGUGG A UGCCACAG | 942 | CTGTGGCA GGCTAGCTACAACGA CCACTGGA | 1930 |
| 4208 | CAGUGGAU G CCACAGCC | 943 | GGCTGTGG GGCTAGCTACAACGA ATCCACTG | 1931 |
| 4211 | UGGAUGCC A CAGCCCAG | 944 | CTGGGCTG GGCTAGCTACAACGA GGCATCCA | 1932 |
| 4214 | AUGCCACA G CCCAGCUU | 945 | AAGCTGGG GGCTAGCTACAACGA TGTGGCAT | 1933 |
| 4219 | ACAGCCCA G CUUGGCCC | 946 | GGGCCAAG GGCTAGCTACAACGA TGGGCTGT | 1934 |
| 4224 | CCAGCUUG G CCCUUUCC | 947 | GGAAAGGG GGCTAGCTACAACGA CAAGCTGG | 1935 |
| 4239 | CCUUCCAG A UCCUGGGU | 948 | ACCCAGGA GGCTAGCTACAACGA CTGGAAGG | 1936 |
| 4246 | GAUCCUGG G UACUGAAA | 949 | TTTCAGTA GGCTAGCTACAACGA CCAGGATC | 1937 |
| 4248 | UCCUGGGU A CUGAAAGC | 950 | GCTTTCAG GGCTAGCTACAACGA ACCCAGGA | 1938 |
| 4255 | UACUGAAA G CCUUAGGG | 951 | CCCTAAGG GGCTAGCTACAACGA TTTCAGTA | 1939 |
| 4266 | UUAGGGAA G CUGGCCUG | 952 | CAGGCCAG GGCTAGCTACAACGA TTCCCTAA | 1940 |
| 4270 | GGAAGCUG G CCUGAGAG | 953 | CTCTCAGG GGCTAGCTACAACGA CAGCTTCC | 1941 |
| 4284 | GAGGGGAA G CGGCCCUA | 954 | TAGGGCCG GGCTAGCTACAACGA TTCCCCTC | 1942 |
| 4287 | GGGAAGCG G CCCUAAGG | 955 | CCTTAGGG GGCTAGCTACAACGA CGCTTCCC | 1943 |
| 4298 | CUAAGGGA G UGUCUAAG | 956 | CTTAGACA GGCTAGCTACAACGA TCCCTTAG | 1944 |
| 4300 | AAGGGAGU G UCUAAGAA | 957 | TTCTTAGA GGCTAGCTACAACGA ACTCCCTT | 1945 |
| 4308 | GUCUAAGA A CAAAAGCG | 958 | CGCTTTTG GGCTAGCTACAACGA TCTTAGAC | 1946 |
| 4314 | GAACAAAA G CGACCCAU | 959 | ATGGGTCG GGCTAGCTACAACGA TTTTGTTC | 1947 |
| 4317 | CAAAAGCG A CCCAUUCA | 960 | TGAATGGG GGCTAGCTACAACGA CGCTTTTG | 1948 |
| 4321 | AGCGACCC A UUCAGAGA | 961 | TCTCTGAA GGCTAGCTACAACGA GGGTCGCT | 1949 |
| 4329 | AUUCAGAG A CUGUCCCU | 962 | AGGGACAG GGCTAGCTACAACGA CTCTGAAT | 1950 |
| 4332 | CAGAGACU G UCCCUGAA | 963 | TTCAGGGA GGCTAGCTACAACGA AGTCTCTG | 1951 |
| 4341 | UCCCUGAA A CCUAGUAC | 964 | GTACTAGG GGCTAGCTACAACGA TTCAGGGA | 1952 |
| 4346 | GAAACCUA G UACUGCCC | 965 | GGGCAGTA GGCTAGCTACAACGA TAGGTTTC | 1953 |
| 4348 | AACCUAGU A CUGCCCCC | 966 | GGGGGCAG GGCTAGCTACAACGA ACTAGGTT | 1954 |
| 4351 | CUAGUACU G CCCCCCAU | 967 | ATGGGGGG GGCTAGCTACAACGA AGTACTAG | 1955 |
| 4358 | UGCCCCCC A UGAGGAAG | 968 | CTTCCTCA GGCTAGCTACAACGA GGGGGGCA | 1956 |
| 4369 | AGGAAGGA A CAGCAAUG | 969 | CATTGCTG GGCTAGCTACAACGA TCCTTCCT | 1957 |
| 4372 | AAGGAACA G CAAUGGUG | 970 | CACCATTG GGCTAGCTACAACGA TGTTCCTT | 1958 |
| 4375 | GAACAGCA A UGGUGUCA | 971 | TGACACCA GGCTAGCTACAACGA TGCTGTTC | 1959 |
| 4378 | CAGCAAUG G UGUCAGUA | 972 | TACTGACA GGCTAGCTACAACGA CATTGCTG | 1960 |
| 4380 | GCAAUGGU G UCAGUAUC | 973 | GATACTGA GGCTAGCTACAACGA ACCATTGC | 1961 |
| 4384 | UGGUGUCA G UAUCCAGG | 974 | CCTGGATA GGCTAGCTACAACGA TGACACCA | 1962 |
| 4386 | GUGUCAGU A UCCAGGCU | 975 | AGCCTGGA GGCTAGCTACAACGA ACTGACAC | 1963 |
| 4392 | GUAUCCAG G CUUUGUAC | 976 | GTACAAAG GGCTAGCTACAACGA CTGGATAC | 1964 |
| 4397 | CAGGCUUU G UACAGAGU | 977 | ACTCTGTA GGCTAGCTACAACGA AAAGCCTG | 1965 |
| 4399 | GGCUUUGU A CAGAGUGC | 978 | GCACTCTG GGCTAGCTACAACGA ACAAAGCC | 1966 |
| 4404 | UGUACAGA G UGCUUUUC | 979 | GAAAAGCA GGCTAGCTACAACGA TCTGTACA | 1967 |
| 4406 | UACAGAGU G CUUUUCUG | 980 | CAGAAAAG GGCTAGCTACAACGA ACTCTGTA | 1968 |
| 4414 | GCUUUUCU G UUUAGUUU | 981 | AAACTAAA GGCTAGCTACAACGA AGAAAAGC | 1969 |
| 4419 | UCUGUUUA G UUUUUACU | 982 | AGTAAAAA GGCTAGCTACAACGA TAAACAGA | 1970 |
| 4425 | UAGUUUUU A CUUUUUUU | 983 | AAAAAAAG GGCTAGCTACAACGA AAAAACTA | 1971 |
| 4434 | CUUUUUUU G UUUUGUUU | 984 | AAACAAAA GGCTAGCTACAACGA AAAAAAAG | 1972 |
| 4439 | UUUGUUUU G UUUUUUUA | 985 | TAAAAAAA GGCTAGCTACAACGA AAAACAAA | 1973 |
| 4451 | UUUUAAAG A UGAAAUAA | 986 | TTATTTCA GGCTAGCTACAACGA CTTTAAAA | 1974 |
| 4456 | AAGAUGAA A UAAAGACC | 987 | GGTCTTTA GGCTAGCTACAACGA TTCATCTT | 1975 |
| 4462 | AAAUAAAG A CCCAGGGG | 988 | CCCCTGGG GGCTAGCTACAACGA CTTTATTT | 1976 |

TABLE IV
Human HER2 Synthetic DNAzyme and Target molecules

| | | | | | | |
|---|---|---|---|---|---|---|
| | | | Seq | | | Seq |
| Gene | Pos | Target | ID | RPI# | DNAzyme | ID |

| | | | | | | |
|---|---|---|---|---|---|---|
| erbB2 | 377 | CCACCA A UGCCAG | 1977 | 24998 | cuggca GGCTAGCTACAACGA uggugg B | 1982 |
| erbB2 | 766 | UUCUCCG A UGUGUAA | 1978 | 24999 | uuacaca GGCTAGCTACAACGA cggagaa B | 1983 |
| erbB2 | 1202 | UGUGCU A UGGUCU | 1979 | 25000 | agacca GGCTAGCTACAACGA agcaca B | 1984 |
| erbB2 | 1444 | CCUCAGC G UCUUCCA | 1980 | 25001 | uggaaga GGCTAGCTACAACGA gcugagg B | 1985 |
| erbB2 | 1583 | AUCCACC A UAACACC | 1981 | 25002 | gguguua GGCTAGCTACAACGA gguggau B | 1986 |

A, G, C, T (italic) = deoxy
lower case = 2′-O-methyl
B = inverted deoxyabasic derivative

What we claim is:

1. A siRNA nucleic acid molecule that modulates expression of a nucleic acid molecule encoding HER2.

2. An enzymatic nucleic acid molecule that modulates expression of a nucleic acid molecule encoding HER2.

3. An enzymatic nucleic acid molecule comprising a sequence selected from the group consisting of SEQ ID NOs: 989-1976 and 1982-1986.

4. An enzymatic nucleic acid molecule comprising at least one binding arm wherein one or more of said binding arms comprises a sequence complementary to a sequence selected from the group consisting of SEQ ID NOs: 1-988 and 1977-1981.

5. A siRNA nucleic acid molecule comprising a sequence complementary to a sequence selected from the group consisting of SEQ ID NOs: 1-988 and 1977-1981.

6. The nucleic acid molecule of any of claims 1-5, wherein said nucleic acid molecule is adapted to treat cancer.

7. The enzymatic nucleic acid molecule of any of claims 2-4, wherein said enzymatic nucleic acid molecule has an endonuclease activity to cleave RNA having HER2 sequence.

8. The enzymatic nucleic acid molecule of claim 2, wherein said enzymatic nucleic acid molecule is a DNAzyme in a 10-23 configuration.

9. The enzymatic nucleic acid molecule of claim 8, wherein said enzymatic nucleic acid molecule comprises a sequence complementary to a sequence selected from the group consisting of SEQ ID NOs: 1-988 and 1977-1981.

10. The enzymatic nucleic acid molecule of claim 8, wherein said enzymatic nucleic acid molecule comprises a sequence selected from the group consisting of SEQ ID NOs: 989-1976 and 1982-1986.

11. The nucleic acid molecule of any of claims 1, 2, 4 or 5, wherein said nucleic acid molecule comprises between 12 and 100 bases complementary to a RNA having HER2 sequence.

12. The nucleic acid molecule of claim of any of claims 1, 2, 4 or 5, wherein said nucleic acid molecule comprises between 14 and 24 bases complementary to a RNA having HER2 sequence.

13. The nucleic acid molecule of any of claims 1-5, wherein said nucleic acid molecule is chemically synthesized.

14. The nucleic acid molecule of any of claims 1-5, wherein said nucleic acid molecule comprises at least one 2′-sugar modification.

15. The nucleic acid molecule of any of claims 1-5, wherein said nucleic acid molecule comprises at least one nucleic acid base modification.

16. The nucleic acid molecule of any of claims 1-5, wherein said nucleic acid molecule comprises at least one phosphate backbone modification.

17. A mammalian cell comprising the nucleic acid molecule of any of claims 1-5.

18. The mammalian cell of claim 17, wherein said mammalian cell is a human cell.

19. A method of reducing HER2 activity in a cell, comprising contacting said cell with the nucleic acid molecule of any of claims 1-5, under conditions suitable for said reduction of HER2 activity.

20. A method of treatment of a subject having a condition associated with the level of HER2, comprising contacting cells of said subject with the nucleic acid molecule of any of claims 1-5, under conditions suitable for said treatment.

21. The method of claim 20 further comprising the use of one or more drug therapies under conditions suitable for said treatment.

22. A method of cleaving RNA having HER2 sequence comprising contacting an enzymatic nucleic acid molecule of any of claims 2-4 with said RNA under conditions suitable for the cleavage.

23. The method of claim 22, wherein said cleavage is carried out in the presence of a divalent cation.

24. The method of claim 23, wherein said divalent cation is Mg²⁺.

25. The nucleic acid molecule of any of claims 1-5, wherein said nucleic acid molecule comprises a cap structure, wherein the cap structure is at the 5′-end, 3′-end, or both the 5′-end and the 3′-end of said nucleic acid molecule.

26. The nucleic acid molecule of claim 25, wherein the cap structure at the 5′-end, 3′-end, or both the 5′-end and the 3′-end comprises a 3′,3′-linked or 5′,5′-linked deoxyabasic ribose derivative.

27. An expression vector comprising a nucleic acid sequence encoding at least one nucleic acid molecule of any of claims 1-5 in a manner that allows expression of the nucleic acid molecule.

28. A mammalian cell comprising an expression vector of claim 27.

29. The mammalian cell of claim 28, wherein said mammalian cell is a human cell.

30. The expression vector of claim 27, wherein said nucleic acid molecule is in a DNAzyme configuration.

31. The expression vector of claim 27, wherein said expression vector further comprises a sequence for a nucleic acid molecule complementary to a nucleic acid molecule having HER2 sequence.

32. The expression vector of claim 27, wherein said expression vector comprises a nucleic acid sequence encoding two or more of said nucleic acid molecules, which may be the same or different.

33. The expression vector of claim 32, wherein said expression vector further comprises a sequence encoding an antisense nucleic acid molecule or siRNA molecule complementary to a nucleic acid molecule having HER2 sequence.

34. A method for treatment of cancer comprising administering to a subject the nucleic acid molecule of any of claims 1-5 under conditions suitable for said treatment.

35. The method of claim 34, wherein said cancer is breast cancer.

36. The method of claim 34, wherein said cancer is ovarian cancer.

37. The method of claim 34, wherein said method further comprises administering to said subject one or more other therapies under conditions suitable for said treatment.

38. The method of claim 21 wherein said other drug therapies are chosen from the group consisting of monoclonal antibody therapy, chemotherapy, radiation therapy, and analgesic therapy.

39. The method of claim 37 wherein said other drug therapies are chosen from the group consisting of monoclonal antibody therapy, chemotherapy, radiation therapy, and analgesic therapy.

40. The method of claim 38, wherein said chemotherapy is selected from the group consisting of paclitaxel (Taxol), docetaxel, cisplatin, methotrexate, cyclophosphamide, doxorubin, fluorouracil carboplatin, edatrexate, gemcitabine, and vinorelbine.

41. The method of claim 38, wherein said monoclonal antibody is Herceptin (trastuzumab).

42. The method of claim 39, wherein said chemotherapy is selected from the group consisting of paclitaxel (Taxol), docetaxel, cisplatin, methotrexate, cyclophosphamide, doxorubin, fluorouracil carboplatin, edatrexate, gemcitabine, and vinorelbine.

43. The method of claim 39, wherein said monoclonal antibody is Herceptin (trastuzumab).

44. A composition comprising a nucleic acid molecule of any of claims 1-5 in a pharmaceutically acceptable carrier.

45. A method of administering to a cell a nucleic acid molecule of any of claims 1-5 comprising contacting said cell with the nucleic acid molecule under conditions suitable for said administration.

46. The method of claim 45, wherein said cell is a mammalian cell.

47. The method of claim 45, wherein said cell is a human cell.

48. The method of claim 45, wherein said administration is in the presence of a delivery reagent.

49. The method of claim 48, wherein said delivery reagent is a lipid.

50. The method of claim 49, wherein said lipid is a cationic lipid.

51. The method of claim 49, wherein said lipid is a phospholipid.

52. The method of claim 48, wherein said delivery reagent is a liposome.


SEQUENCE LISTING (1997 sequences)

SEQ ID NO: 1 - RNA, 17 nt, Homo sapiens
aaggggagguaacccug

SEQ ID NO: 2 - RNA, 17 nt, Homo sapiens
gggagguaacccuggcc

SEQ ID NO: 3 - RNA, 17 nt, Homo sapiens
uaacccuggccccuuug

SEQ ID NO: 4 - RNA, 17 nt, Homo sapiens
ccccuuuggucggggcc

SEQ ID NO: 5 - RNA, 17 nt, Homo sapiens
uggucggggccccgggc

SEQ ID NO: 6 - RNA, 17 nt, Homo sapiens
ggccccgggcagccgcg

SEQ ID NO: 7 - RNA, 17 nt, Homo sapiens
cccgggcagccgcgcgc

SEQ ID NO: 8 - RNA, 17 nt, Homo sapiens
gggcagccgcgcgcccc

SEQ ID NO: 9 - RNA, 17 nt, Homo sapiens
gcagccgcgcgccccuu

SEQ ID NO: 10 - RNA, 17 nt, Homo sapiens
agccgcgcgccccuucc

SEQ ID NO: 11 - RNA, 17 nt, Homo sapiens
cccuucccacggggccc

SEQ ID NO: 12 - RNA, 17 nt, Homo sapiens
cccacggggcccuuuac

SEQ ID NO: 13 - RNA, 17 nt, Homo sapiens
ggcccuuuacugcgccg

SEQ ID NO: 14 - RNA, 17 nt, Homo sapiens
ccuuuacugcgccgcgc

SEQ ID NO: 15 - RNA, 17 nt, Homo sapiens
uuuacugcgccgcgcgc

SEQ ID NO: 16 - RNA, 17 nt, Homo sapiens
acugcgccgcgcgcccg

SEQ ID NO: 17 - RNA, 17 nt, Homo sapiens
ugcgccgcgcgcccggc

SEQ ID NO: 18 - RNA, 17 nt, Homo sapiens
cgccgcgcgcccggccc

SEQ ID NO: 19 - RNA, 17 nt, Homo sapiens
cgcgcccggcccccacc

SEQ ID NO: 20 - RNA, 17 nt, Homo sapiens
cggcccccaccccucgc

SEQ ID NO: 21 - RNA, 17 nt, Homo sapiens
caccccucgcagcaccc

SEQ ID NO: 22 - RNA, 17 nt, Homo sapiens
cccucgcagcaccccgc

SEQ ID NO: 23 - RNA, 17 nt, Homo sapiens
cucgcagcaccccgcgc

SEQ ID NO: 24 - RNA, 17 nt, Homo sapiens
agcaccccgcgccccgc

SEQ ID NO: 25 - RNA, 17 nt, Homo sapiens
caccccgcgccccgcgc

SEQ ID NO: 26 - RNA, 17 nt, Homo sapiens
cgcgccccgcgcccucc

SEQ ID NO: 27 - RNA, 17 nt, Homo sapiens
cgccccgcgcccuccca

SEQ ID NO: 28 - RNA, 17 nt, Homo sapiens
cccucccagccgggucc

SEQ ID NO: 29 - RNA, 17 nt, Homo sapiens
ccagccggguccagccg

SEQ ID NO: 30 - RNA, 17 nt, Homo sapiens
cggguccagccggagcc

SEQ ID NO: 31 - RNA, 17 nt, Homo sapiens
cagccggagccaugggg

SEQ ID NO: 32 - RNA, 17 nt, Homo sapiens
ccggagccauggggccg

SEQ ID NO: 33 - RNA, 17 nt, Homo sapiens
gccauggggccggagcc

SEQ ID NO: 34 - RNA, 17 nt, Homo sapiens
gggccggagccgcagug

SEQ ID NO: 35 - RNA, 17 nt, Homo sapiens
ccggagccgcagugagc

SEQ ID NO: 36 - RNA, 17 nt, Homo sapiens
gagccgcagugagcacc

SEQ ID NO: 37 - RNA, 17 nt, Homo sapiens
cgcagugagcaccaugg

SEQ ID NO: 38 - RNA, 17 nt, Homo sapiens
cagugagcaccauggag

SEQ ID NO: 39 - RNA, 17 nt, Homo sapiens
ugagcaccauggagcug

SEQ ID NO: 40 - RNA, 17 nt, Homo sapiens
accauggagcuggcggc

SEQ ID NO: 41 - RNA, 17 nt, Homo sapiens
uggagcuggcggccuug

SEQ ID NO: 42 - RNA, 17 nt, Homo sapiens
agcuggcggccuugugc

SEQ ID NO: 43 - RNA, 17 nt, Homo sapiens
gcggccuugugccgcug

SEQ ID NO: 44 - RNA, 17 nt, Homo sapiens
ggccuugugccgcuggg

SEQ ID NO: 45 - RNA, 17 nt, Homo sapiens
cuugugccgcugggggc

SEQ ID NO: 46 - RNA, 17 nt, Homo sapiens
cgcugggggcuccuccu

SEQ ID NO: 47 - RNA, 17 nt, Homo sapiens
uccuccucgcccucuug

SEQ ID NO: 48 - RNA, 17 nt, Homo sapiens
gcccucuugccccccgg

SEQ ID NO: 49 - RNA, 17 nt, Homo sapiens
cccccggagccgcgagc

SEQ ID NO: 50 - RNA, 17 nt, Homo sapiens
ccggagccgcgagcacc

SEQ ID NO: 51 - RNA, 17 nt, Homo sapiens
agccgcgagcacccaag

SEQ ID NO: 52 - RNA, 17 nt, Homo sapiens
ccgcgagcacccaagug

SEQ ID NO: 53 - RNA, 17 nt, Homo sapiens
gcacccaagugugcacc

SEQ ID NO: 54 - RNA, 17 nt, Homo sapiens
acccaagugugcaccgg

SEQ ID NO: 55 - RNA, 17 nt, Homo sapiens
ccaagugugcaccggca

SEQ ID NO: 56 - RNA, 17 nt, Homo sapiens
aagugugcaccggcaca

SEQ ID NO: 57 - RNA, 17 nt, Homo sapiens
gugcaccggcacagaca

SEQ ID NO: 58 - RNA, 17 nt, Homo sapiens
gcaccggcacagacaug

SEQ ID NO: 59 - RNA, 17 nt, Homo sapiens
cggcacagacaugaagc

SEQ ID NO: 60 - RNA, 17 nt, Homo sapiens
gcacagacaugaagcug

SEQ ID NO: 61 - RNA, 17 nt, Homo sapiens
gacaugaagcugcggcu

SEQ ID NO: 62 - RNA, 17 nt, Homo sapiens
augaagcugcggcuccc

SEQ ID NO: 63 - RNA, 17 nt, Homo sapiens
aagcugcggcucccugc

SEQ ID NO: 64 - RNA, 17 nt, Homo sapiens
ggcucccugccaguccc

SEQ ID NO: 65 - RNA, 17 nt, Homo sapiens
cccugccagucccgaga

SEQ ID NO: 66 - RNA, 17 nt, Homo sapiens
gucccgagacccaccug

SEQ ID NO: 67 - RNA, 17 nt, Homo sapiens
cgagacccaccuggaca

SEQ ID NO: 68 - RNA, 17 nt, Homo sapiens
ccaccuggacaugcucc

SEQ ID NO: 69 - RNA, 17 nt, Homo sapiens
accuggacaugcuccgc

SEQ ID NO: 70 - RNA, 17 nt, Homo sapiens
cuggacaugcuccgcca

SEQ ID NO: 71 - RNA, 17 nt, Homo sapiens
caugcuccgccaccucu

SEQ ID NO: 72 - RNA, 17 nt, Homo sapiens
gcuccgccaccucuacc

SEQ ID NO: 73 - RNA, 17 nt, Homo sapiens
ccaccucuaccagggcu

SEQ ID NO: 74 - RNA, 17 nt, Homo sapiens
cuaccagggcugccagg

SEQ ID NO: 75 - RNA, 17 nt, Homo sapiens
ccagggcugccaggugg

SEQ ID NO: 76 - RNA, 17 nt, Homo sapiens
gcugccagguggugcag

SEQ ID NO: 77 - RNA, 17 nt, Homo sapiens
gccagguggugcaggga

SEQ ID NO: 78 - RNA, 17 nt, Homo sapiens
cagguggugcagggaaa

SEQ ID NO: 79 - RNA, 17 nt, Homo sapiens
gcagggaaaccuggaac

SEQ ID NO: 80 - RNA, 17 nt, Homo sapiens
aaccuggaacucaccua

SEQ ID NO: 81 - RNA, 17 nt, Homo sapiens
uggaacucaccuaccug

SEQ ID NO: 82 - RNA, 17 nt, Homo sapiens
acucaccuaccugccca

SEQ ID NO: 83 - RNA, 17 nt, Homo sapiens
accuaccugcccaccaa

SEQ ID NO: 84 - RNA, 17 nt, Homo sapiens
accugcccaccaaugcc

SEQ ID NO: 85 - RNA, 17 nt, Homo sapiens
gcccaccaaugccagcc

SEQ ID NO: 86 - RNA, 17 nt, Homo sapiens
ccaccaaugccagccug

SEQ ID NO: 87 - RNA, 17 nt, Homo sapiens
caaugccagccuguccu

SEQ ID NO: 88 - RNA, 17 nt, Homo sapiens
gccagccuguccuuccu

SEQ ID NO: 89 - RNA, 17 nt, Homo sapiens
uccuuccugcaggauau

SEQ ID NO: 90 - RNA, 17 nt, Homo sapiens
ccugcaggauauccagg

SEQ ID NO: 91 - RNA, 17 nt, Homo sapiens
ugcaggauauccaggag

SEQ ID NO: 92 - RNA, 17 nt, Homo sapiens
uccaggaggugcagggc

SEQ ID NO: 93 - RNA, 17 nt, Homo sapiens
caggaggugcagggcua

SEQ ID NO: 94 - RNA, 17 nt, Homo sapiens
ggugcagggcuacgugc

SEQ ID NO: 95 - RNA, 17 nt, Homo sapiens
gcagggcuacgugcuca

SEQ ID NO: 96 - RNA, 17 nt, Homo sapiens
agggcuacgugcucauc

SEQ ID NO: 97 - RNA, 17 nt, Homo sapiens
ggcuacgugcucaucgc

SEQ ID NO: 98 - RNA, 17 nt, Homo sapiens
acgugcucaucgcucac

SEQ ID NO: 99 - RNA, 17 nt, Homo sapiens
ugcucaucgcucacaac

SEQ ID NO: 100 - RNA, 17 nt, Homo sapiens
caucgcucacaaccaag

SEQ ID NO: 101 - RNA, 17 nt, Homo sapiens
cgcucacaaccaaguga

SEQ ID NO: 102 - RNA, 17 nt, Homo sapiens
acaaccaagugaggcag

SEQ ID NO: 103 - RNA, 17 nt, Homo sapiens
caagugaggcagguccc

SEQ ID NO: 104 - RNA, 17 nt, Homo sapiens
ugaggcaggucccacug

SEQ ID NO: 105 - RNA, 17 nt, Homo sapiens
caggucccacugcagag

SEQ ID NO: 106 - RNA, 17 nt, Homo sapiens
gucccacugcagaggcu

SEQ ID NO: 107 - RNA, 17 nt, Homo sapiens
cugcagaggcugcggau

SEQ ID NO: 108 - RNA, 17 nt, Homo sapiens
cagaggcugcggauugu

SEQ ID NO: 109 - RNA, 17 nt, Homo sapiens
ggcugcggauugugcga

SEQ ID NO: 110 - RNA, 17 nt, Homo sapiens
ugcggauugugcgaggc

SEQ ID NO: 111 - RNA, 17 nt, Homo sapiens
cggauugugcgaggcac

SEQ ID NO: 112 - RNA, 17 nt, Homo sapiens
ugugcgaggcacccagc

SEQ ID NO: 113 - RNA, 17 nt, Homo sapiens
ugcgaggcacccagcuc

SEQ ID NO: 114 - RNA, 17 nt, Homo sapiens
ggcacccagcucuuuga

SEQ ID NO: 115 - RNA, 17 nt, Homo sapiens
cuuugaggacaacuaug

SEQ ID NO: 116 - RNA, 17 nt, Homo sapiens
ugaggacaacuaugccc

SEQ ID NO: 117 - RNA, 17 nt, Homo sapiens
ggacaacuaugcccugg

SEQ ID NO: 118 - RNA, 17 nt, Homo sapiens
acaacuaugcccuggcc

SEQ ID NO: 119 - RNA, 17 nt, Homo sapiens
augcccuggccgugcua

SEQ ID NO: 120 - RNA, 17 nt, Homo sapiens
cccuggccgugcuagac

SEQ ID NO: 121 - RNA, 17 nt, Homo sapiens
cuggccgugcuagacaa

SEQ ID NO: 122 - RNA, 17 nt, Homo sapiens
cgugcuagacaauggag

SEQ ID NO: 123 - RNA, 17 nt, Homo sapiens
gcuagacaauggagacc

SEQ ID NO: 124 - RNA, 17 nt, Homo sapiens
caauggagacccgcuga

SEQ ID NO: 125 - RNA, 17 nt, Homo sapiens
ggagacccgcugaacaa

SEQ ID NO: 126 - RNA, 17 nt, Homo sapiens
cccgcugaacaauacca

SEQ ID NO: 127 - RNA, 17 nt, Homo sapiens
gcugaacaauaccaccc

SEQ ID NO: 128 - RNA, 17 nt, Homo sapiens
ugaacaauaccaccccu

SEQ ID NO: 129 - RNA, 17 nt, Homo sapiens
acaauaccaccccuguc

SEQ ID NO: 130 - RNA, 17 nt, Homo sapiens
ccaccccugucacaggg

SEQ ID NO: 131 - RNA, 17 nt, Homo sapiens
ccccugucacaggggcc

SEQ ID NO: 132 - RNA, 17 nt, Homo sapiens
ucacaggggccucccca

SEQ ID NO: 133 - RNA, 17 nt, Homo sapiens
cccaggaggccugcggg

SEQ ID NO: 134 - RNA, 17 nt, Homo sapiens
ggaggccugcgggagcu

SEQ ID NO: 135 - RNA, 17 nt, Homo sapiens
cugcgggagcugcagcu

SEQ ID NO: 136 - RNA, 17 nt, Homo sapiens
cgggagcugcagcuucg

SEQ ID NO: 137 - RNA, 17 nt, Homo sapiens
gagcugcagcuucgaag

SEQ ID NO: 138 - RNA, 17 nt, Homo sapiens
gcuucgaagccucacag

SEQ ID NO: 139 - RNA, 17 nt, Homo sapiens
gaagccucacagagauc

SEQ ID NO: 140 - RNA, 17 nt, Homo sapiens
ucacagagaucuugaaa

SEQ ID NO: 141 - RNA, 17 nt, Homo sapiens
aaggaggggucuugauc

SEQ ID NO: 142 - RNA, 17 nt, Homo sapiens
gggucuugauccagcgg

SEQ ID NO: 143 - RNA, 17 nt, Homo sapiens
uugauccagcggaaccc

SEQ ID NO: 144 - RNA, 17 nt, Homo sapiens
ccagcggaacccccagc

SEQ ID NO: 145 - RNA, 17 nt, Homo sapiens
aacccccagcucugcua

SEQ ID NO: 146 - RNA, 17 nt, Homo sapiens
ccagcucugcuaccagg

SEQ ID NO: 147 - RNA, 17 nt, Homo sapiens
gcucugcuaccaggaca

SEQ ID NO: 148 - RNA, 17 nt, Homo sapiens
cuaccaggacacgauuu

SEQ ID NO: 149 - RNA, 17 nt, Homo sapiens
accaggacacgauuuug

SEQ ID NO: 150 - RNA, 17 nt, Homo sapiens
aggacacgauuuugugg

SEQ ID NO: 151 - RNA, 17 nt, Homo sapiens
acgauuuuguggaagga

SEQ ID NO: 152 - RNA, 17 nt, Homo sapiens
guggaaggacaucuucc

SEQ ID NO: 153 - RNA, 17 nt, Homo sapiens
ggaaggacaucuuccac

SEQ ID NO: 154 - RNA, 17 nt, Homo sapiens
caucuuccacaagaaca

SEQ ID NO: 155 - RNA, 17 nt, Homo sapiens
ccacaagaacaaccagc

SEQ ID NO: 156 - RNA, 17 nt, Homo sapiens
caagaacaaccagcugg

SEQ ID NO: 157 - RNA, 17 nt, Homo sapiens
aacaaccagcuggcucu

SEQ ID NO: 158 - RNA, 17 nt, Homo sapiens
accagcuggcucucaca

SEQ ID NO: 159 - RNA, 17 nt, Homo sapiens
uggcucucacacugaua

SEQ ID NO: 160 - RNA, 17 nt, Homo sapiens
gcucucacacugauaga

SEQ ID NO: 161 - RNA, 17 nt, Homo sapiens
ucacacugauagacacc

SEQ ID NO: 162 - RNA, 17 nt, Homo sapiens
acugauagacaccaacc

SEQ ID NO: 163 - RNA, 17 nt, Homo sapiens
ugauagacaccaaccgc

SEQ ID NO: 164 - RNA, 17 nt, Homo sapiens
agacaccaaccgcucuc

SEQ ID NO: 165 - RNA, 17 nt, Homo sapiens
caccaaccgcucucggg

SEQ ID NO: 166 - RNA, 17 nt, Homo sapiens
gcucucgggccugccac

SEQ ID NO: 167 - RNA, 17 nt, Homo sapiens
ucgggccugccaccccu

SEQ ID NO: 168 - RNA, 17 nt, Homo sapiens
ggccugccaccccuguu

SEQ ID NO: 169 - RNA, 17 nt, Homo sapiens
ccaccccuguucuccga

SEQ ID NO: 170 - RNA, 17 nt, Homo sapiens
guucuccgauguguaag

SEQ ID NO: 171 - RNA, 17 nt, Homo sapiens
ucuccgauguguaaggg

SEQ ID NO: 172 - RNA, 17 nt, Homo sapiens
uccgauguguaagggcu

SEQ ID NO: 173 - RNA, 17 nt, Homo sapiens
guguaagggcucccgcu

SEQ ID NO: 174 - RNA, 17 nt, Homo sapiens
gggcucccgcugcuggg

SEQ ID NO: 175 - RNA, 17 nt, Homo sapiens
cucccgcugcuggggag

SEQ ID NO: 176 - RNA, 17 nt, Homo sapiens
gggagagaguucugagg

SEQ ID NO: 177 - RNA, 17 nt, Homo sapiens
uucugaggauugucaga

SEQ ID NO: 178 - RNA, 17 nt, Homo sapiens
ugaggauugucagagcc

SEQ ID NO: 179 - RNA, 17 nt, Homo sapiens
uugucagagccugacgc

SEQ ID NO: 180 - RNA, 17 nt, Homo sapiens
agagccugacgcgcacu

SEQ ID NO: 181 - RNA, 17 nt, Homo sapiens
agccugacgcgcacugu

SEQ ID NO: 182 - RNA, 17 nt, Homo sapiens
ccugacgcgcacugucu

SEQ ID NO: 183 - RNA, 17 nt, Homo sapiens
ugacgcgcacugucugu

SEQ ID NO: 184 - RNA, 17 nt, Homo sapiens
cgcgcacugucugugcc

SEQ ID NO: 185 - RNA, 17 nt, Homo sapiens
cacugucugugccggug

SEQ ID NO: 186 - RNA, 17 nt, Homo sapiens
cugucugugccgguggc

SEQ ID NO: 187 - RNA, 17 nt, Homo sapiens
cugugccgguggcugug

SEQ ID NO: 188 - RNA, 17 nt, Homo sapiens
ugccgguggcugugccc

SEQ ID NO: 189 - RNA, 17 nt, Homo sapiens
cgguggcugugcccgcu

SEQ ID NO: 190 - RNA, 17 nt, Homo sapiens
guggcugugcccgcugc

SEQ ID NO: 191 - RNA, 17 nt, Homo sapiens
cugugcccgcugcaagg

SEQ ID NO: 192 - RNA, 17 nt, Homo sapiens
ugcccgcugcaaggggc

SEQ ID NO: 193 - RNA, 17 nt, Homo sapiens
ugcaaggggccacugcc

SEQ ID NO: 194 - RNA, 17 nt, Homo sapiens
aaggggccacugcccac

SEQ ID NO: 195 - RNA, 17 nt, Homo sapiens
gggccacugcccacuga

SEQ ID NO: 196 - RNA, 17 nt, Homo sapiens
cacugcccacugacugc

SEQ ID NO: 197 - RNA, 17 nt, Homo sapiens
gcccacugacugcugcc

SEQ ID NO: 198 - RNA, 17 nt, Homo sapiens
cacugacugcugccaug

SEQ ID NO: 199 - RNA, 17 nt, Homo sapiens
ugacugcugccaugagc

SEQ ID NO: 200 - RNA, 17 nt, Homo sapiens
cugcugccaugagcagu

SEQ ID NO: 201 - RNA, 17 nt, Homo sapiens
ugccaugagcagugugc

SEQ ID NO: 202 - RNA, 17 nt, Homo sapiens
caugagcagugugcugc

SEQ ID NO: 203 - RNA, 17 nt, Homo sapiens
ugagcagugugcugccg

SEQ ID NO: 204 - RNA, 17 nt, Homo sapiens
agcagugugcugccggc

SEQ ID NO: 205 - RNA, 17 nt, Homo sapiens
agugugcugccggcugc

SEQ ID NO: 206 - RNA, 17 nt, Homo sapiens
ugcugccggcugcacgg

SEQ ID NO: 207 - RNA, 17 nt, Homo sapiens
ugccggcugcacgggcc

SEQ ID NO: 208 - RNA, 17 nt, Homo sapiens
ccggcugcacgggcccc

SEQ ID NO: 209 - RNA, 17 nt, Homo sapiens
cugcacgggccccaagc

SEQ ID NO: 210 - RNA, 17 nt, Homo sapiens
ggccccaagcacucuga

SEQ ID NO: 211 - RNA, 17 nt, Homo sapiens
ccccaagcacucugacu

SEQ ID NO: 212 - RNA, 17 nt, Homo sapiens
gcacucugacugccugg

SEQ ID NO: 213 - RNA, 17 nt, Homo sapiens
cucugacugccuggccu

SEQ ID NO: 214 - RNA, 17 nt, Homo sapiens
acugccuggccugccuc

SEQ ID NO: 215 - RNA, 17 nt, Homo sapiens
ccuggccugccuccacu

SEQ ID NO: 216 - RNA, 17 nt, Homo sapiens
cugccuccacuucaacc

SEQ ID NO: 217 - RNA, 17 nt, Homo sapiens
ccacuucaaccacagug

SEQ ID NO: 218 - RNA, 17 nt, Homo sapiens
cuucaaccacaguggca

SEQ ID NO: 219 - RNA, 17 nt, Homo sapiens
caaccacaguggcaucu

SEQ ID NO: 220 - RNA, 17 nt, Homo sapiens
ccacaguggcaucugug

SEQ ID NO: 221 - RNA, 17 nt, Homo sapiens
acaguggcaucugugag

SEQ ID NO: 222 - RNA, 17 nt, Homo sapiens
uggcaucugugagcugc

SEQ ID NO: 223 - RNA, 17 nt, Homo sapiens
aucugugagcugcacug

SEQ ID NO: 224 - RNA, 17 nt, Homo sapiens
ugugagcugcacugccc

SEQ ID NO: 225 - RNA, 17 nt, Homo sapiens
ugagcugcacugcccag

SEQ ID NO: 226 - RNA, 17 nt, Homo sapiens
gcugcacugcccagccc

SEQ ID NO: 227 - RNA, 17 nt, Homo sapiens
acugcccagcccugguc

SEQ ID NO: 228 - RNA, 17 nt, Homo sapiens
cagcccuggucaccuac

SEQ ID NO: 229 - RNA, 17 nt, Homo sapiens
cccuggucaccuacaac

SEQ ID NO: 230 - RNA, 17 nt, Homo sapiens
ggucaccuacaacacag

SEQ ID NO: 231 - RNA, 17 nt, Homo sapiens
caccuacaacacagaca

SEQ ID NO: 232 - RNA, 17 nt, Homo sapiens
ccuacaacacagacacg

SEQ ID NO: 233 - RNA, 17 nt, Homo sapiens
caacacagacacguuug

SEQ ID NO: 234 - RNA, 17 nt, Homo sapiens
acacagacacguuugag

SEQ ID NO: 235 - RNA, 17 nt, Homo sapiens
acagacacguuugaguc

SEQ ID NO: 236 - RNA, 17 nt, Homo sapiens
acguuugaguccaugcc

SEQ ID NO: 237 - RNA, 17 nt, Homo sapiens
uugaguccaugcccaau

SEQ ID NO: 238 - RNA, 17 nt, Homo sapiens
gaguccaugcccaaucc

SEQ ID NO: 239 - RNA, 17 nt, Homo sapiens
caugcccaaucccgagg

SEQ ID NO: 240 - RNA, 17 nt, Homo sapiens
ucccgagggccgguaua

SEQ ID NO: 241 - RNA, 17 nt, Homo sapiens
gagggccgguauacauu

SEQ ID NO: 242 - RNA, 17 nt, Homo sapiens
gggccgguauacauucg

SEQ ID NO: 243 - RNA, 17 nt, Homo sapiens
gccgguauacauucggc

SEQ ID NO: 244 - RNA, 17 nt, Homo sapiens
cgguauacauucggcgc

SEQ ID NO: 245 - RNA, 17 nt, Homo sapiens
uacauucggcgccagcu

SEQ ID NO: 246 - RNA, 17 nt, Homo sapiens
cauucggcgccagcugu

SEQ ID NO: 247 - RNA, 17 nt, Homo sapiens
cggcgccagcuguguga

SEQ ID NO: 248 - RNA, 17 nt, Homo sapiens
cgccagcugugugacug

SEQ ID NO: 249 - RNA, 17 nt, Homo sapiens
ccagcugugugacugcc

SEQ ID NO: 250 - RNA, 17 nt, Homo sapiens
gcugugugacugccugu

SEQ ID NO: 251 - RNA, 17 nt, Homo sapiens
gugugacugccuguccc

SEQ ID NO: 252 - RNA, 17 nt, Homo sapiens
gacugccugucccuaca

SEQ ID NO: 253 - RNA, 17 nt, Homo sapiens
cugucccuacaacuacc

SEQ ID NO: 254 - RNA, 17 nt, Homo sapiens
ucccuacaacuaccuuu

SEQ ID NO: 255 - RNA, 17 nt, Homo sapiens
cuacaacuaccuuucua

SEQ ID NO: 256 - RNA, 17 nt, Homo sapiens
accuuucuacggacgug

SEQ ID NO: 257 - RNA, 17 nt, Homo sapiens
uucuacggacgugggau

SEQ ID NO: 258 - RNA, 17 nt, Homo sapiens
cuacggacgugggaucc

SEQ ID NO: 259 - RNA, 17 nt, Homo sapiens
gacgugggauccugcac

SEQ ID NO: 260 - RNA, 17 nt, Homo sapiens
gggauccugcacccucg

SEQ ID NO: 261 - RNA, 17 nt, Homo sapiens
gauccugcacccucguc

SEQ ID NO: 262 - RNA, 17 nt, Homo sapiens
gcacccucgucugcccc

SEQ ID NO: 263 - RNA, 17 nt, Homo sapiens
ccucgucugcccccugc

SEQ ID NO: 264 - RNA, 17 nt, Homo sapiens
ugcccccugcacaacca

SEQ ID NO: 265 - RNA, 17 nt, Homo sapiens
cccccugcacaaccaag

SEQ ID NO: 266 - RNA, 17 nt, Homo sapiens
ccugcacaaccaagagg

SEQ ID NO: 267 - RNA, 17 nt, Homo sapiens
accaagaggugacagca

SEQ ID NO: 268 - RNA, 17 nt, Homo sapiens
aagaggugacagcagag

SEQ ID NO: 269 - RNA, 17 nt, Homo sapiens
aggugacagcagaggau

SEQ ID NO: 270 - RNA, 17 nt, Homo sapiens
agcagaggauggaacac

SEQ ID NO: 271 - RNA, 17 nt, Homo sapiens
aggauggaacacagcgg

SEQ ID NO: 272 - RNA, 17 nt, Homo sapiens
gauggaacacagcggug

SEQ ID NO: 273 - RNA, 17 nt, Homo sapiens
ggaacacagcgguguga

SEQ ID NO: 274 - RNA, 17 nt, Homo sapiens
acacagcggugugagaa

SEQ ID NO: 275 - RNA, 17 nt, Homo sapiens
acagcggugugagaagu

SEQ ID NO: 276 - RNA, 17 nt, Homo sapiens
ugugagaagugcagcaa

SEQ ID NO: 277 - RNA, 17 nt, Homo sapiens
ugagaagugcagcaagc

SEQ ID NO: 278 - RNA, 17 nt, Homo sapiens
gaagugcagcaagcccu

SEQ ID NO: 279 - RNA, 17 nt, Homo sapiens
ugcagcaagcccugugc

SEQ ID NO: 280 - RNA, 17 nt, Homo sapiens
caagcccugugcccgag

SEQ ID NO: 281 - RNA, 17 nt, Homo sapiens
agcccugugcccgagug

SEQ ID NO: 282 - RNA, 17 nt, Homo sapiens
gugcccgagugugcuau

SEQ ID NO: 283 - RNA, 17 nt, Homo sapiens
gcccgagugugcuaugg

SEQ ID NO: 284 - RNA, 17 nt, Homo sapiens
ccgagugugcuaugguc

SEQ ID NO: 285 - RNA, 17 nt, Homo sapiens
agugugcuauggucugg

SEQ ID NO: 286 - RNA, 17 nt, Homo sapiens
gugcuauggucugggca

SEQ ID NO: 287 - RNA, 17 nt, Homo sapiens
uggucugggcauggagc

SEQ ID NO: 288 - RNA, 17 nt, Homo sapiens
gucugggcauggagcac

SEQ ID NO: 289 - RNA, 17 nt, Homo sapiens
ggcauggagcacuugcg

SEQ ID NO: 290 - RNA, 17 nt, Homo sapiens
cauggagcacuugcgag

SEQ ID NO: 291 - RNA, 17 nt, Homo sapiens
gagcacuugcgagaggu

SEQ ID NO: 292 - RNA, 17 nt, Homo sapiens
ugcgagaggugagggca

SEQ ID NO: 293 - RNA, 17 nt, Homo sapiens
aggugagggcaguuacc

SEQ ID NO: 294 - RNA, 17 nt, Homo sapiens
ugagggcaguuaccagu

SEQ ID NO: 295 - RNA, 17 nt, Homo sapiens
gggcaguuaccagugcc

SEQ ID NO: 296 - RNA, 17 nt, Homo sapiens
aguuaccagugccaaua

SEQ ID NO: 297 - RNA, 17 nt, Homo sapiens
uuaccagugccaauauc

SEQ ID NO: 298 - RNA, 17 nt, Homo sapiens
cagugccaauauccagg

SEQ ID NO: 299 - RNA, 17 nt, Homo sapiens
gugccaauauccaggag

SEQ ID NO: 300 - RNA, 17 nt, Homo sapiens
auccaggaguuugcugg

SEQ ID NO: 301 - RNA, 17 nt, Homo sapiens
aggaguuugcuggcugc

SEQ ID NO: 302 - RNA, 17 nt, Homo sapiens
guuugcuggcugcaaga

SEQ ID NO: 303 - RNA, 17 nt, Homo sapiens
ugcuggcugcaagaaga

SEQ ID NO: 304 - RNA, 17 nt, Homo sapiens
gcaagaagaucuuuggg

SEQ ID NO: 305 - RNA, 17 nt, Homo sapiens
cuuugggagccuggcau

SEQ ID NO: 306 - RNA, 17 nt, Homo sapiens
ggagccuggcauuucug

SEQ ID NO: 307 - RNA, 17 nt, Homo sapiens
agccuggcauuucugcc

SEQ ID NO: 308 - RNA, 17 nt, Homo sapiens
gcauuucugccggagag

SEQ ID NO: 309 - RNA, 17 nt, Homo sapiens
gccggagagcuuugaug

SEQ ID NO: 310 - RNA, 17 nt, Homo sapiens
gagcuuugauggggacc

SEQ ID NO: 311 - RNA, 17 nt, Homo sapiens
ugauggggacccagccu

SEQ ID NO: 312 - RNA, 17 nt, Homo sapiens
gggacccagccuccaac

SEQ ID NO: 313 - RNA, 17 nt, Homo sapiens
agccuccaacacugccc

SEQ ID NO: 314 - RNA, 17 nt, Homo sapiens
ccuccaacacugccccg

SEQ ID NO: 315 - RNA, 17 nt, Homo sapiens
ccaacacugccccgcuc

SEQ ID NO: 316 - RNA, 17 nt, Homo sapiens
acugccccgcuccagcc

SEQ ID NO: 317 - RNA, 17 nt, Homo sapiens
ccgcuccagccagagca

SEQ ID NO: 318 - RNA, 17 nt, Homo sapiens
cagccagagcagcucca

SEQ ID NO: 319 - RNA, 17 nt, Homo sapiens
ccagagcagcuccaagu

SEQ ID NO: 320 - RNA, 17 nt, Homo sapiens
agcuccaaguguuugag

SEQ ID NO: 321 - RNA, 17 nt, Homo sapiens
cuccaaguguuugagac

SEQ ID NO: 322 - RNA, 17 nt, Homo sapiens
uguuugagacucuggaa

SEQ ID NO: 323 - RNA, 17 nt, Homo sapiens
uggaagagaucacaggu

SEQ ID NO: 324 - RNA, 17 nt, Homo sapiens
aagagaucacagguuac

SEQ ID NO: 325 - RNA, 17 nt, Homo sapiens
gaucacagguuaccuau

SEQ ID NO: 326 - RNA, 17 nt, Homo sapiens
cacagguuaccuauaca

SEQ ID NO: 327 - RNA, 17 nt, Homo sapiens
gguuaccuauacaucuc

SEQ ID NO: 328 - RNA, 17 nt, Homo sapiens
uuaccuauacaucucag

SEQ ID NO: 329 - RNA, 17 nt, Homo sapiens
accuauacaucucagca

SEQ ID NO: 330 - RNA, 17 nt, Homo sapiens
acaucucagcauggccg

SEQ ID NO: 331 - RNA, 17 nt, Homo sapiens
aucucagcauggccgga

SEQ ID NO: 332 - RNA, 17 nt, Homo sapiens
ucagcauggccggacag

SEQ ID NO: 333 - RNA, 17 nt, Homo sapiens
auggccggacagccugc

SEQ ID NO: 334 - RNA, 17 nt, Homo sapiens
gccggacagccugccug

SEQ ID NO: 335 - RNA, 17 nt, Homo sapiens
gacagccugccugaccu

SEQ ID NO: 336 - RNA, 17 nt, Homo sapiens
ccugccugaccucagcg

SEQ ID NO: 337 - RNA, 17 nt, Homo sapiens
ugaccucagcgucuucc

SEQ ID NO: 338 - RNA, 17 nt, Homo sapiens
accucagcgucuuccag

SEQ ID NO: 339 - RNA, 17 nt, Homo sapiens
cuuccagaaccugcaag

SEQ ID NO: 340 - RNA, 17 nt, Homo sapiens
cagaaccugcaaguaau

SEQ ID NO: 341 - RNA, 17 nt, Homo sapiens
accugcaaguaauccgg

SEQ ID NO: 342 - RNA, 17 nt, Homo sapiens
ugcaaguaauccgggga

SEQ ID NO: 343 - RNA, 17 nt, Homo sapiens
auccggggacgaauucu

SEQ ID NO: 344 - RNA, 17 nt, Homo sapiens
ggggacgaauucugcac

SEQ ID NO: 345 - RNA, 17 nt, Homo sapiens
cgaauucugcacaaugg

SEQ ID NO: 346 - RNA, 17 nt, Homo sapiens
aauucugcacaauggcg

SEQ ID NO: 347 - RNA, 17 nt, Homo sapiens
ucugcacaauggcgccu

SEQ ID NO: 348 - RNA, 17 nt, Homo sapiens
gcacaauggcgccuacu

SEQ ID NO: 349 - RNA, 17 nt, Homo sapiens
acaauggcgccuacucg

SEQ ID NO: 350 - RNA, 17 nt, Homo sapiens
uggcgccuacucgcuga

SEQ ID NO: 351 - RNA, 17 nt, Homo sapiens
gccuacucgcugacccu

SEQ ID NO: 352 - RNA, 17 nt, Homo sapiens
acucgcugacccugcaa

SEQ ID NO: 353 - RNA, 17 nt, Homo sapiens
cugacccugcaagggcu

SEQ ID NO: 354 - RNA, 17 nt, Homo sapiens
cugcaagggcugggcau

SEQ ID NO: 355 - RNA, 17 nt, Homo sapiens
agggcugggcaucagcu

SEQ ID NO: 356 - RNA, 17 nt, Homo sapiens
ggcugggcaucagcugg

SEQ ID NO: 357 - RNA, 17 nt, Homo sapiens
gggcaucagcuggcugg

SEQ ID NO: 358 - RNA, 17 nt, Homo sapiens
aucagcuggcuggggcu

SEQ ID NO: 359 - RNA, 17 nt, Homo sapiens
uggcuggggcugcgcuc

SEQ ID NO: 360 - RNA, 17 nt, Homo sapiens
cuggggcugcgcucacu

SEQ ID NO: 361 - RNA, 17 nt, Homo sapiens
ggggcugcgcucacuga

SEQ ID NO: 362 - RNA, 17 nt, Homo sapiens
cugcgcucacugaggga

SEQ ID NO: 363 - RNA, 17 nt, Homo sapiens
cugagggaacugggcag

SEQ ID NO: 364 - RNA, 17 nt, Homo sapiens
ggaacugggcaguggac

SEQ ID NO: 365 - RNA, 17 nt, Homo sapiens
acugggcaguggacugg

SEQ ID NO: 366 - RNA, 17 nt, Homo sapiens
ggcaguggacuggcccu

SEQ ID NO: 367 - RNA, 17 nt, Homo sapiens
guggacuggcccucauc

SEQ ID NO: 368 - RNA, 17 nt, Homo sapiens
uggcccucauccaccau

SEQ ID NO: 369 - RNA, 17 nt, Homo sapiens
ccucauccaccauaaca

SEQ ID NO: 370 - RNA, 17 nt, Homo sapiens
cauccaccauaacaccc

SEQ ID NO: 371 - RNA, 17 nt, Homo sapiens
ccaccauaacacccacc

SEQ ID NO: 372 - RNA, 17 nt, Homo sapiens
accauaacacccaccuc

SEQ ID NO: 373 - RNA, 17 nt, Homo sapiens
uaacacccaccucugcu

SEQ ID NO: 374 - RNA, 17 nt, Homo sapiens
ccaccucugcuucgugc

SEQ ID NO: 375 - RNA, 17 nt, Homo sapiens
ucugcuucgugcacacg

SEQ ID NO: 376 - RNA, 17 nt, Homo sapiens
ugcuucgugcacacggu

SEQ ID NO: 377 - RNA, 17 nt, Homo sapiens
cuucgugcacacggugc

SEQ ID NO: 378 - RNA, 17 nt, Homo sapiens
ucgugcacacggugccc

SEQ ID NO: 379 - RNA, 17 nt, Homo sapiens
ugcacacggugcccugg

SEQ ID NO: 380 - RNA, 17 nt, Homo sapiens
cacacggugcccuggga

SEQ ID NO: 381 - RNA, 17 nt, Homo sapiens
gcccugggaccagcucu

SEQ ID NO: 382 - RNA, 17 nt, Homo sapiens
ugggaccagcucuuucg

SEQ ID NO: 383 - RNA, 17 nt, Homo sapiens
cuuucggaacccgcacc

SEQ ID NO: 384 - RNA, 17 nt, Homo sapiens
cggaacccgcaccaagc

SEQ ID NO: 385 - RNA, 17 nt, Homo sapiens
gaacccgcaccaagcuc

SEQ ID NO: 386 - RNA, 17 nt, Homo sapiens
cgcaccaagcucugcuc

SEQ ID NO: 387 - RNA, 17 nt, Homo sapiens
caagcucugcuccacac

SEQ ID NO: 388 - RNA, 17 nt, Homo sapiens
ucugcuccacacugcca

SEQ ID NO: 389 - RNA, 17 nt, Homo sapiens
ugcuccacacugccaac

SEQ ID NO: 390 - RNA, 17 nt, Homo sapiens
uccacacugccaaccgg

SEQ ID NO: 391 - RNA, 17 nt, Homo sapiens
cacugccaaccggccag

SEQ ID NO: 392 - RNA, 17 nt, Homo sapiens
gccaaccggccagagga

SEQ ID NO: 393 - RNA, 17 nt, Homo sapiens
gccagaggacgagugug

SEQ ID NO: 394 - RNA, 17 nt, Homo sapiens
gaggacgaguguguggg

SEQ ID NO: 395 - RNA, 17 nt, Homo sapiens
ggacgagugugugggcg

SEQ ID NO: 396 - RNA, 17 nt, Homo sapiens
acgagugugugggcgag

SEQ ID NO: 397 - RNA, 17 nt, Homo sapiens
gugugugggcgagggcc

SEQ ID NO: 398 - RNA, 17 nt, Homo sapiens
gggcgagggccuggccu

SEQ ID NO: 399 - RNA, 17 nt, Homo sapiens
agggccuggccugccac

SEQ ID NO: 400 - RNA, 17 nt, Homo sapiens
ccuggccugccaccagc

SEQ ID NO: 401 - RNA, 17 nt, Homo sapiens
ggccugccaccagcugu

SEQ ID NO: 402 - RNA, 17 nt, Homo sapiens
ugccaccagcugugcgc

SEQ ID NO: 403 - RNA, 17 nt, Homo sapiens
caccagcugugcgcccg

SEQ ID NO: 404 - RNA, 17 nt, Homo sapiens
ccagcugugcgcccgag

SEQ ID NO: 405 - RNA, 17 nt, Homo sapiens
agcugugcgcccgaggg

SEQ ID NO: 406 - RNA, 17 nt, Homo sapiens
gcccgagggcacugcug

SEQ ID NO: 407 - RNA, 17 nt, Homo sapiens
ccgagggcacugcuggg

SEQ ID NO: 408 - RNA, 17 nt, Homo sapiens
agggcacugcugggguc

SEQ ID NO: 409 - RNA, 17 nt, Homo sapiens
cugcugggguccagggc

SEQ ID NO: 410 - RNA, 17 nt, Homo sapiens
gguccagggcccaccca

SEQ ID NO: 411 - RNA, 17 nt, Homo sapiens
cagggcccacccagugu

SEQ ID NO: 412 - RNA, 17 nt, Homo sapiens
cccacccagugugucaa

SEQ ID NO: 413 - RNA, 17 nt, Homo sapiens
cacccagugugucaacu

SEQ ID NO: 414 - RNA, 17 nt, Homo sapiens
cccagugugucaacugc

SEQ ID NO: 415 - RNA, 17 nt, Homo sapiens
gugugucaacugcagcc

SEQ ID NO: 416 - RNA, 17 nt, Homo sapiens
ugucaacugcagccagu

SEQ ID NO: 417 - RNA, 17 nt, Homo sapiens
caacugcagccaguucc

SEQ ID NO: 418 - RNA, 17 nt, Homo sapiens
ugcagccaguuccuucg

SEQ ID NO: 419 - RNA, 17 nt, Homo sapiens
ccuucggggccaggagu

SEQ ID NO: 420 - RNA, 17 nt, Homo sapiens
ggccaggagugcgugga

SEQ ID NO: 421 - RNA, 17 nt, Homo sapiens
ccaggagugcguggagg

SEQ ID NO: 422 - RNA, 17 nt, Homo sapiens
aggagugcguggaggaa

SEQ ID NO: 423 - RNA, 17 nt, Homo sapiens
guggaggaaugccgagu

SEQ ID NO: 424 - RNA, 17 nt, Homo sapiens
ggaggaaugccgaguac

SEQ ID NO: 425 - RNA, 17 nt, Homo sapiens
aaugccgaguacugcag

SEQ ID NO: 426 - RNA, 17 nt, Homo sapiens
ugccgaguacugcaggg

SEQ ID NO: 427 - RNA, 17 nt, Homo sapiens
cgaguacugcaggggcu

SEQ ID NO: 428 - RNA, 17 nt, Homo sapiens
cugcaggggcuccccag

SEQ ID NO: 429 - RNA, 17 nt, Homo sapiens
cccagggaguaugugaa

SEQ ID NO: 430 - RNA, 17 nt, Homo sapiens
cagggaguaugugaaug

SEQ ID NO: 431 - RNA, 17 nt, Homo sapiens
gggaguaugugaaugcc

SEQ ID NO: 432 - RNA, 17 nt, Homo sapiens
guaugugaaugccaggc

SEQ ID NO: 433 - RNA, 17 nt, Homo sapiens
augugaaugccaggcac

SEQ ID NO: 434 - RNA, 17 nt, Homo sapiens
aaugccaggcacuguuu

SEQ ID NO: 435 - RNA, 17 nt, Homo sapiens
ugccaggcacuguuugc

SEQ ID NO: 436 - RNA, 17 nt, Homo sapiens
caggcacuguuugccgu

SEQ ID NO: 437 - RNA, 17 nt, Homo sapiens
cacuguuugccgugcca

SEQ ID NO: 438 - RNA, 17 nt, Homo sapiens
uguuugccgugccaccc

SEQ ID NO: 439 - RNA, 17 nt, Homo sapiens
uuugccgugccacccug

SEQ ID NO: 440 - RNA, 17 nt, Homo sapiens
gccgugccacccugagu

SEQ ID NO: 441 - RNA, 17 nt, Homo sapiens
cacccugagugucagcc

SEQ ID NO: 442 - RNA, 17 nt, Homo sapiens
cccugagugucagcccc

SEQ ID NO: 443 - RNA, 17 nt, Homo sapiens
gagugucagccccagaa

SEQ ID NO: 444 - RNA, 17 nt, Homo sapiens
gccccagaauggcucag

SEQ ID NO: 445 - RNA, 17 nt, Homo sapiens
ccagaauggcucaguga

SEQ ID NO: 446 - RNA, 17 nt, Homo sapiens
auggcucagugaccugu

SEQ ID NO: 447 - RNA, 17 nt, Homo sapiens
gcucagugaccuguuuu

SEQ ID NO: 448 - RNA, 17 nt, Homo sapiens
agugaccuguuuuggac

SEQ ID NO: 449 - RNA, 17 nt, Homo sapiens
uguuuuggaccggaggc

SEQ ID NO: 450 - RNA, 17 nt, Homo sapiens
gaccggaggcugaccag

SEQ ID NO: 451 - RNA, 17 nt, Homo sapiens
ggaggcugaccagugug

SEQ ID NO: 452 - RNA, 17 nt, Homo sapiens
gcugaccaguguguggc

SEQ ID NO: 453 - RNA, 17 nt, Homo sapiens
ugaccaguguguggccu

SEQ ID NO: 454 - RNA, 17 nt, Homo sapiens
accaguguguggccugu

SEQ ID NO: 455 - RNA, 17 nt, Homo sapiens
aguguguggccugugcc

SEQ ID NO: 456 - RNA, 17 nt, Homo sapiens
uguggccugugcccacu

SEQ ID NO: 457 - RNA, 17 nt, Homo sapiens
uggccugugcccacuau

SEQ ID NO: 458 - RNA, 17 nt, Homo sapiens
cugugcccacuauaagg

SEQ ID NO: 459 - RNA, 17 nt, Homo sapiens
ugcccacuauaaggacc

SEQ ID NO: 460 - RNA, 17 nt, Homo sapiens
cuauaaggacccucccu

SEQ ID NO: 461 - RNA, 17 nt, Homo sapiens
ucccuucugcguggccc

SEQ ID NO: 462 - RNA, 17 nt, Homo sapiens
ccuucugcguggcccgc

SEQ ID NO: 463 - RNA, 17 nt, Homo sapiens
ucugcguggcccgcugc

SEQ ID NO: 464 - RNA, 17 nt, Homo sapiens
cguggcccgcugcccca

SEQ ID NO: 465 - RNA, 17 nt, Homo sapiens
ggcccgcugccccagcg

SEQ ID NO: 466 - RNA, 17 nt, Homo sapiens
cugccccagcgguguga

SEQ ID NO: 467 - RNA, 17 nt, Homo sapiens
ccccagcggugugaaac

SEQ ID NO: 468 - RNA, 17 nt, Homo sapiens
ccagcggugugaaaccu

SEQ ID NO: 469 - RNA, 17 nt, Homo sapiens
ggugugaaaccugaccu

SEQ ID NO: 470 - RNA, 17 nt, Homo sapiens
gaaaccugaccucuccu

SEQ ID NO: 471 - RNA, 17 nt, Homo sapiens
ccucuccuacaugccca

SEQ ID NO: 472 - RNA, 17 nt, Homo sapiens
ucuccuacaugcccauc

SEQ ID NO: 473 - RNA, 17 nt, Homo sapiens
uccuacaugcccaucug

SEQ ID NO: 474 - RNA, 17 nt, Homo sapiens
acaugcccaucuggaag

SEQ ID NO: 475 - RNA, 17 nt, Homo sapiens
aucuggaaguuuccaga

SEQ ID NO: 476 - RNA, 17 nt, Homo sapiens
guuuccagaugaggagg

SEQ ID NO: 477 - RNA, 17 nt, Homo sapiens
ugaggagggcgcaugcc

SEQ ID NO: 478 - RNA, 17 nt, Homo sapiens
aggagggcgcaugccag

SEQ ID NO: 479 - RNA, 17 nt, Homo sapiens
gagggcgcaugccagcc

SEQ ID NO: 480 - RNA, 17 nt, Homo sapiens
gggcgcaugccagccuu

SEQ ID NO: 481 - RNA, 17 nt, Homo sapiens
gcaugccagccuugccc

SEQ ID NO: 482 - RNA, 17 nt, Homo sapiens
ccagccuugccccauca

SEQ ID NO: 483 - RNA, 17 nt, Homo sapiens
cuugccccaucaacugc

SEQ ID NO: 484 - RNA, 17 nt, Homo sapiens
ccccaucaacugcaccc

SEQ ID NO: 485 - RNA, 17 nt, Homo sapiens
caucaacugcacccacu

SEQ ID NO: 486 - RNA, 17 nt, Homo sapiens
ucaacugcacccacucc

SEQ ID NO: 487 - RNA, 17 nt, Homo sapiens
cugcacccacuccugug

SEQ ID NO: 488 - RNA, 17 nt, Homo sapiens
ccacuccuguguggacc

SEQ ID NO: 489 - RNA, 17 nt, Homo sapiens
acuccuguguggaccug

SEQ ID NO: 490 - RNA, 17 nt, Homo sapiens
cuguguggaccuggaug

SEQ ID NO: 491 - RNA, 17 nt, Homo sapiens
ggaccuggaugacaagg

SEQ ID NO: 492 - RNA, 17 nt, Homo sapiens
ccuggaugacaagggcu

SEQ ID NO: 493 - RNA, 17 nt, Homo sapiens
ugacaagggcugccccg

SEQ ID NO: 494 - RNA, 17 nt, Homo sapiens
caagggcugccccgccg

SEQ ID NO: 495 - RNA, 17 nt, Homo sapiens
gcugccccgccgagcag

SEQ ID NO: 496 - RNA, 17 nt, Homo sapiens
cccgccgagcagagagc

SEQ ID NO: 497 - RNA, 17 nt, Homo sapiens
agcagagagccagcccu

SEQ ID NO: 498 - RNA, 17 nt, Homo sapiens
gagagccagcccucuga

SEQ ID NO: 499 - RNA, 17 nt, Homo sapiens
gcccucugacguccauc

SEQ ID NO: 500 - RNA, 17 nt, Homo sapiens
ccucugacguccaucau

SEQ ID NO: 501 - RNA, 17 nt, Homo sapiens
ugacguccaucaucucu

SEQ ID NO: 502 - RNA, 17 nt, Homo sapiens
cguccaucaucucugcg

SEQ ID NO: 503 - RNA, 17 nt, Homo sapiens
ucaucucugcggugguu

SEQ ID NO: 504 - RNA, 17 nt, Homo sapiens
ucucugcggugguuggc

SEQ ID NO: 505 - RNA, 17 nt, Homo sapiens
cugcggugguuggcauu

SEQ ID NO: 506 - RNA, 17 nt, Homo sapiens
ggugguuggcauucugc

SEQ ID NO: 507 - RNA, 17 nt, Homo sapiens
ugguuggcauucugcug

SEQ ID NO: 508 - RNA, 17 nt, Homo sapiens
ggcauucugcuggucgu

SEQ ID NO: 509 - RNA, 17 nt, Homo sapiens
uucugcuggucgugguc

SEQ ID NO: 510 - RNA, 17 nt, Homo sapiens
ugcuggucguggucuug

SEQ ID NO: 511 - RNA, 17 nt, Homo sapiens
uggucguggucuugggg

SEQ ID NO: 512 - RNA, 17 nt, Homo sapiens
ucuuggggguggucuuu

SEQ ID NO: 513 - RNA, 17 nt, Homo sapiens
uggggguggucuuuggg

SEQ ID NO: 514 - RNA, 17 nt, Homo sapiens
ucuuugggauccucauc

SEQ ID NO: 515 - RNA, 17 nt, Homo sapiens
ggauccucaucaagcga

SEQ ID NO: 516 - RNA, 17 nt, Homo sapiens
cucaucaagcgacggca

SEQ ID NO: 517 - RNA, 17 nt, Homo sapiens
aucaagcgacggcagca

SEQ ID NO: 518 - RNA, 17 nt, Homo sapiens
aagcgacggcagcagaa

SEQ ID NO: 519 - RNA, 17 nt, Homo sapiens
cgacggcagcagaagau

SEQ ID NO: 520 - RNA, 17 nt, Homo sapiens
agcagaagauccggaag

SEQ ID NO: 521 - RNA, 17 nt, Homo sapiens
auccggaaguacacgau

SEQ ID NO: 522 - RNA, 17 nt, Homo sapiens
ccggaaguacacgaugc

SEQ ID NO: 523 - RNA, 17 nt, Homo sapiens
ggaaguacacgaugcgg

SEQ ID NO: 524 - RNA, 17 nt, Homo sapiens
aguacacgaugcggaga

SEQ ID NO: 525 - RNA, 17 nt, Homo sapiens
uacacgaugcggagacu

SEQ ID NO: 526 - RNA, 17 nt, Homo sapiens
augcggagacugcugca

SEQ ID NO: 527 - RNA, 17 nt, Homo sapiens
cggagacugcugcagga

SEQ ID NO: 528 - RNA, 17 nt, Homo sapiens
agacugcugcaggaaac

SEQ ID NO: 529 - RNA, 17 nt, Homo sapiens
ugcaggaaacggagcug

SEQ ID NO: 530 - RNA, 17 nt, Homo sapiens
gaaacggagcuggugga

SEQ ID NO: 531 - RNA, 17 nt, Homo sapiens
cggagcugguggagccg

SEQ ID NO: 532 - RNA, 17 nt, Homo sapiens
cugguggagccgcugac

SEQ ID NO: 533 - RNA, 17 nt, Homo sapiens
guggagccgcugacacc

SEQ ID NO: 534 - RNA, 17 nt, Homo sapiens
agccgcugacaccuagc

SEQ ID NO: 535 - RNA, 17 nt, Homo sapiens
ccgcugacaccuagcgg

SEQ ID NO: 536 - RNA, 17 nt, Homo sapiens
gacaccuagcggagcga

SEQ ID NO: 537 - RNA, 17 nt, Homo sapiens
cuagcggagcgaugccc

SEQ ID NO: 538 - RNA, 17 nt, Homo sapiens
gcggagcgaugcccaac

SEQ ID NO: 539 - RNA, 17 nt, Homo sapiens
ggagcgaugcccaacca

SEQ ID NO: 540 - RNA, 17 nt, Homo sapiens
gaugcccaaccaggcgc

SEQ ID NO: 541 - RNA, 17 nt, Homo sapiens
ccaaccaggcgcagaug

SEQ ID NO: 542 - RNA, 17 nt, Homo sapiens
aaccaggcgcagaugcg

SEQ ID NO: 543 - RNA, 17 nt, Homo sapiens
aggcgcagaugcggauc

SEQ ID NO: 544 - RNA, 17 nt, Homo sapiens
gcgcagaugcggauccu

SEQ ID NO: 545 - RNA, 17 nt, Homo sapiens
agaugcggauccugaaa

SEQ ID NO: 546 - RNA, 17 nt, Homo sapiens
ugaaagagacggagcug

SEQ ID NO: 547 - RNA, 17 nt, Homo sapiens
gagacggagcugaggaa

SEQ ID NO: 548 - RNA, 17 nt, Homo sapiens
ugaggaaggugaaggug

SEQ ID NO: 549 - RNA, 17 nt, Homo sapiens
aggugaaggugcuugga

SEQ ID NO: 550 - RNA, 17 nt, Homo sapiens
gugaaggugcuuggauc

SEQ ID NO: 551 - RNA, 17 nt, Homo sapiens
gugcuuggaucuggcgc

SEQ ID NO: 552 - RNA, 17 nt, Homo sapiens
uggaucuggcgcuuuug

SEQ ID NO: 553 - RNA, 17 nt, Homo sapiens
gaucuggcgcuuuuggc

SEQ ID NO: 554 - RNA, 17 nt, Homo sapiens
cgcuuuuggcacagucu

SEQ ID NO: 555 - RNA, 17 nt, Homo sapiens
cuuuuggcacagucuac

SEQ ID NO: 556 - RNA, 17 nt, Homo sapiens
uuggcacagucuacaag

SEQ ID NO: 557 - RNA, 17 nt, Homo sapiens
cacagucuacaagggca

SEQ ID NO: 558 - RNA, 17 nt, Homo sapiens
cuacaagggcaucugga

SEQ ID NO: 559 - RNA, 17 nt, Homo sapiens
acaagggcaucuggauc

SEQ ID NO: 560 - RNA, 17 nt, Homo sapiens
gcaucuggaucccugau

SEQ ID NO: 561 - RNA, 17 nt, Homo sapiens
gaucccugauggggaga

SEQ ID NO: 562 - RNA, 17 nt, Homo sapiens
uggggagaaugugaaaa

SEQ ID NO: 563 - RNA, 17 nt, Homo sapiens
gggagaaugugaaaauu

SEQ ID NO: 564 - RNA, 17 nt, Homo sapiens
augugaaaauuccagug

SEQ ID NO: 565 - RNA, 17 nt, Homo sapiens
aaauuccaguggccauc

SEQ ID NO: 566 - RNA, 17 nt, Homo sapiens
uuccaguggccaucaaa

SEQ ID NO: 567 - RNA, 17 nt, Homo sapiens
caguggccaucaaagug

SEQ ID NO: 568 - RNA, 17 nt, Homo sapiens
ccaucaaaguguugagg

SEQ ID NO: 569 - RNA, 17 nt, Homo sapiens
aucaaaguguugaggga

SEQ ID NO: 570 - RNA, 17 nt, Homo sapiens
gagggaaaacacauccc

SEQ ID NO: 571 - RNA, 17 nt, Homo sapiens
gggaaaacacauccccc

SEQ ID NO: 572 - RNA, 17 nt, Homo sapiens
gaaaacacaucccccaa

SEQ ID NO: 573 - RNA, 17 nt, Homo sapiens
cccccaaagccaacaaa

SEQ ID NO: 574 - RNA, 17 nt, Homo sapiens
caaagccaacaaagaaa

SEQ ID NO: 575 - RNA, 17 nt, Homo sapiens
acaaagaaaucuuagac

SEQ ID NO: 576 - RNA, 17 nt, Homo sapiens
aaucuuagacgaagcau

SEQ ID NO: 577 - RNA, 17 nt, Homo sapiens
uagacgaagcauacgug

SEQ ID NO: 578 - RNA, 17 nt, Homo sapiens
gacgaagcauacgugau

SEQ ID NO: 579 - RNA, 17 nt, Homo sapiens
cgaagcauacgugaugg

SEQ ID NO: 580 - RNA, 17 nt, Homo sapiens
aagcauacgugauggcu

SEQ ID NO: 581 - RNA, 17 nt, Homo sapiens
cauacgugauggcuggu

SEQ ID NO: 582 - RNA, 17 nt, Homo sapiens
acgugauggcuggugug

SEQ ID NO: 583 - RNA, 17 nt, Homo sapiens
gauggcuggugugggcu

SEQ ID NO: 584 - RNA, 17 nt, Homo sapiens
uggcuggugugggcucc

SEQ ID NO: 585 - RNA, 17 nt, Homo sapiens
uggugugggcuccccau

SEQ ID NO: 586 - RNA, 17 nt, Homo sapiens
ggcuccccauaugucuc

SEQ ID NO: 587 - RNA, 17 nt, Homo sapiens
cuccccauaugucuccc

SEQ ID NO: 588 - RNA, 17 nt, Homo sapiens
ccccauaugucucccgc

SEQ ID NO: 589 - RNA, 17 nt, Homo sapiens
ugucucccgccuucugg

SEQ ID NO: 590 - RNA, 17 nt, Homo sapiens
ccuucugggcaucugcc

SEQ ID NO: 591 - RNA, 17 nt, Homo sapiens
uucugggcaucugccug

SEQ ID NO: 592 - RNA, 17 nt, Homo sapiens
gggcaucugccugacau

SEQ ID NO: 593 - RNA, 17 nt, Homo sapiens
ucugccugacauccacg

SEQ ID NO: 594 - RNA, 17 nt, Homo sapiens
ugccugacauccacggu

SEQ ID NO: 595 - RNA, 17 nt, Homo sapiens
ugacauccacggugcag

SEQ ID NO: 596 - RNA, 17 nt, Homo sapiens
cauccacggugcagcug

SEQ ID NO: 597 - RNA, 17 nt, Homo sapiens
uccacggugcagcuggu

SEQ ID NO: 598 - RNA, 17 nt, Homo sapiens
acggugcagcuggugac

SEQ ID NO: 599 - RNA, 17 nt, Homo sapiens
ugcagcuggugacacag

SEQ ID NO: 600 - RNA, 17 nt, Homo sapiens
agcuggugacacagcuu

SEQ ID NO: 601 - RNA, 17 nt, Homo sapiens
cuggugacacagcuuau

SEQ ID NO: 602 - RNA, 17 nt, Homo sapiens
gugacacagcuuaugcc

SEQ ID NO: 603 - RNA, 17 nt, Homo sapiens
cacagcuuaugcccuau

SEQ ID NO: 604 - RNA, 17 nt, Homo sapiens
cagcuuaugcccuaugg

SEQ ID NO: 605 - RNA, 17 nt, Homo sapiens
uaugcccuauggcugcc

SEQ ID NO: 606 - RNA, 17 nt, Homo sapiens
gcccuauggcugccucu

SEQ ID NO: 607 - RNA, 17 nt, Homo sapiens
cuauggcugccucuuag

SEQ ID NO: 608 - RNA, 17 nt, Homo sapiens
ccucuuagaccaugucc

SEQ ID NO: 609 - RNA, 17 nt, Homo sapiens
cuuagaccauguccggg

SEQ ID NO: 610 - RNA, 17 nt, Homo sapiens
uagaccauguccgggaa

SEQ ID NO: 611 - RNA, 17 nt, Homo sapiens
ccgggaaaaccgcggac

SEQ ID NO: 612 - RNA, 17 nt, Homo sapiens
ggaaaaccgcggacgcc

SEQ ID NO: 613 - RNA, 17 nt, Homo sapiens
aaccgcggacgccuggg

SEQ ID NO: 614 - RNA, 17 nt, Homo sapiens
ccgcggacgccugggcu

SEQ ID NO: 615 - RNA, 17 nt, Homo sapiens
acgccugggcucccagg

SEQ ID NO: 616 - RNA, 17 nt, Homo sapiens
cucccaggaccugcuga

SEQ ID NO: 617 - RNA, 17 nt, Homo sapiens
caggaccugcugaacug

SEQ ID NO: 618 - RNA, 17 nt, Homo sapiens
ccugcugaacuggugua

SEQ ID NO: 619 - RNA, 17 nt, Homo sapiens
cugaacugguguaugca

SEQ ID NO: 620 - RNA, 17 nt, Homo sapiens
gaacugguguaugcaga

SEQ ID NO: 621 - RNA, 17 nt, Homo sapiens
acugguguaugcagauu

SEQ ID NO: 622 - RNA, 17 nt, Homo sapiens
ugguguaugcagauugc

SEQ ID NO: 623 - RNA, 17 nt, Homo sapiens
guaugcagauugccaag

SEQ ID NO: 624 - RNA, 17 nt, Homo sapiens
ugcagauugccaagggg

SEQ ID NO: 625 - RNA, 17 nt, Homo sapiens
ccaaggggaugagcuac

SEQ ID NO: 626 - RNA, 17 nt, Homo sapiens
ggggaugagcuaccugg

SEQ ID NO: 627 - RNA, 17 nt, Homo sapiens
gaugagcuaccuggagg

SEQ ID NO: 628 - RNA, 17 nt, Homo sapiens
ccuggaggaugugcggc

SEQ ID NO: 629 - RNA, 17 nt, Homo sapiens
uggaggaugugcggcuc

SEQ ID NO: 630 - RNA, 17 nt, Homo sapiens
gaggaugugcggcucgu

SEQ ID NO: 631 - RNA, 17 nt, Homo sapiens
gaugugcggcucguaca

SEQ ID NO: 632 - RNA, 17 nt, Homo sapiens
ugcggcucguacacagg

SEQ ID NO: 633 - RNA, 17 nt, Homo sapiens
cggcucguacacaggga

SEQ ID NO: 634 - RNA, 17 nt, Homo sapiens
gcucguacacagggacu

SEQ ID NO: 635 - RNA, 17 nt, Homo sapiens
acacagggacuuggccg

SEQ ID NO: 636 - RNA, 17 nt, Homo sapiens
gggacuuggccgcucgg

SEQ ID NO: 637 - RNA, 17 nt, Homo sapiens
acuuggccgcucggaac

SEQ ID NO: 638 - RNA, 17 nt, Homo sapiens
cgcucggaacgugcugg

SEQ ID NO: 639 - RNA, 17 nt, Homo sapiens
cucggaacgugcugguc

SEQ ID NO: 640 - RNA, 17 nt, Homo sapiens
cggaacgugcuggucaa

SEQ ID NO: 641 - RNA, 17 nt, Homo sapiens
acgugcuggucaagagu

SEQ ID NO: 642 - RNA, 17 nt, Homo sapiens
ggucaagagucccaacc

SEQ ID NO: 643 - RNA, 17 nt, Homo sapiens
gagucccaaccauguca

SEQ ID NO: 644 - RNA, 17 nt, Homo sapiens
ucccaaccaugucaaaa

SEQ ID NO: 645 - RNA, 17 nt, Homo sapiens
ccaaccaugucaaaauu

SEQ ID NO: 646 - RNA, 17 nt, Homo sapiens
augucaaaauuacagac

SEQ ID NO: 647 - RNA, 17 nt, Homo sapiens
ucaaaauuacagacuuc

SEQ ID NO: 648 - RNA, 17 nt, Homo sapiens
aauuacagacuucgggc

SEQ ID NO: 649 - RNA, 17 nt, Homo sapiens
gacuucgggcuggcucg

SEQ ID NO: 650 - RNA, 17 nt, Homo sapiens
ucgggcuggcucggcug

SEQ ID NO: 651 - RNA, 17 nt, Homo sapiens
cuggcucggcugcugga

SEQ ID NO: 652 - RNA, 17 nt, Homo sapiens
gcucggcugcuggacau

SEQ ID NO: 653 - RNA, 17 nt, Homo sapiens
gcugcuggacauugacg

SEQ ID NO: 654 - RNA, 17 nt, Homo sapiens
ugcuggacauugacgag

SEQ ID NO: 655 - RNA, 17 nt, Homo sapiens
ggacauugacgagacag

SEQ ID NO: 656 - RNA, 17 nt, Homo sapiens
uugacgagacagaguac

SEQ ID NO: 657 - RNA, 17 nt, Homo sapiens
gagacagaguaccaugc

SEQ ID NO: 658 - RNA, 17 nt, Homo sapiens
gacagaguaccaugcag

SEQ ID NO: 659 - RNA, 17 nt, Homo sapiens
agaguaccaugcagaug

SEQ ID NO: 660 - RNA, 17 nt, Homo sapiens
aguaccaugcagauggg

SEQ ID NO: 661 - RNA, 17 nt, Homo sapiens
ccaugcagaugggggca

SEQ ID NO: 662 - RNA, 17 nt, Homo sapiens
agaugggggcaaggugc

SEQ ID NO: 663 - RNA, 17 nt, Homo sapiens
ggggcaaggugcccauc

SEQ ID NO: 664 - RNA, 17 nt, Homo sapiens
ggcaaggugcccaucaa

SEQ ID NO: 665 - RNA, 17 nt, Homo sapiens
aggugcccaucaagugg

SEQ ID NO: 666 - RNA, 17 nt, Homo sapiens
cccaucaaguggauggc

SEQ ID NO: 667 - RNA, 17 nt, Homo sapiens
ucaaguggauggcgcug

SEQ ID NO: 668 - RNA, 17 nt, Homo sapiens
aguggauggcgcuggag

SEQ ID NO: 669 - RNA, 17 nt, Homo sapiens
uggauggcgcuggaguc

SEQ ID NO: 670 - RNA, 17 nt, Homo sapiens
gcgcuggaguccauucu

SEQ ID NO: 671 - RNA, 17 nt, Homo sapiens
uggaguccauucuccgc

SEQ ID NO: 672 - RNA, 17 nt, Homo sapiens
cauucuccgccggcggu

SEQ ID NO: 673 - RNA, 17 nt, Homo sapiens
cuccgccggcgguucac

SEQ ID NO: 674 - RNA, 17 nt, Homo sapiens
cgccggcgguucaccca

SEQ ID NO: 675 - RNA, 17 nt, Homo sapiens
ggcgguucacccaccag

SEQ ID NO: 676 - RNA, 17 nt, Homo sapiens
guucacccaccagagug

SEQ ID NO: 677 - RNA, 17 nt, Homo sapiens
ccaccagagugaugugu

SEQ ID NO: 678 - RNA, 17 nt, Homo sapiens
ccagagugaugugugga

SEQ ID NO: 679 - RNA, 17 nt, Homo sapiens
agagugauguguggagu

SEQ ID NO: 680 - RNA, 17 nt, Homo sapiens
agugauguguggaguua

SEQ ID NO: 681 - RNA, 17 nt, Homo sapiens
uguguggaguuauggug

SEQ ID NO: 682 - RNA, 17 nt, Homo sapiens
guggaguuaugguguga

SEQ ID NO: 683 - RNA, 17 nt, Homo sapiens
gaguuauggugugacug

SEQ ID NO: 684 - RNA, 17 nt, Homo sapiens
guuauggugugacugug

SEQ ID NO: 685 - RNA, 17 nt, Homo sapiens
auggugugacugugugg

SEQ ID NO: 686 - RNA, 17 nt, Homo sapiens
gugugacugugugggag

SEQ ID NO: 687 - RNA, 17 nt, Homo sapiens
gugacugugugggagcu

SEQ ID NO: 688 - RNA, 17 nt, Homo sapiens
gugugggagcugaugac

SEQ ID NO: 689 - RNA, 17 nt, Homo sapiens
gggagcugaugacuuuu

SEQ ID NO: 690 - RNA, 17 nt, Homo sapiens
agcugaugacuuuuggg

SEQ ID NO: 691 - RNA, 17 nt, Homo sapiens
cuuuuggggccaaaccu

SEQ ID NO: 692 - RNA, 17 nt, Homo sapiens
ggggccaaaccuuacga

SEQ ID NO: 693 - RNA, 17 nt, Homo sapiens
caaaccuuacgauggga

SEQ ID NO: 694 - RNA, 17 nt, Homo sapiens
accuuacgaugggaucc

SEQ ID NO: 695 - RNA, 17 nt, Homo sapiens
acgaugggaucccagcc

SEQ ID NO: 696 - RNA, 17 nt, Homo sapiens
ggaucccagcccgggag

SEQ ID NO: 697 - RNA, 17 nt, Homo sapiens
cccgggagaucccugac

SEQ ID NO: 698 - RNA, 17 nt, Homo sapiens
gaucccugaccugcugg

SEQ ID NO: 699 - RNA, 17 nt, Homo sapiens
ccugaccugcuggaaaa

SEQ ID NO: 700 - RNA, 17 nt, Homo sapiens
aagggggagcggcugcc

SEQ ID NO: 701 - RNA, 17 nt, Homo sapiens
ggggagcggcugcccca

SEQ ID NO: 702 - RNA, 17 nt, Homo sapiens
gagcggcugccccagcc

SEQ ID NO: 703 - RNA, 17 nt, Homo sapiens
cugccccagccccccau

SEQ ID NO: 704 - RNA, 17 nt, Homo sapiens
agccccccaucugcacc

SEQ ID NO: 705 - RNA, 17 nt, Homo sapiens
ccccaucugcaccauug

SEQ ID NO: 706 - RNA, 17 nt, Homo sapiens
ccaucugcaccauugau

SEQ ID NO: 707 - RNA, 17 nt, Homo sapiens
ucugcaccauugauguc

SEQ ID NO: 708 - RNA, 17 nt, Homo sapiens
caccauugaugucuaca

SEQ ID NO: 709 - RNA, 17 nt, Homo sapiens
ccauugaugucuacaug

SEQ ID NO: 710 - RNA, 17 nt, Homo sapiens
ugaugucuacaugauca

SEQ ID NO: 711 - RNA, 17 nt, Homo sapiens
augucuacaugaucaug

SEQ ID NO: 712 - RNA, 17 nt, Homo sapiens
ucuacaugaucaugguc

SEQ ID NO: 713 - RNA, 17 nt, Homo sapiens
acaugaucauggucaaa

SEQ ID NO: 714 - RNA, 17 nt, Homo sapiens
ugaucauggucaaaugu

SEQ ID NO: 715 - RNA, 17 nt, Homo sapiens
auggucaaauguuggau

SEQ ID NO: 716 - RNA, 17 nt, Homo sapiens
ggucaaauguuggauga

SEQ ID NO: 717 - RNA, 17 nt, Homo sapiens
aauguuggaugauugac

SEQ ID NO: 718 - RNA, 17 nt, Homo sapiens
guuggaugauugacucu

SEQ ID NO: 719 - RNA, 17 nt, Homo sapiens
gaugauugacucugaau

SEQ ID NO: 720 - RNA, 17 nt, Homo sapiens
gacucugaaugucggcc

SEQ ID NO: 721 - RNA, 17 nt, Homo sapiens
cucugaaugucggccaa

SEQ ID NO: 722 - RNA, 17 nt, Homo sapiens
gaaugucggccaagauu

SEQ ID NO: 723 - RNA, 17 nt, Homo sapiens
cggccaagauuccggga

SEQ ID NO: 724 - RNA, 17 nt, Homo sapiens
uuccgggaguugguguc

SEQ ID NO: 725 - RNA, 17 nt, Homo sapiens
gggaguuggugucugaa

SEQ ID NO: 726 - RNA, 17 nt, Homo sapiens
gaguuggugucugaauu

SEQ ID NO: 727 - RNA, 17 nt, Homo sapiens
gugucugaauucucccg

SEQ ID NO: 728 - RNA, 17 nt, Homo sapiens
auucucccgcauggcca

SEQ ID NO: 729 - RNA, 17 nt, Homo sapiens
ucucccgcauggccagg

SEQ ID NO: 730 - RNA, 17 nt, Homo sapiens
cccgcauggccagggac

SEQ ID NO: 731 - RNA, 17 nt, Homo sapiens
ggccagggacccccagc

SEQ ID NO: 732 - RNA, 17 nt, Homo sapiens
gacccccagcgcuuugu

SEQ ID NO: 733 - RNA, 17 nt, Homo sapiens
cccccagcgcuuugugg

SEQ ID NO: 734 - RNA, 17 nt, Homo sapiens
agcgcuuuguggucauc

SEQ ID NO: 735 - RNA, 17 nt, Homo sapiens
gcuuuguggucauccag

SEQ ID NO: 736 - RNA, 17 nt, Homo sapiens
uuguggucauccagaau

SEQ ID NO: 737 - RNA, 17 nt, Homo sapiens
cauccagaaugaggacu

SEQ ID NO: 738 - RNA, 17 nt, Homo sapiens
gaaugaggacuugggcc

SEQ ID NO: 739 - RNA, 17 nt, Homo sapiens
ggacuugggcccagcca

SEQ ID NO: 740 - RNA, 17 nt, Homo sapiens
ugggcccagccaguccc

SEQ ID NO: 741 - RNA, 17 nt, Homo sapiens
cccagccagucccuugg

SEQ ID NO: 742 - RNA, 17 nt, Homo sapiens
ucccuuggacagcaccu

SEQ ID NO: 743 - RNA, 17 nt, Homo sapiens
cuuggacagcaccuucu

SEQ ID NO: 744 - RNA, 17 nt, Homo sapiens
uggacagcaccuucuac

SEQ ID NO: 745 - RNA, 17 nt, Homo sapiens
caccuucuaccgcucac

SEQ ID NO: 746 - RNA, 17 nt, Homo sapiens
cuucuaccgcucacugc

SEQ ID NO: 747 - RNA, 17 nt, Homo sapiens
uaccgcucacugcugga

SEQ ID NO: 748 - RNA, 17 nt, Homo sapiens
cgcucacugcuggagga

SEQ ID NO: 749 - RNA, 17 nt, Homo sapiens
gcuggaggacgaugaca

SEQ ID NO: 750 - RNA, 17 nt, Homo sapiens
ggaggacgaugacaugg

SEQ ID NO: 751 - RNA, 17 nt, Homo sapiens
ggacgaugacauggggg

SEQ ID NO: 752 - RNA, 17 nt, Homo sapiens
acgaugacaugggggac

SEQ ID NO: 753 - RNA, 17 nt, Homo sapiens
caugggggaccuggugg

SEQ ID NO: 754 - RNA, 17 nt, Homo sapiens
gggaccugguggaugcu

SEQ ID NO: 755 - RNA, 17 nt, Homo sapiens
ccugguggaugcugagg

SEQ ID NO: 756 - RNA, 17 nt, Homo sapiens
ugguggaugcugaggag

SEQ ID NO: 757 - RNA, 17 nt, Homo sapiens
gcugaggaguaucuggu

SEQ ID NO: 758 - RNA, 17 nt, Homo sapiens
ugaggaguaucugguac

SEQ ID NO: 759 - RNA, 17 nt, Homo sapiens
aguaucugguaccccag

SEQ ID NO: 760 - RNA, 17 nt, Homo sapiens
uaucugguaccccagca

SEQ ID NO: 761 - RNA, 17 nt, Homo sapiens
guaccccagcagggcuu

SEQ ID NO: 762 - RNA, 17 nt, Homo sapiens
ccagcagggcuucuucu

SEQ ID NO: 763 - RNA, 17 nt, Homo sapiens
cuucuucuguccagacc

SEQ ID NO: 764 - RNA, 17 nt, Homo sapiens
cuguccagacccugccc

SEQ ID NO: 765 - RNA, 17 nt, Homo sapiens
cagacccugccccgggc

SEQ ID NO: 766 - RNA, 17 nt, Homo sapiens
ugccccgggcgcugggg

SEQ ID NO: 767 - RNA, 17 nt, Homo sapiens
ccccgggcgcugggggc

SEQ ID NO: 768 - RNA, 17 nt, Homo sapiens
cgcugggggcauggucc

SEQ ID NO: 769 - RNA, 17 nt, Homo sapiens
cugggggcaugguccac

SEQ ID NO: 770 - RNA, 17 nt, Homo sapiens
ggggcaugguccaccac

SEQ ID NO: 771 - RNA, 17 nt, Homo sapiens
caugguccaccacaggc

SEQ ID NO: 772 - RNA, 17 nt, Homo sapiens
gguccaccacaggcacc

SEQ ID NO: 773 - RNA, 17 nt, Homo sapiens
caccacaggcaccgcag

SEQ ID NO: 774 - RNA, 17 nt, Homo sapiens
ccacaggcaccgcagcu

SEQ ID NO: 775 - RNA, 17 nt, Homo sapiens
caggcaccgcagcucau

SEQ ID NO: 776 - RNA, 17 nt, Homo sapiens
gcaccgcagcucaucua

SEQ ID NO: 777 - RNA, 17 nt, Homo sapiens
cgcagcucaucuaccag

SEQ ID NO: 778 - RNA, 17 nt, Homo sapiens
gcucaucuaccaggagu

SEQ ID NO: 779 - RNA, 17 nt, Homo sapiens
uaccaggaguggcggug

SEQ ID NO: 780 - RNA, 17 nt, Homo sapiens
caggaguggcggugggg

SEQ ID NO: 781 - RNA, 17 nt, Homo sapiens
gaguggcgguggggacc

SEQ ID NO: 782 - RNA, 17 nt, Homo sapiens
cgguggggaccugacac

SEQ ID NO: 783 - RNA, 17 nt, Homo sapiens
gggaccugacacuaggg

SEQ ID NO: 784 - RNA, 17 nt, Homo sapiens
gaccugacacuagggcu

SEQ ID NO: 785 - RNA, 17 nt, Homo sapiens
acacuagggcuggagcc

SEQ ID NO: 786 - RNA, 17 nt, Homo sapiens
gggcuggagcccucuga

SEQ ID NO: 787 - RNA, 17 nt, Homo sapiens
aagaggaggcccccagg

SEQ ID NO: 788 - RNA, 17 nt, Homo sapiens
gcccccaggucuccacu

SEQ ID NO: 789 - RNA, 17 nt, Homo sapiens
aggucuccacuggcacc

SEQ ID NO: 790 - RNA, 17 nt, Homo sapiens
cuccacuggcacccucc

SEQ ID NO: 791 - RNA, 17 nt, Homo sapiens
ccacuggcacccuccga

SEQ ID NO: 792 - RNA, 17 nt, Homo sapiens
ccgaaggggcuggcucc

SEQ ID NO: 793 - RNA, 17 nt, Homo sapiens
aggggcuggcuccgaug

SEQ ID NO: 794 - RNA, 17 nt, Homo sapiens
uggcuccgauguauuug

SEQ ID NO: 795 - RNA, 17 nt, Homo sapiens
gcuccgauguauuugau

SEQ ID NO: 796 - RNA, 17 nt, Homo sapiens
uccgauguauuugaugg

SEQ ID NO: 797 - RNA, 17 nt, Homo sapiens
uguauuugauggugacc

SEQ ID NO: 798 - RNA, 17 nt, Homo sapiens
auuugauggugaccugg

SEQ ID NO: 799 - RNA, 17 nt, Homo sapiens
ugauggugaccugggaa

SEQ ID NO: 800 - RNA, 17 nt, Homo sapiens
accugggaaugggggca

SEQ ID NO: 801 - RNA, 17 nt, Homo sapiens
gaaugggggcagccaag

SEQ ID NO: 802 - RNA, 17 nt, Homo sapiens
ugggggcagccaagggg

SEQ ID NO: 803 - RNA, 17 nt, Homo sapiens
gccaaggggcugcaaag

SEQ ID NO: 804 - RNA, 17 nt, Homo sapiens
aaggggcugcaaagccu

SEQ ID NO: 805 - RNA, 17 nt, Homo sapiens
gcugcaaagccucccca

SEQ ID NO: 806 - RNA, 17 nt, Homo sapiens
gccuccccacacaugac

SEQ ID NO: 807 - RNA, 17 nt, Homo sapiens
cuccccacacaugaccc

SEQ ID NO: 808 - RNA, 17 nt, Homo sapiens
ccccacacaugacccca

SEQ ID NO: 809 - RNA, 17 nt, Homo sapiens
cacacaugaccccagcc

SEQ ID NO: 810 - RNA, 17 nt, Homo sapiens
ugaccccagcccucuac

SEQ ID NO: 811 - RNA, 17 nt, Homo sapiens
agcccucuacagcggua

SEQ ID NO: 812 - RNA, 17 nt, Homo sapiens
ccucuacagcgguacag

SEQ ID NO: 813 - RNA, 17 nt, Homo sapiens
cuacagcgguacaguga

SEQ ID NO: 814 - RNA, 17 nt, Homo sapiens
acagcgguacagugagg

SEQ ID NO: 815 - RNA, 17 nt, Homo sapiens
gcgguacagugaggacc

SEQ ID NO: 816 - RNA, 17 nt, Homo sapiens
cagugaggaccccacag

SEQ ID NO: 817 - RNA, 17 nt, Homo sapiens
aggaccccacaguaccc

SEQ ID NO: 818 - RNA, 17 nt, Homo sapiens
accccacaguaccccug

SEQ ID NO: 819 - RNA, 17 nt, Homo sapiens
cccacaguaccccugcc

SEQ ID NO: 820 - RNA, 17 nt, Homo sapiens
guaccccugcccucuga

SEQ ID NO: 821 - RNA, 17 nt, Homo sapiens
ccucugagacugauggc

SEQ ID NO: 822 - RNA, 17 nt, Homo sapiens
ugagacugauggcuacg

SEQ ID NO: 823 - RNA, 17 nt, Homo sapiens
gacugauggcuacguug

SEQ ID NO: 824 - RNA, 17 nt, Homo sapiens
ugauggcuacguugccc

SEQ ID NO: 825 - RNA, 17 nt, Homo sapiens
auggcuacguugccccc

SEQ ID NO: 826 - RNA, 17 nt, Homo sapiens
gcuacguugccccccug

SEQ ID NO: 827 - RNA, 17 nt, Homo sapiens
ccccccugaccugcagc

SEQ ID NO: 828 - RNA, 17 nt, Homo sapiens
ccugaccugcagccccc

SEQ ID NO: 829 - RNA, 17 nt, Homo sapiens
gaccugcagcccccagc

SEQ ID NO: 830 - RNA, 17 nt, Homo sapiens
agcccccagccugaaua

SEQ ID NO: 831 - RNA, 17 nt, Homo sapiens
cagccugaauaugugaa

SEQ ID NO: 832 - RNA, 17 nt, Homo sapiens
gccugaauaugugaacc

SEQ ID NO: 833 - RNA, 17 nt, Homo sapiens
cugaauaugugaaccag

SEQ ID NO: 834 - RNA, 17 nt, Homo sapiens
auaugugaaccagccag

SEQ ID NO: 835 - RNA, 17 nt, Homo sapiens
gugaaccagccagaugu

SEQ ID NO: 836 - RNA, 17 nt, Homo sapiens
ccagccagauguucggc

SEQ ID NO: 837 - RNA, 17 nt, Homo sapiens
agccagauguucggccc

SEQ ID NO: 838 - RNA, 17 nt, Homo sapiens
gauguucggccccagcc

SEQ ID NO: 839 - RNA, 17 nt, Homo sapiens
cggccccagcccccuuc

SEQ ID NO: 840 - RNA, 17 nt, Homo sapiens
cccccuucgccccgaga

SEQ ID NO: 841 - RNA, 17 nt, Homo sapiens
ccgagagggcccucugc

SEQ ID NO: 842 - RNA, 17 nt, Homo sapiens
ggcccucugccugcugc

SEQ ID NO: 843 - RNA, 17 nt, Homo sapiens
cucugccugcugcccga

SEQ ID NO: 844 - RNA, 17 nt, Homo sapiens
ugccugcugcccgaccu

SEQ ID NO: 845 - RNA, 17 nt, Homo sapiens
gcugcccgaccugcugg

SEQ ID NO: 846 - RNA, 17 nt, Homo sapiens
cccgaccugcuggugcc

SEQ ID NO: 847 - RNA, 17 nt, Homo sapiens
accugcuggugccacuc

SEQ ID NO: 848 - RNA, 17 nt, Homo sapiens
cugcuggugccacucug

SEQ ID NO: 849 - RNA, 17 nt, Homo sapiens
cuggugccacucuggaa

SEQ ID NO: 850 - RNA, 17 nt, Homo sapiens
cuggaaaggcccaagac

SEQ ID NO: 851 - RNA, 17 nt, Homo sapiens
ggcccaagacucucucc

SEQ ID NO: 852 - RNA, 17 nt, Homo sapiens
agggaagaauggggucg

SEQ ID NO: 853 - RNA, 17 nt, Homo sapiens
agaauggggucgucaaa

SEQ ID NO: 854 - RNA, 17 nt, Homo sapiens
auggggucgucaaagac

SEQ ID NO: 855 - RNA, 17 nt, Homo sapiens
cgucaaagacguuuuug

SEQ ID NO: 856 - RNA, 17 nt, Homo sapiens
ucaaagacguuuuugcc

SEQ ID NO: 857 - RNA, 17 nt, Homo sapiens
acguuuuugccuuuggg

SEQ ID NO: 858 - RNA, 17 nt, Homo sapiens
cuuugggggugccgugg

SEQ ID NO: 859 - RNA, 17 nt, Homo sapiens
uugggggugccguggag

SEQ ID NO: 860 - RNA, 17 nt, Homo sapiens
ggggugccguggagaac

SEQ ID NO: 861 - RNA, 17 nt, Homo sapiens
cguggagaaccccgagu

SEQ ID NO: 862 - RNA, 17 nt, Homo sapiens
aaccccgaguacuugac

SEQ ID NO: 863 - RNA, 17 nt, Homo sapiens
ccccgaguacuugacac

SEQ ID NO: 864 - RNA, 17 nt, Homo sapiens
aguacuugacaccccag

SEQ ID NO: 865 - RNA, 17 nt, Homo sapiens
uacuugacaccccaggg

SEQ ID NO: 866 - RNA, 17 nt, Homo sapiens
agggaggagcugccccu

SEQ ID NO: 867 - RNA, 17 nt, Homo sapiens
gaggagcugccccucag

SEQ ID NO: 868 - RNA, 17 nt, Homo sapiens
gccccucagccccaccc

SEQ ID NO: 869 - RNA, 17 nt, Homo sapiens
ucagccccacccuccuc

SEQ ID NO: 870 - RNA, 17 nt, Homo sapiens
cuccuccugccuucagc

SEQ ID NO: 871 - RNA, 17 nt, Homo sapiens
ugccuucagcccagccu

SEQ ID NO: 872 - RNA, 17 nt, Homo sapiens
ucagcccagccuucgac

SEQ ID NO: 873 - RNA, 17 nt, Homo sapiens
agccuucgacaaccucu

SEQ ID NO: 874 - RNA, 17 nt, Homo sapiens
cuucgacaaccucuauu

SEQ ID NO: 875 - RNA, 17 nt, Homo sapiens
caaccucuauuacuggg

SEQ ID NO: 876 - RNA, 17 nt, Homo sapiens
ccucuauuacugggacc

SEQ ID NO: 877 - RNA, 17 nt, Homo sapiens
uuacugggaccaggacc

SEQ ID NO: 878 - RNA, 17 nt, Homo sapiens
ggaccaggacccaccag

SEQ ID NO: 879 - RNA, 17 nt, Homo sapiens
caggacccaccagagcg

SEQ ID NO: 880 - RNA, 17 nt, Homo sapiens
ccaccagagcggggggc

SEQ ID NO: 881 - RNA, 17 nt, Homo sapiens
agcggggggcuccaccc

SEQ ID NO: 882 - RNA, 17 nt, Homo sapiens
ggggcuccacccagcac

SEQ ID NO: 883 - RNA, 17 nt, Homo sapiens
uccacccagcaccuuca

SEQ ID NO: 884 - RNA, 17 nt, Homo sapiens
cacccagcaccuucaaa

SEQ ID NO: 885 - RNA, 17 nt, Homo sapiens
ucaaagggacaccuacg

SEQ ID NO: 886 - RNA, 17 nt, Homo sapiens
aaagggacaccuacggc

SEQ ID NO: 887 - RNA, 17 nt, Homo sapiens
ggacaccuacggcagag

SEQ ID NO: 888 - RNA, 17 nt, Homo sapiens
caccuacggcagagaac

SEQ ID NO: 889 - RNA, 17 nt, Homo sapiens
ggcagagaacccagagu

SEQ ID NO: 890 - RNA, 17 nt, Homo sapiens
aacccagaguaccuggg

SEQ ID NO: 891 - RNA, 17 nt, Homo sapiens
cccagaguaccuggguc

SEQ ID NO: 892 - RNA, 17 nt, Homo sapiens
guaccugggucuggacg

SEQ ID NO: 893 - RNA, 17 nt, Homo sapiens
gggucuggacgugccag

SEQ ID NO: 894 - RNA, 17 nt, Homo sapiens
gucuggacgugccagug

SEQ ID NO: 895 - RNA, 17 nt, Homo sapiens
cuggacgugccagugug

SEQ ID NO: 896 - RNA, 17 nt, Homo sapiens
acgugccagugugaacc

SEQ ID NO: 897 - RNA, 17 nt, Homo sapiens
gugccagugugaaccag

SEQ ID NO: 898 - RNA, 17 nt, Homo sapiens
cagugugaaccagaagg

SEQ ID NO: 899 - RNA, 17 nt, Homo sapiens
accagaaggccaagucc

SEQ ID NO: 900 - RNA, 17 nt, Homo sapiens
aaggccaaguccgcaga

SEQ ID NO: 901 - RNA, 17 nt, Homo sapiens
ccaaguccgcagaagcc

SEQ ID NO: 902 - RNA, 17 nt, Homo sapiens
ccgcagaagcccugaug

SEQ ID NO: 903 - RNA, 17 nt, Homo sapiens
aagcccugauguguccu

SEQ ID NO: 904 - RNA, 17 nt, Homo sapiens
gcccugauguguccuca

SEQ ID NO: 905 - RNA, 17 nt, Homo sapiens
ccugauguguccucagg

SEQ ID NO: 906 - RNA, 17 nt, Homo sapiens
cucagggagcagggaag

SEQ ID NO: 907 - RNA, 17 nt, Homo sapiens
cagggaaggccugacuu

SEQ ID NO: 908 - RNA, 17 nt, Homo sapiens
aaggccugacuucugcu

SEQ ID NO: 909 - RNA, 17 nt, Homo sapiens
ugacuucugcuggcauc

SEQ ID NO: 910 - RNA, 17 nt, Homo sapiens
uucugcuggcaucaaga

SEQ ID NO: 911 - RNA, 17 nt, Homo sapiens
cugcuggcaucaagagg

SEQ ID NO: 912 - RNA, 17 nt, Homo sapiens
aucaagaggugggaggg

SEQ ID NO: 913 - RNA, 17 nt, Homo sapiens
gugggagggcccuccga

SEQ ID NO: 914 - RNA, 17 nt, Homo sapiens
gcccuccgaccacuucc

SEQ ID NO: 915 - RNA, 17 nt, Homo sapiens
cuccgaccacuuccagg

SEQ ID NO: 916 - RNA, 17 nt, Homo sapiens
ccaggggaaccugccau

SEQ ID NO: 917 - RNA, 17 nt, Homo sapiens
gggaaccugccaugcca

SEQ ID NO: 918 - RNA, 17 nt, Homo sapiens
aaccugccaugccagga

SEQ ID NO: 919 - RNA, 17 nt, Homo sapiens
ccugccaugccaggaac

SEQ ID NO: 920 - RNA, 17 nt, Homo sapiens
ugccaggaaccuguccu

SEQ ID NO: 921 - RNA, 17 nt, Homo sapiens
aggaaccuguccuaagg

SEQ ID NO: 922 - RNA, 17 nt, Homo sapiens
ccuaaggaaccuuccuu

SEQ ID NO: 923 - RNA, 17 nt, Homo sapiens
uccuuccugcuugaguu

SEQ ID NO: 924 - RNA, 17 nt, Homo sapiens
cugcuugaguucccaga

SEQ ID NO: 925 - RNA, 17 nt, Homo sapiens
guucccagauggcugga

SEQ ID NO: 926 - RNA, 17 nt, Homo sapiens
cccagauggcuggaagg

SEQ ID NO: 927 - RNA, 17 nt, Homo sapiens
uggaagggguccagccu

SEQ ID NO: 928 - RNA, 17 nt, Homo sapiens
gggguccagccucguug

SEQ ID NO: 929 - RNA, 17 nt, Homo sapiens
ccagccucguuggaaga

SEQ ID NO: 930 - RNA, 17 nt, Homo sapiens
gaagaggaacagcacug

SEQ ID NO: 931 - RNA, 17 nt, Homo sapiens
gaggaacagcacugggg

SEQ ID NO: 932 - RNA, 17 nt, Homo sapiens
ggaacagcacuggggag

SEQ ID NO: 933 - RNA, 17 nt, Homo sapiens
acuggggagucuuugug

SEQ ID NO: 934 - RNA, 17 nt, Homo sapiens
gagucuuuguggauucu

SEQ ID NO: 935 - RNA, 17 nt, Homo sapiens
cuuuguggauucugagg

SEQ ID NO: 936 - RNA, 17 nt, Homo sapiens
auucugaggcccugccc

SEQ ID NO: 937 - RNA, 17 nt, Homo sapiens
gaggcccugcccaauga

SEQ ID NO: 938 - RNA, 17 nt, Homo sapiens
ccugcccaaugagacuc

SEQ ID NO: 939 - RNA, 17 nt, Homo sapiens
ccaaugagacucuaggg

SEQ ID NO: 940 - RNA, 17 nt, Homo sapiens
acucuaggguccagugg

SEQ ID NO: 941 - RNA, 17 nt, Homo sapiens
aggguccaguggaugcc

SEQ ID NO: 942 - RNA, 17 nt, Homo sapiens
uccaguggaugccacag

SEQ ID NO: 943 - RNA, 17 nt, Homo sapiens
caguggaugccacagcc

SEQ ID NO: 944 - RNA, 17 nt, Homo sapiens
uggaugccacagcccag

SEQ ID NO: 945 - RNA, 17 nt, Homo sapiens
augccacagcccagcuu

SEQ ID NO: 946 - RNA, 17 nt, Homo sapiens
acagcccagcuuggccc

SEQ ID NO: 947 - RNA, 17 nt, Homo sapiens
ccagcuuggcccuuucc

SEQ ID NO: 948 - RNA, 17 nt, Homo sapiens
ccuuccagauccugggu

SEQ ID NO: 949 - RNA, 17 nt, Homo sapiens
gauccuggguacugaaa

SEQ ID NO: 950 - RNA, 17 nt, Homo sapiens
uccuggguacugaaagc

SEQ ID NO: 951 - RNA, 17 nt, Homo sapiens
uacugaaagccuuaggg

SEQ ID NO: 952 - RNA, 17 nt, Homo sapiens
uuagggaagcuggccug

SEQ ID NO: 953 - RNA, 17 nt, Homo sapiens
ggaagcuggccugagag

SEQ ID NO: 954 - RNA, 17 nt, Homo sapiens
gaggggaagcggcccua

SEQ ID NO: 955 - RNA, 17 nt, Homo sapiens
gggaagcggcccuaagg

SEQ ID NO: 956 - RNA, 17 nt, Homo sapiens
cuaagggagugucuaag

SEQ ID NO: 957 - RNA, 17 nt, Homo sapiens
aagggagugucuaagaa

SEQ ID NO: 958 - RNA, 17 nt, Homo sapiens
gucuaagaacaaaagcg

SEQ ID NO: 959 - RNA, 17 nt, Homo sapiens
gaacaaaagcgacccau

SEQ ID NO: 960 - RNA, 17 nt, Homo sapiens
caaaagcgacccauuca

SEQ ID NO: 961 - RNA, 17 nt, Homo sapiens
agcgacccauucagaga

SEQ ID NO: 962 - RNA, 17 nt, Homo sapiens
auucagagacugucccu

SEQ ID NO: 963 - RNA, 17 nt, Homo sapiens
cagagacugucccugaa

SEQ ID NO: 964 - RNA, 17 nt, Homo sapiens
ucccugaaaccuaguac

SEQ ID NO: 965 - RNA, 17 nt, Homo sapiens
gaaaccuaguacugccc

SEQ ID NO: 966 - RNA, 17 nt, Homo sapiens
aaccuaguacugccccc

SEQ ID NO: 967 - RNA, 17 nt, Homo sapiens
cuaguacugccccccau

SEQ ID NO: 968 - RNA, 17 nt, Homo sapiens
ugccccccaugaggaag

SEQ ID NO: 969 - RNA, 17 nt, Homo sapiens
aggaaggaacagcaaug

SEQ ID NO: 970 - RNA, 17 nt, Homo sapiens
aaggaacagcaauggug

SEQ ID NO: 971 - RNA, 17 nt, Homo sapiens
gaacagcaaugguguca

SEQ ID NO: 972 - RNA, 17 nt, Homo sapiens
cagcaauggugucagua

SEQ ID NO: 973 - RNA, 17 nt, Homo sapiens
gcaauggugucaguauc

SEQ ID NO: 974 - RNA, 17 nt, Homo sapiens
uggugucaguauccagg

SEQ ID NO: 975 - RNA, 17 nt, Homo sapiens
gugucaguauccaggcu

SEQ ID NO: 976 - RNA, 17 nt, Homo sapiens
guauccaggcuuuguac

SEQ ID NO: 977 - RNA, 17 nt, Homo sapiens
caggcuuuguacagagu

SEQ ID NO: 978 - RNA, 17 nt, Homo sapiens
ggcuuuguacagagugc

SEQ ID NO: 979 - RNA, 17 nt, Homo sapiens
uguacagagugcuuuuc

SEQ ID NO: 980 - RNA, 17 nt, Homo sapiens
uacagagugcuuuucug

SEQ ID NO: 981 - RNA, 17 nt, Homo sapiens
gcuuuucuguuuaguuu

SEQ ID NO: 982 - RNA, 17 nt, Homo sapiens
ucuguuuaguuuuuacu

SEQ ID NO: 983 - RNA, 17 nt, Homo sapiens
uaguuuuuacuuuuuuu

SEQ ID NO: 984 - RNA, 17 nt, Homo sapiens
cuuuuuuuguuuuguuu

SEQ ID NO: 985 - RNA, 17 nt, Homo sapiens
uuuguuuuguuuuuuua

SEQ ID NO: 986 - RNA, 17 nt, Homo sapiens
uuuuaaagaugaaauaa

SEQ ID NO: 987 - RNA, 17 nt, Homo sapiens
aagaugaaauaaagacc

SEQ ID NO: 988 - RNA, 17 nt, Homo sapiens
aaauaaagacccagggg

SEQ ID NO: 989 - DNA, 31 bp, Artificial Sequence
  Description of Artificial Sequence  Enzymatic Nucleic Acid
cagggttaggctagctacaacgactcccctt

SEQ ID NO: 990 - DNA, 31 bp, Artificial Sequence
  Description of Artificial Sequence  Enzymatic Nucleic Acid
ggccagggggctagctacaacgatacctccc

SEQ ID NO: 991 - DNA, 31 bp, Artificial Sequence
  Description of Artificial Sequence  Enzymatic Nucleic Acid
caaaggggggctagctacaacgacagggtta

SEQ ID NO: 992 - DNA, 31 bp, Artificial Sequence
  Description of Artificial Sequence  Enzymatic Nucleic Acid
ggccccgaggctagctacaacgacaaagggg

SEQ ID NO: 993 - DNA, 31 bp, Artificial Sequence
  Description of Artificial Sequence  Enzymatic Nucleic Acid
gcccggggggctagctacaacgacccgacca

SEQ ID NO: 994 - DNA, 31 bp, Artificial Sequence
  Description of Artificial Sequence  Enzymatic Nucleic Acid
cgcggctgggctagctacaacgaccggggcc

SEQ ID NO: 995 - DNA, 31 bp, Artificial Sequence
  Description of Artificial Sequence  Enzymatic Nucleic Acid
gcgcgcggggctagctacaacgatgcccggg

SEQ ID NO: 996 - DNA, 31 bp, Artificial Sequence
  Description of Artificial Sequence  Enzymatic Nucleic Acid
ggggcgcgggctagctacaacgaggctgccc

SEQ ID NO: 997 - DNA, 31 bp, Artificial Sequence
  Description of Artificial Sequence  Enzymatic Nucleic Acid
aaggggcgggctagctacaacgagcggctgc

SEQ ID NO: 998 - DNA, 31 bp, Artificial Sequence
  Description of Artificial Sequence  Enzymatic Nucleic Acid
ggaaggggggctagctacaacgagcgcggct

SEQ ID NO: 999 - DNA, 31 bp, Artificial Sequence
  Description of Artificial Sequence  Enzymatic Nucleic Acid
gggccccgggctagctacaacgagggaaggg

SEQ ID NO: 1000 - DNA, 31 bp, Artificial Sequence
  Description of Artificial Sequence  Enzymatic Nucleic Acid
gtaaagggggctagctacaacgacccgtggg

SEQ ID NO: 1001 - DNA, 31 bp, Artificial Sequence
  Description of Artificial Sequence  Enzymatic Nucleic Acid
cggcgcagggctagctacaacgaaaagggcc

SEQ ID NO: 1002 - DNA, 31 bp, Artificial Sequence
  Description of Artificial Sequence  Enzymatic Nucleic Acid
gcgcggcgggctagctacaacgaagtaaagg

SEQ ID NO: 1003 - DNA, 31 bp, Artificial Sequence
  Description of Artificial Sequence  Enzymatic Nucleic Acid
gcgcgcggggctagctacaacgagcagtaaa

SEQ ID NO: 1004 - DNA, 31 bp, Artificial Sequence
  Description of Artificial Sequence  Enzymatic Nucleic Acid
cgggcgcgggctagctacaacgaggcgcagt

SEQ ID NO: 1005 - DNA, 31 bp, Artificial Sequence
  Description of Artificial Sequence  Enzymatic Nucleic Acid
gccgggcgggctagctacaacgagcggcgca

SEQ ID NO: 1006 - DNA, 31 bp, Artificial Sequence
  Description of Artificial Sequence  Enzymatic Nucleic Acid
gggccgggggctagctacaacgagcgcggcg

SEQ ID NO: 1007 - DNA, 31 bp, Artificial Sequence
  Description of Artificial Sequence  Enzymatic Nucleic Acid
ggtgggggggctagctacaacgacgggcgcg

SEQ ID NO: 1008 - DNA, 31 bp, Artificial Sequence
  Description of Artificial Sequence  Enzymatic Nucleic Acid
gcgaggggggctagctacaacgagggggccg

SEQ ID NO: 1009 - DNA, 31 bp, Artificial Sequence
  Description of Artificial Sequence  Enzymatic Nucleic Acid
gggtgctgggctagctacaacgagaggggtg

SEQ ID NO: 1010 - DNA, 31 bp, Artificial Sequence
  Description of Artificial Sequence  Enzymatic Nucleic Acid
gcggggtgggctagctacaacgatgcgaggg

SEQ ID NO: 1011 - DNA, 31 bp, Artificial Sequence
  Description of Artificial Sequence  Enzymatic Nucleic Acid
gcgcggggggctagctacaacgagctgcgag

SEQ ID NO: 1012 - DNA, 31 bp, Artificial Sequence
  Description of Artificial Sequence  Enzymatic Nucleic Acid
gcggggcgggctagctacaacgaggggtgct

SEQ ID NO: 1013 - DNA, 31 bp, Artificial Sequence
  Description of Artificial Sequence  Enzymatic Nucleic Acid
gcgcggggggctagctacaacgagcggggtg

SEQ ID NO: 1014 - DNA, 31 bp, Artificial Sequence
  Description of Artificial Sequence  Enzymatic Nucleic Acid
ggagggcgggctagctacaacgaggggcgcg

SEQ ID NO: 1015 - DNA, 31 bp, Artificial Sequence
  Description of Artificial Sequence  Enzymatic Nucleic Acid
tgggagggggctagctacaacgagcggggcg

SEQ ID NO: 1016 - DNA, 31 bp, Artificial Sequence
  Description of Artificial Sequence  Enzymatic Nucleic Acid
ggacccggggctagctacaacgatgggaggg

SEQ ID NO: 1017 - DNA, 31 bp, Artificial Sequence
  Description of Artificial Sequence  Enzymatic Nucleic Acid
cggctggaggctagctacaacgaccggctgg

SEQ ID NO: 1018 - DNA, 31 bp, Artificial Sequence
  Description of Artificial Sequence  Enzymatic Nucleic Acid
ggctccggggctagctacaacgatggacccg

SEQ ID NO: 1019 - DNA, 31 bp, Artificial Sequence
  Description of Artificial Sequence  Enzymatic Nucleic Acid
ccccatggggctagctacaacgatccggctg

SEQ ID NO: 1020 - DNA, 31 bp, Artificial Sequence
  Description of Artificial Sequence  Enzymatic Nucleic Acid
cggccccaggctagctacaacgaggctccgg

SEQ ID NO: 1021 - DNA, 31 bp, Artificial Sequence
  Description of Artificial Sequence  Enzymatic Nucleic Acid
ggctccggggctagctacaacgacccatggc

SEQ ID NO: 1022 - DNA, 31 bp, Artificial Sequence
  Description of Artificial Sequence  Enzymatic Nucleic Acid
cactgcggggctagctacaacgatccggccc

SEQ ID NO: 1023 - DNA, 31 bp, Artificial Sequence
  Description of Artificial Sequence  Enzymatic Nucleic Acid
gctcactgggctagctacaacgaggctccgg

SEQ ID NO: 1024 - DNA, 31 bp, Artificial Sequence
  Description of Artificial Sequence  Enzymatic Nucleic Acid
ggtgctcaggctagctacaacgatgcggctc

SEQ ID NO: 1025 - DNA, 31 bp, Artificial Sequence
  Description of Artificial Sequence  Enzymatic Nucleic Acid
ccatggtgggctagctacaacgatcactgcg

SEQ ID NO: 1026 - DNA, 31 bp, Artificial Sequence
  Description of Artificial Sequence  Enzymatic Nucleic Acid
ctccatggggctagctacaacgagctcactg

SEQ ID NO: 1027 - DNA, 31 bp, Artificial Sequence
  Description of Artificial Sequence  Enzymatic Nucleic Acid
cagctccaggctagctacaacgaggtgctca

SEQ ID NO: 1028 - DNA, 31 bp, Artificial Sequence
  Description of Artificial Sequence  Enzymatic Nucleic Acid
gccgccagggctagctacaacgatccatggt

SEQ ID NO: 1029 - DNA, 31 bp, Artificial Sequence
  Description of Artificial Sequence  Enzymatic Nucleic Acid
caaggccgggctagctacaacgacagctcca

SEQ ID NO: 1030 - DNA, 31 bp, Artificial Sequence
  Description of Artificial Sequence  Enzymatic Nucleic Acid
gcacaaggggctagctacaacgacgccagct

SEQ ID NO: 1031 - DNA, 31 bp, Artificial Sequence
  Description of Artificial Sequence  Enzymatic Nucleic Acid
cagcggcaggctagctacaacgaaaggccgc

SEQ ID NO: 1032 - DNA, 31 bp, Artificial Sequence
  Description of Artificial Sequence  Enzymatic Nucleic Acid
cccagcggggctagctacaacgaacaaggcc

SEQ ID NO: 1033 - DNA, 31 bp, Artificial Sequence
  Description of Artificial Sequence  Enzymatic Nucleic Acid
gcccccagggctagctacaacgaggcacaag

SEQ ID NO: 1034 - DNA, 31 bp, Artificial Sequence
  Description of Artificial Sequence  Enzymatic Nucleic Acid
aggaggagggctagctacaacgaccccagcg

SEQ ID NO: 1035 - DNA, 31 bp, Artificial Sequence
  Description of Artificial Sequence  Enzymatic Nucleic Acid
caagagggggctagctacaacgagaggagga

SEQ ID NO: 1036 - DNA, 31 bp, Artificial Sequence
  Description of Artificial Sequence  Enzymatic Nucleic Acid
ccggggggggctagctacaacgaaagagggc

SEQ ID NO: 1037 - DNA, 31 bp, Artificial Sequence
  Description of Artificial Sequence  Enzymatic Nucleic Acid
gctcgcggggctagctacaacgatccggggg

SEQ ID NO: 1038 - DNA, 31 bp, Artificial Sequence
  Description of Artificial Sequence  Enzymatic Nucleic Acid
ggtgctcgggctagctacaacgaggctccgg

SEQ ID NO: 1039 - DNA, 31 bp, Artificial Sequence
  Description of Artificial Sequence  Enzymatic Nucleic Acid
cttgggtgggctagctacaacgatcgcggct

SEQ ID NO: 1040 - DNA, 31 bp, Artificial Sequence
  Description of Artificial Sequence  Enzymatic Nucleic Acid
cacttgggggctagctacaacgagctcgcgg

SEQ ID NO: 1041 - DNA, 31 bp, Artificial Sequence
  Description of Artificial Sequence  Enzymatic Nucleic Acid
ggtgcacaggctagctacaacgattgggtgc

SEQ ID NO: 1042 - DNA, 31 bp, Artificial Sequence
  Description of Artificial Sequence  Enzymatic Nucleic Acid
ccggtgcaggctagctacaacgaacttgggt

SEQ ID NO: 1043 - DNA, 31 bp, Artificial Sequence
  Description of Artificial Sequence  Enzymatic Nucleic Acid
tgccggtgggctagctacaacgaacacttgg

SEQ ID NO: 1044 - DNA, 31 bp, Artificial Sequence
  Description of Artificial Sequence  Enzymatic Nucleic Acid
tgtgccggggctagctacaacgagcacactt

SEQ ID NO: 1045 - DNA, 31 bp, Artificial Sequence
  Description of Artificial Sequence  Enzymatic Nucleic Acid
tgtctgtgggctagctacaacgacggtgcac

SEQ ID NO: 1046 - DNA, 31 bp, Artificial Sequence
  Description of Artificial Sequence  Enzymatic Nucleic Acid
catgtctgggctagctacaacgagccggtgc

SEQ ID NO: 1047 - DNA, 31 bp, Artificial Sequence
  Description of Artificial Sequence  Enzymatic Nucleic Acid
gcttcatgggctagctacaacgactgtgccg

SEQ ID NO: 1048 - DNA, 31 bp, Artificial Sequence
  Description of Artificial Sequence  Enzymatic Nucleic Acid
cagcttcaggctagctacaacgagtctgtgc

SEQ ID NO: 1049 - DNA, 31 bp, Artificial Sequence
  Description of Artificial Sequence  Enzymatic Nucleic Acid
agccgcagggctagctacaacgattcatgtc

SEQ ID NO: 1050 - DNA, 31 bp, Artificial Sequence
  Description of Artificial Sequence  Enzymatic Nucleic Acid
gggagccgggctagctacaacgaagcttcat

SEQ ID NO: 1051 - DNA, 31 bp, Artificial Sequence
  Description of Artificial Sequence  Enzymatic Nucleic Acid
gcagggagggctagctacaacgacgcagctt

SEQ ID NO: 1052 - DNA, 31 bp, Artificial Sequence
  Description of Artificial Sequence  Enzymatic Nucleic Acid
gggactggggctagctacaacgaagggagcc

SEQ ID NO: 1053 - DNA, 31 bp, Artificial Sequence
  Description of Artificial Sequence  Enzymatic Nucleic Acid
tctcgggaggctagctacaacgatggcaggg

SEQ ID NO: 1054 - DNA, 31 bp, Artificial Sequence
  Description of Artificial Sequence  Enzymatic Nucleic Acid
caggtgggggctagctacaacgactcgggac

SEQ ID NO: 1055 - DNA, 31 bp, Artificial Sequence
  Description of Artificial Sequence  Enzymatic Nucleic Acid
tgtccaggggctagctacaacgagggtctcg

SEQ ID NO: 1056 - DNA, 31 bp, Artificial Sequence
  Description of Artificial Sequence  Enzymatic Nucleic Acid
ggagcatgggctagctacaacgaccaggtgg

SEQ ID NO: 1057 - DNA, 31 bp, Artificial Sequence
  Description of Artificial Sequence  Enzymatic Nucleic Acid
gcggagcaggctagctacaacgagtccaggt

SEQ ID NO: 1058 - DNA, 31 bp, Artificial Sequence
  Description of Artificial Sequence  Enzymatic Nucleic Acid
tggcggagggctagctacaacgaatgtccag

SEQ ID NO: 1059 - DNA, 31 bp, Artificial Sequence
  Description of Artificial Sequence  Enzymatic Nucleic Acid
agaggtggggctagctacaacgaggagcatg

SEQ ID NO: 1060 - DNA, 31 bp, Artificial Sequence
  Description of Artificial Sequence  Enzymatic Nucleic Acid
ggtagaggggctagctacaacgaggcggagc

SEQ ID NO: 1061 - DNA, 31 bp, Artificial Sequence
  Description of Artificial Sequence  Enzymatic Nucleic Acid
agccctggggctagctacaacgaagaggtgg

SEQ ID NO: 1062 - DNA, 31 bp, Artificial Sequence
  Description of Artificial Sequence  Enzymatic Nucleic Acid
cctggcagggctagctacaacgacctggtag

SEQ ID NO: 1063 - DNA, 31 bp, Artificial Sequence
  Description of Artificial Sequence  Enzymatic Nucleic Acid
ccacctggggctagctacaacgaagccctgg

SEQ ID NO: 1064 - DNA, 31 bp, Artificial Sequence
  Description of Artificial Sequence  Enzymatic Nucleic Acid
ctgcaccaggctagctacaacgactggcagc

SEQ ID NO: 1065 - DNA, 31 bp, Artificial Sequence
  Description of Artificial Sequence  Enzymatic Nucleic Acid
tccctgcaggctagctacaacgacacctggc

SEQ ID NO: 1066 - DNA, 31 bp, Artificial Sequence
  Description of Artificial Sequence  Enzymatic Nucleic Acid
tttccctgggctagctacaacgaaccacctg

SEQ ID NO: 1067 - DNA, 31 bp, Artificial Sequence
  Description of Artificial Sequence  Enzymatic Nucleic Acid
gttccaggggctagctacaacgattccctgc

SEQ ID NO: 1068 - DNA, 31 bp, Artificial Sequence
  Description of Artificial Sequence  Enzymatic Nucleic Acid
taggtgagggctagctacaacgatccaggtt

SEQ ID NO: 1069 - DNA, 31 bp, Artificial Sequence
  Description of Artificial Sequence  Enzymatic Nucleic Acid
caggtaggggctagctacaacgagagttcca

SEQ ID NO: 1070 - DNA, 31 bp, Artificial Sequence
  Description of Artificial Sequence  Enzymatic Nucleic Acid
tgggcaggggctagctacaacgaaggtgagt

SEQ ID NO: 1071 - DNA, 31 bp, Artificial Sequence
  Description of Artificial Sequence  Enzymatic Nucleic Acid
ttggtgggggctagctacaacgaaggtaggt

SEQ ID NO: 1072 - DNA, 31 bp, Artificial Sequence
  Description of Artificial Sequence  Enzymatic Nucleic Acid
ggcattggggctagctacaacgagggcaggt

SEQ ID NO: 1073 - DNA, 31 bp, Artificial Sequence
  Description of Artificial Sequence  Enzymatic Nucleic Acid
ggctggcaggctagctacaacgatggtgggc

SEQ ID NO: 1074 - DNA, 31 bp, Artificial Sequence
  Description of Artificial Sequence  Enzymatic Nucleic Acid
caggctggggctagctacaacgaattggtgg

SEQ ID NO: 1075 - DNA, 31 bp, Artificial Sequence
  Description of Artificial Sequence  Enzymatic Nucleic Acid
aggacaggggctagctacaacgatggcattg

SEQ ID NO: 1076 - DNA, 31 bp, Artificial Sequence
  Description of Artificial Sequence  Enzymatic Nucleic Acid
aggaaggaggctagctacaacgaaggctggc

SEQ ID NO: 1077 - DNA, 31 bp, Artificial Sequence
  Description of Artificial Sequence  Enzymatic Nucleic Acid
atatcctgggctagctacaacgaaggaagga

SEQ ID NO: 1078 - DNA, 31 bp, Artificial Sequence
  Description of Artificial Sequence  Enzymatic Nucleic Acid
cctggataggctagctacaacgacctgcagg

SEQ ID NO: 1079 - DNA, 31 bp, Artificial Sequence
  Description of Artificial Sequence  Enzymatic Nucleic Acid
ctcctggaggctagctacaacgaatcctgca

SEQ ID NO: 1080 - DNA, 31 bp, Artificial Sequence
  Description of Artificial Sequence  Enzymatic Nucleic Acid
gccctgcaggctagctacaacgactcctgga

SEQ ID NO: 1081 - DNA, 31 bp, Artificial Sequence
  Description of Artificial Sequence  Enzymatic Nucleic Acid
tagccctgggctagctacaacgaacctcctg

SEQ ID NO: 1082 - DNA, 31 bp, Artificial Sequence
  Description of Artificial Sequence  Enzymatic Nucleic Acid
gcacgtagggctagctacaacgacctgcacc

SEQ ID NO: 1083 - DNA, 31 bp, Artificial Sequence
  Description of Artificial Sequence  Enzymatic Nucleic Acid
tgagcacgggctagctacaacgaagccctgc

SEQ ID NO: 1084 - DNA, 31 bp, Artificial Sequence
  Description of Artificial Sequence  Enzymatic Nucleic Acid
gatgagcaggctagctacaacgagtagccct

SEQ ID NO: 1085 - DNA, 31 bp, Artificial Sequence
  Description of Artificial Sequence  Enzymatic Nucleic Acid
gcgatgagggctagctacaacgaacgtagcc

SEQ ID NO: 1086 - DNA, 31 bp, Artificial Sequence
  Description of Artificial Sequence  Enzymatic Nucleic Acid
gtgagcgaggctagctacaacgagagcacgt

SEQ ID NO: 1087 - DNA, 31 bp, Artificial Sequence
  Description of Artificial Sequence  Enzymatic Nucleic Acid
gttgtgagggctagctacaacgagatgagca

SEQ ID NO: 1088 - DNA, 31 bp, Artificial Sequence
  Description of Artificial Sequence  Enzymatic Nucleic Acid
cttggttgggctagctacaacgagagcgatg

SEQ ID NO: 1089 - DNA, 31 bp, Artificial Sequence
  Description of Artificial Sequence  Enzymatic Nucleic Acid
tcacttggggctagctacaacgatgtgagcg

SEQ ID NO: 1090 - DNA, 31 bp, Artificial Sequence
  Description of Artificial Sequence  Enzymatic Nucleic Acid
ctgcctcaggctagctacaacgattggttgt

SEQ ID NO: 1091 - DNA, 31 bp, Artificial Sequence
  Description of Artificial Sequence  Enzymatic Nucleic Acid
gggacctgggctagctacaacgactcacttg

SEQ ID NO: 1092 - DNA, 31 bp, Artificial Sequence
  Description of Artificial Sequence  Enzymatic Nucleic Acid
cagtgggaggctagctacaacgactgcctca

SEQ ID NO: 1093 - DNA, 31 bp, Artificial Sequence
  Description of Artificial Sequence  Enzymatic Nucleic Acid
ctctgcagggctagctacaacgagggacctg

SEQ ID NO: 1094 - DNA, 31 bp, Artificial Sequence
  Description of Artificial Sequence  Enzymatic Nucleic Acid
agcctctgggctagctacaacgaagtgggac

SEQ ID NO: 1095 - DNA, 31 bp, Artificial Sequence
  Description of Artificial Sequence  Enzymatic Nucleic Acid
atccgcagggctagctacaacgactctgcag

SEQ ID NO: 1096 - DNA, 31 bp, Artificial Sequence
  Description of Artificial Sequence  Enzymatic Nucleic Acid
acaatccgggctagctacaacgaagcctctg

SEQ ID NO: 1097 - DNA, 31 bp, Artificial Sequence
  Description of Artificial Sequence  Enzymatic Nucleic Acid
tcgcacaaggctagctacaacgaccgcagcc

SEQ ID NO: 1098 - DNA, 31 bp, Artificial Sequence
  Description of Artificial Sequence  Enzymatic Nucleic Acid
gcctcgcaggctagctacaacgaaatccgca

SEQ ID NO: 1099 - DNA, 31 bp, Artificial Sequence
  Description of Artificial Sequence  Enzymatic Nucleic Acid
gtgcctcgggctagctacaacgaacaatccg

SEQ ID NO: 1100 - DNA, 31 bp, Artificial Sequence
  Description of Artificial Sequence  Enzymatic Nucleic Acid
gctgggtgggctagctacaacgactcgcaca

SEQ ID NO: 1101 - DNA, 31 bp, Artificial Sequence
  Description of Artificial Sequence  Enzymatic Nucleic Acid
gagctgggggctagctacaacgagcctcgca

SEQ ID NO: 1102 - DNA, 31 bp, Artificial Sequence
  Description of Artificial Sequence  Enzymatic Nucleic Acid
tcaaagagggctagctacaacgatgggtgcc

SEQ ID NO: 1103 - DNA, 31 bp, Artificial Sequence
  Description of Artificial Sequence  Enzymatic Nucleic Acid
catagttgggctagctacaacgacctcaaag

SEQ ID NO: 1104 - DNA, 31 bp, Artificial Sequence
  Description of Artificial Sequence  Enzymatic Nucleic Acid
gggcatagggctagctacaacgatgtcctca

SEQ ID NO: 1105 - DNA, 31 bp, Artificial Sequence
  Description of Artificial Sequence  Enzymatic Nucleic Acid
ccagggcaggctagctacaacgaagttgtcc

SEQ ID NO: 1106 - DNA, 31 bp, Artificial Sequence
  Description of Artificial Sequence  Enzymatic Nucleic Acid
ggccagggggctagctacaacgaatagttgt

SEQ ID NO: 1107 - DNA, 31 bp, Artificial Sequence
  Description of Artificial Sequence  Enzymatic Nucleic Acid
tagcacggggctagctacaacgacagggcat

SEQ ID NO: 1108 - DNA, 31 bp, Artificial Sequence
  Description of Artificial Sequence  Enzymatic Nucleic Acid
gtctagcaggctagctacaacgaggccaggg

SEQ ID NO: 1109 - DNA, 31 bp, Artificial Sequence
  Description of Artificial Sequence  Enzymatic Nucleic Acid
ttgtctagggctagctacaacgaacggccag

SEQ ID NO: 1110 - DNA, 31 bp, Artificial Sequence
  Description of Artificial Sequence  Enzymatic Nucleic Acid
ctccattgggctagctacaacgactagcacg

SEQ ID NO: 1111 - DNA, 31 bp, Artificial Sequence
  Description of Artificial Sequence  Enzymatic Nucleic Acid
ggtctccaggctagctacaacgatgtctagc

SEQ ID NO: 1112 - DNA, 31 bp, Artificial Sequence
  Description of Artificial Sequence  Enzymatic Nucleic Acid
tcagcgggggctagctacaacgactccattg

SEQ ID NO: 1113 - DNA, 31 bp, Artificial Sequence
  Description of Artificial Sequence  Enzymatic Nucleic Acid
ttgttcagggctagctacaacgagggtctcc

SEQ ID NO: 1114 - DNA, 31 bp, Artificial Sequence
  Description of Artificial Sequence  Enzymatic Nucleic Acid
tggtattgggctagctacaacgatcagcggg

SEQ ID NO: 1115 - DNA, 31 bp, Artificial Sequence
  Description of Artificial Sequence  Enzymatic Nucleic Acid
gggtggtaggctagctacaacgatgttcagc

SEQ ID NO: 1116 - DNA, 31 bp, Artificial Sequence
  Description of Artificial Sequence  Enzymatic Nucleic Acid
aggggtggggctagctacaacgaattgttca

SEQ ID NO: 1117 - DNA, 31 bp, Artificial Sequence
  Description of Artificial Sequence  Enzymatic Nucleic Acid
gacaggggggctagctacaacgaggtattgt

SEQ ID NO: 1118 - DNA, 31 bp, Artificial Sequence
  Description of Artificial Sequence  Enzymatic Nucleic Acid
ccctgtgaggctagctacaacgaaggggtgg

SEQ ID NO: 1119 - DNA, 31 bp, Artificial Sequence
  Description of Artificial Sequence  Enzymatic Nucleic Acid
ggcccctgggctagctacaacgagacagggg

SEQ ID NO: 1120 - DNA, 31 bp, Artificial Sequence
  Description of Artificial Sequence  Enzymatic Nucleic Acid
tggggaggggctagctacaacgaccctgtga

SEQ ID NO: 1121 - DNA, 31 bp, Artificial Sequence
  Description of Artificial Sequence  Enzymatic Nucleic Acid
cccgcaggggctagctacaacgactcctggg

SEQ ID NO: 1122 - DNA, 31 bp, Artificial Sequence
  Description of Artificial Sequence  Enzymatic Nucleic Acid
agctcccgggctagctacaacgaaggcctcc

SEQ ID NO: 1123 - DNA, 31 bp, Artificial Sequence
  Description of Artificial Sequence  Enzymatic Nucleic Acid
agctgcagggctagctacaacgatcccgcag

SEQ ID NO: 1124 - DNA, 31 bp, Artificial Sequence
  Description of Artificial Sequence  Enzymatic Nucleic Acid
cgaagctgggctagctacaacgaagctcccg

SEQ ID NO: 1125 - DNA, 31 bp, Artificial Sequence
  Description of Artificial Sequence  Enzymatic Nucleic Acid
cttcgaagggctagctacaacgatgcagctc

SEQ ID NO: 1126 - DNA, 31 bp, Artificial Sequence
  Description of Artificial Sequence  Enzymatic Nucleic Acid
ctgtgaggggctagctacaacgattcgaagc

SEQ ID NO: 1127 - DNA, 31 bp, Artificial Sequence
  Description of Artificial Sequence  Enzymatic Nucleic Acid
gatctctgggctagctacaacgagaggcttc

SEQ ID NO: 1128 - DNA, 31 bp, Artificial Sequence
  Description of Artificial Sequence  Enzymatic Nucleic Acid
tttcaagaggctagctacaacgactctgtga

SEQ ID NO: 1129 - DNA, 31 bp, Artificial Sequence
  Description of Artificial Sequence  Enzymatic Nucleic Acid
gatcaagaggctagctacaacgaccctcctt

SEQ ID NO: 1130 - DNA, 31 bp, Artificial Sequence
  Description of Artificial Sequence  Enzymatic Nucleic Acid
ccgctggaggctagctacaacgacaagaccc

SEQ ID NO: 1131 - DNA, 31 bp, Artificial Sequence
  Description of Artificial Sequence  Enzymatic Nucleic Acid
gggttccgggctagctacaacgatggatcaa

SEQ ID NO: 1132 - DNA, 31 bp, Artificial Sequence
  Description of Artificial Sequence  Enzymatic Nucleic Acid
gctgggggggctagctacaacgatccgctgg

SEQ ID NO: 1133 - DNA, 31 bp, Artificial Sequence
  Description of Artificial Sequence  Enzymatic Nucleic Acid
tagcagagggctagctacaacgatgggggtt

SEQ ID NO: 1134 - DNA, 31 bp, Artificial Sequence
  Description of Artificial Sequence  Enzymatic Nucleic Acid
cctggtagggctagctacaacgaagagctgg

SEQ ID NO: 1135 - DNA, 31 bp, Artificial Sequence
  Description of Artificial Sequence  Enzymatic Nucleic Acid
tgtcctggggctagctacaacgaagcagagc

SEQ ID NO: 1136 - DNA, 31 bp, Artificial Sequence
  Description of Artificial Sequence  Enzymatic Nucleic Acid
aaatcgtgggctagctacaacgacctggtag

SEQ ID NO: 1137 - DNA, 31 bp, Artificial Sequence
  Description of Artificial Sequence  Enzymatic Nucleic Acid
caaaatcgggctagctacaacgagtcctggt

SEQ ID NO: 1138 - DNA, 31 bp, Artificial Sequence
  Description of Artificial Sequence  Enzymatic Nucleic Acid
ccacaaaaggctagctacaacgacgtgtcct

SEQ ID NO: 1139 - DNA, 31 bp, Artificial Sequence
  Description of Artificial Sequence  Enzymatic Nucleic Acid
tccttccaggctagctacaacgaaaaatcgt

SEQ ID NO: 1140 - DNA, 31 bp, Artificial Sequence
  Description of Artificial Sequence  Enzymatic Nucleic Acid
ggaagatgggctagctacaacgaccttccac

SEQ ID NO: 1141 - DNA, 31 bp, Artificial Sequence
  Description of Artificial Sequence  Enzymatic Nucleic Acid
gtggaagaggctagctacaacgagtccttcc

SEQ ID NO: 1142 - DNA, 31 bp, Artificial Sequence
  Description of Artificial Sequence  Enzymatic Nucleic Acid
tgttcttgggctagctacaacgaggaagatg

SEQ ID NO: 1143 - DNA, 31 bp, Artificial Sequence
  Description of Artificial Sequence  Enzymatic Nucleic Acid
gctggttgggctagctacaacgatcttgtgg

SEQ ID NO: 1144 - DNA, 31 bp, Artificial Sequence
  Description of Artificial Sequence  Enzymatic Nucleic Acid
ccagctggggctagctacaacgatgttcttg

SEQ ID NO: 1145 - DNA, 31 bp, Artificial Sequence
  Description of Artificial Sequence  Enzymatic Nucleic Acid
agagccagggctagctacaacgatggttgtt

SEQ ID NO: 1146 - DNA, 31 bp, Artificial Sequence
  Description of Artificial Sequence  Enzymatic Nucleic Acid
tgtgagagggctagctacaacgacagctggt

SEQ ID NO: 1147 - DNA, 31 bp, Artificial Sequence
  Description of Artificial Sequence  Enzymatic Nucleic Acid
tatcagtgggctagctacaacgagagagcca

SEQ ID NO: 1148 - DNA, 31 bp, Artificial Sequence
  Description of Artificial Sequence  Enzymatic Nucleic Acid
tctatcagggctagctacaacgagtgagagc

SEQ ID NO: 1149 - DNA, 31 bp, Artificial Sequence
  Description of Artificial Sequence  Enzymatic Nucleic Acid
ggtgtctaggctagctacaacgacagtgtga

SEQ ID NO: 1150 - DNA, 31 bp, Artificial Sequence
  Description of Artificial Sequence  Enzymatic Nucleic Acid
ggttggtgggctagctacaacgactatcagt

SEQ ID NO: 1151 - DNA, 31 bp, Artificial Sequence
  Description of Artificial Sequence  Enzymatic Nucleic Acid
gcggttggggctagctacaacgagtctatca

SEQ ID NO: 1152 - DNA, 31 bp, Artificial Sequence
  Description of Artificial Sequence  Enzymatic Nucleic Acid
gagagcggggctagctacaacgatggtgtct

SEQ ID NO: 1153 - DNA, 31 bp, Artificial Sequence
  Description of Artificial Sequence  Enzymatic Nucleic Acid
cccgagagggctagctacaacgaggttggtg

SEQ ID NO: 1154 - DNA, 31 bp, Artificial Sequence
  Description of Artificial Sequence  Enzymatic Nucleic Acid
gtggcaggggctagctacaacgaccgagagc

SEQ ID NO: 1155 - DNA, 31 bp, Artificial Sequence
  Description of Artificial Sequence  Enzymatic Nucleic Acid
aggggtggggctagctacaacgaaggcccga

SEQ ID NO: 1156 - DNA, 31 bp, Artificial Sequence
  Description of Artificial Sequence  Enzymatic Nucleic Acid
aacaggggggctagctacaacgaggcaggcc

SEQ ID NO: 1157 - DNA, 31 bp, Artificial Sequence
  Description of Artificial Sequence  Enzymatic Nucleic Acid
tcggagaaggctagctacaacgaaggggtgg

SEQ ID NO: 1158 - DNA, 31 bp, Artificial Sequence
  Description of Artificial Sequence  Enzymatic Nucleic Acid
cttacacaggctagctacaacgacggagaac

SEQ ID NO: 1159 - DNA, 31 bp, Artificial Sequence
  Description of Artificial Sequence  Enzymatic Nucleic Acid
cccttacaggctagctacaacgaatcggaga

SEQ ID NO: 1160 - DNA, 31 bp, Artificial Sequence
  Description of Artificial Sequence  Enzymatic Nucleic Acid
agcccttaggctagctacaacgaacatcgga

SEQ ID NO: 1161 - DNA, 31 bp, Artificial Sequence
  Description of Artificial Sequence  Enzymatic Nucleic Acid
agcgggagggctagctacaacgaccttacac

SEQ ID NO: 1162 - DNA, 31 bp, Artificial Sequence
  Description of Artificial Sequence  Enzymatic Nucleic Acid
cccagcagggctagctacaacgagggagccc

SEQ ID NO: 1163 - DNA, 31 bp, Artificial Sequence
  Description of Artificial Sequence  Enzymatic Nucleic Acid
ctccccagggctagctacaacgaagcgggag

SEQ ID NO: 1164 - DNA, 31 bp, Artificial Sequence
  Description of Artificial Sequence  Enzymatic Nucleic Acid
cctcagaaggctagctacaacgatctctccc

SEQ ID NO: 1165 - DNA, 31 bp, Artificial Sequence
  Description of Artificial Sequence  Enzymatic Nucleic Acid
tctgacaaggctagctacaacgacctcagaa

SEQ ID NO: 1166 - DNA, 31 bp, Artificial Sequence
  Description of Artificial Sequence  Enzymatic Nucleic Acid
ggctctgaggctagctacaacgaaatcctca

SEQ ID NO: 1167 - DNA, 31 bp, Artificial Sequence
  Description of Artificial Sequence  Enzymatic Nucleic Acid
gcgtcaggggctagctacaacgatctgacaa

SEQ ID NO: 1168 - DNA, 31 bp, Artificial Sequence
  Description of Artificial Sequence  Enzymatic Nucleic Acid
agtgcgcgggctagctacaacgacaggctct

SEQ ID NO: 1169 - DNA, 31 bp, Artificial Sequence
  Description of Artificial Sequence  Enzymatic Nucleic Acid
acagtgcgggctagctacaacgagtcaggct

SEQ ID NO: 1170 - DNA, 31 bp, Artificial Sequence
  Description of Artificial Sequence  Enzymatic Nucleic Acid
agacagtgggctagctacaacgagcgtcagg

SEQ ID NO: 1171 - DNA, 31 bp, Artificial Sequence
  Description of Artificial Sequence  Enzymatic Nucleic Acid
acagacagggctagctacaacgagcgcgtca

SEQ ID NO: 1172 - DNA, 31 bp, Artificial Sequence
  Description of Artificial Sequence  Enzymatic Nucleic Acid
ggcacagaggctagctacaacgaagtgcgcg

SEQ ID NO: 1173 - DNA, 31 bp, Artificial Sequence
  Description of Artificial Sequence  Enzymatic Nucleic Acid
caccggcaggctagctacaacgaagacagtg

SEQ ID NO: 1174 - DNA, 31 bp, Artificial Sequence
  Description of Artificial Sequence  Enzymatic Nucleic Acid
gccaccggggctagctacaacgaacagacag

SEQ ID NO: 1175 - DNA, 31 bp, Artificial Sequence
  Description of Artificial Sequence  Enzymatic Nucleic Acid
cacagccaggctagctacaacgacggcacag

SEQ ID NO: 1176 - DNA, 31 bp, Artificial Sequence
  Description of Artificial Sequence  Enzymatic Nucleic Acid
gggcacagggctagctacaacgacaccggca

SEQ ID NO: 1177 - DNA, 31 bp, Artificial Sequence
  Description of Artificial Sequence  Enzymatic Nucleic Acid
agcgggcaggctagctacaacgaagccaccg

SEQ ID NO: 1178 - DNA, 31 bp, Artificial Sequence
  Description of Artificial Sequence  Enzymatic Nucleic Acid
gcagcgggggctagctacaacgaacagccac

SEQ ID NO: 1179 - DNA, 31 bp, Artificial Sequence
  Description of Artificial Sequence  Enzymatic Nucleic Acid
ccttgcagggctagctacaacgagggcacag

SEQ ID NO: 1180 - DNA, 31 bp, Artificial Sequence
  Description of Artificial Sequence  Enzymatic Nucleic Acid
gccccttgggctagctacaacgaagcgggca

SEQ ID NO: 1181 - DNA, 31 bp, Artificial Sequence
  Description of Artificial Sequence  Enzymatic Nucleic Acid
ggcagtggggctagctacaacgacccttgca

SEQ ID NO: 1182 - DNA, 31 bp, Artificial Sequence
  Description of Artificial Sequence  Enzymatic Nucleic Acid
gtgggcagggctagctacaacgaggcccctt

SEQ ID NO: 1183 - DNA, 31 bp, Artificial Sequence
  Description of Artificial Sequence  Enzymatic Nucleic Acid
tcagtgggggctagctacaacgaagtggccc

SEQ ID NO: 1184 - DNA, 31 bp, Artificial Sequence
  Description of Artificial Sequence  Enzymatic Nucleic Acid
gcagtcagggctagctacaacgagggcagtg

SEQ ID NO: 1185 - DNA, 31 bp, Artificial Sequence
  Description of Artificial Sequence  Enzymatic Nucleic Acid
ggcagcagggctagctacaacgacagtgggc

SEQ ID NO: 1186 - DNA, 31 bp, Artificial Sequence
  Description of Artificial Sequence  Enzymatic Nucleic Acid
catggcagggctagctacaacgaagtcagtg

SEQ ID NO: 1187 - DNA, 31 bp, Artificial Sequence
  Description of Artificial Sequence  Enzymatic Nucleic Acid
gctcatggggctagctacaacgaagcagtca

SEQ ID NO: 1188 - DNA, 31 bp, Artificial Sequence
  Description of Artificial Sequence  Enzymatic Nucleic Acid
actgctcaggctagctacaacgaggcagcag

SEQ ID NO: 1189 - DNA, 31 bp, Artificial Sequence
  Description of Artificial Sequence  Enzymatic Nucleic Acid
gcacactgggctagctacaacgatcatggca

SEQ ID NO: 1190 - DNA, 31 bp, Artificial Sequence
  Description of Artificial Sequence  Enzymatic Nucleic Acid
gcagcacaggctagctacaacgatgctcatg

SEQ ID NO: 1191 - DNA, 31 bp, Artificial Sequence
  Description of Artificial Sequence  Enzymatic Nucleic Acid
cggcagcaggctagctacaacgaactgctca

SEQ ID NO: 1192 - DNA, 31 bp, Artificial Sequence
  Description of Artificial Sequence  Enzymatic Nucleic Acid
gccggcagggctagctacaacgaacactgct

SEQ ID NO: 1193 - DNA, 31 bp, Artificial Sequence
  Description of Artificial Sequence  Enzymatic Nucleic Acid
gcagccggggctagctacaacgaagcacact

SEQ ID NO: 1194 - DNA, 31 bp, Artificial Sequence
  Description of Artificial Sequence  Enzymatic Nucleic Acid
ccgtgcagggctagctacaacgacggcagca

SEQ ID NO: 1195 - DNA, 31 bp, Artificial Sequence
  Description of Artificial Sequence  Enzymatic Nucleic Acid
ggcccgtgggctagctacaacgaagccggca

SEQ ID NO: 1196 - DNA, 31 bp, Artificial Sequence
  Description of Artificial Sequence  Enzymatic Nucleic Acid
ggggcccgggctagctacaacgagcagccgg

SEQ ID NO: 1197 - DNA, 31 bp, Artificial Sequence
  Description of Artificial Sequence  Enzymatic Nucleic Acid
gcttggggggctagctacaacgaccgtgcag

SEQ ID NO: 1198 - DNA, 31 bp, Artificial Sequence
  Description of Artificial Sequence  Enzymatic Nucleic Acid
tcagagtgggctagctacaacgattggggcc

SEQ ID NO: 1199 - DNA, 31 bp, Artificial Sequence
  Description of Artificial Sequence  Enzymatic Nucleic Acid
agtcagagggctagctacaacgagcttgggg

SEQ ID NO: 1200 - DNA, 31 bp, Artificial Sequence
  Description of Artificial Sequence  Enzymatic Nucleic Acid
ccaggcagggctagctacaacgacagagtgc

SEQ ID NO: 1201 - DNA, 31 bp, Artificial Sequence
  Description of Artificial Sequence  Enzymatic Nucleic Acid
aggccaggggctagctacaacgaagtcagag

SEQ ID NO: 1202 - DNA, 31 bp, Artificial Sequence
  Description of Artificial Sequence  Enzymatic Nucleic Acid
gaggcaggggctagctacaacgacaggcagt

SEQ ID NO: 1203 - DNA, 31 bp, Artificial Sequence
  Description of Artificial Sequence  Enzymatic Nucleic Acid
agtggaggggctagctacaacgaaggccagg

SEQ ID NO: 1204 - DNA, 31 bp, Artificial Sequence
  Description of Artificial Sequence  Enzymatic Nucleic Acid
ggttgaagggctagctacaacgaggaggcag

SEQ ID NO: 1205 - DNA, 31 bp, Artificial Sequence
  Description of Artificial Sequence  Enzymatic Nucleic Acid
cactgtggggctagctacaacgatgaagtgg

SEQ ID NO: 1206 - DNA, 31 bp, Artificial Sequence
  Description of Artificial Sequence  Enzymatic Nucleic Acid
tgccactgggctagctacaacgaggttgaag

SEQ ID NO: 1207 - DNA, 31 bp, Artificial Sequence
  Description of Artificial Sequence  Enzymatic Nucleic Acid
agatgccaggctagctacaacgatgtggttg

SEQ ID NO: 1208 - DNA, 31 bp, Artificial Sequence
  Description of Artificial Sequence  Enzymatic Nucleic Acid
cacagatgggctagctacaacgacactgtgg

SEQ ID NO: 1209 - DNA, 31 bp, Artificial Sequence
  Description of Artificial Sequence  Enzymatic Nucleic Acid
ctcacagaggctagctacaacgagccactgt

SEQ ID NO: 1210 - DNA, 31 bp, Artificial Sequence
  Description of Artificial Sequence  Enzymatic Nucleic Acid
gcagctcaggctagctacaacgaagatgcca

SEQ ID NO: 1211 - DNA, 31 bp, Artificial Sequence
  Description of Artificial Sequence  Enzymatic Nucleic Acid
cagtgcagggctagctacaacgatcacagat

SEQ ID NO: 1212 - DNA, 31 bp, Artificial Sequence
  Description of Artificial Sequence  Enzymatic Nucleic Acid
gggcagtgggctagctacaacgaagctcaca

SEQ ID NO: 1213 - DNA, 31 bp, Artificial Sequence
  Description of Artificial Sequence  Enzymatic Nucleic Acid
ctgggcagggctagctacaacgagcagctca

SEQ ID NO: 1214 - DNA, 31 bp, Artificial Sequence
  Description of Artificial Sequence  Enzymatic Nucleic Acid
gggctgggggctagctacaacgaagtgcagc

SEQ ID NO: 1215 - DNA, 31 bp, Artificial Sequence
  Description of Artificial Sequence  Enzymatic Nucleic Acid
gaccagggggctagctacaacgatgggcagt

SEQ ID NO: 1216 - DNA, 31 bp, Artificial Sequence
  Description of Artificial Sequence  Enzymatic Nucleic Acid
gtaggtgaggctagctacaacgacagggctg

SEQ ID NO: 1217 - DNA, 31 bp, Artificial Sequence
  Description of Artificial Sequence  Enzymatic Nucleic Acid
gttgtaggggctagctacaacgagaccaggg

SEQ ID NO: 1218 - DNA, 31 bp, Artificial Sequence
  Description of Artificial Sequence  Enzymatic Nucleic Acid
ctgtgttgggctagctacaacgaaggtgacc

SEQ ID NO: 1219 - DNA, 31 bp, Artificial Sequence
  Description of Artificial Sequence  Enzymatic Nucleic Acid
tgtctgtgggctagctacaacgatgtaggtg

SEQ ID NO: 1220 - DNA, 31 bp, Artificial Sequence
  Description of Artificial Sequence  Enzymatic Nucleic Acid
cgtgtctgggctagctacaacgagttgtagg

SEQ ID NO: 1221 - DNA, 31 bp, Artificial Sequence
  Description of Artificial Sequence  Enzymatic Nucleic Acid
caaacgtgggctagctacaacgactgtgttg

SEQ ID NO: 1222 - DNA, 31 bp, Artificial Sequence
  Description of Artificial Sequence  Enzymatic Nucleic Acid
ctcaaacgggctagctacaacgagtctgtgt

SEQ ID NO: 1223 - DNA, 31 bp, Artificial Sequence
  Description of Artificial Sequence  Enzymatic Nucleic Acid
gactcaaaggctagctacaacgagtgtctgt

SEQ ID NO: 1224 - DNA, 31 bp, Artificial Sequence
  Description of Artificial Sequence  Enzymatic Nucleic Acid
ggcatggaggctagctacaacgatcaaacgt

SEQ ID NO: 1225 - DNA, 31 bp, Artificial Sequence
  Description of Artificial Sequence  Enzymatic Nucleic Acid
attgggcaggctagctacaacgaggactcaa

SEQ ID NO: 1226 - DNA, 31 bp, Artificial Sequence
  Description of Artificial Sequence  Enzymatic Nucleic Acid
ggattgggggctagctacaacgaatggactc

SEQ ID NO: 1227 - DNA, 31 bp, Artificial Sequence
  Description of Artificial Sequence  Enzymatic Nucleic Acid
cctcgggaggctagctacaacgatgggcatg

SEQ ID NO: 1228 - DNA, 31 bp, Artificial Sequence
  Description of Artificial Sequence  Enzymatic Nucleic Acid
tataccggggctagctacaacgacctcggga

SEQ ID NO: 1229 - DNA, 31 bp, Artificial Sequence
  Description of Artificial Sequence  Enzymatic Nucleic Acid
aatgtataggctagctacaacgacggccctc

SEQ ID NO: 1230 - DNA, 31 bp, Artificial Sequence
  Description of Artificial Sequence  Enzymatic Nucleic Acid
cgaatgtaggctagctacaacgaaccggccc

SEQ ID NO: 1231 - DNA, 31 bp, Artificial Sequence
  Description of Artificial Sequence  Enzymatic Nucleic Acid
gccgaatgggctagctacaacgaataccggc

SEQ ID NO: 1232 - DNA, 31 bp, Artificial Sequence
  Description of Artificial Sequence  Enzymatic Nucleic Acid
gcgccgaaggctagctacaacgagtataccg

SEQ ID NO: 1233 - DNA, 31 bp, Artificial Sequence
  Description of Artificial Sequence  Enzymatic Nucleic Acid
agctggcgggctagctacaacgacgaatgta

SEQ ID NO: 1234 - DNA, 31 bp, Artificial Sequence
  Description of Artificial Sequence  Enzymatic Nucleic Acid
acagctggggctagctacaacgagccgaatg

SEQ ID NO: 1235 - DNA, 31 bp, Artificial Sequence
  Description of Artificial Sequence  Enzymatic Nucleic Acid
tcacacagggctagctacaacgatggcgccg

SEQ ID NO: 1236 - DNA, 31 bp, Artificial Sequence
  Description of Artificial Sequence  Enzymatic Nucleic Acid
cagtcacaggctagctacaacgaagctggcg

SEQ ID NO: 1237 - DNA, 31 bp, Artificial Sequence
  Description of Artificial Sequence  Enzymatic Nucleic Acid
ggcagtcaggctagctacaacgaacagctgg

SEQ ID NO: 1238 - DNA, 31 bp, Artificial Sequence
  Description of Artificial Sequence  Enzymatic Nucleic Acid
acaggcagggctagctacaacgacacacagc

SEQ ID NO: 1239 - DNA, 31 bp, Artificial Sequence
  Description of Artificial Sequence  Enzymatic Nucleic Acid
gggacaggggctagctacaacgaagtcacac

SEQ ID NO: 1240 - DNA, 31 bp, Artificial Sequence
  Description of Artificial Sequence  Enzymatic Nucleic Acid
tgtagggaggctagctacaacgaaggcagtc

SEQ ID NO: 1241 - DNA, 31 bp, Artificial Sequence
  Description of Artificial Sequence  Enzymatic Nucleic Acid
ggtagttgggctagctacaacgaagggacag

SEQ ID NO: 1242 - DNA, 31 bp, Artificial Sequence
  Description of Artificial Sequence  Enzymatic Nucleic Acid
aaaggtagggctagctacaacgatgtaggga

SEQ ID NO: 1243 - DNA, 31 bp, Artificial Sequence
  Description of Artificial Sequence  Enzymatic Nucleic Acid
tagaaaggggctagctacaacgaagttgtag

SEQ ID NO: 1244 - DNA, 31 bp, Artificial Sequence
  Description of Artificial Sequence  Enzymatic Nucleic Acid
cacgtccgggctagctacaacgaagaaaggt

SEQ ID NO: 1245 - DNA, 31 bp, Artificial Sequence
  Description of Artificial Sequence  Enzymatic Nucleic Acid
atcccacgggctagctacaacgaccgtagaa

SEQ ID NO: 1246 - DNA, 31 bp, Artificial Sequence
  Description of Artificial Sequence  Enzymatic Nucleic Acid
ggatcccaggctagctacaacgagtccgtag

SEQ ID NO: 1247 - DNA, 31 bp, Artificial Sequence
  Description of Artificial Sequence  Enzymatic Nucleic Acid
gtgcaggaggctagctacaacgacccacgtc

SEQ ID NO: 1248 - DNA, 31 bp, Artificial Sequence
  Description of Artificial Sequence  Enzymatic Nucleic Acid
cgagggtgggctagctacaacgaaggatccc

SEQ ID NO: 1249 - DNA, 31 bp, Artificial Sequence
  Description of Artificial Sequence  Enzymatic Nucleic Acid
gacgagggggctagctacaacgagcaggatc

SEQ ID NO: 1250 - DNA, 31 bp, Artificial Sequence
  Description of Artificial Sequence  Enzymatic Nucleic Acid
ggggcagaggctagctacaacgagagggtgc

SEQ ID NO: 1251 - DNA, 31 bp, Artificial Sequence
  Description of Artificial Sequence  Enzymatic Nucleic Acid
gcagggggggctagctacaacgaagacgagg

SEQ ID NO: 1252 - DNA, 31 bp, Artificial Sequence
  Description of Artificial Sequence  Enzymatic Nucleic Acid
tggttgtgggctagctacaacgaagggggca

SEQ ID NO: 1253 - DNA, 31 bp, Artificial Sequence
  Description of Artificial Sequence  Enzymatic Nucleic Acid
cttggttgggctagctacaacgagcaggggg

SEQ ID NO: 1254 - DNA, 31 bp, Artificial Sequence
  Description of Artificial Sequence  Enzymatic Nucleic Acid
cctcttggggctagctacaacgatgtgcagg

SEQ ID NO: 1255 - DNA, 31 bp, Artificial Sequence
  Description of Artificial Sequence  Enzymatic Nucleic Acid
tgctgtcaggctagctacaacgactcttggt

SEQ ID NO: 1256 - DNA, 31 bp, Artificial Sequence
  Description of Artificial Sequence  Enzymatic Nucleic Acid
ctctgctgggctagctacaacgacacctctt

SEQ ID NO: 1257 - DNA, 31 bp, Artificial Sequence
  Description of Artificial Sequence  Enzymatic Nucleic Acid
atcctctgggctagctacaacgatgtcacct

SEQ ID NO: 1258 - DNA, 31 bp, Artificial Sequence
  Description of Artificial Sequence  Enzymatic Nucleic Acid
gtgttccaggctagctacaacgacctctgct

SEQ ID NO: 1259 - DNA, 31 bp, Artificial Sequence
  Description of Artificial Sequence  Enzymatic Nucleic Acid
ccgctgtgggctagctacaacgatccatcct

SEQ ID NO: 1260 - DNA, 31 bp, Artificial Sequence
  Description of Artificial Sequence  Enzymatic Nucleic Acid
caccgctgggctagctacaacgagttccatc

SEQ ID NO: 1261 - DNA, 31 bp, Artificial Sequence
  Description of Artificial Sequence  Enzymatic Nucleic Acid
tcacaccgggctagctacaacgatgtgttcc

SEQ ID NO: 1262 - DNA, 31 bp, Artificial Sequence
  Description of Artificial Sequence  Enzymatic Nucleic Acid
ttctcacaggctagctacaacgacgctgtgt

SEQ ID NO: 1263 - DNA, 31 bp, Artificial Sequence
  Description of Artificial Sequence  Enzymatic Nucleic Acid
acttctcaggctagctacaacgaaccgctgt

SEQ ID NO: 1264 - DNA, 31 bp, Artificial Sequence
  Description of Artificial Sequence  Enzymatic Nucleic Acid
ttgctgcaggctagctacaacgattctcaca

SEQ ID NO: 1265 - DNA, 31 bp, Artificial Sequence
  Description of Artificial Sequence  Enzymatic Nucleic Acid
gcttgctgggctagctacaacgaacttctca

SEQ ID NO: 1266 - DNA, 31 bp, Artificial Sequence
  Description of Artificial Sequence  Enzymatic Nucleic Acid
agggcttgggctagctacaacgatgcacttc

SEQ ID NO: 1267 - DNA, 31 bp, Artificial Sequence
  Description of Artificial Sequence  Enzymatic Nucleic Acid
gcacagggggctagctacaacgattgctgca

SEQ ID NO: 1268 - DNA, 31 bp, Artificial Sequence
  Description of Artificial Sequence  Enzymatic Nucleic Acid
ctcgggcaggctagctacaacgaagggcttg

SEQ ID NO: 1269 - DNA, 31 bp, Artificial Sequence
  Description of Artificial Sequence  Enzymatic Nucleic Acid
cactcgggggctagctacaacgaacagggct

SEQ ID NO: 1270 - DNA, 31 bp, Artificial Sequence
  Description of Artificial Sequence  Enzymatic Nucleic Acid
atagcacaggctagctacaacgatcgggcac

SEQ ID NO: 1271 - DNA, 31 bp, Artificial Sequence
  Description of Artificial Sequence  Enzymatic Nucleic Acid
ccatagcaggctagctacaacgaactcgggc

SEQ ID NO: 1272 - DNA, 31 bp, Artificial Sequence
  Description of Artificial Sequence  Enzymatic Nucleic Acid
gaccatagggctagctacaacgaacactcgg

SEQ ID NO: 1273 - DNA, 31 bp, Artificial Sequence
  Description of Artificial Sequence  Enzymatic Nucleic Acid
ccagaccaggctagctacaacgaagcacact

SEQ ID NO: 1274 - DNA, 31 bp, Artificial Sequence
  Description of Artificial Sequence  Enzymatic Nucleic Acid
tgcccagaggctagctacaacgacatagcac

SEQ ID NO: 1275 - DNA, 31 bp, Artificial Sequence
  Description of Artificial Sequence  Enzymatic Nucleic Acid
gctccatgggctagctacaacgaccagacca

SEQ ID NO: 1276 - DNA, 31 bp, Artificial Sequence
  Description of Artificial Sequence  Enzymatic Nucleic Acid
gtgctccaggctagctacaacgagcccagac

SEQ ID NO: 1277 - DNA, 31 bp, Artificial Sequence
  Description of Artificial Sequence  Enzymatic Nucleic Acid
cgcaagtgggctagctacaacgatccatgcc

SEQ ID NO: 1278 - DNA, 31 bp, Artificial Sequence
  Description of Artificial Sequence  Enzymatic Nucleic Acid
ctcgcaagggctagctacaacgagctccatg

SEQ ID NO: 1279 - DNA, 31 bp, Artificial Sequence
  Description of Artificial Sequence  Enzymatic Nucleic Acid
acctctcgggctagctacaacgaaagtgctc

SEQ ID NO: 1280 - DNA, 31 bp, Artificial Sequence
  Description of Artificial Sequence  Enzymatic Nucleic Acid
tgccctcaggctagctacaacgactctcgca

SEQ ID NO: 1281 - DNA, 31 bp, Artificial Sequence
  Description of Artificial Sequence  Enzymatic Nucleic Acid
ggtaactgggctagctacaacgacctcacct

SEQ ID NO: 1282 - DNA, 31 bp, Artificial Sequence
  Description of Artificial Sequence  Enzymatic Nucleic Acid
actggtaaggctagctacaacgatgccctca

SEQ ID NO: 1283 - DNA, 31 bp, Artificial Sequence
  Description of Artificial Sequence  Enzymatic Nucleic Acid
ggcactggggctagctacaacgaaactgccc

SEQ ID NO: 1284 - DNA, 31 bp, Artificial Sequence
  Description of Artificial Sequence  Enzymatic Nucleic Acid
tattggcaggctagctacaacgatggtaact

SEQ ID NO: 1285 - DNA, 31 bp, Artificial Sequence
  Description of Artificial Sequence  Enzymatic Nucleic Acid
gatattggggctagctacaacgaactggtaa

SEQ ID NO: 1286 - DNA, 31 bp, Artificial Sequence
  Description of Artificial Sequence  Enzymatic Nucleic Acid
cctggataggctagctacaacgatggcactg

SEQ ID NO: 1287 - DNA, 31 bp, Artificial Sequence
  Description of Artificial Sequence  Enzymatic Nucleic Acid
ctcctggaggctagctacaacgaattggcac

SEQ ID NO: 1288 - DNA, 31 bp, Artificial Sequence
  Description of Artificial Sequence  Enzymatic Nucleic Acid
ccagcaaaggctagctacaacgatcctggat

SEQ ID NO: 1289 - DNA, 31 bp, Artificial Sequence
  Description of Artificial Sequence  Enzymatic Nucleic Acid
gcagccagggctagctacaacgaaaactcct

SEQ ID NO: 1290 - DNA, 31 bp, Artificial Sequence
  Description of Artificial Sequence  Enzymatic Nucleic Acid
tcttgcagggctagctacaacgacagcaaac

SEQ ID NO: 1291 - DNA, 31 bp, Artificial Sequence
  Description of Artificial Sequence  Enzymatic Nucleic Acid
tcttcttgggctagctacaacgaagccagca

SEQ ID NO: 1292 - DNA, 31 bp, Artificial Sequence
  Description of Artificial Sequence  Enzymatic Nucleic Acid
cccaaagaggctagctacaacgacttcttgc

SEQ ID NO: 1293 - DNA, 31 bp, Artificial Sequence
  Description of Artificial Sequence  Enzymatic Nucleic Acid
atgccaggggctagctacaacgatcccaaag

SEQ ID NO: 1294 - DNA, 31 bp, Artificial Sequence
  Description of Artificial Sequence  Enzymatic Nucleic Acid
cagaaatgggctagctacaacgacaggctcc

SEQ ID NO: 1295 - DNA, 31 bp, Artificial Sequence
  Description of Artificial Sequence  Enzymatic Nucleic Acid
ggcagaaaggctagctacaacgagccaggct

SEQ ID NO: 1296 - DNA, 31 bp, Artificial Sequence
  Description of Artificial Sequence  Enzymatic Nucleic Acid
ctctccggggctagctacaacgaagaaatgc

SEQ ID NO: 1297 - DNA, 31 bp, Artificial Sequence
  Description of Artificial Sequence  Enzymatic Nucleic Acid
catcaaagggctagctacaacgatctccggc

SEQ ID NO: 1298 - DNA, 31 bp, Artificial Sequence
  Description of Artificial Sequence  Enzymatic Nucleic Acid
ggtccccaggctagctacaacgacaaagctc

SEQ ID NO: 1299 - DNA, 31 bp, Artificial Sequence
  Description of Artificial Sequence  Enzymatic Nucleic Acid
aggctgggggctagctacaacgaccccatca

SEQ ID NO: 1300 - DNA, 31 bp, Artificial Sequence
  Description of Artificial Sequence  Enzymatic Nucleic Acid
gttggaggggctagctacaacgatgggtccc

SEQ ID NO: 1301 - DNA, 31 bp, Artificial Sequence
  Description of Artificial Sequence  Enzymatic Nucleic Acid
gggcagtgggctagctacaacgatggaggct

SEQ ID NO: 1302 - DNA, 31 bp, Artificial Sequence
  Description of Artificial Sequence  Enzymatic Nucleic Acid
cggggcagggctagctacaacgagttggagg

SEQ ID NO: 1303 - DNA, 31 bp, Artificial Sequence
  Description of Artificial Sequence  Enzymatic Nucleic Acid
gagcggggggctagctacaacgaagtgttgg

SEQ ID NO: 1304 - DNA, 31 bp, Artificial Sequence
  Description of Artificial Sequence  Enzymatic Nucleic Acid
ggctggagggctagctacaacgaggggcagt

SEQ ID NO: 1305 - DNA, 31 bp, Artificial Sequence
  Description of Artificial Sequence  Enzymatic Nucleic Acid
tgctctggggctagctacaacgatggagcgg

SEQ ID NO: 1306 - DNA, 31 bp, Artificial Sequence
  Description of Artificial Sequence  Enzymatic Nucleic Acid
tggagctgggctagctacaacgatctggctg

SEQ ID NO: 1307 - DNA, 31 bp, Artificial Sequence
  Description of Artificial Sequence  Enzymatic Nucleic Acid
acttggagggctagctacaacgatgctctgg

SEQ ID NO: 1308 - DNA, 31 bp, Artificial Sequence
  Description of Artificial Sequence  Enzymatic Nucleic Acid
ctcaaacaggctagctacaacgattggagct

SEQ ID NO: 1309 - DNA, 31 bp, Artificial Sequence
  Description of Artificial Sequence  Enzymatic Nucleic Acid
gtctcaaaggctagctacaacgaacttggag

SEQ ID NO: 1310 - DNA, 31 bp, Artificial Sequence
  Description of Artificial Sequence  Enzymatic Nucleic Acid
ttccagagggctagctacaacgactcaaaca

SEQ ID NO: 1311 - DNA, 31 bp, Artificial Sequence
  Description of Artificial Sequence  Enzymatic Nucleic Acid
acctgtgaggctagctacaacgactcttcca

SEQ ID NO: 1312 - DNA, 31 bp, Artificial Sequence
  Description of Artificial Sequence  Enzymatic Nucleic Acid
gtaacctgggctagctacaacgagatctctt

SEQ ID NO: 1313 - DNA, 31 bp, Artificial Sequence
  Description of Artificial Sequence  Enzymatic Nucleic Acid
ataggtaaggctagctacaacgactgtgatc

SEQ ID NO: 1314 - DNA, 31 bp, Artificial Sequence
  Description of Artificial Sequence  Enzymatic Nucleic Acid
tgtataggggctagctacaacgaaacctgtg

SEQ ID NO: 1315 - DNA, 31 bp, Artificial Sequence
  Description of Artificial Sequence  Enzymatic Nucleic Acid
gagatgtaggctagctacaacgaaggtaacc

SEQ ID NO: 1316 - DNA, 31 bp, Artificial Sequence
  Description of Artificial Sequence  Enzymatic Nucleic Acid
ctgagatgggctagctacaacgaataggtaa

SEQ ID NO: 1317 - DNA, 31 bp, Artificial Sequence
  Description of Artificial Sequence  Enzymatic Nucleic Acid
tgctgagaggctagctacaacgagtataggt

SEQ ID NO: 1318 - DNA, 31 bp, Artificial Sequence
  Description of Artificial Sequence  Enzymatic Nucleic Acid
cggccatgggctagctacaacgatgagatgt

SEQ ID NO: 1319 - DNA, 31 bp, Artificial Sequence
  Description of Artificial Sequence  Enzymatic Nucleic Acid
tccggccaggctagctacaacgagctgagat

SEQ ID NO: 1320 - DNA, 31 bp, Artificial Sequence
  Description of Artificial Sequence  Enzymatic Nucleic Acid
ctgtccggggctagctacaacgacatgctga

SEQ ID NO: 1321 - DNA, 31 bp, Artificial Sequence
  Description of Artificial Sequence  Enzymatic Nucleic Acid
gcaggctgggctagctacaacgaccggccat

SEQ ID NO: 1322 - DNA, 31 bp, Artificial Sequence
  Description of Artificial Sequence  Enzymatic Nucleic Acid
caggcaggggctagctacaacgatgtccggc

SEQ ID NO: 1323 - DNA, 31 bp, Artificial Sequence
  Description of Artificial Sequence  Enzymatic Nucleic Acid
aggtcaggggctagctacaacgaaggctgtc

SEQ ID NO: 1324 - DNA, 31 bp, Artificial Sequence
  Description of Artificial Sequence  Enzymatic Nucleic Acid
cgctgaggggctagctacaacgacaggcagg

SEQ ID NO: 1325 - DNA, 31 bp, Artificial Sequence
  Description of Artificial Sequence  Enzymatic Nucleic Acid
ggaagacgggctagctacaacgatgaggtca

SEQ ID NO: 1326 - DNA, 31 bp, Artificial Sequence
  Description of Artificial Sequence  Enzymatic Nucleic Acid
ctggaagaggctagctacaacgagctgaggt

SEQ ID NO: 1327 - DNA, 31 bp, Artificial Sequence
  Description of Artificial Sequence  Enzymatic Nucleic Acid
cttgcaggggctagctacaacgatctggaag

SEQ ID NO: 1328 - DNA, 31 bp, Artificial Sequence
  Description of Artificial Sequence  Enzymatic Nucleic Acid
attacttgggctagctacaacgaaggttctg

SEQ ID NO: 1329 - DNA, 31 bp, Artificial Sequence
  Description of Artificial Sequence  Enzymatic Nucleic Acid
ccggattaggctagctacaacgattgcaggt

SEQ ID NO: 1330 - DNA, 31 bp, Artificial Sequence
  Description of Artificial Sequence  Enzymatic Nucleic Acid
tccccggaggctagctacaacgatacttgca

SEQ ID NO: 1331 - DNA, 31 bp, Artificial Sequence
  Description of Artificial Sequence  Enzymatic Nucleic Acid
agaattcgggctagctacaacgaccccggat

SEQ ID NO: 1332 - DNA, 31 bp, Artificial Sequence
  Description of Artificial Sequence  Enzymatic Nucleic Acid
gtgcagaaggctagctacaacgatcgtcccc

SEQ ID NO: 1333 - DNA, 31 bp, Artificial Sequence
  Description of Artificial Sequence  Enzymatic Nucleic Acid
ccattgtgggctagctacaacgaagaattcg

SEQ ID NO: 1334 - DNA, 31 bp, Artificial Sequence
  Description of Artificial Sequence  Enzymatic Nucleic Acid
cgccattgggctagctacaacgagcagaatt

SEQ ID NO: 1335 - DNA, 31 bp, Artificial Sequence
  Description of Artificial Sequence  Enzymatic Nucleic Acid
aggcgccaggctagctacaacgatgtgcaga

SEQ ID NO: 1336 - DNA, 31 bp, Artificial Sequence
  Description of Artificial Sequence  Enzymatic Nucleic Acid
agtaggcgggctagctacaacgacattgtgc

SEQ ID NO: 1337 - DNA, 31 bp, Artificial Sequence
  Description of Artificial Sequence  Enzymatic Nucleic Acid
cgagtaggggctagctacaacgagccattgt

SEQ ID NO: 1338 - DNA, 31 bp, Artificial Sequence
  Description of Artificial Sequence  Enzymatic Nucleic Acid
tcagcgagggctagctacaacgaaggcgcca

SEQ ID NO: 1339 - DNA, 31 bp, Artificial Sequence
  Description of Artificial Sequence  Enzymatic Nucleic Acid
agggtcagggctagctacaacgagagtaggc

SEQ ID NO: 1340 - DNA, 31 bp, Artificial Sequence
  Description of Artificial Sequence  Enzymatic Nucleic Acid
ttgcagggggctagctacaacgacagcgagt

SEQ ID NO: 1341 - DNA, 31 bp, Artificial Sequence
  Description of Artificial Sequence  Enzymatic Nucleic Acid
agcccttgggctagctacaacgaagggtcag

SEQ ID NO: 1342 - DNA, 31 bp, Artificial Sequence
  Description of Artificial Sequence  Enzymatic Nucleic Acid
atgcccagggctagctacaacgaccttgcag

SEQ ID NO: 1343 - DNA, 31 bp, Artificial Sequence
  Description of Artificial Sequence  Enzymatic Nucleic Acid
agctgatgggctagctacaacgaccagccct

SEQ ID NO: 1344 - DNA, 31 bp, Artificial Sequence
  Description of Artificial Sequence  Enzymatic Nucleic Acid
ccagctgaggctagctacaacgagcccagcc

SEQ ID NO: 1345 - DNA, 31 bp, Artificial Sequence
  Description of Artificial Sequence  Enzymatic Nucleic Acid
ccagccagggctagctacaacgatgatgccc

SEQ ID NO: 1346 - DNA, 31 bp, Artificial Sequence
  Description of Artificial Sequence  Enzymatic Nucleic Acid
agccccagggctagctacaacgacagctgat

SEQ ID NO: 1347 - DNA, 31 bp, Artificial Sequence
  Description of Artificial Sequence  Enzymatic Nucleic Acid
gagcgcagggctagctacaacgacccagcca

SEQ ID NO: 1348 - DNA, 31 bp, Artificial Sequence
  Description of Artificial Sequence  Enzymatic Nucleic Acid
agtgagcgggctagctacaacgaagccccag

SEQ ID NO: 1349 - DNA, 31 bp, Artificial Sequence
  Description of Artificial Sequence  Enzymatic Nucleic Acid
tcagtgagggctagctacaacgagcagcccc

SEQ ID NO: 1350 - DNA, 31 bp, Artificial Sequence
  Description of Artificial Sequence  Enzymatic Nucleic Acid
tccctcagggctagctacaacgagagcgcag

SEQ ID NO: 1351 - DNA, 31 bp, Artificial Sequence
  Description of Artificial Sequence  Enzymatic Nucleic Acid
ctgcccagggctagctacaacgatccctcag

SEQ ID NO: 1352 - DNA, 31 bp, Artificial Sequence
  Description of Artificial Sequence  Enzymatic Nucleic Acid
gtccactgggctagctacaacgaccagttcc

SEQ ID NO: 1353 - DNA, 31 bp, Artificial Sequence
  Description of Artificial Sequence  Enzymatic Nucleic Acid
ccagtccaggctagctacaacgatgcccagt

SEQ ID NO: 1354 - DNA, 31 bp, Artificial Sequence
  Description of Artificial Sequence  Enzymatic Nucleic Acid
agggccagggctagctacaacgaccactgcc

SEQ ID NO: 1355 - DNA, 31 bp, Artificial Sequence
  Description of Artificial Sequence  Enzymatic Nucleic Acid
gatgagggggctagctacaacgacagtccac

SEQ ID NO: 1356 - DNA, 31 bp, Artificial Sequence
  Description of Artificial Sequence  Enzymatic Nucleic Acid
atggtggaggctagctacaacgagagggcca

SEQ ID NO: 1357 - DNA, 31 bp, Artificial Sequence
  Description of Artificial Sequence  Enzymatic Nucleic Acid
tgttatggggctagctacaacgaggatgagg

SEQ ID NO: 1358 - DNA, 31 bp, Artificial Sequence
  Description of Artificial Sequence  Enzymatic Nucleic Acid
gggtgttaggctagctacaacgaggtggatg

SEQ ID NO: 1359 - DNA, 31 bp, Artificial Sequence
  Description of Artificial Sequence  Enzymatic Nucleic Acid
ggtgggtgggctagctacaacgatatggtgg

SEQ ID NO: 1360 - DNA, 31 bp, Artificial Sequence
  Description of Artificial Sequence  Enzymatic Nucleic Acid
gaggtgggggctagctacaacgagttatggt

SEQ ID NO: 1361 - DNA, 31 bp, Artificial Sequence
  Description of Artificial Sequence  Enzymatic Nucleic Acid
agcagaggggctagctacaacgagggtgtta

SEQ ID NO: 1362 - DNA, 31 bp, Artificial Sequence
  Description of Artificial Sequence  Enzymatic Nucleic Acid
gcacgaagggctagctacaacgaagaggtgg

SEQ ID NO: 1363 - DNA, 31 bp, Artificial Sequence
  Description of Artificial Sequence  Enzymatic Nucleic Acid
cgtgtgcaggctagctacaacgagaagcaga

SEQ ID NO: 1364 - DNA, 31 bp, Artificial Sequence
  Description of Artificial Sequence  Enzymatic Nucleic Acid
accgtgtgggctagctacaacgaacgaagca

SEQ ID NO: 1365 - DNA, 31 bp, Artificial Sequence
  Description of Artificial Sequence  Enzymatic Nucleic Acid
gcaccgtgggctagctacaacgagcacgaag

SEQ ID NO: 1366 - DNA, 31 bp, Artificial Sequence
  Description of Artificial Sequence  Enzymatic Nucleic Acid
gggcaccgggctagctacaacgagtgcacga

SEQ ID NO: 1367 - DNA, 31 bp, Artificial Sequence
  Description of Artificial Sequence  Enzymatic Nucleic Acid
ccagggcaggctagctacaacgacgtgtgca

SEQ ID NO: 1368 - DNA, 31 bp, Artificial Sequence
  Description of Artificial Sequence  Enzymatic Nucleic Acid
tcccagggggctagctacaacgaaccgtgtg

SEQ ID NO: 1369 - DNA, 31 bp, Artificial Sequence
  Description of Artificial Sequence  Enzymatic Nucleic Acid
agagctggggctagctacaacgacccagggc

SEQ ID NO: 1370 - DNA, 31 bp, Artificial Sequence
  Description of Artificial Sequence  Enzymatic Nucleic Acid
cgaaagagggctagctacaacgatggtccca

SEQ ID NO: 1371 - DNA, 31 bp, Artificial Sequence
  Description of Artificial Sequence  Enzymatic Nucleic Acid
ggtgcgggggctagctacaacgatccgaaag

SEQ ID NO: 1372 - DNA, 31 bp, Artificial Sequence
  Description of Artificial Sequence  Enzymatic Nucleic Acid
gcttggtgggctagctacaacgagggttccg

SEQ ID NO: 1373 - DNA, 31 bp, Artificial Sequence
  Description of Artificial Sequence  Enzymatic Nucleic Acid
gagcttggggctagctacaacgagcgggttc

SEQ ID NO: 1374 - DNA, 31 bp, Artificial Sequence
  Description of Artificial Sequence  Enzymatic Nucleic Acid
gagcagagggctagctacaacgattggtgcg

SEQ ID NO: 1375 - DNA, 31 bp, Artificial Sequence
  Description of Artificial Sequence  Enzymatic Nucleic Acid
gtgtggagggctagctacaacgaagagcttg

SEQ ID NO: 1376 - DNA, 31 bp, Artificial Sequence
  Description of Artificial Sequence  Enzymatic Nucleic Acid
tggcagtgggctagctacaacgaggagcaga

SEQ ID NO: 1377 - DNA, 31 bp, Artificial Sequence
  Description of Artificial Sequence  Enzymatic Nucleic Acid
gttggcagggctagctacaacgagtggagca

SEQ ID NO: 1378 - DNA, 31 bp, Artificial Sequence
  Description of Artificial Sequence  Enzymatic Nucleic Acid
ccggttggggctagctacaacgaagtgtgga

SEQ ID NO: 1379 - DNA, 31 bp, Artificial Sequence
  Description of Artificial Sequence  Enzymatic Nucleic Acid
ctggccggggctagctacaacgatggcagtg

SEQ ID NO: 1380 - DNA, 31 bp, Artificial Sequence
  Description of Artificial Sequence  Enzymatic Nucleic Acid
tcctctggggctagctacaacgacggttggc

SEQ ID NO: 1381 - DNA, 31 bp, Artificial Sequence
  Description of Artificial Sequence  Enzymatic Nucleic Acid
cacactcgggctagctacaacgacctctggc

SEQ ID NO: 1382 - DNA, 31 bp, Artificial Sequence
  Description of Artificial Sequence  Enzymatic Nucleic Acid
cccacacaggctagctacaacgatcgtcctc

SEQ ID NO: 1383 - DNA, 31 bp, Artificial Sequence
  Description of Artificial Sequence  Enzymatic Nucleic Acid
cgcccacaggctagctacaacgaactcgtcc

SEQ ID NO: 1384 - DNA, 31 bp, Artificial Sequence
  Description of Artificial Sequence  Enzymatic Nucleic Acid
ctcgcccaggctagctacaacgaacactcgt

SEQ ID NO: 1385 - DNA, 31 bp, Artificial Sequence
  Description of Artificial Sequence  Enzymatic Nucleic Acid
ggccctcgggctagctacaacgaccacacac

SEQ ID NO: 1386 - DNA, 31 bp, Artificial Sequence
  Description of Artificial Sequence  Enzymatic Nucleic Acid
aggccaggggctagctacaacgacctcgccc

SEQ ID NO: 1387 - DNA, 31 bp, Artificial Sequence
  Description of Artificial Sequence  Enzymatic Nucleic Acid
gtggcaggggctagctacaacgacaggccct

SEQ ID NO: 1388 - DNA, 31 bp, Artificial Sequence
  Description of Artificial Sequence  Enzymatic Nucleic Acid
gctggtggggctagctacaacgaaggccagg

SEQ ID NO: 1389 - DNA, 31 bp, Artificial Sequence
  Description of Artificial Sequence  Enzymatic Nucleic Acid
acagctggggctagctacaacgaggcaggcc

SEQ ID NO: 1390 - DNA, 31 bp, Artificial Sequence
  Description of Artificial Sequence  Enzymatic Nucleic Acid
gcgcacagggctagctacaacgatggtggca

SEQ ID NO: 1391 - DNA, 31 bp, Artificial Sequence
  Description of Artificial Sequence  Enzymatic Nucleic Acid
cgggcgcaggctagctacaacgaagctggtg

SEQ ID NO: 1392 - DNA, 31 bp, Artificial Sequence
  Description of Artificial Sequence  Enzymatic Nucleic Acid
ctcgggcgggctagctacaacgaacagctgg

SEQ ID NO: 1393 - DNA, 31 bp, Artificial Sequence
  Description of Artificial Sequence  Enzymatic Nucleic Acid
ccctcgggggctagctacaacgagcacagct

SEQ ID NO: 1394 - DNA, 31 bp, Artificial Sequence
  Description of Artificial Sequence  Enzymatic Nucleic Acid
cagcagtgggctagctacaacgacctcgggc

SEQ ID NO: 1395 - DNA, 31 bp, Artificial Sequence
  Description of Artificial Sequence  Enzymatic Nucleic Acid
cccagcagggctagctacaacgagccctcgg

SEQ ID NO: 1396 - DNA, 31 bp, Artificial Sequence
  Description of Artificial Sequence  Enzymatic Nucleic Acid
gaccccagggctagctacaacgaagtgccct

SEQ ID NO: 1397 - DNA, 31 bp, Artificial Sequence
  Description of Artificial Sequence  Enzymatic Nucleic Acid
gccctggaggctagctacaacgacccagcag

SEQ ID NO: 1398 - DNA, 31 bp, Artificial Sequence
  Description of Artificial Sequence  Enzymatic Nucleic Acid
tgggtgggggctagctacaacgacctggacc

SEQ ID NO: 1399 - DNA, 31 bp, Artificial Sequence
  Description of Artificial Sequence  Enzymatic Nucleic Acid
acactgggggctagctacaacgagggccctg

SEQ ID NO: 1400 - DNA, 31 bp, Artificial Sequence
  Description of Artificial Sequence  Enzymatic Nucleic Acid
ttgacacaggctagctacaacgatgggtggg

SEQ ID NO: 1401 - DNA, 31 bp, Artificial Sequence
  Description of Artificial Sequence  Enzymatic Nucleic Acid
agttgacaggctagctacaacgaactgggtg

SEQ ID NO: 1402 - DNA, 31 bp, Artificial Sequence
  Description of Artificial Sequence  Enzymatic Nucleic Acid
gcagttgaggctagctacaacgaacactggg

SEQ ID NO: 1403 - DNA, 31 bp, Artificial Sequence
  Description of Artificial Sequence  Enzymatic Nucleic Acid
ggctgcagggctagctacaacgatgacacac

SEQ ID NO: 1404 - DNA, 31 bp, Artificial Sequence
  Description of Artificial Sequence  Enzymatic Nucleic Acid
actggctgggctagctacaacgaagttgaca

SEQ ID NO: 1405 - DNA, 31 bp, Artificial Sequence
  Description of Artificial Sequence  Enzymatic Nucleic Acid
ggaactggggctagctacaacgatgcagttg

SEQ ID NO: 1406 - DNA, 31 bp, Artificial Sequence
  Description of Artificial Sequence  Enzymatic Nucleic Acid
cgaaggaaggctagctacaacgatggctgca

SEQ ID NO: 1407 - DNA, 31 bp, Artificial Sequence
  Description of Artificial Sequence  Enzymatic Nucleic Acid
actcctggggctagctacaacgacccgaagg

SEQ ID NO: 1408 - DNA, 31 bp, Artificial Sequence
  Description of Artificial Sequence  Enzymatic Nucleic Acid
tccacgcaggctagctacaacgatcctggcc

SEQ ID NO: 1409 - DNA, 31 bp, Artificial Sequence
  Description of Artificial Sequence  Enzymatic Nucleic Acid
cctccacgggctagctacaacgaactcctgg

SEQ ID NO: 1410 - DNA, 31 bp, Artificial Sequence
  Description of Artificial Sequence  Enzymatic Nucleic Acid
ttcctccaggctagctacaacgagcactcct

SEQ ID NO: 1411 - DNA, 31 bp, Artificial Sequence
  Description of Artificial Sequence  Enzymatic Nucleic Acid
actcggcaggctagctacaacgatcctccac

SEQ ID NO: 1412 - DNA, 31 bp, Artificial Sequence
  Description of Artificial Sequence  Enzymatic Nucleic Acid
gtactcggggctagctacaacgaattcctcc

SEQ ID NO: 1413 - DNA, 31 bp, Artificial Sequence
  Description of Artificial Sequence  Enzymatic Nucleic Acid
ctgcagtaggctagctacaacgatcggcatt

SEQ ID NO: 1414 - DNA, 31 bp, Artificial Sequence
  Description of Artificial Sequence  Enzymatic Nucleic Acid
ccctgcagggctagctacaacgaactcggca

SEQ ID NO: 1415 - DNA, 31 bp, Artificial Sequence
  Description of Artificial Sequence  Enzymatic Nucleic Acid
agcccctgggctagctacaacgaagtactcg

SEQ ID NO: 1416 - DNA, 31 bp, Artificial Sequence
  Description of Artificial Sequence  Enzymatic Nucleic Acid
ctggggagggctagctacaacgaccctgcag

SEQ ID NO: 1417 - DNA, 31 bp, Artificial Sequence
  Description of Artificial Sequence  Enzymatic Nucleic Acid
ttcacataggctagctacaacgatccctggg

SEQ ID NO: 1418 - DNA, 31 bp, Artificial Sequence
  Description of Artificial Sequence  Enzymatic Nucleic Acid
cattcacaggctagctacaacgaactccctg

SEQ ID NO: 1419 - DNA, 31 bp, Artificial Sequence
  Description of Artificial Sequence  Enzymatic Nucleic Acid
ggcattcaggctagctacaacgaatactccc

SEQ ID NO: 1420 - DNA, 31 bp, Artificial Sequence
  Description of Artificial Sequence  Enzymatic Nucleic Acid
gcctggcaggctagctacaacgatcacatac

SEQ ID NO: 1421 - DNA, 31 bp, Artificial Sequence
  Description of Artificial Sequence  Enzymatic Nucleic Acid
gtgcctggggctagctacaacgaattcacat

SEQ ID NO: 1422 - DNA, 31 bp, Artificial Sequence
  Description of Artificial Sequence  Enzymatic Nucleic Acid
aaacagtgggctagctacaacgactggcatt

SEQ ID NO: 1423 - DNA, 31 bp, Artificial Sequence
  Description of Artificial Sequence  Enzymatic Nucleic Acid
gcaaacagggctagctacaacgagcctggca

SEQ ID NO: 1424 - DNA, 31 bp, Artificial Sequence
  Description of Artificial Sequence  Enzymatic Nucleic Acid
acggcaaaggctagctacaacgaagtgcctg

SEQ ID NO: 1425 - DNA, 31 bp, Artificial Sequence
  Description of Artificial Sequence  Enzymatic Nucleic Acid
tggcacggggctagctacaacgaaaacagtg

SEQ ID NO: 1426 - DNA, 31 bp, Artificial Sequence
  Description of Artificial Sequence  Enzymatic Nucleic Acid
gggtggcaggctagctacaacgaggcaaaca

SEQ ID NO: 1427 - DNA, 31 bp, Artificial Sequence
  Description of Artificial Sequence  Enzymatic Nucleic Acid
cagggtggggctagctacaacgaacggcaaa

SEQ ID NO: 1428 - DNA, 31 bp, Artificial Sequence
  Description of Artificial Sequence  Enzymatic Nucleic Acid
actcagggggctagctacaacgaggcacggc

SEQ ID NO: 1429 - DNA, 31 bp, Artificial Sequence
  Description of Artificial Sequence  Enzymatic Nucleic Acid
ggctgacaggctagctacaacgatcagggtg

SEQ ID NO: 1430 - DNA, 31 bp, Artificial Sequence
  Description of Artificial Sequence  Enzymatic Nucleic Acid
ggggctgaggctagctacaacgaactcaggg

SEQ ID NO: 1431 - DNA, 31 bp, Artificial Sequence
  Description of Artificial Sequence  Enzymatic Nucleic Acid
ttctggggggctagctacaacgatgacactc

SEQ ID NO: 1432 - DNA, 31 bp, Artificial Sequence
  Description of Artificial Sequence  Enzymatic Nucleic Acid
ctgagccaggctagctacaacgatctggggc

SEQ ID NO: 1433 - DNA, 31 bp, Artificial Sequence
  Description of Artificial Sequence  Enzymatic Nucleic Acid
tcactgagggctagctacaacgacattctgg

SEQ ID NO: 1434 - DNA, 31 bp, Artificial Sequence
  Description of Artificial Sequence  Enzymatic Nucleic Acid
acaggtcaggctagctacaacgatgagccat

SEQ ID NO: 1435 - DNA, 31 bp, Artificial Sequence
  Description of Artificial Sequence  Enzymatic Nucleic Acid
aaaacaggggctagctacaacgacactgagc

SEQ ID NO: 1436 - DNA, 31 bp, Artificial Sequence
  Description of Artificial Sequence  Enzymatic Nucleic Acid
gtccaaaaggctagctacaacgaaggtcact

SEQ ID NO: 1437 - DNA, 31 bp, Artificial Sequence
  Description of Artificial Sequence  Enzymatic Nucleic Acid
gcctccggggctagctacaacgaccaaaaca

SEQ ID NO: 1438 - DNA, 31 bp, Artificial Sequence
  Description of Artificial Sequence  Enzymatic Nucleic Acid
ctggtcagggctagctacaacgactccggtc

SEQ ID NO: 1439 - DNA, 31 bp, Artificial Sequence
  Description of Artificial Sequence  Enzymatic Nucleic Acid
cacactggggctagctacaacgacagcctcc

SEQ ID NO: 1440 - DNA, 31 bp, Artificial Sequence
  Description of Artificial Sequence  Enzymatic Nucleic Acid
gccacacaggctagctacaacgatggtcagc

SEQ ID NO: 1441 - DNA, 31 bp, Artificial Sequence
  Description of Artificial Sequence  Enzymatic Nucleic Acid
aggccacaggctagctacaacgaactggtca

SEQ ID NO: 1442 - DNA, 31 bp, Artificial Sequence
  Description of Artificial Sequence  Enzymatic Nucleic Acid
acaggccaggctagctacaacgaacactggt

SEQ ID NO: 1443 - DNA, 31 bp, Artificial Sequence
  Description of Artificial Sequence  Enzymatic Nucleic Acid
ggcacaggggctagctacaacgacacacact

SEQ ID NO: 1444 - DNA, 31 bp, Artificial Sequence
  Description of Artificial Sequence  Enzymatic Nucleic Acid
agtgggcaggctagctacaacgaaggccaca

SEQ ID NO: 1445 - DNA, 31 bp, Artificial Sequence
  Description of Artificial Sequence  Enzymatic Nucleic Acid
atagtgggggctagctacaacgaacaggcca

SEQ ID NO: 1446 - DNA, 31 bp, Artificial Sequence
  Description of Artificial Sequence  Enzymatic Nucleic Acid
ccttatagggctagctacaacgagggcacag

SEQ ID NO: 1447 - DNA, 31 bp, Artificial Sequence
  Description of Artificial Sequence  Enzymatic Nucleic Acid
ggtccttaggctagctacaacgaagtgggca

SEQ ID NO: 1448 - DNA, 31 bp, Artificial Sequence
  Description of Artificial Sequence  Enzymatic Nucleic Acid
agggagggggctagctacaacgaccttatag

SEQ ID NO: 1449 - DNA, 31 bp, Artificial Sequence
  Description of Artificial Sequence  Enzymatic Nucleic Acid
gggccacgggctagctacaacgaagaaggga

SEQ ID NO: 1450 - DNA, 31 bp, Artificial Sequence
  Description of Artificial Sequence  Enzymatic Nucleic Acid
gcgggccaggctagctacaacgagcagaagg

SEQ ID NO: 1451 - DNA, 31 bp, Artificial Sequence
  Description of Artificial Sequence  Enzymatic Nucleic Acid
gcagcgggggctagctacaacgacacgcaga

SEQ ID NO: 1452 - DNA, 31 bp, Artificial Sequence
  Description of Artificial Sequence  Enzymatic Nucleic Acid
tggggcagggctagctacaacgagggccacg

SEQ ID NO: 1453 - DNA, 31 bp, Artificial Sequence
  Description of Artificial Sequence  Enzymatic Nucleic Acid
cgctggggggctagctacaacgaagcgggcc

SEQ ID NO: 1454 - DNA, 31 bp, Artificial Sequence
  Description of Artificial Sequence  Enzymatic Nucleic Acid
tcacaccgggctagctacaacgatggggcag

SEQ ID NO: 1455 - DNA, 31 bp, Artificial Sequence
  Description of Artificial Sequence  Enzymatic Nucleic Acid
gtttcacaggctagctacaacgacgctgggg

SEQ ID NO: 1456 - DNA, 31 bp, Artificial Sequence
  Description of Artificial Sequence  Enzymatic Nucleic Acid
aggtttcaggctagctacaacgaaccgctgg

SEQ ID NO: 1457 - DNA, 31 bp, Artificial Sequence
  Description of Artificial Sequence  Enzymatic Nucleic Acid
aggtcaggggctagctacaacgattcacacc

SEQ ID NO: 1458 - DNA, 31 bp, Artificial Sequence
  Description of Artificial Sequence  Enzymatic Nucleic Acid
aggagaggggctagctacaacgacaggtttc

SEQ ID NO: 1459 - DNA, 31 bp, Artificial Sequence
  Description of Artificial Sequence  Enzymatic Nucleic Acid
tgggcatgggctagctacaacgaaggagagg

SEQ ID NO: 1460 - DNA, 31 bp, Artificial Sequence
  Description of Artificial Sequence  Enzymatic Nucleic Acid
gatgggcaggctagctacaacgagtaggaga

SEQ ID NO: 1461 - DNA, 31 bp, Artificial Sequence
  Description of Artificial Sequence  Enzymatic Nucleic Acid
cagatgggggctagctacaacgaatgtagga

SEQ ID NO: 1462 - DNA, 31 bp, Artificial Sequence
  Description of Artificial Sequence  Enzymatic Nucleic Acid
cttccagaggctagctacaacgagggcatgt

SEQ ID NO: 1463 - DNA, 31 bp, Artificial Sequence
  Description of Artificial Sequence  Enzymatic Nucleic Acid
tctggaaaggctagctacaacgattccagat

SEQ ID NO: 1464 - DNA, 31 bp, Artificial Sequence
  Description of Artificial Sequence  Enzymatic Nucleic Acid
cctcctcaggctagctacaacgactggaaac

SEQ ID NO: 1465 - DNA, 31 bp, Artificial Sequence
  Description of Artificial Sequence  Enzymatic Nucleic Acid
ggcatgcgggctagctacaacgacctcctca

SEQ ID NO: 1466 - DNA, 31 bp, Artificial Sequence
  Description of Artificial Sequence  Enzymatic Nucleic Acid
ctggcatgggctagctacaacgagccctcct

SEQ ID NO: 1467 - DNA, 31 bp, Artificial Sequence
  Description of Artificial Sequence  Enzymatic Nucleic Acid
ggctggcaggctagctacaacgagcgccctc

SEQ ID NO: 1468 - DNA, 31 bp, Artificial Sequence
  Description of Artificial Sequence  Enzymatic Nucleic Acid
aaggctggggctagctacaacgaatgcgccc

SEQ ID NO: 1469 - DNA, 31 bp, Artificial Sequence
  Description of Artificial Sequence  Enzymatic Nucleic Acid
gggcaaggggctagctacaacgatggcatgc

SEQ ID NO: 1470 - DNA, 31 bp, Artificial Sequence
  Description of Artificial Sequence  Enzymatic Nucleic Acid
tgatggggggctagctacaacgaaaggctgg

SEQ ID NO: 1471 - DNA, 31 bp, Artificial Sequence
  Description of Artificial Sequence  Enzymatic Nucleic Acid
gcagttgaggctagctacaacgaggggcaag

SEQ ID NO: 1472 - DNA, 31 bp, Artificial Sequence
  Description of Artificial Sequence  Enzymatic Nucleic Acid
gggtgcagggctagctacaacgatgatgggg

SEQ ID NO: 1473 - DNA, 31 bp, Artificial Sequence
  Description of Artificial Sequence  Enzymatic Nucleic Acid
agtgggtgggctagctacaacgaagttgatg

SEQ ID NO: 1474 - DNA, 31 bp, Artificial Sequence
  Description of Artificial Sequence  Enzymatic Nucleic Acid
ggagtgggggctagctacaacgagcagttga

SEQ ID NO: 1475 - DNA, 31 bp, Artificial Sequence
  Description of Artificial Sequence  Enzymatic Nucleic Acid
cacaggagggctagctacaacgagggtgcag

SEQ ID NO: 1476 - DNA, 31 bp, Artificial Sequence
  Description of Artificial Sequence  Enzymatic Nucleic Acid
ggtccacaggctagctacaacgaaggagtgg

SEQ ID NO: 1477 - DNA, 31 bp, Artificial Sequence
  Description of Artificial Sequence  Enzymatic Nucleic Acid
caggtccaggctagctacaacgaacaggagt

SEQ ID NO: 1478 - DNA, 31 bp, Artificial Sequence
  Description of Artificial Sequence  Enzymatic Nucleic Acid
catccaggggctagctacaacgaccacacag

SEQ ID NO: 1479 - DNA, 31 bp, Artificial Sequence
  Description of Artificial Sequence  Enzymatic Nucleic Acid
ccttgtcaggctagctacaacgaccaggtcc

SEQ ID NO: 1480 - DNA, 31 bp, Artificial Sequence
  Description of Artificial Sequence  Enzymatic Nucleic Acid
agcccttgggctagctacaacgacatccagg

SEQ ID NO: 1481 - DNA, 31 bp, Artificial Sequence
  Description of Artificial Sequence  Enzymatic Nucleic Acid
cggggcagggctagctacaacgaccttgtca

SEQ ID NO: 1482 - DNA, 31 bp, Artificial Sequence
  Description of Artificial Sequence  Enzymatic Nucleic Acid
cggcggggggctagctacaacgaagcccttg

SEQ ID NO: 1483 - DNA, 31 bp, Artificial Sequence
  Description of Artificial Sequence  Enzymatic Nucleic Acid
ctgctcggggctagctacaacgaggggcagc

SEQ ID NO: 1484 - DNA, 31 bp, Artificial Sequence
  Description of Artificial Sequence  Enzymatic Nucleic Acid
gctctctgggctagctacaacgatcggcggg

SEQ ID NO: 1485 - DNA, 31 bp, Artificial Sequence
  Description of Artificial Sequence  Enzymatic Nucleic Acid
agggctggggctagctacaacgatctctgct

SEQ ID NO: 1486 - DNA, 31 bp, Artificial Sequence
  Description of Artificial Sequence  Enzymatic Nucleic Acid
tcagagggggctagctacaacgatggctctc

SEQ ID NO: 1487 - DNA, 31 bp, Artificial Sequence
  Description of Artificial Sequence  Enzymatic Nucleic Acid
gatggacgggctagctacaacgacagagggc

SEQ ID NO: 1488 - DNA, 31 bp, Artificial Sequence
  Description of Artificial Sequence  Enzymatic Nucleic Acid
atgatggaggctagctacaacgagtcagagg

SEQ ID NO: 1489 - DNA, 31 bp, Artificial Sequence
  Description of Artificial Sequence  Enzymatic Nucleic Acid
agagatgaggctagctacaacgaggacgtca

SEQ ID NO: 1490 - DNA, 31 bp, Artificial Sequence
  Description of Artificial Sequence  Enzymatic Nucleic Acid
cgcagagaggctagctacaacgagatggacg

SEQ ID NO: 1491 - DNA, 31 bp, Artificial Sequence
  Description of Artificial Sequence  Enzymatic Nucleic Acid
aaccaccgggctagctacaacgaagagatga

SEQ ID NO: 1492 - DNA, 31 bp, Artificial Sequence
  Description of Artificial Sequence  Enzymatic Nucleic Acid
gccaaccaggctagctacaacgacgcagaga

SEQ ID NO: 1493 - DNA, 31 bp, Artificial Sequence
  Description of Artificial Sequence  Enzymatic Nucleic Acid
aatgccaaggctagctacaacgacaccgcag

SEQ ID NO: 1494 - DNA, 31 bp, Artificial Sequence
  Description of Artificial Sequence  Enzymatic Nucleic Acid
gcagaatgggctagctacaacgacaaccacc

SEQ ID NO: 1495 - DNA, 31 bp, Artificial Sequence
  Description of Artificial Sequence  Enzymatic Nucleic Acid
cagcagaaggctagctacaacgagccaacca

SEQ ID NO: 1496 - DNA, 31 bp, Artificial Sequence
  Description of Artificial Sequence  Enzymatic Nucleic Acid
acgaccagggctagctacaacgaagaatgcc

SEQ ID NO: 1497 - DNA, 31 bp, Artificial Sequence
  Description of Artificial Sequence  Enzymatic Nucleic Acid
gaccacgaggctagctacaacgacagcagaa

SEQ ID NO: 1498 - DNA, 31 bp, Artificial Sequence
  Description of Artificial Sequence  Enzymatic Nucleic Acid
caagaccaggctagctacaacgagaccagca

SEQ ID NO: 1499 - DNA, 31 bp, Artificial Sequence
  Description of Artificial Sequence  Enzymatic Nucleic Acid
ccccaagaggctagctacaacgacacgacca

SEQ ID NO: 1500 - DNA, 31 bp, Artificial Sequence
  Description of Artificial Sequence  Enzymatic Nucleic Acid
aaagaccaggctagctacaacgaccccaaga

SEQ ID NO: 1501 - DNA, 31 bp, Artificial Sequence
  Description of Artificial Sequence  Enzymatic Nucleic Acid
cccaaagaggctagctacaacgacaccccca

SEQ ID NO: 1502 - DNA, 31 bp, Artificial Sequence
  Description of Artificial Sequence  Enzymatic Nucleic Acid
gatgaggaggctagctacaacgacccaaaga

SEQ ID NO: 1503 - DNA, 31 bp, Artificial Sequence
  Description of Artificial Sequence  Enzymatic Nucleic Acid
tcgcttgaggctagctacaacgagaggatcc

SEQ ID NO: 1504 - DNA, 31 bp, Artificial Sequence
  Description of Artificial Sequence  Enzymatic Nucleic Acid
tgccgtcgggctagctacaacgattgatgag

SEQ ID NO: 1505 - DNA, 31 bp, Artificial Sequence
  Description of Artificial Sequence  Enzymatic Nucleic Acid
tgctgccgggctagctacaacgacgcttgat

SEQ ID NO: 1506 - DNA, 31 bp, Artificial Sequence
  Description of Artificial Sequence  Enzymatic Nucleic Acid
ttctgctgggctagctacaacgacgtcgctt

SEQ ID NO: 1507 - DNA, 31 bp, Artificial Sequence
  Description of Artificial Sequence  Enzymatic Nucleic Acid
atcttctgggctagctacaacgatgccgtcg

SEQ ID NO: 1508 - DNA, 31 bp, Artificial Sequence
  Description of Artificial Sequence  Enzymatic Nucleic Acid
cttccggaggctagctacaacgacttctgct

SEQ ID NO: 1509 - DNA, 31 bp, Artificial Sequence
  Description of Artificial Sequence  Enzymatic Nucleic Acid
atcgtgtaggctagctacaacgattccggat

SEQ ID NO: 1510 - DNA, 31 bp, Artificial Sequence
  Description of Artificial Sequence  Enzymatic Nucleic Acid
gcatcgtgggctagctacaacgaacttccgg

SEQ ID NO: 1511 - DNA, 31 bp, Artificial Sequence
  Description of Artificial Sequence  Enzymatic Nucleic Acid
ccgcatcgggctagctacaacgagtacttcc

SEQ ID NO: 1512 - DNA, 31 bp, Artificial Sequence
  Description of Artificial Sequence  Enzymatic Nucleic Acid
tctccgcaggctagctacaacgacgtgtact

SEQ ID NO: 1513 - DNA, 31 bp, Artificial Sequence
  Description of Artificial Sequence  Enzymatic Nucleic Acid
agtctccgggctagctacaacgaatcgtgta

SEQ ID NO: 1514 - DNA, 31 bp, Artificial Sequence
  Description of Artificial Sequence  Enzymatic Nucleic Acid
tgcagcagggctagctacaacgactccgcat

SEQ ID NO: 1515 - DNA, 31 bp, Artificial Sequence
  Description of Artificial Sequence  Enzymatic Nucleic Acid
tcctgcagggctagctacaacgaagtctccg

SEQ ID NO: 1516 - DNA, 31 bp, Artificial Sequence
  Description of Artificial Sequence  Enzymatic Nucleic Acid
gtttcctgggctagctacaacgaagcagtct

SEQ ID NO: 1517 - DNA, 31 bp, Artificial Sequence
  Description of Artificial Sequence  Enzymatic Nucleic Acid
cagctccgggctagctacaacgattcctgca

SEQ ID NO: 1518 - DNA, 31 bp, Artificial Sequence
  Description of Artificial Sequence  Enzymatic Nucleic Acid
tccaccagggctagctacaacgatccgtttc

SEQ ID NO: 1519 - DNA, 31 bp, Artificial Sequence
  Description of Artificial Sequence  Enzymatic Nucleic Acid
cggctccaggctagctacaacgacagctccg

SEQ ID NO: 1520 - DNA, 31 bp, Artificial Sequence
  Description of Artificial Sequence  Enzymatic Nucleic Acid
gtcagcggggctagctacaacgatccaccag

SEQ ID NO: 1521 - DNA, 31 bp, Artificial Sequence
  Description of Artificial Sequence  Enzymatic Nucleic Acid
ggtgtcagggctagctacaacgaggctccac

SEQ ID NO: 1522 - DNA, 31 bp, Artificial Sequence
  Description of Artificial Sequence  Enzymatic Nucleic Acid
gctaggtgggctagctacaacgacagcggct

SEQ ID NO: 1523 - DNA, 31 bp, Artificial Sequence
  Description of Artificial Sequence  Enzymatic Nucleic Acid
ccgctaggggctagctacaacgagtcagcgg

SEQ ID NO: 1524 - DNA, 31 bp, Artificial Sequence
  Description of Artificial Sequence  Enzymatic Nucleic Acid
tcgctccgggctagctacaacgataggtgtc

SEQ ID NO: 1525 - DNA, 31 bp, Artificial Sequence
  Description of Artificial Sequence  Enzymatic Nucleic Acid
gggcatcgggctagctacaacgatccgctag

SEQ ID NO: 1526 - DNA, 31 bp, Artificial Sequence
  Description of Artificial Sequence  Enzymatic Nucleic Acid
gttgggcaggctagctacaacgacgctccgc

SEQ ID NO: 1527 - DNA, 31 bp, Artificial Sequence
  Description of Artificial Sequence  Enzymatic Nucleic Acid
tggttgggggctagctacaacgaatcgctcc

SEQ ID NO: 1528 - DNA, 31 bp, Artificial Sequence
  Description of Artificial Sequence  Enzymatic Nucleic Acid
gcgcctggggctagctacaacgatgggcatc

SEQ ID NO: 1529 - DNA, 31 bp, Artificial Sequence
  Description of Artificial Sequence  Enzymatic Nucleic Acid
catctgcgggctagctacaacgactggttgg

SEQ ID NO: 1530 - DNA, 31 bp, Artificial Sequence
  Description of Artificial Sequence  Enzymatic Nucleic Acid
cgcatctgggctagctacaacgagcctggtt

SEQ ID NO: 1531 - DNA, 31 bp, Artificial Sequence
  Description of Artificial Sequence  Enzymatic Nucleic Acid
gatccgcaggctagctacaacgactgcgcct

SEQ ID NO: 1532 - DNA, 31 bp, Artificial Sequence
  Description of Artificial Sequence  Enzymatic Nucleic Acid
aggatccgggctagctacaacgaatctgcgc

SEQ ID NO: 1533 - DNA, 31 bp, Artificial Sequence
  Description of Artificial Sequence  Enzymatic Nucleic Acid
tttcaggaggctagctacaacgaccgcatct

SEQ ID NO: 1534 - DNA, 31 bp, Artificial Sequence
  Description of Artificial Sequence  Enzymatic Nucleic Acid
cagctccgggctagctacaacgactctttca

SEQ ID NO: 1535 - DNA, 31 bp, Artificial Sequence
  Description of Artificial Sequence  Enzymatic Nucleic Acid
ttcctcagggctagctacaacgatccgtctc

SEQ ID NO: 1536 - DNA, 31 bp, Artificial Sequence
  Description of Artificial Sequence  Enzymatic Nucleic Acid
caccttcaggctagctacaacgacttcctca

SEQ ID NO: 1537 - DNA, 31 bp, Artificial Sequence
  Description of Artificial Sequence  Enzymatic Nucleic Acid
tccaagcaggctagctacaacgacttcacct

SEQ ID NO: 1538 - DNA, 31 bp, Artificial Sequence
  Description of Artificial Sequence  Enzymatic Nucleic Acid
gatccaagggctagctacaacgaaccttcac

SEQ ID NO: 1539 - DNA, 31 bp, Artificial Sequence
  Description of Artificial Sequence  Enzymatic Nucleic Acid
gcgccagaggctagctacaacgaccaagcac

SEQ ID NO: 1540 - DNA, 31 bp, Artificial Sequence
  Description of Artificial Sequence  Enzymatic Nucleic Acid
caaaagcgggctagctacaacgacagatcca

SEQ ID NO: 1541 - DNA, 31 bp, Artificial Sequence
  Description of Artificial Sequence  Enzymatic Nucleic Acid
gccaaaagggctagctacaacgagccagatc

SEQ ID NO: 1542 - DNA, 31 bp, Artificial Sequence
  Description of Artificial Sequence  Enzymatic Nucleic Acid
agactgtgggctagctacaacgacaaaagcg

SEQ ID NO: 1543 - DNA, 31 bp, Artificial Sequence
  Description of Artificial Sequence  Enzymatic Nucleic Acid
gtagactgggctagctacaacgagccaaaag

SEQ ID NO: 1544 - DNA, 31 bp, Artificial Sequence
  Description of Artificial Sequence  Enzymatic Nucleic Acid
cttgtagaggctagctacaacgatgtgccaa

SEQ ID NO: 1545 - DNA, 31 bp, Artificial Sequence
  Description of Artificial Sequence  Enzymatic Nucleic Acid
tgcccttgggctagctacaacgaagactgtg

SEQ ID NO: 1546 - DNA, 31 bp, Artificial Sequence
  Description of Artificial Sequence  Enzymatic Nucleic Acid
tccagatgggctagctacaacgaccttgtag

SEQ ID NO: 1547 - DNA, 31 bp, Artificial Sequence
  Description of Artificial Sequence  Enzymatic Nucleic Acid
gatccagaggctagctacaacgagcccttgt

SEQ ID NO: 1548 - DNA, 31 bp, Artificial Sequence
  Description of Artificial Sequence  Enzymatic Nucleic Acid
atcagggaggctagctacaacgaccagatgc

SEQ ID NO: 1549 - DNA, 31 bp, Artificial Sequence
  Description of Artificial Sequence  Enzymatic Nucleic Acid
tctccccaggctagctacaacgacagggatc

SEQ ID NO: 1550 - DNA, 31 bp, Artificial Sequence
  Description of Artificial Sequence  Enzymatic Nucleic Acid
ttttcacaggctagctacaacgatctcccca

SEQ ID NO: 1551 - DNA, 31 bp, Artificial Sequence
  Description of Artificial Sequence  Enzymatic Nucleic Acid
aattttcaggctagctacaacgaattctccc

SEQ ID NO: 1552 - DNA, 31 bp, Artificial Sequence
  Description of Artificial Sequence  Enzymatic Nucleic Acid
cactggaaggctagctacaacgatttcacat

SEQ ID NO: 1553 - DNA, 31 bp, Artificial Sequence
  Description of Artificial Sequence  Enzymatic Nucleic Acid
gatggccaggctagctacaacgatggaattt

SEQ ID NO: 1554 - DNA, 31 bp, Artificial Sequence
  Description of Artificial Sequence  Enzymatic Nucleic Acid
tttgatggggctagctacaacgacactggaa

SEQ ID NO: 1555 - DNA, 31 bp, Artificial Sequence
  Description of Artificial Sequence  Enzymatic Nucleic Acid
cactttgaggctagctacaacgaggccactg

SEQ ID NO: 1556 - DNA, 31 bp, Artificial Sequence
  Description of Artificial Sequence  Enzymatic Nucleic Acid
cctcaacaggctagctacaacgatttgatgg

SEQ ID NO: 1557 - DNA, 31 bp, Artificial Sequence
  Description of Artificial Sequence  Enzymatic Nucleic Acid
tccctcaaggctagctacaacgaactttgat

SEQ ID NO: 1558 - DNA, 31 bp, Artificial Sequence
  Description of Artificial Sequence  Enzymatic Nucleic Acid
gggatgtgggctagctacaacgatttccctc

SEQ ID NO: 1559 - DNA, 31 bp, Artificial Sequence
  Description of Artificial Sequence  Enzymatic Nucleic Acid
gggggatgggctagctacaacgagttttccc

SEQ ID NO: 1560 - DNA, 31 bp, Artificial Sequence
  Description of Artificial Sequence  Enzymatic Nucleic Acid
ttgggggaggctagctacaacgagtgttttc

SEQ ID NO: 1561 - DNA, 31 bp, Artificial Sequence
  Description of Artificial Sequence  Enzymatic Nucleic Acid
tttgttggggctagctacaacgatttggggg

SEQ ID NO: 1562 - DNA, 31 bp, Artificial Sequence
  Description of Artificial Sequence  Enzymatic Nucleic Acid
tttctttgggctagctacaacgatggctttg

SEQ ID NO: 1563 - DNA, 31 bp, Artificial Sequence
  Description of Artificial Sequence  Enzymatic Nucleic Acid
gtctaagaggctagctacaacgattctttgt

SEQ ID NO: 1564 - DNA, 31 bp, Artificial Sequence
  Description of Artificial Sequence  Enzymatic Nucleic Acid
atgcttcgggctagctacaacgactaagatt

SEQ ID NO: 1565 - DNA, 31 bp, Artificial Sequence
  Description of Artificial Sequence  Enzymatic Nucleic Acid
cacgtatgggctagctacaacgattcgtcta

SEQ ID NO: 1566 - DNA, 31 bp, Artificial Sequence
  Description of Artificial Sequence  Enzymatic Nucleic Acid
atcacgtaggctagctacaacgagcttcgtc

SEQ ID NO: 1567 - DNA, 31 bp, Artificial Sequence
  Description of Artificial Sequence  Enzymatic Nucleic Acid
ccatcacgggctagctacaacgaatgcttcg

SEQ ID NO: 1568 - DNA, 31 bp, Artificial Sequence
  Description of Artificial Sequence  Enzymatic Nucleic Acid
agccatcaggctagctacaacgagtatgctt

SEQ ID NO: 1569 - DNA, 31 bp, Artificial Sequence
  Description of Artificial Sequence  Enzymatic Nucleic Acid
accagccaggctagctacaacgacacgtatg

SEQ ID NO: 1570 - DNA, 31 bp, Artificial Sequence
  Description of Artificial Sequence  Enzymatic Nucleic Acid
cacaccagggctagctacaacgacatcacgt

SEQ ID NO: 1571 - DNA, 31 bp, Artificial Sequence
  Description of Artificial Sequence  Enzymatic Nucleic Acid
agcccacaggctagctacaacgacagccatc

SEQ ID NO: 1572 - DNA, 31 bp, Artificial Sequence
  Description of Artificial Sequence  Enzymatic Nucleic Acid
ggagcccaggctagctacaacgaaccagcca

SEQ ID NO: 1573 - DNA, 31 bp, Artificial Sequence
  Description of Artificial Sequence  Enzymatic Nucleic Acid
atggggagggctagctacaacgaccacacca

SEQ ID NO: 1574 - DNA, 31 bp, Artificial Sequence
  Description of Artificial Sequence  Enzymatic Nucleic Acid
gagacataggctagctacaacgaggggagcc

SEQ ID NO: 1575 - DNA, 31 bp, Artificial Sequence
  Description of Artificial Sequence  Enzymatic Nucleic Acid
gggagacaggctagctacaacgaatggggag

SEQ ID NO: 1576 - DNA, 31 bp, Artificial Sequence
  Description of Artificial Sequence  Enzymatic Nucleic Acid
gcgggagaggctagctacaacgaatatgggg

SEQ ID NO: 1577 - DNA, 31 bp, Artificial Sequence
  Description of Artificial Sequence  Enzymatic Nucleic Acid
ccagaaggggctagctacaacgagggagaca

SEQ ID NO: 1578 - DNA, 31 bp, Artificial Sequence
  Description of Artificial Sequence  Enzymatic Nucleic Acid
ggcagatgggctagctacaacgaccagaagg

SEQ ID NO: 1579 - DNA, 31 bp, Artificial Sequence
  Description of Artificial Sequence  Enzymatic Nucleic Acid
caggcagaggctagctacaacgagcccagaa

SEQ ID NO: 1580 - DNA, 31 bp, Artificial Sequence
  Description of Artificial Sequence  Enzymatic Nucleic Acid
atgtcaggggctagctacaacgaagatgccc

SEQ ID NO: 1581 - DNA, 31 bp, Artificial Sequence
  Description of Artificial Sequence  Enzymatic Nucleic Acid
cgtggatgggctagctacaacgacaggcaga

SEQ ID NO: 1582 - DNA, 31 bp, Artificial Sequence
  Description of Artificial Sequence  Enzymatic Nucleic Acid
accgtggaggctagctacaacgagtcaggca

SEQ ID NO: 1583 - DNA, 31 bp, Artificial Sequence
  Description of Artificial Sequence  Enzymatic Nucleic Acid
ctgcaccgggctagctacaacgaggatgtca

SEQ ID NO: 1584 - DNA, 31 bp, Artificial Sequence
  Description of Artificial Sequence  Enzymatic Nucleic Acid
cagctgcaggctagctacaacgacgtggatg

SEQ ID NO: 1585 - DNA, 31 bp, Artificial Sequence
  Description of Artificial Sequence  Enzymatic Nucleic Acid
accagctgggctagctacaacgaaccgtgga

SEQ ID NO: 1586 - DNA, 31 bp, Artificial Sequence
  Description of Artificial Sequence  Enzymatic Nucleic Acid
gtcaccagggctagctacaacgatgcaccgt

SEQ ID NO: 1587 - DNA, 31 bp, Artificial Sequence
  Description of Artificial Sequence  Enzymatic Nucleic Acid
ctgtgtcaggctagctacaacgacagctgca

SEQ ID NO: 1588 - DNA, 31 bp, Artificial Sequence
  Description of Artificial Sequence  Enzymatic Nucleic Acid
aagctgtgggctagctacaacgacaccagct

SEQ ID NO: 1589 - DNA, 31 bp, Artificial Sequence
  Description of Artificial Sequence  Enzymatic Nucleic Acid
ataagctgggctagctacaacgagtcaccag

SEQ ID NO: 1590 - DNA, 31 bp, Artificial Sequence
  Description of Artificial Sequence  Enzymatic Nucleic Acid
ggcataagggctagctacaacgatgtgtcac

SEQ ID NO: 1591 - DNA, 31 bp, Artificial Sequence
  Description of Artificial Sequence  Enzymatic Nucleic Acid
atagggcaggctagctacaacgaaagctgtg

SEQ ID NO: 1592 - DNA, 31 bp, Artificial Sequence
  Description of Artificial Sequence  Enzymatic Nucleic Acid
ccatagggggctagctacaacgaataagctg

SEQ ID NO: 1593 - DNA, 31 bp, Artificial Sequence
  Description of Artificial Sequence  Enzymatic Nucleic Acid
ggcagccaggctagctacaacgaagggcata

SEQ ID NO: 1594 - DNA, 31 bp, Artificial Sequence
  Description of Artificial Sequence  Enzymatic Nucleic Acid
agaggcagggctagctacaacgacatagggc

SEQ ID NO: 1595 - DNA, 31 bp, Artificial Sequence
  Description of Artificial Sequence  Enzymatic Nucleic Acid
ctaagaggggctagctacaacgaagccatag

SEQ ID NO: 1596 - DNA, 31 bp, Artificial Sequence
  Description of Artificial Sequence  Enzymatic Nucleic Acid
ggacatggggctagctacaacgactaagagg

SEQ ID NO: 1597 - DNA, 31 bp, Artificial Sequence
  Description of Artificial Sequence  Enzymatic Nucleic Acid
cccggacaggctagctacaacgaggtctaag

SEQ ID NO: 1598 - DNA, 31 bp, Artificial Sequence
  Description of Artificial Sequence  Enzymatic Nucleic Acid
ttcccggaggctagctacaacgaatggtcta

SEQ ID NO: 1599 - DNA, 31 bp, Artificial Sequence
  Description of Artificial Sequence  Enzymatic Nucleic Acid
gtccgcggggctagctacaacgatttcccgg

SEQ ID NO: 1600 - DNA, 31 bp, Artificial Sequence
  Description of Artificial Sequence  Enzymatic Nucleic Acid
ggcgtccgggctagctacaacgaggttttcc

SEQ ID NO: 1601 - DNA, 31 bp, Artificial Sequence
  Description of Artificial Sequence  Enzymatic Nucleic Acid
cccaggcgggctagctacaacgaccgcggtt

SEQ ID NO: 1602 - DNA, 31 bp, Artificial Sequence
  Description of Artificial Sequence  Enzymatic Nucleic Acid
agcccaggggctagctacaacgagtccgcgg

SEQ ID NO: 1603 - DNA, 31 bp, Artificial Sequence
  Description of Artificial Sequence  Enzymatic Nucleic Acid
cctgggagggctagctacaacgaccaggcgt

SEQ ID NO: 1604 - DNA, 31 bp, Artificial Sequence
  Description of Artificial Sequence  Enzymatic Nucleic Acid
tcagcaggggctagctacaacgacctgggag

SEQ ID NO: 1605 - DNA, 31 bp, Artificial Sequence
  Description of Artificial Sequence  Enzymatic Nucleic Acid
cagttcagggctagctacaacgaaggtcctg

SEQ ID NO: 1606 - DNA, 31 bp, Artificial Sequence
  Description of Artificial Sequence  Enzymatic Nucleic Acid
tacaccagggctagctacaacgatcagcagg

SEQ ID NO: 1607 - DNA, 31 bp, Artificial Sequence
  Description of Artificial Sequence  Enzymatic Nucleic Acid
tgcatacaggctagctacaacgacagttcag

SEQ ID NO: 1608 - DNA, 31 bp, Artificial Sequence
  Description of Artificial Sequence  Enzymatic Nucleic Acid
tctgcataggctagctacaacgaaccagttc

SEQ ID NO: 1609 - DNA, 31 bp, Artificial Sequence
  Description of Artificial Sequence  Enzymatic Nucleic Acid
aatctgcaggctagctacaacgaacaccagt

SEQ ID NO: 1610 - DNA, 31 bp, Artificial Sequence
  Description of Artificial Sequence  Enzymatic Nucleic Acid
gcaatctgggctagctacaacgaatacacca

SEQ ID NO: 1611 - DNA, 31 bp, Artificial Sequence
  Description of Artificial Sequence  Enzymatic Nucleic Acid
cttggcaaggctagctacaacgactgcatac

SEQ ID NO: 1612 - DNA, 31 bp, Artificial Sequence
  Description of Artificial Sequence  Enzymatic Nucleic Acid
ccccttggggctagctacaacgaaatctgca

SEQ ID NO: 1613 - DNA, 31 bp, Artificial Sequence
  Description of Artificial Sequence  Enzymatic Nucleic Acid
gtagctcaggctagctacaacgaccccttgg

SEQ ID NO: 1614 - DNA, 31 bp, Artificial Sequence
  Description of Artificial Sequence  Enzymatic Nucleic Acid
ccaggtagggctagctacaacgatcatcccc

SEQ ID NO: 1615 - DNA, 31 bp, Artificial Sequence
  Description of Artificial Sequence  Enzymatic Nucleic Acid
cctccaggggctagctacaacgaagctcatc

SEQ ID NO: 1616 - DNA, 31 bp, Artificial Sequence
  Description of Artificial Sequence  Enzymatic Nucleic Acid
gccgcacaggctagctacaacgacctccagg

SEQ ID NO: 1617 - DNA, 31 bp, Artificial Sequence
  Description of Artificial Sequence  Enzymatic Nucleic Acid
gagccgcaggctagctacaacgaatcctcca

SEQ ID NO: 1618 - DNA, 31 bp, Artificial Sequence
  Description of Artificial Sequence  Enzymatic Nucleic Acid
acgagccgggctagctacaacgaacatcctc

SEQ ID NO: 1619 - DNA, 31 bp, Artificial Sequence
  Description of Artificial Sequence  Enzymatic Nucleic Acid
tgtacgagggctagctacaacgacgcacatc

SEQ ID NO: 1620 - DNA, 31 bp, Artificial Sequence
  Description of Artificial Sequence  Enzymatic Nucleic Acid
cctgtgtaggctagctacaacgagagccgca

SEQ ID NO: 1621 - DNA, 31 bp, Artificial Sequence
  Description of Artificial Sequence  Enzymatic Nucleic Acid
tccctgtgggctagctacaacgaacgagccg

SEQ ID NO: 1622 - DNA, 31 bp, Artificial Sequence
  Description of Artificial Sequence  Enzymatic Nucleic Acid
agtccctgggctagctacaacgagtacgagc

SEQ ID NO: 1623 - DNA, 31 bp, Artificial Sequence
  Description of Artificial Sequence  Enzymatic Nucleic Acid
cggccaagggctagctacaacgaccctgtgt

SEQ ID NO: 1624 - DNA, 31 bp, Artificial Sequence
  Description of Artificial Sequence  Enzymatic Nucleic Acid
ccgagcggggctagctacaacgacaagtccc

SEQ ID NO: 1625 - DNA, 31 bp, Artificial Sequence
  Description of Artificial Sequence  Enzymatic Nucleic Acid
gttccgagggctagctacaacgaggccaagt

SEQ ID NO: 1626 - DNA, 31 bp, Artificial Sequence
  Description of Artificial Sequence  Enzymatic Nucleic Acid
ccagcacgggctagctacaacgatccgagcg

SEQ ID NO: 1627 - DNA, 31 bp, Artificial Sequence
  Description of Artificial Sequence  Enzymatic Nucleic Acid
gaccagcaggctagctacaacgagttccgag

SEQ ID NO: 1628 - DNA, 31 bp, Artificial Sequence
  Description of Artificial Sequence  Enzymatic Nucleic Acid
ttgaccagggctagctacaacgaacgttccg

SEQ ID NO: 1629 - DNA, 31 bp, Artificial Sequence
  Description of Artificial Sequence  Enzymatic Nucleic Acid
actcttgaggctagctacaacgacagcacgt

SEQ ID NO: 1630 - DNA, 31 bp, Artificial Sequence
  Description of Artificial Sequence  Enzymatic Nucleic Acid
ggttgggaggctagctacaacgatcttgacc

SEQ ID NO: 1631 - DNA, 31 bp, Artificial Sequence
  Description of Artificial Sequence  Enzymatic Nucleic Acid
tgacatggggctagctacaacgatgggactc

SEQ ID NO: 1632 - DNA, 31 bp, Artificial Sequence
  Description of Artificial Sequence  Enzymatic Nucleic Acid
ttttgacaggctagctacaacgaggttggga

SEQ ID NO: 1633 - DNA, 31 bp, Artificial Sequence
  Description of Artificial Sequence  Enzymatic Nucleic Acid
aattttgaggctagctacaacgaatggttgg

SEQ ID NO: 1634 - DNA, 31 bp, Artificial Sequence
  Description of Artificial Sequence  Enzymatic Nucleic Acid
gtctgtaaggctagctacaacgatttgacat

SEQ ID NO: 1635 - DNA, 31 bp, Artificial Sequence
  Description of Artificial Sequence  Enzymatic Nucleic Acid
gaagtctgggctagctacaacgaaattttga

SEQ ID NO: 1636 - DNA, 31 bp, Artificial Sequence
  Description of Artificial Sequence  Enzymatic Nucleic Acid
gcccgaagggctagctacaacgactgtaatt

SEQ ID NO: 1637 - DNA, 31 bp, Artificial Sequence
  Description of Artificial Sequence  Enzymatic Nucleic Acid
cgagccagggctagctacaacgaccgaagtc

SEQ ID NO: 1638 - DNA, 31 bp, Artificial Sequence
  Description of Artificial Sequence  Enzymatic Nucleic Acid
cagccgagggctagctacaacgacagcccga

SEQ ID NO: 1639 - DNA, 31 bp, Artificial Sequence
  Description of Artificial Sequence  Enzymatic Nucleic Acid
tccagcagggctagctacaacgacgagccag

SEQ ID NO: 1640 - DNA, 31 bp, Artificial Sequence
  Description of Artificial Sequence  Enzymatic Nucleic Acid
atgtccagggctagctacaacgaagccgagc

SEQ ID NO: 1641 - DNA, 31 bp, Artificial Sequence
  Description of Artificial Sequence  Enzymatic Nucleic Acid
cgtcaatgggctagctacaacgaccagcagc

SEQ ID NO: 1642 - DNA, 31 bp, Artificial Sequence
  Description of Artificial Sequence  Enzymatic Nucleic Acid
ctcgtcaaggctagctacaacgagtccagca

SEQ ID NO: 1643 - DNA, 31 bp, Artificial Sequence
  Description of Artificial Sequence  Enzymatic Nucleic Acid
ctgtctcgggctagctacaacgacaatgtcc

SEQ ID NO: 1644 - DNA, 31 bp, Artificial Sequence
  Description of Artificial Sequence  Enzymatic Nucleic Acid
gtactctgggctagctacaacgactcgtcaa

SEQ ID NO: 1645 - DNA, 31 bp, Artificial Sequence
  Description of Artificial Sequence  Enzymatic Nucleic Acid
gcatggtaggctagctacaacgatctgtctc

SEQ ID NO: 1646 - DNA, 31 bp, Artificial Sequence
  Description of Artificial Sequence  Enzymatic Nucleic Acid
ctgcatggggctagctacaacgaactctgtc

SEQ ID NO: 1647 - DNA, 31 bp, Artificial Sequence
  Description of Artificial Sequence  Enzymatic Nucleic Acid
catctgcaggctagctacaacgaggtactct

SEQ ID NO: 1648 - DNA, 31 bp, Artificial Sequence
  Description of Artificial Sequence  Enzymatic Nucleic Acid
cccatctgggctagctacaacgaatggtact

SEQ ID NO: 1649 - DNA, 31 bp, Artificial Sequence
  Description of Artificial Sequence  Enzymatic Nucleic Acid
tgcccccaggctagctacaacgactgcatgg

SEQ ID NO: 1650 - DNA, 31 bp, Artificial Sequence
  Description of Artificial Sequence  Enzymatic Nucleic Acid
gcaccttgggctagctacaacgaccccatct

SEQ ID NO: 1651 - DNA, 31 bp, Artificial Sequence
  Description of Artificial Sequence  Enzymatic Nucleic Acid
gatgggcaggctagctacaacgacttgcccc

SEQ ID NO: 1652 - DNA, 31 bp, Artificial Sequence
  Description of Artificial Sequence  Enzymatic Nucleic Acid
ttgatgggggctagctacaacgaaccttgcc

SEQ ID NO: 1653 - DNA, 31 bp, Artificial Sequence
  Description of Artificial Sequence  Enzymatic Nucleic Acid
ccacttgaggctagctacaacgagggcacct

SEQ ID NO: 1654 - DNA, 31 bp, Artificial Sequence
  Description of Artificial Sequence  Enzymatic Nucleic Acid
gccatccaggctagctacaacgattgatggg

SEQ ID NO: 1655 - DNA, 31 bp, Artificial Sequence
  Description of Artificial Sequence  Enzymatic Nucleic Acid
cagcgccaggctagctacaacgaccacttga

SEQ ID NO: 1656 - DNA, 31 bp, Artificial Sequence
  Description of Artificial Sequence  Enzymatic Nucleic Acid
ctccagcgggctagctacaacgacatccact

SEQ ID NO: 1657 - DNA, 31 bp, Artificial Sequence
  Description of Artificial Sequence  Enzymatic Nucleic Acid
gactccagggctagctacaacgagccatcca

SEQ ID NO: 1658 - DNA, 31 bp, Artificial Sequence
  Description of Artificial Sequence  Enzymatic Nucleic Acid
agaatggaggctagctacaacgatccagcgc

SEQ ID NO: 1659 - DNA, 31 bp, Artificial Sequence
  Description of Artificial Sequence  Enzymatic Nucleic Acid
gcggagaaggctagctacaacgaggactcca

SEQ ID NO: 1660 - DNA, 31 bp, Artificial Sequence
  Description of Artificial Sequence  Enzymatic Nucleic Acid
accgccggggctagctacaacgaggagaatg

SEQ ID NO: 1661 - DNA, 31 bp, Artificial Sequence
  Description of Artificial Sequence  Enzymatic Nucleic Acid
gtgaaccgggctagctacaacgacggcggag

SEQ ID NO: 1662 - DNA, 31 bp, Artificial Sequence
  Description of Artificial Sequence  Enzymatic Nucleic Acid
tgggtgaaggctagctacaacgacgccggcg

SEQ ID NO: 1663 - DNA, 31 bp, Artificial Sequence
  Description of Artificial Sequence  Enzymatic Nucleic Acid
ctggtgggggctagctacaacgagaaccgcc

SEQ ID NO: 1664 - DNA, 31 bp, Artificial Sequence
  Description of Artificial Sequence  Enzymatic Nucleic Acid
cactctggggctagctacaacgagggtgaac

SEQ ID NO: 1665 - DNA, 31 bp, Artificial Sequence
  Description of Artificial Sequence  Enzymatic Nucleic Acid
acacatcaggctagctacaacgatctggtgg

SEQ ID NO: 1666 - DNA, 31 bp, Artificial Sequence
  Description of Artificial Sequence  Enzymatic Nucleic Acid
tccacacaggctagctacaacgacactctgg

SEQ ID NO: 1667 - DNA, 31 bp, Artificial Sequence
  Description of Artificial Sequence  Enzymatic Nucleic Acid
actccacaggctagctacaacgaatcactct

SEQ ID NO: 1668 - DNA, 31 bp, Artificial Sequence
  Description of Artificial Sequence  Enzymatic Nucleic Acid
taactccaggctagctacaacgaacatcact

SEQ ID NO: 1669 - DNA, 31 bp, Artificial Sequence
  Description of Artificial Sequence  Enzymatic Nucleic Acid
caccataaggctagctacaacgatccacaca

SEQ ID NO: 1670 - DNA, 31 bp, Artificial Sequence
  Description of Artificial Sequence  Enzymatic Nucleic Acid
tcacaccaggctagctacaacgaaactccac

SEQ ID NO: 1671 - DNA, 31 bp, Artificial Sequence
  Description of Artificial Sequence  Enzymatic Nucleic Acid
cagtcacaggctagctacaacgacataactc

SEQ ID NO: 1672 - DNA, 31 bp, Artificial Sequence
  Description of Artificial Sequence  Enzymatic Nucleic Acid
cacagtcaggctagctacaacgaaccataac

SEQ ID NO: 1673 - DNA, 31 bp, Artificial Sequence
  Description of Artificial Sequence  Enzymatic Nucleic Acid
ccacacagggctagctacaacgacacaccat

SEQ ID NO: 1674 - DNA, 31 bp, Artificial Sequence
  Description of Artificial Sequence  Enzymatic Nucleic Acid
ctcccacaggctagctacaacgaagtcacac

SEQ ID NO: 1675 - DNA, 31 bp, Artificial Sequence
  Description of Artificial Sequence  Enzymatic Nucleic Acid
agctcccaggctagctacaacgaacagtcac

SEQ ID NO: 1676 - DNA, 31 bp, Artificial Sequence
  Description of Artificial Sequence  Enzymatic Nucleic Acid
gtcatcagggctagctacaacgatcccacac

SEQ ID NO: 1677 - DNA, 31 bp, Artificial Sequence
  Description of Artificial Sequence  Enzymatic Nucleic Acid
aaaagtcaggctagctacaacgacagctccc

SEQ ID NO: 1678 - DNA, 31 bp, Artificial Sequence
  Description of Artificial Sequence  Enzymatic Nucleic Acid
cccaaaagggctagctacaacgacatcagct

SEQ ID NO: 1679 - DNA, 31 bp, Artificial Sequence
  Description of Artificial Sequence  Enzymatic Nucleic Acid
aggtttggggctagctacaacgacccaaaag

SEQ ID NO: 1680 - DNA, 31 bp, Artificial Sequence
  Description of Artificial Sequence  Enzymatic Nucleic Acid
tcgtaaggggctagctacaacgattggcccc

SEQ ID NO: 1681 - DNA, 31 bp, Artificial Sequence
  Description of Artificial Sequence  Enzymatic Nucleic Acid
tcccatcgggctagctacaacgaaaggtttg

SEQ ID NO: 1682 - DNA, 31 bp, Artificial Sequence
  Description of Artificial Sequence  Enzymatic Nucleic Acid
ggatcccaggctagctacaacgacgtaaggt

SEQ ID NO: 1683 - DNA, 31 bp, Artificial Sequence
  Description of Artificial Sequence  Enzymatic Nucleic Acid
ggctgggaggctagctacaacgacccatcgt

SEQ ID NO: 1684 - DNA, 31 bp, Artificial Sequence
  Description of Artificial Sequence  Enzymatic Nucleic Acid
ctcccgggggctagctacaacgatgggatcc

SEQ ID NO: 1685 - DNA, 31 bp, Artificial Sequence
  Description of Artificial Sequence  Enzymatic Nucleic Acid
gtcagggaggctagctacaacgactcccggg

SEQ ID NO: 1686 - DNA, 31 bp, Artificial Sequence
  Description of Artificial Sequence  Enzymatic Nucleic Acid
ccagcaggggctagctacaacgacagggatc

SEQ ID NO: 1687 - DNA, 31 bp, Artificial Sequence
  Description of Artificial Sequence  Enzymatic Nucleic Acid
ttttccagggctagctacaacgaaggtcagg

SEQ ID NO: 1688 - DNA, 31 bp, Artificial Sequence
  Description of Artificial Sequence  Enzymatic Nucleic Acid
ggcagccgggctagctacaacgatccccctt

SEQ ID NO: 1689 - DNA, 31 bp, Artificial Sequence
  Description of Artificial Sequence  Enzymatic Nucleic Acid
tggggcagggctagctacaacgacgctcccc

SEQ ID NO: 1690 - DNA, 31 bp, Artificial Sequence
  Description of Artificial Sequence  Enzymatic Nucleic Acid
ggctggggggctagctacaacgaagccgctc

SEQ ID NO: 1691 - DNA, 31 bp, Artificial Sequence
  Description of Artificial Sequence  Enzymatic Nucleic Acid
atggggggggctagctacaacgatggggcag

SEQ ID NO: 1692 - DNA, 31 bp, Artificial Sequence
  Description of Artificial Sequence  Enzymatic Nucleic Acid
ggtgcagaggctagctacaacgaggggggct

SEQ ID NO: 1693 - DNA, 31 bp, Artificial Sequence
  Description of Artificial Sequence  Enzymatic Nucleic Acid
caatggtgggctagctacaacgaagatgggg

SEQ ID NO: 1694 - DNA, 31 bp, Artificial Sequence
  Description of Artificial Sequence  Enzymatic Nucleic Acid
atcaatggggctagctacaacgagcagatgg

SEQ ID NO: 1695 - DNA, 31 bp, Artificial Sequence
  Description of Artificial Sequence  Enzymatic Nucleic Acid
gacatcaaggctagctacaacgaggtgcaga

SEQ ID NO: 1696 - DNA, 31 bp, Artificial Sequence
  Description of Artificial Sequence  Enzymatic Nucleic Acid
tgtagacaggctagctacaacgacaatggtg

SEQ ID NO: 1697 - DNA, 31 bp, Artificial Sequence
  Description of Artificial Sequence  Enzymatic Nucleic Acid
catgtagaggctagctacaacgaatcaatgg

SEQ ID NO: 1698 - DNA, 31 bp, Artificial Sequence
  Description of Artificial Sequence  Enzymatic Nucleic Acid
tgatcatgggctagctacaacgaagacatca

SEQ ID NO: 1699 - DNA, 31 bp, Artificial Sequence
  Description of Artificial Sequence  Enzymatic Nucleic Acid
catgatcaggctagctacaacgagtagacat

SEQ ID NO: 1700 - DNA, 31 bp, Artificial Sequence
  Description of Artificial Sequence  Enzymatic Nucleic Acid
gaccatgaggctagctacaacgacatgtaga

SEQ ID NO: 1701 - DNA, 31 bp, Artificial Sequence
  Description of Artificial Sequence  Enzymatic Nucleic Acid
tttgaccaggctagctacaacgagatcatgt

SEQ ID NO: 1702 - DNA, 31 bp, Artificial Sequence
  Description of Artificial Sequence  Enzymatic Nucleic Acid
acatttgaggctagctacaacgacatgatca

SEQ ID NO: 1703 - DNA, 31 bp, Artificial Sequence
  Description of Artificial Sequence  Enzymatic Nucleic Acid
atccaacaggctagctacaacgattgaccat

SEQ ID NO: 1704 - DNA, 31 bp, Artificial Sequence
  Description of Artificial Sequence  Enzymatic Nucleic Acid
tcatccaaggctagctacaacgaatttgacc

SEQ ID NO: 1705 - DNA, 31 bp, Artificial Sequence
  Description of Artificial Sequence  Enzymatic Nucleic Acid
gtcaatcaggctagctacaacgaccaacatt

SEQ ID NO: 1706 - DNA, 31 bp, Artificial Sequence
  Description of Artificial Sequence  Enzymatic Nucleic Acid
agagtcaaggctagctacaacgacatccaac

SEQ ID NO: 1707 - DNA, 31 bp, Artificial Sequence
  Description of Artificial Sequence  Enzymatic Nucleic Acid
attcagagggctagctacaacgacaatcatc

SEQ ID NO: 1708 - DNA, 31 bp, Artificial Sequence
  Description of Artificial Sequence  Enzymatic Nucleic Acid
ggccgacaggctagctacaacgatcagagtc

SEQ ID NO: 1709 - DNA, 31 bp, Artificial Sequence
  Description of Artificial Sequence  Enzymatic Nucleic Acid
ttggccgaggctagctacaacgaattcagag

SEQ ID NO: 1710 - DNA, 31 bp, Artificial Sequence
  Description of Artificial Sequence  Enzymatic Nucleic Acid
aatcttggggctagctacaacgacgacattc

SEQ ID NO: 1711 - DNA, 31 bp, Artificial Sequence
  Description of Artificial Sequence  Enzymatic Nucleic Acid
tcccggaaggctagctacaacgacttggccg

SEQ ID NO: 1712 - DNA, 31 bp, Artificial Sequence
  Description of Artificial Sequence  Enzymatic Nucleic Acid
gacaccaaggctagctacaacgatcccggaa

SEQ ID NO: 1713 - DNA, 31 bp, Artificial Sequence
  Description of Artificial Sequence  Enzymatic Nucleic Acid
ttcagacaggctagctacaacgacaactccc

SEQ ID NO: 1714 - DNA, 31 bp, Artificial Sequence
  Description of Artificial Sequence  Enzymatic Nucleic Acid
aattcagaggctagctacaacgaaccaactc

SEQ ID NO: 1715 - DNA, 31 bp, Artificial Sequence
  Description of Artificial Sequence  Enzymatic Nucleic Acid
cgggagaaggctagctacaacgatcagacac

SEQ ID NO: 1716 - DNA, 31 bp, Artificial Sequence
  Description of Artificial Sequence  Enzymatic Nucleic Acid
tggccatgggctagctacaacgagggagaat

SEQ ID NO: 1717 - DNA, 31 bp, Artificial Sequence
  Description of Artificial Sequence  Enzymatic Nucleic Acid
cctggccaggctagctacaacgagcgggaga

SEQ ID NO: 1718 - DNA, 31 bp, Artificial Sequence
  Description of Artificial Sequence  Enzymatic Nucleic Acid
gtccctggggctagctacaacgacatgcggg

SEQ ID NO: 1719 - DNA, 31 bp, Artificial Sequence
  Description of Artificial Sequence  Enzymatic Nucleic Acid
gctgggggggctagctacaacgaccctggcc

SEQ ID NO: 1720 - DNA, 31 bp, Artificial Sequence
  Description of Artificial Sequence  Enzymatic Nucleic Acid
acaaagcgggctagctacaacgatgggggtc

SEQ ID NO: 1721 - DNA, 31 bp, Artificial Sequence
  Description of Artificial Sequence  Enzymatic Nucleic Acid
ccacaaagggctagctacaacgagctggggg

SEQ ID NO: 1722 - DNA, 31 bp, Artificial Sequence
  Description of Artificial Sequence  Enzymatic Nucleic Acid
gatgaccaggctagctacaacgaaaagcgct

SEQ ID NO: 1723 - DNA, 31 bp, Artificial Sequence
  Description of Artificial Sequence  Enzymatic Nucleic Acid
ctggatgaggctagctacaacgacacaaagc

SEQ ID NO: 1724 - DNA, 31 bp, Artificial Sequence
  Description of Artificial Sequence  Enzymatic Nucleic Acid
attctggaggctagctacaacgagaccacaa

SEQ ID NO: 1725 - DNA, 31 bp, Artificial Sequence
  Description of Artificial Sequence  Enzymatic Nucleic Acid
agtcctcaggctagctacaacgatctggatg

SEQ ID NO: 1726 - DNA, 31 bp, Artificial Sequence
  Description of Artificial Sequence  Enzymatic Nucleic Acid
ggcccaagggctagctacaacgacctcattc

SEQ ID NO: 1727 - DNA, 31 bp, Artificial Sequence
  Description of Artificial Sequence  Enzymatic Nucleic Acid
tggctgggggctagctacaacgaccaagtcc

SEQ ID NO: 1728 - DNA, 31 bp, Artificial Sequence
  Description of Artificial Sequence  Enzymatic Nucleic Acid
gggactggggctagctacaacgatgggccca

SEQ ID NO: 1729 - DNA, 31 bp, Artificial Sequence
  Description of Artificial Sequence  Enzymatic Nucleic Acid
ccaagggaggctagctacaacgatggctggg

SEQ ID NO: 1730 - DNA, 31 bp, Artificial Sequence
  Description of Artificial Sequence  Enzymatic Nucleic Acid
aggtgctgggctagctacaacgaccaaggga

SEQ ID NO: 1731 - DNA, 31 bp, Artificial Sequence
  Description of Artificial Sequence  Enzymatic Nucleic Acid
agaaggtgggctagctacaacgatgtccaag

SEQ ID NO: 1732 - DNA, 31 bp, Artificial Sequence
  Description of Artificial Sequence  Enzymatic Nucleic Acid
gtagaaggggctagctacaacgagctgtcca

SEQ ID NO: 1733 - DNA, 31 bp, Artificial Sequence
  Description of Artificial Sequence  Enzymatic Nucleic Acid
gtgagcggggctagctacaacgaagaaggtg

SEQ ID NO: 1734 - DNA, 31 bp, Artificial Sequence
  Description of Artificial Sequence  Enzymatic Nucleic Acid
gcagtgagggctagctacaacgaggtagaag

SEQ ID NO: 1735 - DNA, 31 bp, Artificial Sequence
  Description of Artificial Sequence  Enzymatic Nucleic Acid
tccagcagggctagctacaacgagagcggta

SEQ ID NO: 1736 - DNA, 31 bp, Artificial Sequence
  Description of Artificial Sequence  Enzymatic Nucleic Acid
tcctccagggctagctacaacgaagtgagcg

SEQ ID NO: 1737 - DNA, 31 bp, Artificial Sequence
  Description of Artificial Sequence  Enzymatic Nucleic Acid
tgtcatcgggctagctacaacgacctccagc

SEQ ID NO: 1738 - DNA, 31 bp, Artificial Sequence
  Description of Artificial Sequence  Enzymatic Nucleic Acid
ccatgtcaggctagctacaacgacgtcctcc

SEQ ID NO: 1739 - DNA, 31 bp, Artificial Sequence
  Description of Artificial Sequence  Enzymatic Nucleic Acid
cccccatgggctagctacaacgacatcgtcc

SEQ ID NO: 1740 - DNA, 31 bp, Artificial Sequence
  Description of Artificial Sequence  Enzymatic Nucleic Acid
gtcccccaggctagctacaacgagtcatcgt

SEQ ID NO: 1741 - DNA, 31 bp, Artificial Sequence
  Description of Artificial Sequence  Enzymatic Nucleic Acid
ccaccaggggctagctacaacgacccccatg

SEQ ID NO: 1742 - DNA, 31 bp, Artificial Sequence
  Description of Artificial Sequence  Enzymatic Nucleic Acid
agcatccaggctagctacaacgacaggtccc

SEQ ID NO: 1743 - DNA, 31 bp, Artificial Sequence
  Description of Artificial Sequence  Enzymatic Nucleic Acid
cctcagcaggctagctacaacgaccaccagg

SEQ ID NO: 1744 - DNA, 31 bp, Artificial Sequence
  Description of Artificial Sequence  Enzymatic Nucleic Acid
ctcctcagggctagctacaacgaatccacca

SEQ ID NO: 1745 - DNA, 31 bp, Artificial Sequence
  Description of Artificial Sequence  Enzymatic Nucleic Acid
accagataggctagctacaacgatcctcagc

SEQ ID NO: 1746 - DNA, 31 bp, Artificial Sequence
  Description of Artificial Sequence  Enzymatic Nucleic Acid
gtaccagaggctagctacaacgaactcctca

SEQ ID NO: 1747 - DNA, 31 bp, Artificial Sequence
  Description of Artificial Sequence  Enzymatic Nucleic Acid
ctggggtaggctagctacaacgacagatact

SEQ ID NO: 1748 - DNA, 31 bp, Artificial Sequence
  Description of Artificial Sequence  Enzymatic Nucleic Acid
tgctggggggctagctacaacgaaccagata

SEQ ID NO: 1749 - DNA, 31 bp, Artificial Sequence
  Description of Artificial Sequence  Enzymatic Nucleic Acid
aagccctgggctagctacaacgatggggtac

SEQ ID NO: 1750 - DNA, 31 bp, Artificial Sequence
  Description of Artificial Sequence  Enzymatic Nucleic Acid
agaagaagggctagctacaacgacctgctgg

SEQ ID NO: 1751 - DNA, 31 bp, Artificial Sequence
  Description of Artificial Sequence  Enzymatic Nucleic Acid
ggtctggaggctagctacaacgaagaagaag

SEQ ID NO: 1752 - DNA, 31 bp, Artificial Sequence
  Description of Artificial Sequence  Enzymatic Nucleic Acid
gggcagggggctagctacaacgactggacag

SEQ ID NO: 1753 - DNA, 31 bp, Artificial Sequence
  Description of Artificial Sequence  Enzymatic Nucleic Acid
gcccggggggctagctacaacgaagggtctg

SEQ ID NO: 1754 - DNA, 31 bp, Artificial Sequence
  Description of Artificial Sequence  Enzymatic Nucleic Acid
ccccagcgggctagctacaacgaccggggca

SEQ ID NO: 1755 - DNA, 31 bp, Artificial Sequence
  Description of Artificial Sequence  Enzymatic Nucleic Acid
gcccccagggctagctacaacgagcccgggg

SEQ ID NO: 1756 - DNA, 31 bp, Artificial Sequence
  Description of Artificial Sequence  Enzymatic Nucleic Acid
ggaccatgggctagctacaacgaccccagcg

SEQ ID NO: 1757 - DNA, 31 bp, Artificial Sequence
  Description of Artificial Sequence  Enzymatic Nucleic Acid
gtggaccaggctagctacaacgagcccccag

SEQ ID NO: 1758 - DNA, 31 bp, Artificial Sequence
  Description of Artificial Sequence  Enzymatic Nucleic Acid
gtggtggaggctagctacaacgacatgcccc

SEQ ID NO: 1759 - DNA, 31 bp, Artificial Sequence
  Description of Artificial Sequence  Enzymatic Nucleic Acid
gcctgtggggctagctacaacgaggaccatg

SEQ ID NO: 1760 - DNA, 31 bp, Artificial Sequence
  Description of Artificial Sequence  Enzymatic Nucleic Acid
ggtgcctgggctagctacaacgaggtggacc

SEQ ID NO: 1761 - DNA, 31 bp, Artificial Sequence
  Description of Artificial Sequence  Enzymatic Nucleic Acid
ctgcggtgggctagctacaacgactgtggtg

SEQ ID NO: 1762 - DNA, 31 bp, Artificial Sequence
  Description of Artificial Sequence  Enzymatic Nucleic Acid
agctgcggggctagctacaacgagcctgtgg

SEQ ID NO: 1763 - DNA, 31 bp, Artificial Sequence
  Description of Artificial Sequence  Enzymatic Nucleic Acid
atgagctgggctagctacaacgaggtgcctg

SEQ ID NO: 1764 - DNA, 31 bp, Artificial Sequence
  Description of Artificial Sequence  Enzymatic Nucleic Acid
tagatgagggctagctacaacgatgcggtgc

SEQ ID NO: 1765 - DNA, 31 bp, Artificial Sequence
  Description of Artificial Sequence  Enzymatic Nucleic Acid
ctggtagaggctagctacaacgagagctgcg

SEQ ID NO: 1766 - DNA, 31 bp, Artificial Sequence
  Description of Artificial Sequence  Enzymatic Nucleic Acid
actcctggggctagctacaacgaagatgagc

SEQ ID NO: 1767 - DNA, 31 bp, Artificial Sequence
  Description of Artificial Sequence  Enzymatic Nucleic Acid
caccgccaggctagctacaacgatcctggta

SEQ ID NO: 1768 - DNA, 31 bp, Artificial Sequence
  Description of Artificial Sequence  Enzymatic Nucleic Acid
ccccaccgggctagctacaacgacactcctg

SEQ ID NO: 1769 - DNA, 31 bp, Artificial Sequence
  Description of Artificial Sequence  Enzymatic Nucleic Acid
ggtccccaggctagctacaacgacgccactc

SEQ ID NO: 1770 - DNA, 31 bp, Artificial Sequence
  Description of Artificial Sequence  Enzymatic Nucleic Acid
gtgtcaggggctagctacaacgaccccaccg

SEQ ID NO: 1771 - DNA, 31 bp, Artificial Sequence
  Description of Artificial Sequence  Enzymatic Nucleic Acid
ccctagtgggctagctacaacgacaggtccc

SEQ ID NO: 1772 - DNA, 31 bp, Artificial Sequence
  Description of Artificial Sequence  Enzymatic Nucleic Acid
agccctagggctagctacaacgagtcaggtc

SEQ ID NO: 1773 - DNA, 31 bp, Artificial Sequence
  Description of Artificial Sequence  Enzymatic Nucleic Acid
ggctccagggctagctacaacgacctagtgt

SEQ ID NO: 1774 - DNA, 31 bp, Artificial Sequence
  Description of Artificial Sequence  Enzymatic Nucleic Acid
tcagagggggctagctacaacgatccagccc

SEQ ID NO: 1775 - DNA, 31 bp, Artificial Sequence
  Description of Artificial Sequence  Enzymatic Nucleic Acid
cctgggggggctagctacaacgactcctctt

SEQ ID NO: 1776 - DNA, 31 bp, Artificial Sequence
  Description of Artificial Sequence  Enzymatic Nucleic Acid
agtggagaggctagctacaacgactgggggc

SEQ ID NO: 1777 - DNA, 31 bp, Artificial Sequence
  Description of Artificial Sequence  Enzymatic Nucleic Acid
ggtgccagggctagctacaacgaggagacct

SEQ ID NO: 1778 - DNA, 31 bp, Artificial Sequence
  Description of Artificial Sequence  Enzymatic Nucleic Acid
ggagggtgggctagctacaacgacagtggag

SEQ ID NO: 1779 - DNA, 31 bp, Artificial Sequence
  Description of Artificial Sequence  Enzymatic Nucleic Acid
tcggagggggctagctacaacgagccagtgg

SEQ ID NO: 1780 - DNA, 31 bp, Artificial Sequence
  Description of Artificial Sequence  Enzymatic Nucleic Acid
ggagccagggctagctacaacgacccttcgg

SEQ ID NO: 1781 - DNA, 31 bp, Artificial Sequence
  Description of Artificial Sequence  Enzymatic Nucleic Acid
catcggagggctagctacaacgacagcccct

SEQ ID NO: 1782 - DNA, 31 bp, Artificial Sequence
  Description of Artificial Sequence  Enzymatic Nucleic Acid
caaatacaggctagctacaacgacggagcca

SEQ ID NO: 1783 - DNA, 31 bp, Artificial Sequence
  Description of Artificial Sequence  Enzymatic Nucleic Acid
atcaaataggctagctacaacgaatcggagc

SEQ ID NO: 1784 - DNA, 31 bp, Artificial Sequence
  Description of Artificial Sequence  Enzymatic Nucleic Acid
ccatcaaaggctagctacaacgaacatcgga

SEQ ID NO: 1785 - DNA, 31 bp, Artificial Sequence
  Description of Artificial Sequence  Enzymatic Nucleic Acid
ggtcaccaggctagctacaacgacaaataca

SEQ ID NO: 1786 - DNA, 31 bp, Artificial Sequence
  Description of Artificial Sequence  Enzymatic Nucleic Acid
ccaggtcaggctagctacaacgacatcaaat

SEQ ID NO: 1787 - DNA, 31 bp, Artificial Sequence
  Description of Artificial Sequence  Enzymatic Nucleic Acid
ttcccaggggctagctacaacgacaccatca

SEQ ID NO: 1788 - DNA, 31 bp, Artificial Sequence
  Description of Artificial Sequence  Enzymatic Nucleic Acid
tgcccccaggctagctacaacgatcccaggt

SEQ ID NO: 1789 - DNA, 31 bp, Artificial Sequence
  Description of Artificial Sequence  Enzymatic Nucleic Acid
cttggctgggctagctacaacgaccccattc

SEQ ID NO: 1790 - DNA, 31 bp, Artificial Sequence
  Description of Artificial Sequence  Enzymatic Nucleic Acid
ccccttggggctagctacaacgatgccccca

SEQ ID NO: 1791 - DNA, 31 bp, Artificial Sequence
  Description of Artificial Sequence  Enzymatic Nucleic Acid
ctttgcagggctagctacaacgacccttggc

SEQ ID NO: 1792 - DNA, 31 bp, Artificial Sequence
  Description of Artificial Sequence  Enzymatic Nucleic Acid
aggctttgggctagctacaacgaagcccctt

SEQ ID NO: 1793 - DNA, 31 bp, Artificial Sequence
  Description of Artificial Sequence  Enzymatic Nucleic Acid
tggggaggggctagctacaacgatttgcagc

SEQ ID NO: 1794 - DNA, 31 bp, Artificial Sequence
  Description of Artificial Sequence  Enzymatic Nucleic Acid
gtcatgtgggctagctacaacgaggggaggc

SEQ ID NO: 1795 - DNA, 31 bp, Artificial Sequence
  Description of Artificial Sequence  Enzymatic Nucleic Acid
gggtcatgggctagctacaacgagtggggag

SEQ ID NO: 1796 - DNA, 31 bp, Artificial Sequence
  Description of Artificial Sequence  Enzymatic Nucleic Acid
tggggtcaggctagctacaacgagtgtgggg

SEQ ID NO: 1797 - DNA, 31 bp, Artificial Sequence
  Description of Artificial Sequence  Enzymatic Nucleic Acid
ggctggggggctagctacaacgacatgtgtg

SEQ ID NO: 1798 - DNA, 31 bp, Artificial Sequence
  Description of Artificial Sequence  Enzymatic Nucleic Acid
gtagagggggctagctacaacgatggggtca

SEQ ID NO: 1799 - DNA, 31 bp, Artificial Sequence
  Description of Artificial Sequence  Enzymatic Nucleic Acid
taccgctgggctagctacaacgaagagggct

SEQ ID NO: 1800 - DNA, 31 bp, Artificial Sequence
  Description of Artificial Sequence  Enzymatic Nucleic Acid
ctgtaccgggctagctacaacgatgtagagg

SEQ ID NO: 1801 - DNA, 31 bp, Artificial Sequence
  Description of Artificial Sequence  Enzymatic Nucleic Acid
tcactgtaggctagctacaacgacgctgtag

SEQ ID NO: 1802 - DNA, 31 bp, Artificial Sequence
  Description of Artificial Sequence  Enzymatic Nucleic Acid
cctcactgggctagctacaacgaaccgctgt

SEQ ID NO: 1803 - DNA, 31 bp, Artificial Sequence
  Description of Artificial Sequence  Enzymatic Nucleic Acid
ggtcctcaggctagctacaacgatgtaccgc

SEQ ID NO: 1804 - DNA, 31 bp, Artificial Sequence
  Description of Artificial Sequence  Enzymatic Nucleic Acid
ctgtggggggctagctacaacgacctcactg

SEQ ID NO: 1805 - DNA, 31 bp, Artificial Sequence
  Description of Artificial Sequence  Enzymatic Nucleic Acid
gggtactgggctagctacaacgaggggtcct

SEQ ID NO: 1806 - DNA, 31 bp, Artificial Sequence
  Description of Artificial Sequence  Enzymatic Nucleic Acid
caggggtaggctagctacaacgatgtggggt

SEQ ID NO: 1807 - DNA, 31 bp, Artificial Sequence
  Description of Artificial Sequence  Enzymatic Nucleic Acid
ggcaggggggctagctacaacgaactgtggg

SEQ ID NO: 1808 - DNA, 31 bp, Artificial Sequence
  Description of Artificial Sequence  Enzymatic Nucleic Acid
tcagagggggctagctacaacgaaggggtac

SEQ ID NO: 1809 - DNA, 31 bp, Artificial Sequence
  Description of Artificial Sequence  Enzymatic Nucleic Acid
gccatcagggctagctacaacgactcagagg

SEQ ID NO: 1810 - DNA, 31 bp, Artificial Sequence
  Description of Artificial Sequence  Enzymatic Nucleic Acid
cgtagccaggctagctacaacgacagtctca

SEQ ID NO: 1811 - DNA, 31 bp, Artificial Sequence
  Description of Artificial Sequence  Enzymatic Nucleic Acid
caacgtagggctagctacaacgacatcagtc

SEQ ID NO: 1812 - DNA, 31 bp, Artificial Sequence
  Description of Artificial Sequence  Enzymatic Nucleic Acid
gggcaacgggctagctacaacgaagccatca

SEQ ID NO: 1813 - DNA, 31 bp, Artificial Sequence
  Description of Artificial Sequence  Enzymatic Nucleic Acid
gggggcaaggctagctacaacgagtagccat

SEQ ID NO: 1814 - DNA, 31 bp, Artificial Sequence
  Description of Artificial Sequence  Enzymatic Nucleic Acid
caggggggggctagctacaacgaaacgtagc

SEQ ID NO: 1815 - DNA, 31 bp, Artificial Sequence
  Description of Artificial Sequence  Enzymatic Nucleic Acid
gctgcaggggctagctacaacgacagggggg

SEQ ID NO: 1816 - DNA, 31 bp, Artificial Sequence
  Description of Artificial Sequence  Enzymatic Nucleic Acid
gggggctgggctagctacaacgaaggtcagg

SEQ ID NO: 1817 - DNA, 31 bp, Artificial Sequence
  Description of Artificial Sequence  Enzymatic Nucleic Acid
gctgggggggctagctacaacgatgcaggtc

SEQ ID NO: 1818 - DNA, 31 bp, Artificial Sequence
  Description of Artificial Sequence  Enzymatic Nucleic Acid
tattcaggggctagctacaacgatgggggct

SEQ ID NO: 1819 - DNA, 31 bp, Artificial Sequence
  Description of Artificial Sequence  Enzymatic Nucleic Acid
ttcacataggctagctacaacgatcaggctg

SEQ ID NO: 1820 - DNA, 31 bp, Artificial Sequence
  Description of Artificial Sequence  Enzymatic Nucleic Acid
ggttcacaggctagctacaacgaattcaggc

SEQ ID NO: 1821 - DNA, 31 bp, Artificial Sequence
  Description of Artificial Sequence  Enzymatic Nucleic Acid
ctggttcaggctagctacaacgaatattcag

SEQ ID NO: 1822 - DNA, 31 bp, Artificial Sequence
  Description of Artificial Sequence  Enzymatic Nucleic Acid
ctggctggggctagctacaacgatcacatat

SEQ ID NO: 1823 - DNA, 31 bp, Artificial Sequence
  Description of Artificial Sequence  Enzymatic Nucleic Acid
acatctggggctagctacaacgatggttcac

SEQ ID NO: 1824 - DNA, 31 bp, Artificial Sequence
  Description of Artificial Sequence  Enzymatic Nucleic Acid
gccgaacaggctagctacaacgactggctgg

SEQ ID NO: 1825 - DNA, 31 bp, Artificial Sequence
  Description of Artificial Sequence  Enzymatic Nucleic Acid
gggccgaaggctagctacaacgaatctggct

SEQ ID NO: 1826 - DNA, 31 bp, Artificial Sequence
  Description of Artificial Sequence  Enzymatic Nucleic Acid
ggctggggggctagctacaacgacgaacatc

SEQ ID NO: 1827 - DNA, 31 bp, Artificial Sequence
  Description of Artificial Sequence  Enzymatic Nucleic Acid
gaagggggggctagctacaacgatggggccg

SEQ ID NO: 1828 - DNA, 31 bp, Artificial Sequence
  Description of Artificial Sequence  Enzymatic Nucleic Acid
tctcggggggctagctacaacgagaaggggg

SEQ ID NO: 1829 - DNA, 31 bp, Artificial Sequence
  Description of Artificial Sequence  Enzymatic Nucleic Acid
gcagagggggctagctacaacgacctctcgg

SEQ ID NO: 1830 - DNA, 31 bp, Artificial Sequence
  Description of Artificial Sequence  Enzymatic Nucleic Acid
gcagcaggggctagctacaacgaagagggcc

SEQ ID NO: 1831 - DNA, 31 bp, Artificial Sequence
  Description of Artificial Sequence  Enzymatic Nucleic Acid
tcgggcagggctagctacaacgaaggcagag

SEQ ID NO: 1832 - DNA, 31 bp, Artificial Sequence
  Description of Artificial Sequence  Enzymatic Nucleic Acid
aggtcgggggctagctacaacgaagcaggca

SEQ ID NO: 1833 - DNA, 31 bp, Artificial Sequence
  Description of Artificial Sequence  Enzymatic Nucleic Acid
ccagcaggggctagctacaacgacgggcagc

SEQ ID NO: 1834 - DNA, 31 bp, Artificial Sequence
  Description of Artificial Sequence  Enzymatic Nucleic Acid
ggcaccagggctagctacaacgaaggtcggg

SEQ ID NO: 1835 - DNA, 31 bp, Artificial Sequence
  Description of Artificial Sequence  Enzymatic Nucleic Acid
gagtggcaggctagctacaacgacagcaggt

SEQ ID NO: 1836 - DNA, 31 bp, Artificial Sequence
  Description of Artificial Sequence  Enzymatic Nucleic Acid
cagagtggggctagctacaacgaaccagcag

SEQ ID NO: 1837 - DNA, 31 bp, Artificial Sequence
  Description of Artificial Sequence  Enzymatic Nucleic Acid
ttccagagggctagctacaacgaggcaccag

SEQ ID NO: 1838 - DNA, 31 bp, Artificial Sequence
  Description of Artificial Sequence  Enzymatic Nucleic Acid
gtcttgggggctagctacaacgactttccag

SEQ ID NO: 1839 - DNA, 31 bp, Artificial Sequence
  Description of Artificial Sequence  Enzymatic Nucleic Acid
ggagagagggctagctacaacgacttgggcc

SEQ ID NO: 1840 - DNA, 31 bp, Artificial Sequence
  Description of Artificial Sequence  Enzymatic Nucleic Acid
cgaccccaggctagctacaacgatcttccct

SEQ ID NO: 1841 - DNA, 31 bp, Artificial Sequence
  Description of Artificial Sequence  Enzymatic Nucleic Acid
tttgacgaggctagctacaacgacccattct

SEQ ID NO: 1842 - DNA, 31 bp, Artificial Sequence
  Description of Artificial Sequence  Enzymatic Nucleic Acid
gtctttgaggctagctacaacgagaccccat

SEQ ID NO: 1843 - DNA, 31 bp, Artificial Sequence
  Description of Artificial Sequence  Enzymatic Nucleic Acid
caaaaacgggctagctacaacgactttgacg

SEQ ID NO: 1844 - DNA, 31 bp, Artificial Sequence
  Description of Artificial Sequence  Enzymatic Nucleic Acid
ggcaaaaaggctagctacaacgagtctttga

SEQ ID NO: 1845 - DNA, 31 bp, Artificial Sequence
  Description of Artificial Sequence  Enzymatic Nucleic Acid
cccaaaggggctagctacaacgaaaaaacgt

SEQ ID NO: 1846 - DNA, 31 bp, Artificial Sequence
  Description of Artificial Sequence  Enzymatic Nucleic Acid
ccacggcaggctagctacaacgaccccaaag

SEQ ID NO: 1847 - DNA, 31 bp, Artificial Sequence
  Description of Artificial Sequence  Enzymatic Nucleic Acid
ctccacggggctagctacaacgaacccccaa

SEQ ID NO: 1848 - DNA, 31 bp, Artificial Sequence
  Description of Artificial Sequence  Enzymatic Nucleic Acid
gttctccaggctagctacaacgaggcacccc

SEQ ID NO: 1849 - DNA, 31 bp, Artificial Sequence
  Description of Artificial Sequence  Enzymatic Nucleic Acid
actcggggggctagctacaacgatctccacg

SEQ ID NO: 1850 - DNA, 31 bp, Artificial Sequence
  Description of Artificial Sequence  Enzymatic Nucleic Acid
gtcaagtaggctagctacaacgatcggggtt

SEQ ID NO: 1851 - DNA, 31 bp, Artificial Sequence
  Description of Artificial Sequence  Enzymatic Nucleic Acid
gtgtcaagggctagctacaacgaactcgggg

SEQ ID NO: 1852 - DNA, 31 bp, Artificial Sequence
  Description of Artificial Sequence  Enzymatic Nucleic Acid
ctggggtgggctagctacaacgacaagtact

SEQ ID NO: 1853 - DNA, 31 bp, Artificial Sequence
  Description of Artificial Sequence  Enzymatic Nucleic Acid
ccctggggggctagctacaacgagtcaagta

SEQ ID NO: 1854 - DNA, 31 bp, Artificial Sequence
  Description of Artificial Sequence  Enzymatic Nucleic Acid
aggggcagggctagctacaacgatcctccct

SEQ ID NO: 1855 - DNA, 31 bp, Artificial Sequence
  Description of Artificial Sequence  Enzymatic Nucleic Acid
ctgaggggggctagctacaacgaagctcctc

SEQ ID NO: 1856 - DNA, 31 bp, Artificial Sequence
  Description of Artificial Sequence  Enzymatic Nucleic Acid
gggtggggggctagctacaacgatgaggggc

SEQ ID NO: 1857 - DNA, 31 bp, Artificial Sequence
  Description of Artificial Sequence  Enzymatic Nucleic Acid
gaggagggggctagctacaacgaggggctga

SEQ ID NO: 1858 - DNA, 31 bp, Artificial Sequence
  Description of Artificial Sequence  Enzymatic Nucleic Acid
gctgaaggggctagctacaacgaaggaggag

SEQ ID NO: 1859 - DNA, 31 bp, Artificial Sequence
  Description of Artificial Sequence  Enzymatic Nucleic Acid
aggctgggggctagctacaacgatgaaggca

SEQ ID NO: 1860 - DNA, 31 bp, Artificial Sequence
  Description of Artificial Sequence  Enzymatic Nucleic Acid
gtcgaaggggctagctacaacgatgggctga

SEQ ID NO: 1861 - DNA, 31 bp, Artificial Sequence
  Description of Artificial Sequence  Enzymatic Nucleic Acid
agaggttgggctagctacaacgacgaaggct

SEQ ID NO: 1862 - DNA, 31 bp, Artificial Sequence
  Description of Artificial Sequence  Enzymatic Nucleic Acid
aatagaggggctagctacaacgatgtcgaag

SEQ ID NO: 1863 - DNA, 31 bp, Artificial Sequence
  Description of Artificial Sequence  Enzymatic Nucleic Acid
cccagtaaggctagctacaacgaagaggttg

SEQ ID NO: 1864 - DNA, 31 bp, Artificial Sequence
  Description of Artificial Sequence  Enzymatic Nucleic Acid
ggtcccagggctagctacaacgaaatagagg

SEQ ID NO: 1865 - DNA, 31 bp, Artificial Sequence
  Description of Artificial Sequence  Enzymatic Nucleic Acid
ggtcctggggctagctacaacgacccagtaa

SEQ ID NO: 1866 - DNA, 31 bp, Artificial Sequence
  Description of Artificial Sequence  Enzymatic Nucleic Acid
ctggtgggggctagctacaacgacctggtcc

SEQ ID NO: 1867 - DNA, 31 bp, Artificial Sequence
  Description of Artificial Sequence  Enzymatic Nucleic Acid
cgctctggggctagctacaacgagggtcctg

SEQ ID NO: 1868 - DNA, 31 bp, Artificial Sequence
  Description of Artificial Sequence  Enzymatic Nucleic Acid
gccccccgggctagctacaacgatctggtgg

SEQ ID NO: 1869 - DNA, 31 bp, Artificial Sequence
  Description of Artificial Sequence  Enzymatic Nucleic Acid
gggtggagggctagctacaacgacccccgct

SEQ ID NO: 1870 - DNA, 31 bp, Artificial Sequence
  Description of Artificial Sequence  Enzymatic Nucleic Acid
gtgctgggggctagctacaacgaggagcccc

SEQ ID NO: 1871 - DNA, 31 bp, Artificial Sequence
  Description of Artificial Sequence  Enzymatic Nucleic Acid
tgaaggtgggctagctacaacgatgggtgga

SEQ ID NO: 1872 - DNA, 31 bp, Artificial Sequence
  Description of Artificial Sequence  Enzymatic Nucleic Acid
tttgaaggggctagctacaacgagctgggtg

SEQ ID NO: 1873 - DNA, 31 bp, Artificial Sequence
  Description of Artificial Sequence  Enzymatic Nucleic Acid
cgtaggtgggctagctacaacgaccctttga

SEQ ID NO: 1874 - DNA, 31 bp, Artificial Sequence
  Description of Artificial Sequence  Enzymatic Nucleic Acid
gccgtaggggctagctacaacgagtcccttt

SEQ ID NO: 1875 - DNA, 31 bp, Artificial Sequence
  Description of Artificial Sequence  Enzymatic Nucleic Acid
ctctgccgggctagctacaacgaaggtgtcc

SEQ ID NO: 1876 - DNA, 31 bp, Artificial Sequence
  Description of Artificial Sequence  Enzymatic Nucleic Acid
gttctctgggctagctacaacgacgtaggtg

SEQ ID NO: 1877 - DNA, 31 bp, Artificial Sequence
  Description of Artificial Sequence  Enzymatic Nucleic Acid
actctgggggctagctacaacgatctctgcc

SEQ ID NO: 1878 - DNA, 31 bp, Artificial Sequence
  Description of Artificial Sequence  Enzymatic Nucleic Acid
cccaggtaggctagctacaacgatctgggtt

SEQ ID NO: 1879 - DNA, 31 bp, Artificial Sequence
  Description of Artificial Sequence  Enzymatic Nucleic Acid
gacccaggggctagctacaacgaactctggg

SEQ ID NO: 1880 - DNA, 31 bp, Artificial Sequence
  Description of Artificial Sequence  Enzymatic Nucleic Acid
cgtccagaggctagctacaacgaccaggtac

SEQ ID NO: 1881 - DNA, 31 bp, Artificial Sequence
  Description of Artificial Sequence  Enzymatic Nucleic Acid
ctggcacgggctagctacaacgaccagaccc

SEQ ID NO: 1882 - DNA, 31 bp, Artificial Sequence
  Description of Artificial Sequence  Enzymatic Nucleic Acid
cactggcaggctagctacaacgagtccagac

SEQ ID NO: 1883 - DNA, 31 bp, Artificial Sequence
  Description of Artificial Sequence  Enzymatic Nucleic Acid
cacactggggctagctacaacgaacgtccag

SEQ ID NO: 1884 - DNA, 31 bp, Artificial Sequence
  Description of Artificial Sequence  Enzymatic Nucleic Acid
ggttcacaggctagctacaacgatggcacgt

SEQ ID NO: 1885 - DNA, 31 bp, Artificial Sequence
  Description of Artificial Sequence  Enzymatic Nucleic Acid
ctggttcaggctagctacaacgaactggcac

SEQ ID NO: 1886 - DNA, 31 bp, Artificial Sequence
  Description of Artificial Sequence  Enzymatic Nucleic Acid
ccttctggggctagctacaacgatcacactg

SEQ ID NO: 1887 - DNA, 31 bp, Artificial Sequence
  Description of Artificial Sequence  Enzymatic Nucleic Acid
ggacttggggctagctacaacgacttctggt

SEQ ID NO: 1888 - DNA, 31 bp, Artificial Sequence
  Description of Artificial Sequence  Enzymatic Nucleic Acid
tctgcggaggctagctacaacgattggcctt

SEQ ID NO: 1889 - DNA, 31 bp, Artificial Sequence
  Description of Artificial Sequence  Enzymatic Nucleic Acid
ggcttctgggctagctacaacgaggacttgg

SEQ ID NO: 1890 - DNA, 31 bp, Artificial Sequence
  Description of Artificial Sequence  Enzymatic Nucleic Acid
catcagggggctagctacaacgattctgcgg

SEQ ID NO: 1891 - DNA, 31 bp, Artificial Sequence
  Description of Artificial Sequence  Enzymatic Nucleic Acid
aggacacaggctagctacaacgacagggctt

SEQ ID NO: 1892 - DNA, 31 bp, Artificial Sequence
  Description of Artificial Sequence  Enzymatic Nucleic Acid
tgaggacaggctagctacaacgaatcagggc

SEQ ID NO: 1893 - DNA, 31 bp, Artificial Sequence
  Description of Artificial Sequence  Enzymatic Nucleic Acid
cctgaggaggctagctacaacgaacatcagg

SEQ ID NO: 1894 - DNA, 31 bp, Artificial Sequence
  Description of Artificial Sequence  Enzymatic Nucleic Acid
cttccctgggctagctacaacgatccctgag

SEQ ID NO: 1895 - DNA, 31 bp, Artificial Sequence
  Description of Artificial Sequence  Enzymatic Nucleic Acid
aagtcaggggctagctacaacgacttccctg

SEQ ID NO: 1896 - DNA, 31 bp, Artificial Sequence
  Description of Artificial Sequence  Enzymatic Nucleic Acid
agcagaagggctagctacaacgacaggcctt

SEQ ID NO: 1897 - DNA, 31 bp, Artificial Sequence
  Description of Artificial Sequence  Enzymatic Nucleic Acid
gatgccagggctagctacaacgaagaagtca

SEQ ID NO: 1898 - DNA, 31 bp, Artificial Sequence
  Description of Artificial Sequence  Enzymatic Nucleic Acid
tcttgatgggctagctacaacgacagcagaa

SEQ ID NO: 1899 - DNA, 31 bp, Artificial Sequence
  Description of Artificial Sequence  Enzymatic Nucleic Acid
cctcttgaggctagctacaacgagccagcag

SEQ ID NO: 1900 - DNA, 31 bp, Artificial Sequence
  Description of Artificial Sequence  Enzymatic Nucleic Acid
ccctcccaggctagctacaacgactcttgat

SEQ ID NO: 1901 - DNA, 31 bp, Artificial Sequence
  Description of Artificial Sequence  Enzymatic Nucleic Acid
tcggagggggctagctacaacgacctcccac

SEQ ID NO: 1902 - DNA, 31 bp, Artificial Sequence
  Description of Artificial Sequence  Enzymatic Nucleic Acid
ggaagtggggctagctacaacgacggagggc

SEQ ID NO: 1903 - DNA, 31 bp, Artificial Sequence
  Description of Artificial Sequence  Enzymatic Nucleic Acid
cctggaagggctagctacaacgaggtcggag

SEQ ID NO: 1904 - DNA, 31 bp, Artificial Sequence
  Description of Artificial Sequence  Enzymatic Nucleic Acid
atggcaggggctagctacaacgatcccctgg

SEQ ID NO: 1905 - DNA, 31 bp, Artificial Sequence
  Description of Artificial Sequence  Enzymatic Nucleic Acid
tggcatggggctagctacaacgaaggttccc

SEQ ID NO: 1906 - DNA, 31 bp, Artificial Sequence
  Description of Artificial Sequence  Enzymatic Nucleic Acid
tcctggcaggctagctacaacgaggcaggtt

SEQ ID NO: 1907 - DNA, 31 bp, Artificial Sequence
  Description of Artificial Sequence  Enzymatic Nucleic Acid
gttcctggggctagctacaacgaatggcagg

SEQ ID NO: 1908 - DNA, 31 bp, Artificial Sequence
  Description of Artificial Sequence  Enzymatic Nucleic Acid
aggacaggggctagctacaacgatcctggca

SEQ ID NO: 1909 - DNA, 31 bp, Artificial Sequence
  Description of Artificial Sequence  Enzymatic Nucleic Acid
ccttaggaggctagctacaacgaaggttcct

SEQ ID NO: 1910 - DNA, 31 bp, Artificial Sequence
  Description of Artificial Sequence  Enzymatic Nucleic Acid
aaggaaggggctagctacaacgatccttagg

SEQ ID NO: 1911 - DNA, 31 bp, Artificial Sequence
  Description of Artificial Sequence  Enzymatic Nucleic Acid
aactcaagggctagctacaacgaaggaagga

SEQ ID NO: 1912 - DNA, 31 bp, Artificial Sequence
  Description of Artificial Sequence  Enzymatic Nucleic Acid
tctgggaaggctagctacaacgatcaagcag

SEQ ID NO: 1913 - DNA, 31 bp, Artificial Sequence
  Description of Artificial Sequence  Enzymatic Nucleic Acid
tccagccaggctagctacaacgactgggaac

SEQ ID NO: 1914 - DNA, 31 bp, Artificial Sequence
  Description of Artificial Sequence  Enzymatic Nucleic Acid
ccttccagggctagctacaacgacatctggg

SEQ ID NO: 1915 - DNA, 31 bp, Artificial Sequence
  Description of Artificial Sequence  Enzymatic Nucleic Acid
aggctggaggctagctacaacgacccttcca

SEQ ID NO: 1916 - DNA, 31 bp, Artificial Sequence
  Description of Artificial Sequence  Enzymatic Nucleic Acid
caacgaggggctagctacaacgatggacccc

SEQ ID NO: 1917 - DNA, 31 bp, Artificial Sequence
  Description of Artificial Sequence  Enzymatic Nucleic Acid
tcttccaaggctagctacaacgagaggctgg

SEQ ID NO: 1918 - DNA, 31 bp, Artificial Sequence
  Description of Artificial Sequence  Enzymatic Nucleic Acid
cagtgctgggctagctacaacgatcctcttc

SEQ ID NO: 1919 - DNA, 31 bp, Artificial Sequence
  Description of Artificial Sequence  Enzymatic Nucleic Acid
ccccagtgggctagctacaacgatgttcctc

SEQ ID NO: 1920 - DNA, 31 bp, Artificial Sequence
  Description of Artificial Sequence  Enzymatic Nucleic Acid
ctccccagggctagctacaacgagctgttcc

SEQ ID NO: 1921 - DNA, 31 bp, Artificial Sequence
  Description of Artificial Sequence  Enzymatic Nucleic Acid
cacaaagaggctagctacaacgatccccagt

SEQ ID NO: 1922 - DNA, 31 bp, Artificial Sequence
  Description of Artificial Sequence  Enzymatic Nucleic Acid
agaatccaggctagctacaacgaaaagactc

SEQ ID NO: 1923 - DNA, 31 bp, Artificial Sequence
  Description of Artificial Sequence  Enzymatic Nucleic Acid
cctcagaaggctagctacaacgaccacaaag

SEQ ID NO: 1924 - DNA, 31 bp, Artificial Sequence
  Description of Artificial Sequence  Enzymatic Nucleic Acid
gggcagggggctagctacaacgactcagaat

SEQ ID NO: 1925 - DNA, 31 bp, Artificial Sequence
  Description of Artificial Sequence  Enzymatic Nucleic Acid
tcattgggggctagctacaacgaagggcctc

SEQ ID NO: 1926 - DNA, 31 bp, Artificial Sequence
  Description of Artificial Sequence  Enzymatic Nucleic Acid
gagtctcaggctagctacaacgatgggcagg

SEQ ID NO: 1927 - DNA, 31 bp, Artificial Sequence
  Description of Artificial Sequence  Enzymatic Nucleic Acid
ccctagagggctagctacaacgactcattgg

SEQ ID NO: 1928 - DNA, 31 bp, Artificial Sequence
  Description of Artificial Sequence  Enzymatic Nucleic Acid
ccactggaggctagctacaacgacctagagt

SEQ ID NO: 1929 - DNA, 31 bp, Artificial Sequence
  Description of Artificial Sequence  Enzymatic Nucleic Acid
ggcatccaggctagctacaacgatggaccct

SEQ ID NO: 1930 - DNA, 31 bp, Artificial Sequence
  Description of Artificial Sequence  Enzymatic Nucleic Acid
ctgtggcaggctagctacaacgaccactgga

SEQ ID NO: 1931 - DNA, 31 bp, Artificial Sequence
  Description of Artificial Sequence  Enzymatic Nucleic Acid
ggctgtggggctagctacaacgaatccactg

SEQ ID NO: 1932 - DNA, 31 bp, Artificial Sequence
  Description of Artificial Sequence  Enzymatic Nucleic Acid
ctgggctgggctagctacaacgaggcatcca

SEQ ID NO: 1933 - DNA, 31 bp, Artificial Sequence
  Description of Artificial Sequence  Enzymatic Nucleic Acid
aagctgggggctagctacaacgatgtggcat

SEQ ID NO: 1934 - DNA, 31 bp, Artificial Sequence
  Description of Artificial Sequence  Enzymatic Nucleic Acid
gggccaagggctagctacaacgatgggctgt

SEQ ID NO: 1935 - DNA, 31 bp, Artificial Sequence
  Description of Artificial Sequence  Enzymatic Nucleic Acid
ggaaagggggctagctacaacgacaagctgg

SEQ ID NO: 1936 - DNA, 31 bp, Artificial Sequence
  Description of Artificial Sequence  Enzymatic Nucleic Acid
acccaggaggctagctacaacgactggaagg

SEQ ID NO: 1937 - DNA, 31 bp, Artificial Sequence
  Description of Artificial Sequence  Enzymatic Nucleic Acid
tttcagtaggctagctacaacgaccaggatc

SEQ ID NO: 1938 - DNA, 31 bp, Artificial Sequence
  Description of Artificial Sequence  Enzymatic Nucleic Acid
gctttcagggctagctacaacgaacccagga

SEQ ID NO: 1939 - DNA, 31 bp, Artificial Sequence
  Description of Artificial Sequence  Enzymatic Nucleic Acid
ccctaaggggctagctacaacgatttcagta

SEQ ID NO: 1940 - DNA, 31 bp, Artificial Sequence
  Description of Artificial Sequence  Enzymatic Nucleic Acid
caggccagggctagctacaacgattccctaa

SEQ ID NO: 1941 - DNA, 31 bp, Artificial Sequence
  Description of Artificial Sequence  Enzymatic Nucleic Acid
ctctcaggggctagctacaacgacagcttcc

SEQ ID NO: 1942 - DNA, 31 bp, Artificial Sequence
  Description of Artificial Sequence  Enzymatic Nucleic Acid
tagggccgggctagctacaacgattcccctc

SEQ ID NO: 1943 - DNA, 31 bp, Artificial Sequence
  Description of Artificial Sequence  Enzymatic Nucleic Acid
ccttagggggctagctacaacgacgcttccc

SEQ ID NO: 1944 - DNA, 31 bp, Artificial Sequence
  Description of Artificial Sequence  Enzymatic Nucleic Acid
cttagacaggctagctacaacgatcccttag

SEQ ID NO: 1945 - DNA, 31 bp, Artificial Sequence
  Description of Artificial Sequence  Enzymatic Nucleic Acid
ttcttagaggctagctacaacgaactccctt

SEQ ID NO: 1946 - DNA, 31 bp, Artificial Sequence
  Description of Artificial Sequence  Enzymatic Nucleic Acid
cgcttttgggctagctacaacgatcttagac

SEQ ID NO: 1947 - DNA, 31 bp, Artificial Sequence
  Description of Artificial Sequence  Enzymatic Nucleic Acid
atgggtcgggctagctacaacgattttgttc

SEQ ID NO: 1948 - DNA, 31 bp, Artificial Sequence
  Description of Artificial Sequence  Enzymatic Nucleic Acid
tgaatgggggctagctacaacgacgcttttg

SEQ ID NO: 1949 - DNA, 31 bp, Artificial Sequence
  Description of Artificial Sequence  Enzymatic Nucleic Acid
tctctgaaggctagctacaacgagggtcgct

SEQ ID NO: 1950 - DNA, 31 bp, Artificial Sequence
  Description of Artificial Sequence  Enzymatic Nucleic Acid
agggacagggctagctacaacgactctgaat

SEQ ID NO: 1951 - DNA, 31 bp, Artificial Sequence
  Description of Artificial Sequence  Enzymatic Nucleic Acid
ttcagggaggctagctacaacgaagtctctg

SEQ ID NO: 1952 - DNA, 31 bp, Artificial Sequence
  Description of Artificial Sequence  Enzymatic Nucleic Acid
gtactaggggctagctacaacgattcaggga

SEQ ID NO: 1953 - DNA, 31 bp, Artificial Sequence
  Description of Artificial Sequence  Enzymatic Nucleic Acid
gggcagtaggctagctacaacgataggtttc

SEQ ID NO: 1954 - DNA, 31 bp, Artificial Sequence
  Description of Artificial Sequence  Enzymatic Nucleic Acid
gggggcagggctagctacaacgaactaggtt

SEQ ID NO: 1955 - DNA, 31 bp, Artificial Sequence
  Description of Artificial Sequence  Enzymatic Nucleic Acid
atggggggggctagctacaacgaagtactag

SEQ ID NO: 1956 - DNA, 31 bp, Artificial Sequence
  Description of Artificial Sequence  Enzymatic Nucleic Acid
cttcctcaggctagctacaacgaggggggca

SEQ ID NO: 1957 - DNA, 31 bp, Artificial Sequence
  Description of Artificial Sequence  Enzymatic Nucleic Acid
cattgctgggctagctacaacgatccttcct

SEQ ID NO: 1958 - DNA, 31 bp, Artificial Sequence
  Description of Artificial Sequence  Enzymatic Nucleic Acid
caccattgggctagctacaacgatgttcctt

SEQ ID NO: 1959 - DNA, 31 bp, Artificial Sequence
  Description of Artificial Sequence  Enzymatic Nucleic Acid
tgacaccaggctagctacaacgatgctgttc

SEQ ID NO: 1960 - DNA, 31 bp, Artificial Sequence
  Description of Artificial Sequence  Enzymatic Nucleic Acid
tactgacaggctagctacaacgacattgctg

SEQ ID NO: 1961 - DNA, 31 bp, Artificial Sequence
  Description of Artificial Sequence  Enzymatic Nucleic Acid
gatactgaggctagctacaacgaaccattgc

SEQ ID NO: 1962 - DNA, 31 bp, Artificial Sequence
  Description of Artificial Sequence  Enzymatic Nucleic Acid
cctggataggctagctacaacgatgacacca

SEQ ID NO: 1963 - DNA, 31 bp, Artificial Sequence
  Description of Artificial Sequence  Enzymatic Nucleic Acid
agcctggaggctagctacaacgaactgacac

SEQ ID NO: 1964 - DNA, 31 bp, Artificial Sequence
  Description of Artificial Sequence  Enzymatic Nucleic Acid
gtacaaagggctagctacaacgactggatac

SEQ ID NO: 1965 - DNA, 31 bp, Artificial Sequence
  Description of Artificial Sequence  Enzymatic Nucleic Acid
actctgtaggctagctacaacgaaaagcctg

SEQ ID NO: 1966 - DNA, 31 bp, Artificial Sequence
  Description of Artificial Sequence  Enzymatic Nucleic Acid
gcactctgggctagctacaacgaacaaagcc

SEQ ID NO: 1967 - DNA, 31 bp, Artificial Sequence
  Description of Artificial Sequence  Enzymatic Nucleic Acid
gaaaagcaggctagctacaacgatctgtaca

SEQ ID NO: 1968 - DNA, 31 bp, Artificial Sequence
  Description of Artificial Sequence  Enzymatic Nucleic Acid
cagaaaagggctagctacaacgaactctgta

SEQ ID NO: 1969 - DNA, 31 bp, Artificial Sequence
  Description of Artificial Sequence  Enzymatic Nucleic Acid
aaactaaaggctagctacaacgaagaaaagc

SEQ ID NO: 1970 - DNA, 31 bp, Artificial Sequence
  Description of Artificial Sequence  Enzymatic Nucleic Acid
agtaaaaaggctagctacaacgataaacaga

SEQ ID NO: 1971 - DNA, 31 bp, Artificial Sequence
  Description of Artificial Sequence  Enzymatic Nucleic Acid
aaaaaaagggctagctacaacgaaaaaacta

SEQ ID NO: 1972 - DNA, 31 bp, Artificial Sequence
  Description of Artificial Sequence  Enzymatic Nucleic Acid
aaacaaaaggctagctacaacgaaaaaaaag

SEQ ID NO: 1973 - DNA, 31 bp, Artificial Sequence
  Description of Artificial Sequence  Enzymatic Nucleic Acid
taaaaaaaggctagctacaacgaaaaacaaa

SEQ ID NO: 1974 - DNA, 31 bp, Artificial Sequence
  Description of Artificial Sequence  Enzymatic Nucleic Acid
ttatttcaggctagctacaacgactttaaaa

SEQ ID NO: 1975 - DNA, 31 bp, Artificial Sequence
  Description of Artificial Sequence  Enzymatic Nucleic Acid
ggtctttaggctagctacaacgattcatctt

SEQ ID NO: 1976 - DNA, 31 bp, Artificial Sequence
  Description of Artificial Sequence  Enzymatic Nucleic Acid
cccctgggggctagctacaacgactttattt

SEQ ID NO: 1977 - RNA, 13 nt, Homo sapiens
ccaccaaugccag

SEQ ID NO: 1978 - RNA, 15 nt, Homo sapiens
uucuccgauguguaa

SEQ ID NO: 1979 - RNA, 13 nt, Homo sapiens
ugugcuauggucu

SEQ ID NO: 1980 - RNA, 15 nt, Homo sapiens
ccucagcgucuucca

SEQ ID NO: 1981 - RNA, 15 nt, Homo sapiens
auccaccauaacacc

SEQ ID NO: 1982 - DNA, 28 bp, Artificial Sequence
  Description of Artificial Sequence  Enzymatic Nucleic Acid
cuggcaggctagctacaacgaugguggn

SEQ ID NO: 1983 - DNA, 30 bp, Artificial Sequence
  Description of Artificial Sequence  Enzymatic Nucleic Acid
uuacacaggctagctacaacgacggagaan

SEQ ID NO: 1984 - DNA, 28 bp, Artificial Sequence
  Description of Artificial Sequence  Enzymatic Nucleic Acid
agaccaggctagctacaacgaagcacan

SEQ ID NO: 1985 - DNA, 30 bp, Artificial Sequence
  Description of Artificial Sequence  Enzymatic Nucleic Acid
uggaagaggctagctacaacgagcugaggn

SEQ ID NO: 1986 - DNA, 30 bp, Artificial Sequence
  Description of Artificial Sequence  Enzymatic Nucleic Acid
gguguuaggctagctacaacgagguggaun

SEQ ID NO: 1987 - RNA, 15 nt, Artificial Sequence
  Description of Artificial Sequence  Generic substrate sequence
nnnnnnuhnnnnnnn

SEQ ID NO: 1988 - RNA, 36 nt, Artificial Sequence
  Description of Artificial Sequence  Enzymatic Nucleic Acid
nnnnnnncugaugagnnngaaannncgaaannnnnn

SEQ ID NO: 1989 - RNA, 14 nt, Artificial Sequence
  Description of Artificial Sequence  Generic Substrate Sequence
nnnnnchnnnnnnn

SEQ ID NO: 1990 - RNA, 35 nt, Artificial Sequence
  Description of Artificial Sequence  Enzymatic Nucleic Acid
nnnnnnncugaugagnnngaaannncgaannnnnn

SEQ ID NO: 1991 - RNA, 15 nt, Artificial Sequence
  Description of Artificial Sequence  Generic Substrate Sequence
nnnnnnygnnnnnnn

SEQ ID NO: 1992 - RNA, 35 nt, Artificial Sequence
  Description of Artificial Sequence  Enzymatic Nucleic Acid
nnnnnnnugauggcaugcacuaugcgcgnnnnnnn

SEQ ID NO: 1993 - RNA, 48 nt, Artificial Sequence
  Description of Artificial Sequence  Enzymatic Nucleic Acid
gugugcaaccggaggaaacucccuucaaggacgaaaguccgggacggg

SEQ ID NO: 1994 - RNA, 16 nt, Artificial Sequence
  Description of Artificial Sequence  Substrate Sequence
gccguggguugcacac

SEQ ID NO: 1995 - RNA, 36 nt, Artificial Sequence
  Description of Artificial Sequence  Enzymatic Nucleic Acid
gugccuggccgaaaggcgagugaggucugccgcgcn

SEQ ID NO: 1996 - RNA, 15 nt, Artificial Sequence
  Description of Artificial Sequence  Substrate Sequence
gcgcggcgcaggcac

SEQ ID NO: 1997 - DNA, 16 bp, Artificial Sequence
  Description of Artificial Sequence  Substrate Sequence
rggctagctacaacga